(19) United States
(12) Patent Application Publication
(10) Pub. No.: US 2006/0003953 A1
(43) Pub. Date: Jan. 5, 2006

(54) Compositions and their uses directed to bone growth modulators

(76) Inventors: C. Frank Bennett, Carlsbad, CA (US); Madeline M. Butler, Rancho Santa Fe, CA (US); Nicholas M. Dean, Olivenhain, CA (US); Kenneth W. Dobie, Del Mar, CA (US); Joshua Finger, San Marcos, CA (US); Ravi Jain, Carlsbad, CA (US); Robert McKay, Poway, CA (US); Brett P. Monia, Encinitas, CA (US); Kathleen Myers, Oceanside, CA (US)

Correspondence Address: Isis Pharmaceuticals, Inc., 1896 Rtherford Road, Carlsbad, CA, 92008, US

(21) Appl. No.: 11/004,762

(22) Filed: Dec. 3, 2004

Related U.S. Application Data
(60) Provisional application No. 60/527,370, filed on Dec. 4, 2003.
(60) Provisional application No. 60/527,173, filed on Dec. 4, 2003.
(60) Provisional application No. 60/527,172, filed on Dec. 4, 2003.
(60) Provisional application No. 60/527,420, filed on Dec. 4, 2003.
(60) Provisional application No. 60/527,174, filed on Dec. 4, 2003.
(60) Provisional application No. 60/527,397, filed on Dec. 4, 2003.

(51) Int. Cl. A61K 48/00; C07H 21/04

(52) U.S. Cl. 514/44; 536/23.5

(57) ABSTRACT

Disclosed herein are compounds, compositions and methods for modulating the expression of a bone growth modulator in a cell, tissue or animal. Also provided are methods of target validation. Also provided are uses of disclosed compounds and compositions in the manufacture of a medicament for treatment of diseases and disorders.

### FIELD OF THE INVENTION

Disclosed herein are compounds, compositions and methods for modulating the expression of a bone growth modulator in a cell, tissue or animal.

### BACKGROUND OF THE INVENTION

Targeting disease-causing gene sequences was first suggested more than thirty years ago (Belikova et al., Tet. Lett., 1967, 37, 3557-3562), and antisense activity was demonstrated in cell culture more than a decade later (Zamecnik et al., Proc. Natl. Acad. Sci. U.S.A., 1978, 75, 280-284). One advantage of antisense technology in the treatment of a disease or condition that stems from a disease-causing gene is that it is a direct genetic approach that has the ability to modulate (increase or decrease) the expression of specific disease-causing genes. Another advantage is that validation of a target using antisense compounds results in direct and immediate discovery of the drug candidate; in that the antisense compound is the potential therapeutic agent.

Generally, the principle behind antisense technology is that an antisense compound hybridizes to a target nucleic acid and effects the modulation of gene expression activity, or function, such as transcription or translation. The modulation of gene expression can be achieved by, for example, target degradation or occupancy-based inhibition. An example of modulation of RNA target function by degradation is RNase H-based degradation of the target RNA upon hybridization with a DNA-like antisense compound. Another example of modulation of gene expression by target degradation is RNA interference (RNAi). RNAi is a form of antisense-mediated gene silencing involving the introduction of dsRNA leading to the sequence-specific reduction of targeted endogenous mRNA levels. This sequence-specificity makes antisense compounds extremely attractive as tools for target validation and gene functionalization, as well as therapeutics to selectively modulate the expression of genes involved in the pathogenesis of malignancies and other diseases.

Antisense compounds have been employed as therapeutic agents in the treatment of disease states in animals, including humans. Antisense oligonucleotide drugs are being safely and effectively administered to humans in numerous clinical trials. In 1998, the antisense compound, Vitravene® (fomivirsen; developed by Isis Pharmaceuticals Inc., Carlsbad, Calif.) was the first antisense drug to achieve marketing clearance from the U.S. Food and Drug Administration (FDA), and is currently used in the treatment of cytomegalovirus (CMV)-induced retinitis in AIDS patients. A New Drug Application (NDA) for Genasense™ (oblimersen sodium; developed by Genta, Inc., Berkeley Heights, N.J.), an antisense compound which targets the Bcl-2 mRNA overexpressed in many cancers, was accepted by the FDA. Many other antisense compounds are in clinical trials, including those targeting c-myc (NeuGene® AVI-4126, AVI BioPharma, Ridgefield Park, N.J.), TNF-alpha (ISIS 104838, developed by Isis Pharmaceuticals, Inc.), VLA4 (ATL1102, Antisense Therapeutics Ltd., Toorak, Victoria, Australia) and DNA methyltransferase (MG98, developed by MGI Pharma, Bloomington, Minn.).

Chemical modifications have improved the potency and efficacy of antisense compounds, uncovering the potential for oral delivery as well as enhancing subcutaneous administration, decreasing potential for side effects, and leading to improvements in patient convenience.

Chemical modifications which increase the potency of antisense compounds allow administration of lower doses, which reduces the potential for toxicity, as well as decreasing overall cost of therapy. Modifications which increase the resistance to degradation result in slower clearance from the body, allowing for less frequent dosing. Various chemical modifications can be combined in one compound to further optimize the compound's efficacy.

Morphogenesis and remodeling of bone are accomplished by the coordinated actions of bone-resorbing osteoclasts and bone-forming osteoblasts, which metabolize and remodel bone structure throughout development and adult life. Bone is constantly being resorbed and formed at specific sites in the skeleton called basic multicellular units. An estimated 10% of the total bone mass in the human body is remodeled each year. Upon activation, osteoclasts, which differentiate from hematopoietic monocyte/macrophage precursors, migrate to the basic multicellular unit, resorb a portion of bone and finally undergo apoptosis. Subsequently, newly generated osteoblasts, arising from preosteoblastic/stromal cells, form bone at the site of resorption. The development of osteoclasts is controlled by preosteoblastic cells, so that the processes of bone resorption and formation are tightly coordinated, thus allowing for a wave of bone formation to follow each cycle of bone resorption. Imbalances between osteoclast and osteoblast activities can result in skeletal abnormalities characterized by decreased (osteoporosis) or increased (osteopetrosis) bone mass (Khosla, Endocrinology, 2001, 142, 5050-5055; Nakashima et al., Curr. Opin. Rheumatol., 2003, 15, 280-287).

Wnt proteins are extracellular signaling molecules that play a key role in a variety of developmental processes ranging from cell lineage decisions to control of differentiation of the central nervous system in higher vertebrates (Fedi et al., J. Biol. Chem., 1999, 274, 19465-19472). Wnts act through the cytoplasmic protein disheveled to inhibit glyocogen synthase kinase-3 activity, which in turn leads to beta-catenin stabilization of its non-phosphorylated form. Correspondingly, the non-phosphorylated beta-catenin interacts with T-cell factor/LEF transcription factors, which upon translocation to the nucleus, activate target genes.

There are at least two families of WNT signalling inhibitors: the secreted frizzled-related protein family and the Dickkopf (German for “big head” or “stubborn”) family.

Partial protein sequence determination of a 36-kD heparin-binding protein that copurified with hepatocyte growth factor led to the identification of a cDNA from human embryonic lung fibroblasts homologous to the frizzled transmembrane protein family but lacking the transmembrane domain, hence the designation “frizzled-related protein.” The sFRP-1 gene was localized to chromosome 8 pl1.1-12. In adult tissues, highest mRNA expression is in heart, followed by kidney, ovary, prostate, testis, small intestine and colon. Lower levels are observed in placenta, spleen and brain, while barely detectable in skeletal muscle and pancreas. Expression is undetectable in lung, liver, thymus, and peripheral blood leukocytes. In fetal tissues, mRNA expression was highest in kidney, then brain, then lung, and undetectable in liver. sFRP-1 was found to be a Wnt antagonist in a Xenopus embryo assay (Finch et al., Proc. Natl. Acad. Sci. U.S.A., 1997, 94, 6770-6775).

Studies of Xenopus laevis embryos also lead to the discovery of a novel molecule, designated dickkopf-1 (dkk-1). Dkk-1's role in developmental regulation was demonstrated when originally cloned, together with bone morphogenetic protein (BMP) inhibitors, which is able to induce the formation of ectopic heads in Xenopus. Dkk-1 null mutant embryos lack head structures anterior of the midbrain and show duplications and fusions of limb digits (Mukhopadhyay et al., Dev. Cell, 2001, 1, 423-434).

The human dickkopf related protein 1 (Dkk-1), where the protein and the gene was isolated from SK-LMS-1 cells, (also known as SK) is a member of the Dkk protein family that includes Dkk-1, -2, -3, and 4. Fetal expression of Dkk-1 is highest in kidney and lower in liver and brain. Expression in fetal lung was undetectable. Highest expression in adult tissues was in placenta and prostate and expression was detectable in colon and spleen (Fedi et al., J. Biol. Chem., 1999, 274, 19465-19472). The DKK-1 gene was mapped to chromosome 10q 1.2 (Roessler et al., Cytogenet. Cell Genet., 2000, 89, 220-224).

Dkk-1 has been shown to block both the early and late effects of ectopic Xwnt-8 in Xenopus embryos and inhibit Wnt-induced stabilization of b-catenin (Fedi et al., J. Biol. Chem., 1999, 274, 19465-19472).

Unlike other Wnt antagonists, Dkk-1 prevents activation of the Wnt signaling pathway by binding to LRP5/6 rather than to Wnt proteins. In addition to LRP5/6, Dkk-1 also interacts with Kremen1 and Kremen2. Krm, Dkk-1 and LRP6 form a ternary complex that disrupts Wnt/LRP6 signaling by promoting endocytosis and removal of the Wnt receptor (Frizzled) from the plasma membrane.

Interruption of the Wnt signaling pathway has been correlated with neoplastic processes including human colon cancer, melanomas and hepatocellular carcinomas (Fedi et al., J. Biol. Chem., 1999, 274, 19465-19472). Furthermore, osteoporosis-pseudoglioma, an autosomal recessive disease characterized by low bone mass, with childhood fractures and abnormal eye development, has been shown to be due to LRP5 loss of function mutation (Boyden et al., N. Engl. J. Med., 2002, 346, 1513-1521). Consequently, modulation of the Wnt pathway via Dkk-1 or sFRP-1 can affect bone development.

One of the principal mechanisms by which cellular regulation is effected is through the transduction of extracellular signals across the membrane that in turn modulate biochemical pathways within the cell. Protein phosphorylation, orchestrated by enzymes known as kinases, represents one course by which intracellular signals are propagated from molecule to molecule resulting in a cellular response. These signal transduction cascades are highly regulated and often overlapping as evidenced by the existence of many protein kinases as well as phosphatases, which remove phosphate moieties. It is currently believed that a number of disease states and/or disorders are a result of either aberrant activation or functional mutations in the molecular components of kinase cascades. Consequently, considerable attention has been devoted to the characterization of kinases, especially those involved in energy metabolism. One such kinase is glycogen synthase kinase 3.

Two different mammalian isoforms of glycogen synthase kinase 3 have been identified and each is encoded by a separate gene (Shaw et al., Genome, 1998, 41, 720-727; Woodgett, Embo J, 1990, 9, 2431-2438). These isoforms, designated alpha and beta are expressed in different cell types and in different proportions. In some cells, the expression of these isoforms is under developmental control.

Glycogen synthase kinase 3 beta (also known as tau protein kinase I and GSK3B) is a serine/threonine protein kinase first described as a factor involved in glycogen synthesis. In this pathway, glycogen synthase kinase 3 phosphorylates select residues of glycogen synthase, the rate-limiting enzyme of glycogen deposition, thereby inactivating the enzyme. Therefore, glycogen synthase kinase 3 plays a predominant role in glycogen metabolism and has consequently been investigated as a potential therapeutic target in disease conditions such as diabetes and insulin regulation disorders (Cross et al., FEBS Lett., 1997, 406, 211-215; Eldar-Finkelman et al., Proc. Natl. Acad. Sci. U.S.A., 1996, 93, 10228-10233; Eldar-Finkelman and Krebs, Proc. Natl. Acad. Sci. U.S.A., 1997, 94, 9660-9664; Eldar-Finkelman et al., Diabetes, 1999, 48, 1662-1666). Upstream, glycogen synthase kinase 3 beta is regulated by protein kinase C (Goode et al., J. Biol. Chem., 1992, 267, 16878-16882).

It has been demonstrated that glycogen synthase kinase 3 beta is identical to a previously identified protein known as tau protein kinase-I, which phosphorylates tau, a protein component of paired helical filaments (PHF) found in Alzheimer's brains (Ishiguro et al., FEBS Lett., 1993, 325, 167-172; Lovestone et al., Neuroscience, 1996, 73, 1145-1157; Yamaguchi et al., Acta. Neuropathol. (Berl), 1996, 92, 232-241). The accumulation of these filaments is implicated in the pathological change in brain tissue (Ishiguro et al., FEBS Lett., 1993, 325, 167-172). Glycogen synthase kinase 3 beta is enriched in brain and due to its ability to phosphorylate the tau protein, has been suggested to play a critical role in the development of Alzheimer's disease (Pei et al., J Neuropathol. Exp. Neurol., 1997, 56, 70-78). Increased synthesis of the enzyme has been shown to increase the cellular maturation of another protein related to Alzheimer's disease, APP (Aplin et al., Neuroreport., 1997, 8, 639-643). It is the aberrant processing of APP that leads to deposition of a beta amyloid in neuritic plaques.

Currently, there are no known therapeutic agents which effectively inhibit the synthesis of glycogen synthase kinase 3 beta and to date, investigative strategies aimed at modulating glycogen synthase kinase 3 beta function have involved the use of antibodies, antisense technology and chemical inhibitors. Disclosed in U.S. Pat. No. 5,837,853 are antisense oligonucleotides targeting the nucleotides which encode the first six amino acids of human glycogen synthase kinase beta intended for use in the treatment of Alzheimer's disease and the prevention of neuronal cell death (Takashima et al., 1998). Disclosed in the PCT publication WO 97/41854 are methods to identify inhibitors of glycogen synthase kinase 3 and the use of these inhibitors for the treatment of bipolar disorders, mania, Alzheimer's disease, diabetes and leukopenia (Klein and Melton, 1997). Other inhibitory compounds are disclosed in WO 99/21859. These heterocyclic compounds are intended for the treatment of a disease mediated by a protein kinase, one of which is glycogen synthase kinase 3 (Cheung et al., 1999). Two other compounds, lithium and valproate, both used in the treatment of bipolar disorders, have been shown to inhibit glycogen synthase kinase 3 beta activity (Chen et al., J. Neurochem., 1999, 72, 1327-1330; Hong et al., J. Biol. Chem., 1997, 272, 25326-25332).

Sclerostin was first identified by linkage analysis as the protein whose mutation results in sclerosteosis, a rare bone disease in Afrikaner families. The gene was mapped to chromosome 17q12-q21. Sclerostin gene expression is relatively low overall, but there is significant expression in whole long bone, cartilage, kidney, and liver and lower expression in placenta and fetal skin (Brunkow et al., Am. J. Hum. Genet., 2001, 68, 577-589). Sclerostin gene expression was also detected in bone marrow and osteoblasts (Balemans et al., Hum. Mol. Genet., 2001, 10, 537-543). However, more recent in situ hybridization studies suggest that in vivo sclerostin is secreted by osteoclasts, but not by osteoblasts (Kusu et al., J. Biol. Chem., 2003, 278, 24113-24117).

The sclerostin protein contains a cystine knot motif with high similarity to the dan set of secreted glcoproteins which include dan, cerberus, gremlin, and caronte, shown to act as antagonists of the members of the transforming growth factor superfamily, including bone morphogenetic proteins (BMPs). BMPs are involved in bone development and osteoblast differentiation. Sclerostin inhibits BMP6 and BMP7, but not BMP2 or BMP4, by binding these ligands extracellularly (Kusu et al., J. Biol. Chem., 2003, 278, 24113-24117). Sclerostin plays a pivotal role in prenatal and postnatal bone development. Sclerosteosis is a rare, progressive sclerosing bone dysplasia that results in massive bone overgrowth throughout life leading to gigantism, distortion of the facies, and entrapment of the seventh and eighth cranial nerves. Raised intracranial pressure can lead to sudden death (Balemans et al., Hum. Mol. Genet., 2001, 10, 537-543). Furthermore, direct regulation of BMPs by sclerostin predict a role in embryogenesis (Kusu et al., J. Biol. Chem., 2003, 278, 24113-24117).

Bone morphogenetic proteins (BMPs), members of the transforming growth factor beta (TGF-beta) superfamily, control osteoblast proliferation and differentiation. Smad proteins play a critical role in mediating BMP-induced signaling (Yoshida et al., Cell, 2000, 103, 1085-1097).

Transducer of ERBB2 (also known as TOB, TOB1, TROB, TROB1, APRO6, MGC34446 and transducer of erb-2) is a member of a novel antiproliferative family of proteins, initially demonstrated to suppress cell growth when expressed exogenously in NIH3T3 cells. The cDNA for Transducer of ERBB2 protein was isolated by virtue of the protein's interaction with the c-erbB-2 gene product. The gene was localized to chromosome 17q21 (Matsuda et al., Oncogene, 1996, 12, 705-713).

Transducer of ERBB2 deficient mice demonstrated increased bone formation due to increased osteoblast numbers. Mouse transducer of ERBB2 inhibits BMP-induced, Smad-dependent transcription in osteoblasts, thereby regulating bone growth by inhibiting osteoblast proliferation (Yoshida et al., Cell, 2000, 103, 1085-1097).

The v-src gene encoded by the Rous sarcoma virus was the first discovered as a transmissible agent found to induce tumors in chickens. The protein product of this gene, v-src, is a tyrosine kinase with a cellular homolog known as src-c (also known as src-c, SRC and pp6osrc-c). The structure of the two proteins is similar but the regulatory carboxyl-terminus of v-src is truncated. Found in normal cells and presumed to be a proto-oncogene, src-c is a tyrosine kinase which regulates cell growth via phosphorylation of transcription factors, members of signal transduction cascades and growth factor receptors (Irby and Yeatman, Oncogene, 2000, 19, 5636-5642).

While elevation of src-c protein levels is common to a large number of cancers, this elevation is often modest when compared to the increases in src-c kinase activity that have been observed (Irby and Yeatman, Oncogene, 2000, 19, 5636-5642). These data indicate the importance of src-c activation in human tumor development and progression.

Examples of inhibition of human src-c expression by vectors containing antisense src-c fragments of src-c have been described in a mouse models (Karni et al., Oncogene, 1999, 18, 4654-4662; Wiener et al., Clin. Cancer Res., 1999, 5, 2164-2170), colon cancer cell lines (Ellis et al., J Biol. Chem., 1998, 273, 1052-1057; Fleming et al., Surgery, 1997, 122, 501-507; Rajala et al., Biochem. Biophys. Res. Commun., 2000, 273, 1116-1120; Staley et al., Cell Growth Differ., 1997, 8, 269-274) and leukemia cells lines (Kitanaka et al., Biochem. Biophys. Res. Commun., 1994, 201, 1534-1540; Waki et al., Biochem. Biophys. Res. Commun., 1994, 201, 1001-1007; Yamaguchi et al., Leukemia, 1997, 11, 497-503).

Investigations of src-c null mice indicate that src-c is not required for general cell viability but does have an essential role in osteoclast function and bone remodeling (Soriano et al., Cell, 1991, 64, 693-702). Inhibition of expression of src-c by antisense phosphorothioate oligonucleotides targeting the start codon of human and mouse src-c has been observed in osteoclasts, osteoblasts and vascular endothelial cells (Chellaiah et al., J. Biol. Chem., 1998, 273, 11908-11916.; Marzia et al., J. Cell Biol., 2000, 151, 311-320; Naruse et al., FEBS Lett., 1998, 441, 111-115; Tanaka et al., Nature, 1996, 383, 528-531).

A 60-mer oligonucleotide targeting the 18-nucleotide brain-specific insert of rat src-c was used to map the expression levels of brain-specific src-c in various brain structures (Ross et al., Proc. Natl. Acad. Sci. U.S.A., 1988, 85, 9831-9835).

An expression construct comprising a tumor supressor gene and an antisense src-c gene directed to the use of genetic therapy is claimed in PCT publication WO 99/47690 (Almond et al., 1999).

An antisense molecule inhibiting the expression of src-c in combination with a lipid formulation containing other compounds used for treatment of hyperproliferative disease in humans is claimed in PCT WO/71096 (Ramesh et al., 2000).

A therapeutic composition including an antisense oligonucleotide specific for src-c and at least one second antisense oligonucleotide specific for a nuclear oncogene is claimed in U.S. Pat. No. 5,734,039 (Calabretta and Skorski, 1998).

Antisense oligonucleotides corresponding to src-c and in combination with at least one other antisense oligonucleotide corresponding to a different gene are claimed in PCT publication WO 99/13886 (Nyce, 1999).

A therapeutic agent composed of a nucleic acid construct containing antisense RNA for disrupting expression of src-c is claimed in PCT publication WO 01/00791 (Lee, 2001).

Methods for producing recombinant viral vectors containing antisense constructs of src-c are claimed in PCT publication WO 99/27123 and WO 00/32754 (Fang et al., 1999; Zhang et al., 2000).

Antisense technology is an effective means for modulating the expression of one or more specific gene products and is uniquely useful in a number of therapeutic, diagnostic, and research applications.

Disclosed herein are antisense compounds useful for modulating gene expression and associated pathways via antisense mechanisms of action such as RNaseH, RNAi and dsRNA enzymes, as well as other antisense mechanisms based on target degradation or target occupancy. One having skill in the art, once armed with this disclosure will be able, without undue experimentation, to identify, prepare and exploit antisense compounds for these uses.

### SUMMARY OF THE INVENTION

Provided herein are oligomeric compounds, especially nucleic acid and nucleic acid-like oligomers, which are targeted to a nucleic acid encoding a bone growth modulator. Further provided are antisense compounds which are oligomeric compounds that modulate the expression of a bone growth modulator. Bone growth modulators disclosed herein include DKK-1, GSK3 beta, sFRP-1, sclerostin, transducer of ERRB1, and src-c. Also contemplated is a method of making an oligomeric compound comprising specifically hybridizing in vitro a first oligomeric strand comprising a sequence of at least 8 contiguous nucleobases of any of the sequences set forth in Table 6 to a second oligomeric strand comprising a sequence substantially complementary to said first strand.

Further provided are methods of modulating the expression of a bone growth modulator in cells, tissues or animals comprising contacting said cells, tissues or animals with one or more of the compounds or compositions of the present invention. For example, in one embodiment, the compounds or compositions of the present invention can be used to inhibit the expression of a bone growth modulator in cells, tissues or animals. Further contemplated are one or more antisense compounds or compositions to modulate the expression of more than one bone growth modulator.

Further provided are methods of identifying the relationship between a bone growth modulator and a disease state, phenotype, or condition by detecting or modulating said bone growth modulator comprising contacting a sample, tissue, cell, or organism with one or more oligomeric compounds, measuring the nucleic acid or protein level of said bone growth modulator and/or a related phenotypic or chemical endpoint coincident with or at some time after treatment, and optionally comparing the measured value to a non-treated sample or sample treated with a further compound, wherein a change in said nucleic acid or protein level of said bone growth modulator coincident with said related phenotypic or chemical endpoint indicates the existence or presence of a predisposition to a disease state, phenotype, or condition.

Further provided are methods of screening for modulators of a bone growth modulator expression by contacting a target segment of a nucleic acid molecule encoding said bone growth modulator with one or more candidate modulators, and selecting for one or more candidate modulators which decrease or increase the expression of a nucleic acid molecule encoding said bone growth modulator.

Further provided are methods of screening for additional modulators of a bone growth modulator expression by contacting a validated target segment of a nucleic acid molecule encoding said bone growth modulator with one or more candidate modulators, and selecting for one or more candidate modulators which decrease or increase the expression of a nucleic acid molecule encoding said bone growth modulator.

Pharmaceutical, therapeutic and other compositions comprising the compounds of the present invention are also provided.

Also provided is the use of the compounds or compositions of the invention in the manufacture of a medicament for the treatment of one or more conditions associated with a target of the invention. Further contemplated are methods where cells or tissues are contacted in vivo with an effective amount of one or more of the compounds or compositions of the invention. Also provided are ex vivo methods of treatment that include contacting cells or tissues with an effective amount of one or more of the compounds or compositions of the invention and then introducing said cells or tissues into an animal.

Methods of treating an animal, particularly a human, suspected of having or at risk for a disease or condition associated with expression of a bone growth modulator, such as bone density loss, are also set forth herein. Such methods include administering a therapeutically or prophylactically effective amount of one or more of the compounds or compositions of the present invention to an animal, particularly a human, in order to cause bone growth.

### DETAILED DESCRIPTION OF THE INVENTION

Overview

Disclosed herein are oligomeric compounds, including antisense oligonucleotides and other antisense compounds for use in modulating the expression of nucleic acid molecules encoding a bone growth modulator. Inhibition of a “bone growth modulator” leads to increased bone mass through increased osteoblast proliferation and activity. This is distinct from inhibition of bone remodelers or anti-resorptives which inhibit osteoclast mediated bone loss. This is accomplished by providing oligomeric compounds which hybridize with one or more target nucleic acid molecules encoding a bone growth modulator. As used herein, the terms “target nucleic acid” and “nucleic acid molecule encoding a bone growth modulator” have been used for convenience to encompass DNA encoding a bone growth modulator, RNA (including pre-mRNA and mRNA or portions thereof) transcribed from such DNA, and also cDNA derived from such RNA.

The principle behind antisense technology is that an antisense compound, which hybridizes to a target nucleic acid, modulates gene expression activities such as transcription or translation. This sequence specificity makes antisense compounds extremely attractive as tools for target validation and gene functionalization, as well as therapeutics to selectively modulate the expression of genes involved in disease.

Antisense Mechanisms

Antisense mechanisms are all those involving the hybridization of a compound with target nucleic acid, wherein the outcome or effect of the hybridization is either target degradation or target occupancy with concomitatnt stalling of the cellular machinery involving, for example, transcription or splicing.

Target degradation can include an RNase H. RNase H is a cellular endonuclease which cleaves the RNA strand of an RNA:DNA duplex. It is known in the art that single-stranded antisense compounds which are “DNA-like” elicit RNAse H. Activation of RNase H, therefore, results in cleavage of the RNA target, thereby greatly enhancing the efficiency of DNA-like oligonucleotide-mediated inhibition of gene expression.

Target degradation can include RNA interference (RNAi). RNAi is a form of posttranscriptional gene silencing that was initially defined in the nematode, Caenorhabditis elegans, resulting from exposure to double-stranded RNA (dsRNA). In many species the introduction of double-stranded structures, such as double-stranded RNA (dsRNA) molecules, has been shown to induce potent and specific antisense-mediated reduction of the function of a gene or its associated gene products. The RNAi compounds are often referred to as short interfering RNAs or siRNAs. Recently, it has been shown that it is, in fact, the single-stranded RNA oligomers of antisense polarity of the siRNAs which are the potent inducers of RNAi (Tijsterman et al., Science, 2002, 295, 694-697).

Both RNAi compounds (i.e., single- or double-stranded RNA or RNA-like compounds) and single-stranded RNase H-dependent antisense compounds bind to their RNA target by base pairing (i.e., hybridization) and induce site-specific cleavage of the target RNA by specific RNAses; i.e., both are antisense mechanisms (Vickers et al., 2003, J. Biol. Chem., 278, 7108-7118). Double-stranded ribonucleases (dsRNases) such as those in the RNase III and ribonuclease L family of enzymes also play a role in RNA target degradation. Double-stranded ribonucleases and oligomeric compounds that trigger them are further described in U.S. Pat. Nos. 5,898,031 and 6,107,094.

Nonlimiting examples of an occupancy-based antisense mechanism whereby antisense compounds hybridize yet do not elicit cleavage of the target include inhibition of translation, modulation of splicing, modulation of poly(A) site selection and disruption of regulatory RNA structure. A method of controlling the behavior of a cell through modulation of the processing of an mRNA target by contacting the cell with an antisense compound acting via a non-cleavage event is disclosed in U.S. Pat. No. 6,210,892 and U.S. Pre-Grant Publication 20020049173.

Certain types of antisense compounds which specifically hybridize to the 5′ cap region of their target mRNA can interfere with translation of the target mRNA into protein. Such oligomers include peptide-nucleic acid (PNA) oligomers, morpholino oligomers and oligonucleosides (such as those having an MMI or amide internucleoside linkage) and oligonucleotides having modifications at the 2′ position of the sugar when such oligomers are targeted to the 5′ cap region of their target mRNA. This is believed to occur via interference with ribosome assembly on the target mRNA. Methods for inhibiting the translation of a selected capped target mRNA by contacting target mRNA with an antisense compound are disclosed in U.S. Pat. No. 5,789,573.

Antisense compounds targeted to a specific poly(A) site of mRNA can be used to modulate the populations of alternatively polyadenylated transcripts. In addition, antisense compounds can be used to disrupt RNA regulatory structure thereby affecting, for example, the stability of the targeted RNA and its subsequent expression. Methods directed to such modulation are disclosed in U.S. Pat. No. 6,210,892 and Pre-Grant Publication 20020049173.

Compounds

The term “oligomeric compound” refers to a polymeric structure capable of hybridizing to a region of a nucleic acid molecule. This term includes oligonucleotides, oligonucleosides, oligonucleotide analogs, oligonucleotide mimetics and chimeric combinations of these. Oligomeric compounds are routinely prepared linearly but can be joined or otherwise prepared to be circular. Moreover, branched structures are known in the art. An “antisense compound” or “antisense oligomeric compound” refers to an oligomeric compound that is at least partially complementary to the region of a nucleic acid molecule to which it hybridizes and which modulates (increases or decreases) its expression. Consequently, while all antisense compounds can be said to be oligomeric compounds, not all oligomeric compounds are antisense compounds. An “antisense oligonucleotide” is an antisense compound that is a nucleic acid-based oligomer. An antisense oligonucleotide can be chemically modified. Nonlimiting examples of oligomeric compounds include primers, probes, antisense compounds, antisense oligonucleotides, external guide sequence (EGS) oligonucleotides, alternate splicers, and siRNAs. As such, these compounds can be introduced in the form of single-stranded, double-stranded, circular, branched or hairpins and can contain structural elements such as internal or terminal bulges or loops. Oligomeric double-stranded compounds can be two strands hybridized to form double-stranded compounds or a single strand with sufficient self complementarity to allow for hybridization and formation of a fully or partially double-stranded compound.

In one embodiment of the invention, double-stranded antisense compounds encompass short interfering RNAs (siRNAs). As used herein, the term “siRNA” is defined as a double-stranded compound having a first and second strand and comprises a central complementary portion between said first and second strands and terminal portions that are optionally complementary between said first and second strands or with the target mRNA. The ends of the strands may be modified by the addition of one or more natural or modified nucleobases to form an overhang. In one nonlimiting example, the first strand of the siRNA is antisense to the target nucleic acid, while the second strand is complementary to the first strand. Once the antisense strand is designed to target a particular nucleic acid target, the sense strand of the siRNA can then be designed and synthesized as the complement of the antisense strand and either strand may contain modifications or additions to either terminus. For example, in one embodiment, both strands of the siRNA duplex would be complementary over the central nucleobases, each having overhangs at one or both termini. It is possible for one end of a duplex to be blunt and the other to have overhanging nucleobases. In one embodiment, the number of overhanging nucleobases is from 1 to 6 on the 3′ end of each strand of the duplex. In another embodiment, the number of overhanging nucleobases is from 1 to 6 on the 3′ end of only one strand of the duplex. In a further embodiment, the number of overhanging nucleobases is from 1 to 6 on one or both 5′ ends of the duplexed strands. In another embodiment, the number of overhanging nucleobases is zero.

In one embodiment of the invention, double-stranded antisense compounds are canonical siRNAs. As used herein, the term “canonical siRNA” is defined as a double-stranded oligomeric compound having a first strand and a second strand each strand being 21 nucleobases in length with the strands being complementary over 19 nucleobases and having on each 3′ termini of each strand a deoxy thymidine dimer (dTdT) which in the double-stranded compound acts as a 3′ overhang.

Each strand of the siRNA duplex may be from about 8 to about 80 nucleobases, 10 to 50, 13 to 80, 13 to 50, 13 to 30, 13 to 24, 19 to 23, 20 to 80, 20 to 50, 20 to 30, or 20 to 24 nucleobases. The central complementary portion may be from about 8 to about 80 nucleobases in length, 10 to 50, 13 to 80, 13 to 50, 13 to 30, 13 to 24, 19 to 23, 20 to 80, 20 to 50, 20 to 30, or 20 to 24 nucleobases. The terminal portions can be from 1 to 6 nucleobases. The siRNAs may also have no terminal portions. The two strands of an siRNA can be linked internally leaving free 3′ or 5′ termini or can be linked to form a continuous hairpin structure or loop. The hairpin structure may contain an overhang on either the 5′ or 3′ terminus producing an extension of single-stranded character.

In another embodiment, the double-stranded antisense compounds are blunt-ended siRNAs. As used herein the term “blunt-ended siRNA” is defined as an siRNA having no terminal overhangs. That is, at least one end of the double-stranded compound is blunt. siRNAs whether canonical or blunt act to elicit dsRNAse enzymes and trigger the recruitment or activation of the RNAi antisense mechanism. In a further embodiment, single-stranded RNAi (ssRNAi) compounds that act via the RNAi antisense mechanism are contemplated.

Further modifications can be made to the double-stranded compounds and may include conjugate groups attached to one of the termini, selected nucleobase positions, sugar positions or to one of the internucleoside linkages. Alternatively, the two strands can be linked via a non-nucleic acid moiety or linker group. When formed from only one strand, the compounds can take the form of a self-complementary hairpin-type molecule that doubles back on itself to form a duplex. Thus, the compounds can be fully or partially double-stranded. When formed from two strands, or a single strand that takes the form of a self-complementary hairpin-type molecule doubled back on itself to form a duplex, the two strands (or duplex-forming regions of a single strand) are complementary when they base pair in Watson-Crick fashion.

The oligomeric compounds in accordance with this invention may comprise a complementary oligomeric compound from about 8 to about 80 nucleobases (i.e. from about 8 to about 80 linked nucleosides). In other words, a single-stranded compound of the invention comprises from 8 to about 80 nucleobases, and a double-stranded antisense compound of the invention (such as a siRNA, for example) comprises two strands, each of which is from about 8 to about 80 nucleobases. Contained within the oligomeric compounds of the invention (whether single or double stranded and on at least one strand) are antisense portions. The “antisense portion” is that part of the oligomeric compound that is designed to work by one of the aforementioned antisense mechanisms. One of ordinary skill in the art will appreciate that this comprehends antisense portions of 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases.

In one embodiment, the antisense compounds of the invention have antisense portions of 10 to 50 nucleobases. One having ordinary skill in the art will appreciate that this embodies antisense compounds having antisense portions of 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobases.

In one embodiment, the antisense compounds of the invention have antisense portions of 13 to 80 nucleobases. One having ordinary skill in the art will appreciate that this embodies antisense compounds having antisense portions of 13, 14, 15, 16, 17, 18, 19, 20, 21, 22, 23, 24, 25, 26, 27, 28, 2930, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases.

In one embodiment, the antisense compounds of the invention have antisense portions of 13 to 50 nucleobases. One having ordinary skill in the art will appreciate that this embodies antisense compounds having antisense portions of 13, 14, 15, 16, 17, 18, 19, 20, 21, 22, 23, 24, 25, 26, 27, 28, 2930, 31, 32, 33, 34, 35, 36, 37, 38, 39, 40, 41, 42, 43, 44, 45, 46, 47, 48, 49, or 50 nucleobases.

In one embodiment, the antisense compounds of the invention have antisense portions of 13 to 30 nucleobases. One having ordinary skill in the art will appreciate that this embodies antisense compounds having antisense portions of 13, 14, 15, 16, 17, 18, 19, 20, 21, 22, 23, 24, 25, 26, 27, 28, 29 or 30 nucleobases.

In some embodiments, the antisense compounds of the invention have antisense portions of 13 to 24 nucleobases. One having ordinary skill in the art will appreciate that this embodies antisense compounds having antisense portions of 13, 14, 15, 16, 17, 18, 19, 20, 21, 22, 23 or 24 nucleobases.

In one embodiment, the antisense compounds of the invention have antisense portions of 19 to 23 nucleobases. One having ordinary skill in the art will appreciate that this embodies antisense compounds having antisense portions of 19, 20, 21, 22 or 23 nucleobases.

In one embodiment, the antisense compounds of the invention have antisense portions of 20 to 80 nucleobases. One having ordinary skill in the art will appreciate that this embodies antisense compounds having antisense portions of 20, 21, 22, 23, 24, 25, 26, 27, 28, 29 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 nucleobases.

In one embodiment, the antisense compounds of the invention have antisense portions of 20 to 50 nucleobases. One having ordinary skill in the art will appreciate that this embodies antisense compounds having antisense portions of 20, 21, 22, 23, 24, 25, 26, 27, 28, 29 30, 31, 32, 33, 34, 35, 36, 37, 38, 39, 40, 41, 42, 43, 44, 45, 46, 47, 48, 49, or 50 nucleobases.

In one embodiment, the antisense compounds of the invention have antisense portions of 20 to 30 nucleobases. One having ordinary skill in the art will appreciate that this embodies antisense compounds having antisense portions of 20, 21, 22, 23, 24, 25, 26, 27, 28, 29, or 30 nucleobases.

In one embodiment, the antisense compounds of the invention have antisense portions of 20 to 24 nucleobases. One having ordinary skill in the art will appreciate that this embodies antisense compounds having antisense portions of 20, 21, 22, 23, or 24 nucleobases.

In one embodiment, the antisense compounds of the invention have antisense portions of 20 nucleobases.

In one embodiment, the antisense compounds of the invention have antisense portions of 19 nucleobases.

In one embodiment, the antisense compounds of the invention have antisense portions of 18 nucleobases.

In one embodiment, the antisense compounds of the invention have antisense portions of 17 nucleobases.

In one embodiment, the antisense compounds of the invention have antisense portions of 16 nucleobases.

In one embodiment, the antisense compounds of the invention have antisense portions of 15 nucleobases.

In one embodiment, the antisense compounds of the invention have antisense portions of 14 nucleobases.

In one embodiment, the antisense compounds of the invention have antisense portions of 13 nucleobases.

Antisense compounds 8-80 nucleobases in length comprising a stretch of at least eight (8) consecutive nucleobases selected from within the illustrative antisense compounds are considered to be suitable antisense compounds as well.

Compounds of the invention include oligonucleotide sequences that comprise at least the 8 consecutive nucleobases from the 5′-terminus of one of the illustrative antisense compounds (the remaining nucleobases being a consecutive stretch of the same oligonucleotide beginning immediately upstream of the 5′-terminus of the antisense compound which is specifically hybridizable to the target nucleic acid and continuing until the oligonucleotide contains about 8 to about 80 nucleobases). Other compounds are represented by oligonucleotide sequences that comprise at least the 8 consecutive nucleobases from the 3′-terminus of one of the illustrative antisense compounds (the remaining nucleobases being a consecutive stretch of the same oligonucleotide beginning immediately downstream of the 3′-terminus of the antisense compound which is specifically hybridizable to the target nucleic acid and continuing until the oligonucleotide contains about 8 to about 80 nucleobases). It is also understood that compounds may be represented by oligonucleotide sequences that comprise at least 8 consecutive nucleobases from an internal portion of the sequence of an illustrative compound, and may extend in either or both directions until the oligonucleotide contains about 8 about 80 nucleobases.

One having skill in the art armed with the antisense compounds illustrated herein will be able, without undue experimentation, to identify further antisense compounds.

Chemical Modifications

As is known in the art, a nucleoside is a base-sugar combination. The base portion of the nucleoside is normally a heterocyclic base (sometimes referred to as a “nucleobase” or simply a “base”). The two most common classes of such heterocyclic bases are the purines and the pyrimidines. Nucleotides are nucleosides that further include a phosphate group covalently linked to the sugar portion of the nucleoside. For those nucleosides that include a pentofuranosyl sugar, the phosphate group can be linked to either the 2′, 3′ or 5′ hydroxyl moiety of the sugar. In forming oligonucleotides, the phosphate groups covalently link adjacent nucleosides to one another to form a linear polymeric compound. In turn, the respective ends of this linear polymeric compound can be further joined to form a circular compound. In addition, linear compounds may have internal nucleobase complementarity and may therefore fold in a manner as to produce a fully or partially double-stranded compound. Within oligonucleotides, the phosphate groups are commonly referred to as forming the internucleoside backbone of the oligonucleotide. The normal linkage or backbone of RNA and DNA is a 3′ to 5′ phosphodiester linkage.

Modified Internucleoside Linkages

Specific examples of oligomeric compounds useful of the present invention include oligonucleotides containing modified e.g. non-naturally occurring internucleoside linkages. As defined in this specification, oligonucleotides having modified internucleoside linkages include internucleoside linkages that retain a phosphorus atom and internucleoside linkages that do not have a phosphorus atom. For the purposes of this specification, and as sometimes referenced in the art, modified oligonucleotides that do not have a phosphorus atom in their internucleoside backbone can also be considered to be oligonucleosides.

Oligomeric compounds can have one or more modified internucleoside linkages. Modified oligonucleotide backbones containing a phosphorus atom therein include, for example, phosphorothioates, chiral phosphorothioates, phosphorodithioates, phosphotriesters, aminoalkyl-phosphotriesters, methyl and other alkyl phosphonates including 3′-alkylene phosphonates, 5′-alkylene phosphonates and chiral phosphonates, phosphinates, phosphoramidates including 3′-amino phosphoramidate and aminoalkylphosphoramidates, thionophosphoramidates, thiono-alkylphosphonates, thionoalkylphosphotriesters, phosphonoacetate and thiophosphonoacetate (see Sheehan et al., Nucleic Acids Research, 2003, 31(14), 4109-4118 and Dellinger et al., J. Am. Chem. Soc., 2003, 125, 940-950), selenophosphates and boranophosphates having normal 3′-5′ linkages, 2′-5′ linked analogs of these, and those having inverted polarity wherein one or more internucleotide linkages is a 3′ to 3′, 5′ to 5′ or 2′ to 2′ linkage. Oligonucleotides having inverted polarity comprise a single 3′ to 3′ linkage at the 3′-most internucleotide linkage i.e. a single inverted nucleoside residue which may be abasic (the nucleobase is missing or has a hydroxyl group in place thereof). Various salts, mixed salts and free acid forms are also included.

N3′-P5′-phosphoramidates have been reported to exhibit both a high affinity towards a complementary RNA strand and nuclease resistance (Gryaznov et al., J. Am. Chem. Soc., 1994, 116, 3143-3144). N3′-P5′-phosphoramidates have been studied with some success in vivo to specifically down regulate the expression of the c-myc gene (Skorski et al., Proc. Natl. Acad. Sci., 1997, 94, 3966-3971; and Faira et al., Nat. Biotechnol., 2001, 19, 40-44).

Representative United States patents that teach the preparation of the above phosphorus-containing linkages include, but are not limited to, U.S. Pat. Nos. 3,687,808; 4,469,863; 4,476,301; 5,023,243; 5,177,196; 5,188,897; 5,264,423; 5,276,019; 5,278,302; 5,286,717; 5,321,131; 5,399,676; 5,405,939; 5,453,496; 5,455,233; 5,466,677; 5,476,925; 5,519,126; 5,536,821; 5,541,306; 5,550,111; 5,563,253; 5,571,799; 5,587,361; 5,194,599; 5,565,555; 5,527,899; 5,721,218; 5,672,697 and 5,625,050.

In some embodiments of the invention, oligomeric compounds may have one or more phosphorothioate and/or heteroatom internucleoside linkages, in particular —CH₂—NH—O—CH₂—, —CH₂—N(CH₃)—O—CH₂— (known as a methylene (methylimino) or MMI backbone), —CH₂—O—N(CH₃)—CH₂—, —CH₂—N(CH₃)—N(CH₃)—CH₂— and —O—N(CH₃)—CH₂—CH₂— (wherein the native phosphodiester internucleotide linkage is represented as —O—P(═O)(OH)—O—CH₂—). The MMI type internucleoside linkages are disclosed in the above referenced U.S. Pat. No. 5,489,677. Amide internucleoside linkages are disclosed in the above referenced U.S. Pat. No. 5,602,240.

Some oligonucleotide backbones that do not include a phosphorus atom therein have backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

Representative United States patents that teach the preparation of the above oligonucleosides include, but are not limited to, U.S. Pat. Nos. 5,034,506; 5,166,315; 5,185,444; 5,214,134; 5,216,141; 5,235,033; 5,264,562; 5,264,564; 5,405,938; 5,434,257; 5,466,677; 5,470,967; 5,489,677; 5,541,307; 5,561,225; 5,596,086; 5,602,240; 5,610,289; 5,602,240; 5,608,046; 5,610,289; 5,618,704; 5,623,070; 5,663,312; 5,633,360; 5,677,437; 5,792,608; 5,646,269 and 5,677,439.

Modified Sugars

Oligomeric compounds may also contain one or more substituted sugar moieties. Suitable compounds can comprise one of the following at the 2′ position: OH; F; O—, S—, or N-alkyl; O—, S—, or N-alkenyl; O—, S— or N-alkynyl; or O-alkyl-O-alkyl, wherein the alkyl, alkenyl and alkynyl may be substituted or unsubstituted C₁ to C₁₀ alkyl or C₂ to C₁₀ alkenyl and alkynyl. Also suitable are O(CH₂)ₙO)ₘCH₃, O(CH₂)ₙOCH₃, O(CH₂)ₙNH₂, O(CH₂)ₙCH₃, O(CH₂)ₙONH₂, and O(CH₂)ₙON((CH₂)ₙCH₃)₂, where n and m are from 1 to about 10. Other oligonucleotides comprise one of the following at the 2′ position: C₁ to C₁₀ lower alkyl, substituted lower alkyl, alkenyl, alkynyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. One modification includes 2′-methoxyethoxy (2′-O—CH₂CH₂OCH₃, also known as 2′-O-(2-methoxyethyl) or 2′-MOE) (Martin et al., Helv. Chim. Acta, 1995, 78, 486-504) i.e., an alkoxyalkoxy group. A further modification includes 2′-dimethylaminooxyethoxy, i.e., a O(CH₂)₂ON(CH₃)₂ group, also known as 2′-DMAOE, as described in examples hereinbelow, and 2′-dimethylaminoethoxyethoxy (also known in the art as 2′-O-dimethyl-amino-ethoxy-ethyl or 2′-DMAEOE), i.e., 2′-O—(CH₂—O—(CH₂)₂—N(CH₃₎₂, also described in examples hereinbelow.

Other modifications include 2′-methoxy (2′-O—CH₃), 2′-aminopropoxy (2′-OCH₂CH₂CH₂NH₂), 2′-allyl (2′-CH₂—CH═CH₂), 2′-O-allyl (2′-O—CH₂—CH═CH₂) and 2′-fluoro (2′-F). The 2′-modification may be in the arabino (up) position or ribo (down) position. One 2′-arabino modification is 2′-F. Similar modifications may also be made at other positions on the oligonucleotide, particularly the 3′ position of the sugar on the 3′ terminal nucleotide or in 2′-5′ linked oligonucleotides and the 5′ position of 5′ terminal nucleotide. Antisense compounds may also have sugar mimetics such as cyclobutyl moieties in place of the pentofuranosyl sugar. Representative United States patents that teach the preparation of such modified sugar structures include, but are not limited to, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,658,873; 5,670,633; 5,792,747; 5,700,920; and, 6,147,200.

DNA-Like and RNA-Like Conformations

The terms used to describe the conformational geometry of homoduplex nucleic acids are “A Form” for RNA and “B Form” for DNA. In general, RNA:RNA duplexes are more stable and have higher melting temperatures (Tm's) than DNA:DNA duplexes (Sanger et al., Principles of Nucleic Acid Structure, 1984, Springer-Verlag; New York, N.Y.; Lesnik et al., Biochemistry, 1995, 34, 10807-10815; Conte et al., Nucleic Acids Res., 1997, 25, 2627-2634). The increased stability of RNA has been attributed to several structural features, most notably the improved base stacking interactions that result from an A-form geometry (Searle et al., Nucleic Acids Res., 1993, 21, 2051-2056). The presence of the 2′ hydroxyl in RNA biases the sugar toward a C3′ endo pucker, i.e., also designated as Northern pucker, which causes the duplex to favor the A-form geometry. In addition, the 2′ hydroxyl groups of RNA can form a network of water mediated hydrogen bonds that help stabilize the RNA duplex (Egli et al., Biochemistry, 1996, 35, 8489-8494). On the other hand, deoxy nucleic acids prefer a C2′ endo sugar pucker, i.e., also known as Southern pucker, which is thought to impart a less stable B-form geometry (Sanger et al., Principles of Nucleic Acid Structure, 1984, Springer-Verlag; New York, N.Y.). As used herein, B-form geometry is inclusive of both C2′-endo pucker and O4′-endo pucker.

The structure of a hybrid duplex is intermediate between A- and B-form geometries, which may result in poor stacking interactions (Lane et al., Eur. J. Biochem., 1993, 215, 297-306; Fedoroff et al, J. Mol. Biol., 1993, 233, 509-523; Gonzalez et al., Biochemistry, 1995, 34, 4969-4982; Horton et al, J. Mol. Biol., 1996, 264, 521-533). Consequently, compounds that favor an A-form geometry can enhance stacking interactions, thereby increasing the relative Tm and potentially enhancing a compound's antisense effect.

In one aspect of the present invention oligomeric compounds include nucleosides synthetically modified to induce a 3′-endo sugar conformation. A nucleoside can incorporate synthetic modifications of the heterocyclic base, the sugar moiety or both to induce a desired 3′-endo sugar conformation. These modified nucleosides are used to mimic RNA-like nucleosides so that particular properties of an oligomeric compound can be enhanced while maintaining the desirable 3′-endo conformational geometry.

There is an apparent preference for an RNA type duplex (A form helix, predominantly 3′-endo) as a requirement (e.g. trigger) of RNA interference which is supported in part by the fact that duplexes composed of 2′-deoxy-2′-F-nucleosides appears efficient in triggering RNAi response in the C elegans system. Properties that are enhanced by using more stable 3′-endo nucleosides include but are not limited to: modulation of pharmacokinetic properties through modification of protein binding, protein off-rate, absorption and clearance; modulation of nuclease stability as well as chemical stability; modulation of the binding affinity and specificity of the oligomer (affinity and specificity for enzymes as well as for complementary sequences); and increasing efficacy of RNA cleavage. Also provided herein are oligomeric triggers of RNAi having one or more nucleosides modified in such a way as to favor a C3′-endo type conformation.

Nucleoside conformation is influenced by various factors including substitution at the 2′, 3′ or 4′-positions of the pentofuranosyl sugar. Electronegative substituents generally prefer the axial positions, while sterically demanding substituents generally prefer the equatorial positions (Principles of Nucleic Acid Structure, Wolfgang Sanger, 1984, Springer-Verlag.) Modification of the 2′ position to favor the 3′-endo conformation can be achieved while maintaining the 2′-OH as a recognition element (Gallo et al., Tetrahedron (2001), 57, 5707-5713. Harry-O'kuru et al., J. Org. Chem., (1997), 62(6), 1754-1759 and Tang et al., J. Org. Chem. (1999), 64, 747-754.) Alternatively, preference for the 3′-endo conformation can be achieved by deletion of the 2′-OH as exemplified by 2′deoxy-2′F-nucleosides (Kawasaki et al., J. Med. Chem. (1993), 36, 831-841), which adopts the 3′-endo conformation positioning the electronegative fluorine atom in the axial position. Representative 2′-substituent groups amenable to the present invention that give A-form conformational properties (3′-endo) to the resultant duplexes include 2′-O-alkyl, 2′-O-substituted alkyl and 2′-fluoro substituent groups. Other suitable substituent groups are various alkyl and aryl ethers and thioethers, amines and monoalkyl and dialkyl substituted amines.

Other modifications of the ribose ring, for example substitution at the 4′-position to give 4′-F modified nucleosides (Guillerm et al., Bioorganic and Medicinal Chemistry Letters (1995), 5, 1455-1460 and Owen et al., J. Org. Chem. (1976), 41, 3010-3017), or for example modification to yield methanocarba nucleoside analogs (Jacobson et al., J. Med. Chem. Lett. (2000), 43, 2196-2203 and Lee et al., Bioorganic and Medicinal Chemistry Letters (2001), 11, 1333-1337) also induce preference for the 3′-endo conformation. Along similar lines, triggers of RNAi response might be composed of one or more nucleosides modified in such a way that conformation is locked into a C3′-endo type conformation, i.e. Locked Nucleic Acid (LNA, Singh et al, Chem. Commun. (1998), 4, 455-456), and ethylene bridged Nucleic Acids (ENA™, Morita et al, Bioorganic & Medicinal Chemistry Letters (2002), 12, 73-76.)

It is further intended that multiple modifications can be made to one or more of the oligomeric compounds of the invention at multiple sites of one or more monomeric subunits (nucleosides are suitable) and or internucleoside linkages to enhance properties such as but not limited to activity in a selected application.

The synthesis of numerous of the modified nucleosides amenable to the present invention are known in the art (see for example, Chemistry of Nucleosides and Nucleotides Vol 1-3, ed. Leroy B. Townsend, 1988, Plenum press). The conformation of modified nucleosides and their oligomers can be estimated by various methods routine to those skilled in the art such as molecular dynamics calculations, nuclear magnetic resonance spectroscopy and CD measurements.

Oligonucleotide Mimetics

Another group of oligomeric compounds includes oligonucleotide mimetics. The term “mimetic” as it is applied to oligonucleotides includes oligomeric compounds wherein the furanose ring or the furanose ring and the internucleotide linkage are replaced with novel groups, replacement of only the furanose ring is also referred to in the art as being a sugar surrogate. The heterocyclic base moiety or a modified heterocyclic base moiety is maintained for hybridization with an appropriate target nucleic acid.

One such oligomeric compound, an oligonucleotide mimetic that has been shown to have excellent hybridization properties, is referred to as a peptide nucleic acid (PNA) (Nielsen et al., Science, 1991, 254, 1497-1500). PNAs have favorable hybridization properties, high biological stability and are electrostatically neutral molecules. PNA compounds have been used to correct aberrant splicing in a transgenic mouse model (Sazani et al., Nat. Biotechnol., 2002, 20, 1228-1233). In PNA oligomeric compounds, the sugar-backbone of an oligonucleotide is replaced with an amide containing backbone, in particular an aminoethylglycine backbone. The nucleobases are bound directly or indirectly to aza nitrogen atoms of the amide portion of the backbone. Representative United States patents that teach the preparation of PNA oligomeric compounds include, but are not limited to, U.S. Pat. Nos. 5,539,082; 5,714,331; and 5,719,262. PNA compounds can be obtained commercially from Applied Biosystems (Foster City, Calif., USA). Numerous modifications to the basic PNA backbone are known in the art; particularly useful are PNA compounds with one or more amino acids conjugated to one or both termini. For example, 1-8 lysine or arginine residues are useful when conjugated to the end of a PNA molecule.

Another class of oligonucleotide mimetic that has been studied is based on linked morpholino units (morpholino nucleic acid) having heterocyclic bases attached to the morpholino ring. A number of linking groups have been reported that link the morpholino monomeric units in a morpholino nucleic acid. One class of linking groups have been selected to give a non-ionic oligomeric compound. Morpholino-based oligomeric compounds are non-ionic mimetics of oligonucleotides which are less likely to form undesired interactions with cellular proteins (Dwaine A. Braasch and David R. Corey, Biochemistry, 2002, 41(14), 4503-4510). Morpholino-based oligomeric compounds have been studied in zebrafish embryos (see: Genesis, volume 30, issue 3, 2001 and Heasman, J., Dev. Biol., 2002, 243, 209-214). Further studies of morpholino-based oligomeric compounds have also been reported (Nasevicius et al., Nat. Genet., 2000, 26, 216-220; and Lacerra et al., Proc. Natl. Acad. Sci., 2000, 97, 9591-9596). Morpholino-based oligomeric compounds are disclosed in U.S. Pat. No. 5,034,506. The morpholino class of oligomeric compounds have been prepared having a variety of different linking groups joining the monomeric subunits. Linking groups can be varied from chiral to achiral, and from charged to neutral. U.S. Pat. No. 5,166,315 discloses linkages including —O—P(═O)(N(CH₃)₂)—O—; U.S. Pat. No. 5,034,506 discloses achiral intermorpholino linkages; and U.S. Pat. No. 5,185,444 discloses phosphorus containing chiral intermorpholino linkages.

A further class of oligonucleotide mimetic is referred to as cyclohexene nucleic acids (CeNA). In CeNA oligonucleotides, the furanose ring normally present in a DNA or RNA molecule is replaced with a cyclohexenyl ring. CeNA DMT protected phosphoramidite monomers have been prepared and used for oligomeric compound synthesis following classical phosphoramidite chemistry. Fully modified CeNA oligomeric compounds and oligonucleotides having specific positions modified with CeNA have been prepared and studied (Wang et al., J. Am. Chem. Soc., 2000, 122, 8595-8602). In general the incorporation of CeNA monomers into a DNA chain increases its stability of a DNA/RNA hybrid. CeNA oligoadenylates formed complexes with RNA and DNA complements with similar stability to the native complexes. The study of incorporating CeNA structures into natural nucleic acid structures was shown by NMR and circular dichroism to proceed with easy conformational adaptation. Furthermore the incorporation of CeNA into a sequence targeting RNA was stable to serum and able to activate E. coli RNase H resulting in cleavage of the target RNA strand.

A further modification includes bicyclic sugar moieties such as “Locked Nucleic Acids” (LNAs) in which the 2′-hydroxyl group of the ribosyl sugar ring is linked to the 4′ carbon atom of the sugar ring thereby forming a 2′-C, 4′-C-oxymethylene linkage to form the bicyclic sugar moiety (reviewed in Elayadi et al., Curr. Opinion Invens. Drugs, 2001, 2, 558-561; Braasch et al., Chem. Biol., 2001, 8 1-7; and Orum et al., Curr. Opinion Mol. Ther., 2001, 3, 239-243; see also U.S. Pat. Nos. 6,268,490 and 6,670,461). The linkage can be a methylene (—CH₂—) group bridging the 2′ oxygen atom and the 4′ carbon atom, for which the term LNA is used for the bicyclic moiety; in the case of an ethylene group in this position, the term ENA™ is used (Singh et al., Chem. Commun., 1998, 4, 455-456; ENA™: Morita et al., Bioorganic Medicinal Chemistry, 2003, 11, 2211-2226). LNA and other bicyclic sugar analogs display very high duplex thermal stabilities with complementary DNA and RNA (Tm=+3 to +10 C), stability towards 3′-exonucleolytic degradation and good solubility properties. LNA's are commercially available from ProLigo (Paris, France and Boulder, Colo., USA).

An isomer of LNA that has also been studied is alpha-L-LNA which has been shown to have superior stability against a 3′-exonuclease. The alpha-L-LNA's were incorporated into antisense gapmers and chimeras that showed potent antisense activity (Frieden et al., Nucleic Acids Research, 2003, 21, 6365-6372).

Another similar bicyclic sugar moiety that has been prepared and studied has the bridge going from the 3′-hydroxyl group via a single methylene group to the 4′ carbon atom of the sugar ring thereby forming a 3′-C, 4′-C-oxymethylene linkage (see U.S. Pat. No. 6,043,060).

LNA has been shown to form exceedingly stable LNA:LNA duplexes (Koshkin et al., J. Am. Chem. Soc., 1998, 120, 13252-13253). LNA:LNA hybridization was shown to be the most thermally stable nucleic acid type duplex system, and the RNA-mimicking character of LNA was established at the duplex level. Introduction of 3 LNA monomers (T or A) significantly increased melting points (Tm=+15/+11) toward DNA complements. The universality of LNA-mediated hybridization has been stressed by the formation of exceedingly stable LNA:LNA duplexes. The RNA-mimicking of LNA was reflected with regard to the N-type conformational restriction of the monomers and to the secondary structure of the LNA:RNA duplex.

LNAs also form duplexes with complementary DNA, RNA or LNA with high thermal affinities. Circular dichroism (CD) spectra show that duplexes involving fully modified LNA (esp. LNA:RNA) structurally resemble an A-form RNA:RNA duplex. Nuclear magnetic resonance (NMR) examination of an LNA:DNA duplex confirmed the 3′-endo conformation of an LNA monomer. Recognition of double-stranded DNA has also been demonstrated suggesting strand invasion by LNA. Studies of mismatched sequences show that LNAs obey the Watson-Crick base pairing rules with generally improved selectivity compared to the corresponding unmodified reference strands. DNA LNA chimeras have been shown to efficiently inhibit gene expression when targeted to a variety of regions (5′-untranslated region, region of the start codon or coding region) within the luciferase mRNA (Braasch et al., Nucleic Acids Research, 2002, 30, 5160-5167).

Potent and nontoxic antisense oligonucleotides containing LNAs have been described (Wahlestedt et al., Proc. Natl. Acad. Sci. U.S.A., 2000, 97, 5633-5638). The authors have demonstrated that LNAs confer several desired properties. LNA/DNA copolymers were not degraded readily in blood serum and cell extracts. LNA/DNA copolymers exhibited potent antisense activity in assay systems as disparate as G-protein-coupled receptor signaling in living rat brain and detection of reporter genes in Escherichia coli. Lipofectin-mediated efficient delivery of LNA into living human breast cancer cells has also been accomplished. Further successful in vivo studies involving LNA's have shown knock-down of the rat delta opioid receptor without toxicity (Wahlestedt et al., Proc. Natl. Acad. Sci., 2000, 97, 5633-5638) and in another study showed a blockage of the translation of the large subunit of RNA polymerase II (Fluiter et al., Nucleic Acids Res., 2003, 31, 953-962).

The synthesis and preparation of the LNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). LNAs and preparation thereof are also described in WO 98/39352 and WO 99/14226.

Analogs of LNA, phosphorothioate-LNA and 2′-thio-LNAs, have also been prepared (Kumar et al., Bioorg. Med. Chem. Lett., 1998, 8, 2219-2222). Preparation of locked nucleoside analogs containing oligodeoxyribonucleotide duplexes as substrates for nucleic acid polymerases has also been described (Wengel et al., WO 99/14226). Furthermore, synthesis of 2′-amino-LNA, a novel conformationally restricted high-affinity oligonucleotide analog has been described in the art (Singh et al., J. Org. Chem., 1998, 63, 10035-10039). In addition, 2′-Amino- and 2′-methylamino-LNA’s have been prepared and the thermal stability of their duplexes with complementary RNA and DNA strands has been previously reported.

Another oligonucleotide mimetic that has been prepared and studied is threose nucleic acid. This oligonucleotide mimetic is based on threose nucleosides instead of ribose nucleosides. Initial interest in (3′,2′)-alpha-L-threose nucleic acid (TNA) was directed to the question of whether a DNA polymerase existed that would copy the TNA. It was found that certain DNA polymerases are able to copy limited stretches of a TNA template (reported in Chemical and Engineering News, 2003, 81, 9). In another study it was determined that TNA is capable of antiparallel Watson-Crick base pairing with complementary DNA, RNA and TNA oligonucleotides (Chaput et al., J. Am. Chem. Soc., 2003, 125, 856-857).

In one study (3′,2′)-alpha-L-threose nucleic acid was prepared and compared to the 2′ and 3′ amidate analogs (Wu et al., Organic Letters, 2002, 4(8), 1279-1282). The amidate analogs were shown to bind to RNA and DNA with comparable strength to that of RNA/DNA.

Further oligonucleotide mimetics have been prepared to include bicyclic and tricyclic nucleoside analogs (see Steffens et al., Helv. Chim. Acta, 1997, 80, 2426-2439; Steffens et al., J. Am. Chem. Soc., 1999, 121, 3249-3255; Renneberg et al., J. Am. Chem. Soc., 2002, 124, 5993-6002; and Renneberg et al., Nucleic acids res., 2002, 30, 2751-2757). These modified nucleoside analogs have been oligomerized using the phosphoramidite approach and the resulting oligomeric compounds containing tricyclic nucleoside analogs have shown increased thermal stabilities (Tm's) when hybridized to DNA, RNA and itself Oligomeric compounds containing bicyclic nucleoside analogs have shown thermal stabilities approaching that of DNA duplexes.

Another class of oligonucleotide mimetic is referred to as phosphonomonoester nucleic acids which incorporate a phosphorus group in the backbone. This class of olignucleotide mimetic is reported to have useful physical and biological and pharmacological properties in the areas of inhibiting gene expression (antisense oligonucleotides, sense oligonucleotides and triplex-forming oligonucleotides), as probes for the detection of nucleic acids and as auxiliaries for use in molecular biology. Further oligonucleotide mimetics amenable to the present invention have been prepared wherein a cyclobutyl ring replaces the naturally occurring furanosyl ring.

Modified and Alternate Nucleobases

Oligomeric compounds can also include nucleobase (often referred to in the art as heterocyclic base or simply as “base”) modifications or substitutions. As used herein, “unmodified” or “natural” nucleobases include the purine bases adenine (A) and guanine (G), and the pyrimidine bases thymine (T), cytosine (C) and uracil (U). A “substitution” is the replacement of an unmodified or natural base with another unmodified or natural base. “Modified” nucleobases mean other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C≡C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified nucleobases include tricyclic pyrimidines such as phenoxazine cytidine(1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g. 9-(2-aminoethoxy)-H-pyrimido(5,4-b)(1,4)benzoxazin-2(3H)-one), carbazole cytidine (2H-pyrimido(4,5-b)indol-2-one), pyridoindole cytidine (H-pyrido(3′,2′:4,5)pyrrolo(2,3-d)pyrimidin-2-one). Modified nucleobases may also include those in which the purine or pyrimidine base is replaced with other heterocycles, for example 7-deaza-adenine, 7-deazaguanosine, 2-aminopyridine and 2-pyridone. Further nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al, Angewandte Chemie, International Edition, 1991, 30, 613, and those disclosed by Sanghvi, Y. S., Chapter 15, Antisense Research and Applications, pages 289-302, Crooke, S. T. and Lebleu, B., ed., CRC Press, 1993. Certain of these nucleobases are known to those skilled in ther art as suitable for increasing the binding affinity of the compounds of the invention. These include 5-substituted pyrimidines, 6-azapyrimidines and N-2, N-6 and O-6 substituted purines, including 2-aminopropyladenine, 5-propynyluracil and 5-propynylcytosine. 5-methylcytosine substitutions have been shown to increase nucleic acid duplex stability by 0.6-1.2° C. and are presently suitable base substitutions, even more particularly when combined with 2′-O-methoxyethyl sugar modifications. It is understood in the art that modification of the base does not entail such chemical modifications as to produce substitutions in a nucleic acid sequence.

Representative United States patents that teach the preparation of certain of the above noted modified nucleobases as well as other modified nucleobases include, but are not limited to, the above noted U.S. Pat. No. 3,687,808, as well as U.S. Pat. Nos. 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,681,941; and 5,750,692.

Oligomeric compounds of the present invention can also include polycyclic heterocyclic compounds in place of one or more of the naturally-occurring heterocyclic base moieties. A number of tricyclic heterocyclic compounds have been previously reported. These compounds are routinely used in antisense applications to increase the binding properties of the modified strand to a target strand. The most studied modifications are targeted to guanosines hence they have been termed G-clamps or cytidine analogs. Representative cytosine analogs that make 3 hydrogen bonds with a guanosine in a second strand include 1,3-diazaphenoxazine-2-one (Kurchavov, et al., Nucleosides and Nucleotides, 1997, 16, 1837-1846), 1,3-diazaphenothiazine-2-one, (Lin, K. -Y.; Jones, R. J.; Matteucci, M. J. Am. Chem. Soc. 1995, 117, 3873-3874) and 6,7,8,9-tetrafluoro-1,3-diazaphenoxazine-2-one (Wang, J.; Lin, K. -Y., Matteucci, M. Tetrahedron Lett. 1998, 39, 8385-8388). Incorporated into oligonucleotides these base modifications were shown to hybridize with complementary guanine and the latter was also shown to hybridize with adenine and to enhance helical thermal stability by extended stacking interactions (also see U.S. Pre-Crant Publications 20030207804 and 20030175906).

Further helix-stabilizing properties have been observed when a cytosine analog/substitute has an aminoethoxy moiety attached to the rigid 1,3-diazaphenoxazine-2-one scaffold (Lin, K. -Y.; Matteucci, M. J. Am. Chem. Soc. 1998, 120, 8531-8532). Binding studies demonstrated that a single incorporation could enhance the binding affinity of a model oligonucleotide to its complementary target DNA or RNA with a ΔTₘ of up to 18° relative to 5-methyl cytosine (dC5^{me}), which is a high affinity enhancement for a single modification. On the other hand, the gain in helical stability does not compromise the specificity of the oligonucleotides.

Further tricyclic heterocyclic compounds and methods of using them that are amenable to use in the present invention are disclosed in U.S. Pat. Nos. 6,028,183, and 6,007,992.

The enhanced binding affinity of the phenoxazine derivatives together with their uncompromised sequence specificity makes them valuable nucleobase analogs for the development of more potent antisense-based drugs. In fact, promising data have been derived from in vitro experiments demonstrating that heptanucleotides containing phenoxazine substitutions are capable to activate RNase H, enhance cellular uptake and exhibit an increased antisense activity (Lin, K -Y; Matteucci, M. J. Am. Chem. Soc. 1998, 120, 8531-8532). The activity enhancement was even more pronounced in case of G-clamp, as a single substitution was shown to significantly improve the in vitro potency of a 20mer 2′-deoxyphosphorothioate oligonucleotides (Flanagan, W. M.; Wolf, J. J.; Olson, P.; Grant, D.; Lin, K. -Y.; Wagner, R. W.; Matteucci, M. Proc. Natl. Acad. Sci. USA, 1999, 96, 3513-3518).

Further modified polycyclic heterocyclic compounds useful as heterocyclic bases are disclosed in but not limited to, the above noted U.S. Pat. No. 3,687,808, as well as U.S. Pat. Nos. 4,845,205; 5,130,302; 5,134,066; 5,175,273; 5,367,066; 5,432,272; 5,434,257; 5,457,187; 5,459,255; 5,484,908; 5,502,177; 5,525,711; 5,552,540; 5,587,469; 5,594,121, 5,596,091; 5,614,617; 5,645,985; 5,646,269; 5,750,692; 5,830,653; 5,763,588; 6,005,096; and 5,681,941, and U.S. Pre-Grant Publication 20030158403.

Conjugates

Another modification of the oligomeric compounds of the invention involves chemically linking to the oligomeric compound one or more moieties or conjugates which enhance the properties of the oligomeric compound, such as to enhance the activity, cellular distribution or cellular uptake of the oligomeric compound. These moieties or conjugates can include conjugate groups covalently bound to functional groups such as primary or secondary hydroxyl groups. Conjugate groups of the invention include intercalators, reporter molecules, polyamines, polyamides, polyethylene glycols, polyethers, groups that enhance the pharmaco-dynamic properties of oligomers, and groups that enhance the pharmacokinetic properties of oligomers. Typical conjugate groups include cholesterols, lipids, phospholipids, biotin, phenazine, folate, phenanthridine, anthraquinone, acridine, fluoresceins, rhodamines, coumarins, and dyes. Groups that enhance the pharmacodynamic properties, in the context of this invention, include groups that improve uptake, enhance resistance to degradation, and/or strengthen sequence-specific hybridization with the target nucleic acid. Groups that enhance the pharmaco-kinetic properties, in the context of this invention, include groups that improve uptake, distribution, metabolism or excretion of the compounds of the present invention. Representative conjugate groups are disclosed in International Patent Application PCT/US92/09196, filed Oct. 23, 1992, and U.S. Pat. Nos. 6,287,860 and 6,762,169.

Conjugate moieties include but are not limited to lipid moieties such as a cholesterol moiety, cholic acid, a thioether, e.g., hexyl-5-tritylthiol, a thiocholesterol, an aliphatic chain, e.g., dodecandiol or undecyl residues, a phospholipid, e.g., di-hexadecyl-rac-glycerol or triethyl-ammonium 1,2-di-O-hexadecyl-rac-glycero-3-H-phosphonate, a polyamine or a polyethylene glycol chain, or adamantane acetic acid, a palmityl moiety, or an octadecylamine or hexylamino-carbonyl-oxycholesterol moiety. Oligomeric compounds of the invention may also be conjugated to drug substances, for example, aspirin, warfarin, phenylbutazone, ibuprofen, suprofen, fenbufen, ketoprofen, (S)-(+)-pranoprofen, carprofen, dansylsarcosine, 2,3,5-triiodo-benzoic acid, flufenamic acid, folinic acid, a benzothiadiazide, chlorothiazide, a diazepine, indo-methicin, a barbiturate, a cephalosporin, a sulfa drug, an antidiabetic, an antibacterial or an antibiotic. Oligonucleotide-drug conjugates and their preparation are described in U.S. Pat. No. 6,656,730.

Representative United States patents that teach the preparation of such oligonucleotide conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941.

Oligomeric compounds can also be modified to have one or more stabilizing groups that are generally attached to one or both termini of an oligomeric compound to enhance properties such as for example nuclease stability. Included in stabilizing groups are cap structures. By “cap structure or terminal cap moiety” is meant chemical modifications, which have been incorporated at either terminus of oligonucleotides (see for example Wincott et al., WO 97/26270). These terminal modifications protect the oligomeric compounds having terminal nucleic acid molecules from exonuclease degradation, and can improve delivery and/or localization within a cell. The cap can be present at either the 5′-terminus (5′-cap) or at the 3′-terminus (3′-cap) or can be present on both termini of a single strand, or one or more termini of both strands of a double-stranded compound. This cap structure is not to be confused with the inverted methylguanosine “5′cap” present at the 5′ end of native mRNA molecules. In non-limiting examples, the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl riucleotide, 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety (for more details see Wincott et al., International PCT publication No. WO 97/26270). For siRNA constructs, the 5′ end (5′ cap) is commonly but not limited to 5′-hydroxyl or 5′-phosphate.

Particularly suitable 3′-cap structures include, for example 4′,5′-methylene nucleotide; 1-(beta-D-erythrofuranosyl) nucleotide; 4′-thio nucleotide, carbocyclic nucleotide; 5′-amino-alkyl phosphate; 1,3-diamino-2-propyl phosphate, 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threo-pentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties (for more details see Beaucage and Tyer, 1993, Tetrahedron 49, 1925).

Further 3′ and 5′-stabilizing groups that can be used to cap one or both ends of an oligomeric compound to impart nuclease stability include those disclosed in WO 03/004602 published on Jan. 16, 2003.

Chimeric Compounds

It is not necessary for all positions in a given oligomeric compound to be uniformly modified, and in fact more than one of the aforementioned modifications may be incorporated in a single compound or even within a single nucleoside within an oligomeric compound.

The present invention also includes oligomeric compounds which are chimeric compounds. “Chimeric” oligomeric compounds or “chimeras,” in the context of this invention, are single- or double-stranded oligomeric compounds, such as oligonucleotides, which contain two or more chemically distinct regions, each comprising at least one monomer unit, i.e., a nucleotide in the case of an oligonucleotide compound. Chimeric antisense oligonucleotides are one form of oligomeric compound. These oligonucleotides typically contain at least one region which is modified so as to confer upon the oligonucleotide increased resistance to nuclease degradation, increased cellular uptake, alteration of charge, increased stability and/or increased binding affinity for the target nucleic acid. An additional region of the oligonucleotide may serve as a substrate for RNAses or other enzymes. By way of example, RNAse H is a cellular endonuclease which cleaves the RNA strand of an RNA:DNA duplex. Activation of RNase H, therefore, results in cleavage of the RNA target when bound by a DNA-like oligomeric compound, thereby greatly enhancing the efficiency of oligonucleotide-mediated inhibition of gene expression. The cleavage of RNA:RNA hybrids can, in like fashion, be accomplished through the actions of endoribonucleases, such as RNase III or RNAseL which cleaves both cellular and viral RNA. Cleavage products of the RNA target can be routinely detected by gel electrophoresis and, if necessary, associated nucleic acid hybridization techniques known in the art.

Chimeric oligomeric compounds of the invention can be formed as composite structures of two or more oligonucleotides, modified oligonucleotides, oligonucleosides, oligonucleotide mimetics, or regions or portions thereof. Such compounds have also been referred to in the art as hybrids or gapmers. Representative United States patents that teach the preparation of such hybrid structures include, but are not limited to, U.S. Pat. Nos. 5,013,830; 5,149,797; 5,220,007; 5,256,775; 5,366,878; 5,403,711; 5,491,133; 5,565,350; 5,623,065; 5,652,355; 5,652,356; and 5,700,922.

A “gapmer” is defined as an oligomeric compound, generally an oligonucleotide, having a 2′-deoxyoligonucleotide region flanked by non-deoxyoligonucleotide segments. The central region is referred to as the “gap.” The flanking segments are referred to as “wings.” While not wishing to be bound by theory, the gap of the gapmer presents a substrate recognizable by RNase H when bound to the RNA target whereas the wings do not provide such a substrate but can confer other properties such as contributing to duplex stability or advantageous pharmacokinetic effects. Each wing can be one or more non-deoxyoligonucleotide monomers (if one of the wings has zero non-deoxyoligonucleotide monomers, a “hemimer” is described). In one embodiment, the gapmer is a ten deoxynucleotide gap flanked by five non-deoxynucleotide wings. This is refered to as a 5-10-5 gapmer. Other configurations are readily recognized by those skilled in the art. In one embodiment the wings comprise 2′-MOE modified nucleotides. In another embodiment the gapmer has a phosphorothioate backbone. In another embodiment the gapmer has 2′-MOE wings and a phosphorothioate backbone. Other suitable modifications are readily recognizable by those skilled in the art.

Oligomer Synthesis

Oligomerization of modified and unmodified nucleosides can be routinely performed according to literature procedures for DNA (Protocols for Oligonucleotides and Analogs, Ed. Agrawal (1993), Humana Press) and/or RNA (Scaringe, Methods (2001), 23, 206-217. Gait et al., Applications of Chemically synthesized RNA in RNA: Protein Interactions, Ed. Smith (1998), 1-36. Gallo et al., Tetrahedron (2001), 57, 5707-5713).

Oligomeric compounds of the present invention can be conveniently and routinely made through the well-known technique of solid phase synthesis. Equipment for such synthesis is sold by several vendors including, for example, Applied Biosystems (Foster City, Calif.). Any other means for such synthesis known in the art may additionally or alternatively be employed. It is well known to use similar techniques to prepare oligonucleotides such as the phosphorothioates and alkylated derivatives.

Precursor Compounds

The following precursor compounds, including amidites and their intermediates can be prepared by methods routine to those skilled in the art; 5′-O-Dimethoxytrityl-thymidine intermediate for 5-methyl dC amidite, 5′-O-Dimethoxytrityl-2′-deoxy-5-methylcytidine intermediate for 5-methyl-dC amidite, 5′-O-Dimethoxytrityl-2′-deoxy-N-4-benzoyl-5-methylcytidine penultimate intermediate for 5-methyl dC amidite, (5′-O-(4,4′-Dimethoxytriphenylmethyl)-2′-deoxy-N⁴-benzoyl-5-methylcytidin-3′-O-yl)-2-cyanoethyl-N,N-diisopropylphosphoramidite (5-methyl dC amidite), 2′-Fluorodeoxyadenosine, 2′-Fluorodeoxyguanosine, 2′-Fluorouridine, 2′-Fluorodeoxycytidine, 2′-O-(2-Methoxyethyl) modified amidites, 2′-O-(2-methoxyethyl)-5-methyluridine intermediate, 5′-O-DMT-2′-O-(2-methoxyethyl)-5-methyluridine penultimate intermediate, (5′-O-(4,4′-Dimethoxytriphenylmethyl)-2′-O-(2-methoxyethyl)-5-methyluridin-3′-O-yl)-2-cyanoethyl-N,N-diisopropylphosphoramidite (MOE T amidite), 5′-O-Dimethoxytrityl-2′-O-(2-methoxyethyl)-5-methylcytidine intermediate, 5′-O-dimethoxytrityl-2′-O-(2-methoxyethyl)-N⁴-benzoyl-5-methyl-cytidine penultimate intermediate, (5′-O-(4,4′-Dimethoxytriphenylmethyl)-2′-O-(2-methoxyethyl)-N⁴-benzoyl-5-methylcytidin-3′-O-yl)-2-cyanoethyl-N,N-diisopropylphosphoramidite (MOE 5-Me-C amidite), (5′-O-(4,4′-Dimethoxytriphenylmethyl)-2′-O-(2-methoxyethyl)-N⁶-benzoyladenosin-3′-O-yl)-2-cyanoethyl-N,N-diisopropylphosphoramidite (MOE A amdite), (5′-O-(4,4′-Dimethoxytriphenylmethyl)-2′-O-(2-methoxyethyl)-N⁴-isobutyrylguanosin-3′-O-yl)-2-cyanoethyl-N,N-diisopropylphosphoramidite (MOE G amidite), 2′-O-(Aminooxyethyl) nucleoside amidites and 2′-O-(dimethylaminooxy-ethyl) nucleoside amidites, 2′-(Dimethylaminooxyethoxy) nucleoside amidites, 5′-O-tert-Butyldiphenylsilyl-O₂-2′-anhydro-5-methyluridine, 5′-O-tert-Butyldiphenylsilyl-2′-O-(2-hydroxyethyl)-5-methyluridine, 2′-O-((2-phthalimidoxy)ethyl)-5′-t-butyldiphenylsilyl-5-methyluridine, 5′-O-tert-butyldiphenylsilyl-2′-O-((2-formadoximinooxy)ethyl)-5-methyluridine, 5′-O-tert-Butyldiphenylsilyl-2′-O-(N,N dimethylaminooxyethyl)-5-methyluridine, 2′-O-(dimethylaminooxyethyl)-5-methyluridine, 5′-O-DMT-2′-O-(dimethylaminooxyethyl)-5-methyluridine, 5′-O-DMT-2′-O-(2-N,N-dimethylaminooxyethyl)-5-methyluridine-3′-((2-cyanoethyl)-N,N-diisopropylphosphoramidite), 2′-(Aminooxyethoxy) nucleoside amidites, N2-isobutyryl-6-O-diphenylcarbamoyl-2′-O-(2-ethylacetyl)-5′-O-(4,4′-dimethoxytrityl)guanosine-3′-((2-cyanoethyl)-N,N-diisopropylphosphoramidite), 2′-dimethylaminoethoxyethoxy (2′-DMAEOE) nucleoside amidites, 2′-O-(2(2-N,N-dimethylaminoethoxy)ethyl)-5-methyl uridine, 5′-O-dimethoxytrityl-2′-O-(2(2-N,N-dimethylaminoethoxy)-ethyl))-5-methyl uridine and 5′-O-Dimethoxytrityl-2′-O-(2(2-N,N-dimethylaminoethoxy)-ethyl))-5-methyl uridine-3′-O-(cyanoethyl-N,N-diisopropyl)phosphoramidite.

The preparation of such precursor compounds for oligonucleotide synthesis are routine in the art and disclosed in U.S. Pat. No. 6,426,220 and published PCT WO 02/36743.

2′-Deoxy and 2′-methoxy beta-cyanoethyldiisopropyl phosphoramidites can be purchased from commercial sources (e.g. Chemgenes, Needham, Mass. or Glen Research, Inc. Sterling, Va.). Other 2′-O-alkoxy substituted nucleoside amidites can be prepared as described in U.S. Pat. No. 5,506,351.

Oligonucleotides containing 5-methyl-2′-deoxycytidine (5-Me-C) nucleotides can be synthesized routinely according to published methods (Sanghvi, et. al., Nucleic Acids Research, 1993, 21, 3197-3203) using commercially available phosphoramidites (Glen Research, Sterling Va. or ChemGenes, Needham, Mass.).

2′-fluoro oligonucleotides can be synthesized routinely as described (Kawasaki, et. al., J. Med. Chem., 1993, 36, 831-841) and U.S. Pat. No. 5,670,633.

2′-O-Methoxyethyl-substituted nucleoside amidites can be prepared routinely as per the methods of Martin, P., Helvetica Chimica Acta, 1995, 78, 486-504.

Aminooxyethyl and dimethylaminooxyethyl amidites can be prepared routinely as per the methods of U.S. Pat. No. 6,127,533.

Oligonucleotide Synthesis

Phosphorothioate-containing oligonucleotides (P═S) can be synthesized by methods routine to those skilled in the art (see, for example, Protocols for Oligonucleotides and Analogs, Ed. Agrawal (1993), Humana Press). Phosphinate oligonucleotides can be prepared as described in U.S. Pat. No. 5,508,270.

Alkyl phosphonate oligonucleotides can be prepared as described in U.S. Pat. No. 4,469,863.

3′-Deoxy-3′-methylene phosphonate oligonucleotides can be prepared as described in U.S. Pat. Nos. 5,610,289 or 5,625,050.

Phosphoramidite oligonucleotides can be prepared as described in U.S. Pat. No. 5,256,775 or U.S. Pat. No. 5,366,878.

Alkylphosphonothioate oligonucleotides can be prepared as described in published PCT applications PCT/US94/00902 and PCT/US93/06976 (published as WO 94/17093 and WO 94/02499, respectively).

3′-Deoxy-3′-amino phosphoramidate oligonucleotides can be prepared as described in U.S. Pat. No. 5,476,925.

Phosphotriester oligonucleotides can be prepared as described in U.S. Pat. No. 5,023,243.

Borano phosphate oligonucleotides can be prepared as described in U.S. Pat. Nos. 5,130,302 and 5,177,198.

4′-thio-containing oligonucleotides can be synthesized as described in U.S. Pat. No. 5,639,873.

Oligonucleoside Synthesis

Methylenemethylimino linked oligonucleosides, also identified as MMI linked oligonucleosides, methylenedimethylhydrazo linked oligonucleosides, also identified as MDH linked oligonucleosides, and methylenecarbonylamino linked oligonucleosides, also identified as amide-3 linked oligonucleosides, and methyleneaminocarbonyl linked oligonucleosides, also identified as amide-4 linked oligonucleosides, as well as mixed backbone compounds having, for instance, alternating MMI and P═O or P═S linkages can be prepared as described in U.S. Pat. Nos. 5,378,825, 5,386,023, 5,489,677, 5,602,240 and 5,610,289.

Formacetal and thioformacetal linked oligonucleosides can be prepared as described in U.S. Pat. Nos. 5,264,562 and 5,264,564.

Ethylene oxide linked oligonucleosides can be prepared as described in U.S. Pat. No. 5,223,618.

Peptide Nucleic Acid Synthesis

Peptide nucleic acids (PNAs) can be prepared in accordance with any of the various procedures referred to in Peptide Nucleic Acids (PNA): Synthesis, Properties and Potential Applications, Bioorganic & Medicinal Chemistry, 1996, 4, 5-23. They may also be prepared in accordance with U.S. Pat. Nos. 5,539,082, 5,700,922, 5,719,262, 6,559,279 and 6,762,281.

Synthesis of 2′-O-Protected Oligomers/RNA Synthesis

Oligomeric compounds incorporating at least one 2′-O-protected nucleoside by methods routine in the art. After incorporation and appropriate deprotection the 2′-O-protected nucleoside will be converted to a ribonucleoside at the position of incorporation. The number and position of the 2-ribonucleoside units in the final oligomeric compound can vary from one at any site or the strategy can be used to prepare up to a full 2′-OH modified oligomeric compound.

A large number of 2′-O-protecting groups have been used for the synthesis of oligoribo-nucleotides and any can be used. Some of the protecting groups used initially for oligoribonucleotide synthesis included tetrahydropyran-1-yl and 4-methoxytetrahydropyran-4-yl. These two groups are not compatible with all 5′-O-protecting groups so modified versions were used with 5′-DMT groups such as 1-(2-fluorophenyl)-4-methoxypiperidin-4-yl (Fpmp). Reese et al. have identified a number of piperidine derivatives (like Fpmp) that are useful in the synthesis of oligoribonucleotides including 1-[(chloro-4-methyl)phenyl]-4′-methoxypiperidin-4-yl (Reese et al., Tetrahedron Lett., 1986, (27), 2291). Another approach is to replace the standard 5′-DMT (dimethoxytrityl) group with protecting groups that were removed under non-acidic conditions such as levulinyl and 9-fluorenylmethoxycarbonyl. Such groups enable the use of acid labile 2′-protecting groups for oligoribonucleotide synthesis. Another more widely used protecting group, initially used for the synthesis of oligoribonucleotides, is the t-butyldimethylsilyl group (Ogilvie et al., Tetrahedron Lett., 1974, 2861; Hakimelahi et al., Tetrahedron Lett., 1981, (22), 2543; and Jones et al., J. Chem. Soc: Perkin I., 2762). The 2′-O-protecting groups can require special reagents for their removal. For example, the t-butyldimethylsilyl group is normally removed after all other cleaving/deprotecting steps by treatment of the oligomeric compound with tetrabutylammonium fluoride (TBAF).

One group of researchers examined a number of 2′-O-protecting groups (Pitsch, S., Chimia, 2001, (55), 320-324.) The group examined fluoride labile and photolabile protecting groups that are removed using moderate conditions. One photolabile group that was examined was the [2-(nitrobenzyl)oxy]methyl (nbm) protecting group (Schwartz et al., Bioorg. Med. Chem. Lett., 1992, (2), 1019.) Other groups examined included a number structurally related formaldehyde acetal-derived, 2′-O-protecting groups. Also prepared were a number of related protecting groups for preparing 2′-O-alkylated nucleoside phosphoramidites including 2′-O-[(triisopropylsilyl)oxy]methyl (2′-O—CH₂—O—Si(iPr)₃, TOM). One 2′-O-protecting group that was prepared to be used orthogonally to the TOM group was 2′-O-[(R)-1-(2-nitrophenyl)ethyloxy)methyl] ((R)-mnbm).

Another strategy using a fluoride labile 5′-O-protecting group (non-acid labile) and an acid labile 2′-O-protecting group has been reported (Scaringe, Stephen A., Methods, 2001, (23) 206-217). A number of possible silyl ethers were examined for 5′-O-protection and a number of acetals and orthoesters were examined for 2′-O-protection. The protection scheme that gave the best results was 5′-O-silyl ether-2′-ACE (5′-O-bis(trimethylsiloxy)cyclododecyloxysilyl ether (DOD)-2′-O-bis(2-acetoxyethoxy)methyl (ACE). This approach uses a modified phosphoramidite synthesis approach in that some different reagents are required that are not routinely used for RNA/DNA synthesis.

The main RNA synthesis strategies that are presently being used commercially include 5′-O-DMT-2′-O-t-butyldimethylsilyl (TBDMS), 5′-O-DMT-2′-O-[1 (2-fluorophenyl)₄-methoxypiperidin-4-yl] (FPMP), 2′-O-[(triisopropylsilyl)oxy]methyl (2′-O—CH₂—O—Si(iPr)₃ (TOM), and the 5′-O-silyl ether-2′-ACE (5′-O-bis(trimethylsiloxy)cyclododecyloxysilyl ether (DOD)-2′-O-bis(2-acetoxyethoxy)methyl (ACE). Some companies currently offering RNA products include Pierce Nucleic Acid Technologies (Milwaukee, Wis.), Dharmacon Research Inc. (a subsidiary of Fisher Scientific, Lafayette, Colo.), and Integrated DNA Technologies, Inc. (Coralville, Iowa). One company, Princeton Separations, markets an RNA synthesis activator advertised to reduce coupling times especially with TOM and TBDMS chemistries. Such an activator would also be amenable to the oligomeric compounds of the present invention.

All of the aforementioned RNA synthesis strategies are amenable to the oligomeric compounds of the present invention. Strategies that would be a hybrid of the above e.g. using a 5′-protecting group from one strategy with a 2′-O-protecting from another strategy is also contemplated herein.

Synthesis of Chimeric Oligomeric Compounds

### (2′-O-Me)-(2′-deoxy)-(2′-O-Me) Chimeric Phosphorothioate Oligonucleotides

Chimeric oligonucleotides having 2′-O-alkyl phosphorothioate and 2′-deoxy phosphorothioate oligonucleotide segments can be routinely synthesized by one skilled in the art, using, for example, an Applied Biosystems automated DNA synthesizer Model 394. Oligonucleotides can be synthesized using an automated synthesizer and 2′-deoxy-5′-dimethoxytrityl-3′-O-phosphoramidite for the DNA portion and 5′-dimethoxytrityl-2′-O-methyl-3′-O-phosphoramidite for the 2′-O-alkyl portion. In one nonlimiting example, the standard synthesis cycle is modified by incorporating coupling steps with increased reaction times for the 5′-dimethoxytrityl-2′-O-methyl-3′-O-phosphoramidite. The fully protected oligonucleotide is cleaved from the support and deprotected in concentrated ammonia (NH₄OH) for 12-16 hr at 55° C. The deprotected oligonucleotide is then recovered by an appropriate method (precipitation, column chromatography, volume reduced in vacuo) and analyzed by methods routine in the art.

### (2′-O-(2-Methoxyethyl))-(2′-deoxy)-(2′-O-(2-Methoxyethyl)) Chimeric Phosphorothioate Oligonucleotides

(2′-O-(2-methoxyethyl))—(2′-deoxy)—(-2′-O-(2-methoxyethyl)) chimeric phosphorothioate oligonucleotides can be prepared as per the procedure above for the 2′-O-methyl chimeric oligonucleotide, with the substitution of 2′-O-(methoxyethyl) amidites for the 2′-O-methyl amidites.

### (2′-O-(2-Methoxyethyl)Phosphodiester)—(2′-deoxy Phosphorothioate)—(2′-O-(2-Methoxyethyl) Phosphodiester) Chimeric Oligonucleotides

(2′-O-(2-methoxyethyl phosphodiester)—(2′-deoxy phosphorothioate)—(2′-O-(methoxyethyl) phosphodiester) chimeric oligonucleotides can be prepared as per the above procedure for the 2′-O-methyl chimeric oligonucleotide with the substitution of 2′-O-(methoxyethyl) amidites for the 2′-O-methyl amidites, oxidation with iodine to generate the phosphodiester internucleotide linkages within the wing portions of the chimeric structures and sulfurization utilizing 3,H-1,2 benzodithiole-3-one 1,1 dioxide (Beaucage Reagent) to generate the phosphorothioate internucleotide linkages for the center gap.

Other chimeric oligonucleotides, chimeric oligonucleosides and mixed chimeric oligonucleotides/oligonucleosides can be synthesized according to U.S. Pat. No. 5,623,065.

Oligomer Purification and Analysis

Methods of oligonucleotide purification and analysis are known to those skilled in the art. Analysis methods include capillary electrophoresis (CE) and electrospray-mass spectroscopy. Such synthesis and analysis methods can be performed in multi-well plates.

Hybridization

“Hybridization” means the pairing of complementary strands of oligomeric compounds. While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases) of the strands of oligomeric compounds. For example, adenine and thymine are complementary nucleobases which pair through the formation of hydrogen bonds. Hybridization can occur under varying circumstances.

An oligomeric compound is specifically hybridizable when there is a sufficient degree of complementarity to avoid non-specific binding of the oligomeric compound to non-target nucleic acid sequences under conditions in which specific binding is desired, i.e., under physiological conditions in the case of in vivo assays or therapeutic treatment, and under conditions in which assays are performed in the case of in vitro assays.

“Stringent hybridization conditions” or “stringent conditions” refers to conditions under which an oligomeric compound will hybridize to its target sequence, but to a minimal number of other sequences. Stringent conditions are sequence-dependent and will be different in different circumstances, and “stringent conditions” under which oligomeric compounds hybridize to a target sequence are determined by the nature and composition of the oligomeric compounds and the assays in which they are being investigated.

Complementarity

“Complementarity,” as used herein, refers to the capacity for precise pairing between two nucleobases on one or two oligomeric compound strands. For example, if a nucleobase at a certain position of an antisense compound is capable of hydrogen bonding with a nucleobase at a certain position of a target nucleic acid, then the position of hydrogen bonding between the oligonucleotide and the target nucleic acid is considered to be a complementary position. The oligomeric compound and the further DNA or RNA are complementary to each other when a sufficient number of complementary positions in each molecule are occupied by nucleobases which can hydrogen bond with each other. Thus, “specifically hybridizable” and “complementary” are terms which are used to indicate a sufficient degree of precise pairing or complementarity over a sufficient number of nucleobases such that stable and specific binding occurs between the oligomeric compound and a target nucleic acid.

It is understood in the art that the sequence of an oligomeric compound need not be 100% complementary to that of its target nucleic acid to be specifically hybridizable. Moreover, an oligonucleotide may hybridize over one or more segments such that intervening or adjacent segments are not involved in the hybridization event (e.g., a loop structure, mismatch or hairpin structure). The oligomeric compounds of the present invention comprise at least 70%, or at least 75%, or at least 80%, or at least 85%, or at least 90%, or at least 92%, or at least 95%, or at least 97%, or at least 98%, or at least 99% sequence complementarity to a target region within the target nucleic acid sequence to which they are targeted. For example, an oligomeric compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an oligomeric compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an oligomeric compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489).

The oligomeric compounds of the invention also include variants in which a different base is present at one or more of the nucleotide positions in the compound. For example, if the first nucleotide is an adenosine, variants may be produced which contain thymidine, guanosine or cytidine at this position. This may be done at any of the positions of the oligomeric compound. These compounds are then tested using the methods described herein to determine their ability to inhibit expression of a bone growth modulator mRNA.

Target Nucleic Acids “Targeting” an oligomeric compound to a particular target nucleic acid molecule can be a multistep process. The process usually begins with the identification of a target nucleic acid whose expression is to be modulated. As used herein, the terms “target nucleic acid” and “nucleic acid encoding a bone growth modulator” encompass DNA encoding a bone growth modulator, RNA (including pre-mRNA and mRNA) transcribed from such DNA, and also cDNA derived from such RNA. For example, the target nucleic acid can be a cellular gene (or mRNA transcribed from the gene) whose expression is associated with a particular disorder or disease state, or a nucleic acid molecule from an infectious agent. As disclosed herein, the target nucleic acid encodes a bone growth modulator.

Target Regions, Segments, and Sites

The targeting process usually also includes determination of at least one target region, segment, or site within the target nucleic acid for the antisense interaction to occur such that the desired effect, e.g., modulation of expression, will result. “Region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as unique nucleobase positions within a target nucleic acid.

Start Codons

Since, as is known in the art, the translation initiation codon is typically 5′ AUG (in transcribed mRNA molecules; 5′ ATG in the corresponding DNA molecule), the translation initiation codon is also referred to as the “AUG codon,” the “start codon” or the “AUG start codon.” A minority of genes have a translation initiation codon having the RNA sequence 5′ GUG, 5′ UUG or 5′CUG, and 5′ AUA, 5′ ACG and 5′CUG have been shown to function in vivo. Thus, the terms “translation initiation codon” and “start codon” can encompass many codon sequences, even though the initiator amino acid in each instance is typically methionine (in eukaryotes) or formylmethionine (in prokaryotes). It is also known in the art that eukaryotic and prokaryotic genes may have two or more alternative start codons, any one of which may be preferentially utilized for translation initiation in a particular cell type or tissue, or under a particular set of conditions. “Start codon” and “translation initiation codon” refer to the codon or codons that are used in vivo to initiate translation of an mRNA transcribed from a gene encoding a protein, regardless of the sequence(s) of such codons. It is also known in the art that a translation termination codon (or “stop codon”) of a gene may have one of three sequences, i.e., 5′ UAA, 5′ UAG and 5′ UGA (the corresponding DNA sequences are 5′ TAA, 5′ TAG and 5′ TGA, respectively).

The terms “start codon region” and “translation initiation codon region” refer to a portion of such an mRNA or gene that encompasses from about 25 to about 50 contiguous nucleotides in either direction (i.e., 5′ or 3′) from a translation initiation codon. Similarly, the terms “stop codon region” and “translation termination codon region” refer to a portion of such an mRNA or gene that encompasses from about 25 to about 50 contiguous nucleotides in either direction (i.e., 5′ or 3′) from a translation termination codon. Consequently, the “start codon region” (or “translation initiation codon region”) and the “stop codon region” (or “translation termination codon region”) are all regions which may be targeted effectively with oligomeric compounds of the invention.

Coding Regions

The open reading frame (ORF) or “coding region,” which is known in the art to refer to the region between the translation initiation codon and the translation termination codon, is also a region which may be targeted effectively. Within the context of the present invention, one region is the intragenic region encompassing the translation initiation or termination codon of the open reading frame (ORF) of a gene.

Untranslated Regions

Other target regions include the 5′ untranslated region (5′UTR), known in the art to refer to the portion of an mRNA in the 5′ direction from the translation initiation codon, and thus including nucleotides between the 5′ cap site and the translation initiation codon of an mRNA (or corresponding nucleotides on the gene), and the 3′ untranslated region (3′UTR), known in the art to refer to the portion of an mRNA in the 3′ direction from the translation termination codon, and thus including nucleotides between the translation termination codon and 3′ end of an mRNA (or corresponding nucleotides on the gene). The 5′ cap site of an mRNA comprises an N7-methylated guanosine residue joined to the 5′-most residue of the mRNA via a 5′-5′ triphosphate linkage. The 5′ cap region of an mRNA is considered to include the 5′ cap structure itself as well as the first 50 nucleotides adjacent to the cap site. The 5′ cap region is also a target.

Introns and Exons

Although some eukaryotic mRNA transcripts are directly translated, many contain one or more regions, known as “introns,” which are excised from a transcript before it is translated. The remaining (and therefore translated) regions are known as “exons” and are spliced together to form a continuous mRNA sequence, resulting in exon-exon junctions at the site where exons are joined. Targeting exon-exon junctions can be useful in situations where aberrant levels of a normal splice product is implicated in disease, or where aberrant levels of an aberrant splice product is implicated in disease. Targeting splice sites, i.e., intron-exon junctions or exon-intron junctions can also be particularly useful in situations where aberrant splicing is implicated in disease, or where an overproduction of a particular splice product is implicated in disease. Aberrant fusion junctions due to rearrangements or deletions are also suitable targets. mRNA transcripts produced via the process of splicing of two (or more) mRNAs from different gene sources are known as “fusion transcripts” and are also suitable targets. It is also known that introns can be effectively targeted using antisense compounds targeted to, for example, DNA or pre-mRNA. Single-stranded antisense compounds such as oligonucleotide compounds that work via an RNase H mechanism are effective for targeting pre-mRNA. Antisense compounds that function via an occupancy-based mechanism are effective for redirecting splicing as they do not, for example, elicit RNase H cleavage of the mRNA, but rather leave the mRNA intact and promote the yield of desired splice product(s).

Variants

It is also known in the art that alternative RNA transcripts can be produced from the same genomic region of DNA. These alternative transcripts are generally known as “variants.” More specifically, “pre-mRNA variants” are transcripts produced from the same genomic DNA that differ from other transcripts produced from the same genomic DNA in either their start or stop position and contain both intronic and exonic sequence. Upon excision of one or more exon or intron regions, or portions thereof during splicing, pre-mRNA variants produce smaller “mRNA variants.” Consequently, mRNA variants are processed pre-mRNA variants and each unique pre-mRNA variant must always produce a unique mRNA variant as a result of splicing. These mRNA variants are also known as “alternative splice variants.” If no splicing of the pre-mRNA variant occurs then the pre-mRNA variant is identical to the mRNA variant.

It is also known in the art that variants can be produced through the use of alternative signals to start or stop transcription and that pre-mRNAs and mRNAs can possess more that one start codon or stop codon. Variants that originate from a pre-mRNA or mRNA that use alternative start codons are known as “alternative start variants” of that pre-mRNA or mRNA. Those transcripts that use an alternative stop codon are known as “alternative stop variants” of that pre-mRNA or mRNA. One specific type of alternative stop variant is the “polyA variant” in which the multiple transcripts produced result from the alternative selection of one of the “polyA stop signals” by the transcription machinery, thereby producing transcripts that terminate at unique polyA sites. Consequently, the types of variants described herein are also suitable target nucleic acids.

Target Names, Synonyms, Features

In accordance with the present invention are compositions and methods for modulating the expression of genes which are presented in Table 1. Table 1 lists the gene target names and their respective synonyms, as well as GenBank accession numbers used to design oligomeric compounds targeted to each gene. Table 1 also describes features contained within the gene target nucleic acid sequences of the invention. Representative features include 5′UTR, start codon, coding sequence (CDS), stop codon, 3′UTR, exon, intron, exon:exon junction, intron:exon junction and exon:intron junction. “Feature start site” and “feature end site” refer to the first (5′-most) and last (3′-most) nucleotide numbers, respectively, of the described feature with respect to the designated sequence. For example, for a sequence containing a start codon comprising the first three nucleotides, “feature start site” is “1” and “feature end site” is “3”.

TABLE 1
Gene Targets, Synonyms and Features

| | | | | | Feature | Feature | SEQ |
|---|---|---|---|---|---|---|---|
| Target | | | | | Start | End | ID |
| Name | Synonyms | Species | Genbank # | Feature | Site | Site | NO |
| c-src | src-c; SRC | Rat | AA875131.1 | exon:exon | 81 | 82 | 1 |
| | | | | junction | | | |
| c-src | src-c; SRC | Rat | AA875131.1 | exon | 82 | 181 | 1 |
| c-src | src-c; SRC | Rat | AA875131.1 | exon:exon | 181 | 182 | 1 |
| | | | | junction | | | |
| c-src | src-c; SRC | Rat | AA875131.1 | exon | 182 | 280 | 1 |
| c-src | src-c; SRC | Rat | AA875131.1 | exon:exon | 280 | 281 | 1 |
| | | | | junction | | | |
| c-src | src-c; SRC | Rat | AA875131.1 | exon | 281 | 384 | 1 |
| c-src | src-c; SRC | Rat | AA875131.1 | exon:exon | 384 | 385 | 1 |
| | | | | junction | | | |
| c-src | src-c; SRC | Rat | AF130457.1 | start codon | 1 | 3 | 2 |
| c-src | src-c; SRC | Rat | AF130457.1 | CDS | 1 | 1611 | 2 |
| c-src | src-c; SRC | Rat | AF130457.1 | exon | 1 | 1611 | 2 |
| c-src | src-c; SRC | Rat | AF130457.1 | exon:exon | 250 | 251 | 2 |
| | | | | junction | | | |
| c-src | src-c; SRC | Rat | AF130457.1 | exon:exon | 350 | 351 | 2 |
| | | | | junction | | | |
| c-src | src-c; SRC | Rat | AF130457.1 | exon:exon | 449 | 450 | 2 |
| | | | | junction | | | |
| c-src | src-c; SRC | Rat | AF130457.1 | exon:exon | 553 | 554 | 2 |
| | | | | junction | | | |
| c-src | src-c; SRC | Rat | AF130457.1 | exon:exon | 703 | 704 | 2 |
| | | | | junction | | | |
| c-src | src-c; SRC | Rat | AF130457.1 | exon:exon | 859 | 860 | 2 |
| | | | | junction | | | |
| c-src | src-c; SRC | Rat | AF130457.1 | exon:exon | 1039 | 1040 | 2 |
| | | | | junction | | | |
| c-src | src-c; SRC | Rat | AF130457.1 | exon:exon | 1116 | 1117 | 2 |
| | | | | junction | | | |
| c-src | src-c; SRC | Rat | AF130457.1 | exon:exon | 1270 | 1271 | 2 |
| | | | | junction | | | |
| c-src | src-c; SRC | Rat | AF130457.1 | exon:exon | 1402 | 1403 | 2 |
| | | | | junction | | | |
| c-src | src-c; SRC | Rat | AF130457.1 | stop codon | 1609 | 1611 | 2 |
| c-src | src-c; SRC | Rat | CB720604.1 | 3′UTR | 1 | 523 | 3 |
| c-src | src-c; SRC | Rat | NM_031977.1 | start codon | 1 | 3 | 4 |
| c-src | src-c; SRC | Rat | NM_031977.1 | exon | 1 | 250 | 4 |
| c-src | src-c; SRC | Rat | NM_031977.1 | CDS | 1 | 1629 | 4 |
| c-src | src-c; SRC | Rat | NM_031977.1 | exon:exon | 250 | 251 | 4 |
| | | | | junction | | | |
| c-src | src-c; SRC | Rat | NM_031977.1 | exon | 251 | 350 | 4 |
| c-src | src-c; SRC | Rat | NM_031977.1 | exon | 369 | 467 | 4 |
| c-src | src-c; SRC | Rat | NM_031977.1 | exon:exon | 467 | 468 | 4 |
| | | | | junction | | | |
| c-src | src-c; SRC | Rat | NM_031977.1 | exon | 468 | 571 | 4 |
| c-src | src-c; SRC | Rat | NM_031977.1 | exon:exon | 571 | 572 | 4 |
| | | | | junction | | | |
| c-src | src-c; SRC | Rat | NM_031977.1 | exon | 572 | 721 | 4 |
| c-src | src-c; SRC | Rat | NM_031977.1 | exon:exon | 721 | 722 | 4 |
| | | | | junction | | | |
| c-src | src-c; SRC | Rat | NM_031977.1 | exon | 722 | 877 | 4 |
| c-src | src-c; SRC | Rat | NM_031977.1 | exon:exon | 877 | 878 | 4 |
| | | | | junction | | | |
| c-src | src-c; SRC | Rat | NM_031977.1 | exon | 878 | 1057 | 4 |
| c-src | src-c; SRC | Rat | NM_031977.1 | exon:exon | 1057 | 1058 | 4 |
| | | | | junction | | | |
| c-src | src-c; SRC | Rat | NM_031977.1 | exon | 1058 | 1134 | 4 |
| c-src | src-c; SRC | Rat | NM_031977.1 | exon:exon | 1134 | 1135 | 4 |
| | | | | junction | | | |
| c-src | src-c; SRC | Rat | NM_031977.1 | exon | 1135 | 1288 | 4 |
| c-src | src-c; SRC | Rat | NM_031977.1 | exon:exon | 1288 | 1289 | 4 |
| | | | | junction | | | |
| c-src | src-c; SRC | Rat | NM_031977.1 | exon | 1289 | 1420 | 4 |
| c-src | src-c; SRC | Rat | NM_031977.1 | exon:exon | 1420 | 1421 | 4 |
| | | | | junction | | | |
| c-src | src-c; SRC | Rat | NM_031977.1 | stop codon | 1627 | 1629 | 4 |
| c-src | src-c; SRC | Rat | NM_031977.1 | 3′UTR | 1630 | 2001 | 4 |
| c-src | src-c; SRC | Rat | nucleotides 2038000 | start codon | 1149 | 1151 | 5 |
| | | | to 2054000 of | | | | |
| | | | NW_043651.1 | | | | |
| c-src | src-c; SRC | Rat | nucleotides 2038000 | exon | 1149 | 1398 | 5 |
| | | | to 2054000 of | | | | |
| | | | NW_043651.1 | | | | |
| c-src | src-c; SRC | Rat | nucleotides 2038000 | intron:exon | 1398 | 1399 | 5 |
| | | | to 2054000 of | junction | | | |
| | | | NW_043651.1 | | | | |
| c-src | src-c; SRC | Rat | nucleotides 2038000 | intron | 1399 | 2554 | 5 |
| | | | to 2054000 of | | | | |
| | | | NW_043651.1 | | | | |
| c-src | src-c; SRC | Rat | nucleotides 2038000 | intron:exon | 2554 | 2555 | 5 |
| | | | to 2054000 of | junction | | | |
| | | | NW_043651.1 | | | | |
| c-src | src-c; SRC | Rat | nucleotides 2038000 | exon | 2555 | 2654 | 5 |
| | | | to 2054000 of | | | | |
| | | | NW_043651.1 | | | | |
| c-src | src-c; SRC | Rat | nucleotides 2038000 | intron | 2655 | 7049 | 5 |
| | | | to 2054000 of | | | | |
| | | | NW_043651.1 | | | | |
| c-src | src-c; SRC | Rat | nucleotides 2038000 | intron:exon | 7049 | 7050 | 5 |
| | | | to 2054000 of | junction | | | |
| | | | NW_043651.1 | | | | |
| c-src | src-c; SRC | Rat | nucleotides 2038000 | exon | 7050 | 7148 | 5 |
| | | | to 2054000 of | | | | |
| | | | NW_043651.1 | | | | |
| c-src | src-c; SRC | Rat | nucleotides 2038000 | intron:exon | 7148 | 7149 | 5 |
| | | | to 2054000 of | junction | | | |
| | | | NW_043651.1 | | | | |
| c-src | src-c; SRC | Rat | nucleotides 2038000 | intron | 7149 | 7316 | 5 |
| | | | to 2054000 of | | | | |
| | | | NW_043651.1 | | | | |
| c-src | src-c; SRC | Rat | nucleotides 2038000 | intron:exon | 7316 | 7317 | 5 |
| | | | to 2054000 of | junction | | | |
| | | | NW_043651.1 | | | | |
| c-src | src-c; SRC | Rat | nucleotides 2038000 | exon | 7317 | 7420 | 5 |
| | | | to 2054000 of | | | | |
| | | | NW_043651.1 | | | | |
| c-src | src-c; SRC | Rat | nucleotides 2038000 | intron:exon | 7420 | 7421 | 5 |
| | | | to 2054000 of | junction | | | |
| | | | NW_043651.1 | | | | |
| c-src | src-c; SRC | Rat | nucleotides 2038000 | intron | 7421 | 8854 | 5 |
| | | | to 2054000 of | | | | |
| | | | NW_043651.1 | | | | |
| c-src | src-c; SRC | Rat | nucleotides 2038000 | intron:exon | 8854 | 8855 | 5 |
| | | | to 2054000 of | junction | | | |
| | | | NW_043651.1 | | | | |
| c-src | src-c; SRC | Rat | nucleotides 2038000 | exon | 8855 | 9004 | 5 |
| | | | to 2054000 of | | | | |
| | | | NW_043651.1 | | | | |
| c-src | src-c; SRC | Rat | nucleotides 2038000 | intron:exon | 9004 | 9005 | 5 |
| | | | to 2054000 of | junction | | | |
| | | | NW_043651.1 | | | | |
| c-src | src-c; SRC | Rat | nucleotides 2038000 | intron | 9005 | 10050 | 5 |
| | | | to 2054000 of | | | | |
| | | | NW_043651.1 | | | | |
| c-src | src-c; SRC | Rat | nucleotides 2038000 | intron:exon | 10050 | 10051 | 5 |
| | | | to 2054000 of | junction | | | |
| | | | NW_043651.1 | | | | |
| c-src | src-c; SRC | Rat | nucleotides 2038000 | exon | 10051 | 10206 | 5 |
| | | | to 2054000 of | | | | |
| | | | NW_043651.1 | | | | |
| c-src | src-c; SRC | Rat | nucleotides 2038000 | intron:exon | 10206 | 10207 | 5 |
| | | | to 2054000 of | junction | | | |
| | | | NW_043651.1 | | | | |
| c-src | src-c; SRC | Rat | nucleotides 2038000 | intron | 10207 | 11531 | 5 |
| | | | to 2054000 of | | | | |
| | | | NW_043651.1 | | | | |
| c-src | src-c; SRC | Rat | nucleotides 2038000 | intron:exon | 11531 | 11532 | 5 |
| | | | to 2054000 of | junction | | | |
| | | | NW_043651.1 | | | | |
| c-src | src-c; SRC | Rat | nucleotides 2038000 | exon | 11532 | 11711 | 5 |
| | | | to 2054000 of | | | | |
| | | | NW_043651.1 | | | | |
| c-src | src-c; SRC | Rat | nucleotides 2038000 | intron:exon | 11711 | 11712 | 5 |
| | | | to 2054000 of | junction | | | |
| | | | NW_043651.1 | | | | |
| c-src | src-c; SRC | Rat | nucleotides 2038000 | intron | 11712 | 12569 | 5 |
| | | | to 2054000 of | | | | |
| | | | NW_043651.1 | | | | |
| c-src | src-c; SRC | Rat | nucleotides 2038000 | intron:exon | 12569 | 12570 | 5 |
| | | | to 2054000 of | junction | | | |
| | | | NW_043651.1 | | | | |
| c-src | src-c; SRC | Rat | nucleotides 2038000 | exon | 12570 | 12646 | 5 |
| | | | to 2054000 of | | | | |
| | | | NW_043651.1 | | | | |
| c-src | src-c; SRC | Rat | nucleotides 2038000 | intron:exon | 12646 | 12647 | 5 |
| | | | to 2054000 of | junction | | | |
| | | | NW_043651.1 | | | | |
| c-src | src-c; SRC | Rat | nucleotides 2038000 | intron | 12647 | 13173 | 5 |
| | | | to 2054000 of | | | | |
| | | | NW_043651.1 | | | | |
| c-src | src-c; SRC | Rat | nucleotides 2038000 | intron:exon | 13173 | 13174 | 5 |
| | | | to 2054000 of | junction | | | |
| | | | NW_043651.1 | | | | |
| c-src | src-c; SRC | Rat | nucleotides 2038000 | exon | 13174 | 13327 | 5 |
| | | | to 2054000 of | | | | |
| | | | NW_043651.1 | | | | |
| c-src | src-c; SRC | Rat | nucleotides 2038000 | intron:exon | 13327 | 13328 | 5 |
| | | | to 2054000 of | junction | | | |
| | | | NW_043651.1 | | | | |
| c-src | src-c; SRC | Rat | nucleotides 2038000 | intron | 13328 | 13434 | 5 |
| | | | to 2054000 of | | | | |
| | | | NW_043651.1 | | | | |
| c-src | src-c; SRC | Rat | nucleotides 2038000 | intron:exon | 13434 | 13435 | 5 |
| | | | to 2054000 of | junction | | | |
| | | | NW_043651.1 | | | | |
| c-src | src-c; SRC | Rat | nucleotides 2038000 | exon | 13435 | 13566 | 5 |
| | | | to 2054000 of | | | | |
| | | | NW_043651.1 | | | | |
| c-src | src-c; SRC | Rat | nucleotides 2038000 | intron:exon | 13566 | 13567 | 5 |
| | | | to 2054000 of | junction | | | |
| | | | NW_043651.1 | | | | |
| c-src | src-c; SRC | Rat | nucleotides 2038000 | intron | 13567 | 13836 | 5 |
| | | | to 2054000 of | | | | |
| | | | NW_043651.1 | | | | |
| c-src | src-c; SRC | Rat | nucleotides 2038000 | intron:exon | 13836 | 13837 | 5 |
| | | | to 2054000 of | junction | | | |
| | | | NW_043651.1 | | | | |
| c-src | src-c; SRC | Rat | nucleotides 2038000 | exon | 13837 | 14608 | 5 |
| | | | to 2054000 of | | | | |
| | | | NW_043651.1 | | | | |
| c-src | src-c; SRC | Rat | nucleotides 2038000 | stop codon | 14043 | 14045 | 5 |
| | | | to 2054000 of | | | | |
| | | | NW_043651.1 | | | | |
| c-src | src-c; SRC | Rat | nucleotides 2038000 | 3′UTR | 14046 | 14417 | 5 |
| | | | to 2054000 of | | | | |
| | | | NW_043651.1 | | | | |
| c-src | src-c; SRC | Rat | nucleotides 2038000 | 3′UTR | 14086 | 14608 | 5 |
| | | | to 2054000 of | | | | |
| | | | NW_043651.1 | | | | |
| c-src | src-c; SRC | Rat | the complement of | start codon | 246 | 248 | 6 |
| | | | AA956919.1 | | | | |
| DKK-1 | dickkopf (Xenopus | Human | BX378125.1 | CDS | 212 | 1012 | 7 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | BX378125.1 | exon:exon | 454 | 455 | 7 |
| | laevis) homolog 1; | | | junction | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | BX378125.1 | exon | 455 | 617 | 7 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | BX378125.1 | exon:exon | 617 | 618 | 7 |
| | laevis) homolog 1; | | | junction | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | BX378125.1 | exon | 618 | 758 | 7 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | BX378125.1 | exon:exon | 758 | 759 | 7 |
| | laevis) homolog 1; | | | junction | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | BX378125.1 | stop codon | 1011 | 1013 | 7 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | CA489765.1 | intron:exon | 75 | 76 | 8 |
| | laevis) homolog 1; | | | junction | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | CA489765.1 | exon | 76 | 216 | 8 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | CA489765.1 | intron:exon | 216 | 217 | 8 |
| | laevis) homolog 1; | | | junction | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | CA489765.1 | intron | 217 | 334 | 8 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | CA489765.1 | intron:exon | 334 | 335 | 8 |
| | laevis) homolog 1; | | | junction | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | CA489765.1 | stop codon | 586 | 588 | 8 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | NM_012242.1 | 5′UTR | 1 | 139 | 9 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | NM_012242.1 | start codon | 140 | 142 | 9 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | NM_012242.1 | CDS | 140 | 940 | 9 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | NM_012242.1 | exon:exon | 382 | 383 | 9 |
| | laevis) homolog 1; | | | junction | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | NM_012242.1 | exon | 383 | 545 | 9 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | NM_012242.1 | exon:exon | 545 | 546 | 9 |
| | laevis) homolog 1; | | | junction | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | NM_012242.1 | exon | 546 | 686 | 9 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | NM_012242.1 | exon:exon | 686 | 687 | 9 |
| | laevis) homolog 1; | | | junction | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | NM_012242.1 | exon | 687 | 1519 | 9 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | NM_012242.1 | stop codon | 938 | 940 | 9 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | NM_012242.1 | 3′UTR | 941 | 1554 | 9 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | exon | 373 | 760 | 10 |
| | laevis) homolog 1; | | to 2628701 of | | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | start codon | 518 | 520 | 10 |
| | laevis) homolog 1; | | to 2628701 of | | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | intron:exon | 760 | 761 | 10 |
| | laevis) homolog 1; | | to 2628701 of | junction | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | intron | 761 | 1005 | 10 |
| | laevis) homolog 1; | | to 2628701 of | | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | intron:exon | 1005 | 1006 | 10 |
| | laevis) homolog 1; | | to 2628701 of | junction | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | exon | 1006 | 1168 | 10 |
| | laevis) homolog 1; | | to 2628701 of | | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | intron:exon | 1168 | 1169 | 10 |
| | laevis) homolog 1; | | to 2628701 of | junction | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | intron | 1169 | 2377 | 10 |
| | laevis) homolog 1; | | to 2628701 of | | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | intron:exon | 2377 | 2378 | 10 |
| | laevis) homolog 1; | | to 2628701 of | junction | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | exon | 2378 | 2518 | 10 |
| | laevis) homolog 1; | | to 2628701 of | | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | intron:exon | 2518 | 2519 | 10 |
| | laevis) homolog 1; | | to 2628701 of | junction | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | intron | 2519 | 2636 | 10 |
| | laevis) homolog 1; | | to 2628701 of | | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | intron:exon | 2636 | 2637 | 10 |
| | laevis) homolog 1; | | to 2628701 of | junction | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | exon | 2637 | 3469 | 10 |
| | laevis) homolog 1; | | to 2628701 of | | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | stop codon | 2888 | 2890 | 10 |
| | laevis) homolog 1; | | to 2628701 of | | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Human | nucleotides 2624833 | 3'UTR | 2891 | 3504 | 10 |
| | laevis) homolog 1; | | to 2628701 of | | | | |
| | DKK1; SK; | | NT_008583.16 | | | | |
| | dickkopf homolog 1 | | | | | | |
| | (Xenopus laevis); | | | | | | |
| | dickkopf-1 like | | | | | | |
| DKK-1 | dickkopf (Xenopus | Rat | the complement of | exon | 3320 | 3582 | 11 |
| | laevis) homolog 1; | | nucleotides 3415000 | | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | start codon | 3337 | 3339 | 11 |
| | laevis) homolog 1; | | nucleotides 3415000 | | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | intron:exon | 3582 | 3583 | 11 |
| | laevis) homolog 1; | | nucleotides 3415000 | junction | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | intron | 3583 | 3799 | 11 |
| | laevis) homolog 1; | | nucleotides 3415000 | | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | intron:exon | 3799 | 3800 | 11 |
| | laevis) homolog 1; | | nucleotides 3415000 | junction | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | exon | 3800 | 3968 | 11 |
| | laevis) homolog 1; | | nucleotides 3415000 | | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | intron:exon | 3968 | 3969 | 11 |
| | laevis) homolog 1; | | nucleotides 3415000 | junction | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | intron | 3969 | 5018 | 11 |
| | laevis) homolog 1; | | nucleotides 3415000 | | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | intron:exon | 5018 | 5019 | 11 |
| | laevis) homolog 1; | | nucleotides 3415000 | junction | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | exon | 5019 | 5162 | 11 |
| | laevis) homolog 1; | | nucleotides 3415000 | | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | intron:exon | 5162 | 5163 | 11 |
| | laevis) homolog 1; | | nucleotides 3415000 | junction | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | intron | 5163 | 5285 | 11 |
| | laevis) homolog 1; | | nucleotides 3415000 | | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | intron:exon | 5285 | 5286 | 11 |
| | laevis) homolog 1; | | nucleotides 3415000 | junction | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | exon | 5286 | 5728 | 11 |
| | laevis) homolog 1; | | nucleotides 3415000 | | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | stop codon | 5537 | 5539 | 11 |
| | laevis) homolog 1; | | nucleotides 3415000 | | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | the complement of | 3′UTR | 5540 | 5728 | 11 |
| | laevis) homolog 1; | | nucleotides 3415000 | | | | |
| | DKK1; SK; | | to 3424000 of | | | | |
| | dickkopf homolog 1 | | NW_043411.1 | | | | |
| | (Xenopus laevis); | | | | | | |
| | dickkopf-1 like | | | | | | |
| DKK-1 | dickkopf (Xenopus | Rat | XM_219804.1 | start codon | 1 | 3 | 12 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Rat | XM_219804.1 | CDS | 1 | 813 | 12 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Rat | XM_219804.1 | exon:exon | 246 | 247 | 12 |
| | laevis) homolog 1; | | | junction | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Rat | XM_219804.1 | exon | 247 | 415 | 12 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Rat | XM_219804.1 | exon:exon | 415 | 416 | 12 |
| | laevis) homolog 1; | | | junction | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Rat | XM_219804.1 | exon | 416 | 559 | 12 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Rat | XM_219804.1 | exon:exon | 559 | 560 | 12 |
| | laevis) homolog 1; | | | junction | | | |
| | DKK1; SK; | | | | | | |
| | dickkopf homolog 1 | | | | | | |
| | (Xenopus laevis); | | | | | | |
| | dickkopf-1 like | | | | | | |
| DKK-1 | dickkopf (Xenopus | Rat | XM_219804.1 | stop codon | 811 | 813 | 12 |
| | laevis) homolog 1; | | | | | | |
| | DKK1; SK; | | | | | | |
| | dickkopf homolog 1 | | | | | | |
| | (Xenopus laevis); | | | | | | |
| | dickkopf-1 like | | | | | | |
| GSK3 | glycogen synthase | Rat | AW919724.1 | exon:exon | 27 | 28 | 13 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | AW919724.1 | exon | 28 | 138 | 13 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | AW919724.1 | exon:exon | 138 | 139 | 13 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | AW919724.1 | exon | 139 | 269 | 13 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | AW919724.1 | exon:exon | 269 | 270 | 13 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | AW919724.1 | exon:exon | 377 | 378 | 13 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | AW919724.1 | intron:exon | 377 | 378 | 13 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | AW919724.1 | exon | 378 | 458 | 13 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | AW919724.1 | exon:exon | 458 | 459 | 13 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | AW919724.1 | exon | 459 | 557 | 13 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | AW919724.1 | exon:exon | 557 | 558 | 13 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | AW919724.1 | stop codon | 623 | 625 | 13 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | BF564221.1 | exon | 8 | 40 | 14 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | BF564221.1 | exon:exon | 40 | 41 | 14 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | BF564221.1 | exon | 41 | 227 | 14 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | BF564221.1 | exon:exon | 146 | 147 | 14 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | BF564221.1 | intron:exon | 146 | 147 | 14 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | BF564221.1 | exon:exon | 227 | 228 | 14 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | BF564221.1 | exon | 228 | 326 | 14 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | BF564221.1 | exon:exon | 326 | 327 | 14 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | BF564221.1 | stop codon | 392 | 394 | 14 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | 5′UTR | 1 | 139 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon | 1 | 227 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | start codon | 140 | 142 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | CDS | 140 | 1402 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon:exon | 227 | 228 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon | 228 | 421 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon:exon | 421 | 422 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon | 422 | 505 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon:exon | 505 | 506 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon | 506 | 616 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon:exon | 616 | 617 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon | 617 | 747 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon:exon | 747 | 748 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon | 748 | 854 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon:exon | 854 | 855 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon | 855 | 952 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon:exon | 952 | 953 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon | 953 | 1048 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon:exon | 1048 | 1049 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon | 1049 | 1235 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | intron:exon | 1154 | 1155 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon:exon | 1154 | 1155 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon:exon | 1235 | 1236 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon | 1236 | 1334 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon:exon | 1334 | 1335 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | exon | 1335 | 1525 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | stop codon | 1400 | 1402 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | NM_032080.1 | 3′UTR | 1403 | 1525 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | nucleotides 5224679 | 5′UTR | 401 | 539 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 401 | 627 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | start codon | 540 | 542 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 627 | 628 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron | 628 | 52675 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 52675 | 52676 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 52676 | 52869 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 52869 | 52870 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron | 52870 | 74674 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 74674 | 74675 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 74675 | 74758 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 74758 | 74759 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron | 74759 | 90199 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 90199 | 90200 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 90200 | 90310 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 90310 | 90311 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron | 90311 | 93517 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 93517 | 93518 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 93518 | 93648 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 93648 | 93649 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron | 93649 | 97257 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 97257 | 97258 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 97258 | 97364 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 97364 | 97365 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron | 97365 | 99921 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron | 97365 | 126536 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 99921 | 99922 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 99922 | 100019 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 100019 | 100020 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron | 100020 | 113143 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 113143 | 113144 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 113144 | 113239 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 113239 | 113240 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron | 113240 | 126430 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 123815 | 123847 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 123847 | 123848 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron | 123848 | 126430 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 126430 | 126431 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 126431 | 126617 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 126536 | 126537 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon:exon | 126536 | 126537 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 126537 | 126617 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 126617 | 126618 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron | 126618 | 134134 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | intron:exon | 134134 | 134135 | 16 |
| beta | kinase 3 beta; | | to 5369202 of | junction | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 134135 | 134233 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | exon | 143934 | 144124 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | stop codon | 143999 | 144001 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | nucleotides 5224679 | 3′UTR | 144002 | 144124 | 16 |
| beta | kinase 3 beta; | | to 5369202 of | | | | |
| | GSK3B; GSK3beta; | | NW_042728.1 | | | | |
| | TPKI; Tau kinase I; | | | | | | |
| | tau protein kinase I | | | | | | |
| GSK3 | glycogen synthase | Rat | X53428.1 | start codon | 115 | 117 | 17 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | CDS | 115 | 1377 | 17 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon:exon | 202 | 203 | 17 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon | 203 | 396 | 17 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon:exon | 396 | 397 | 17 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon | 397 | 480 | 17 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon:exon | 480 | 481 | 17 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon | 481 | 591 | 17 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon:exon | 591 | 592 | 17 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon | 592 | 722 | 17 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon:exon | 722 | 723 | 17 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon | 723 | 829 | 17 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon | 830 | 927 | 17 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon:exon | 927 | 928 | 17 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon | 928 | 1023 | 17 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon:exon | 1023 | 1024 | 17 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon | 1024 | 1210 | 17 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon:exon | 1129 | 1130 | 17 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | intron:exon | 1129 | 1130 | 17 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon:exon | 1210 | 1211 | 17 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon | 1211 | 1309 | 17 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | exon:exon | 1309 | 1310 | 17 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X53428.1 | stop codon | 1375 | 1377 | 17 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | 5′UTR | 1 | 139 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon | 1 | 227 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | start codon | 140 | 142 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | CDS | 140 | 1402 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon:exon | 227 | 228 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon | 228 | 421 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon:exon | 421 | 422 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon | 422 | 505 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon:exon | 505 | 506 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon | 506 | 616 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon:exon | 616 | 617 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon | 617 | 747 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon:exon | 747 | 748 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon | 748 | 854 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon:exon | 854 | 855 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon | 855 | 952 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon:exon | 952 | 953 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon | 953 | 1048 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon:exon | 1048 | 1049 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon | 1049 | 1235 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon:exon | 1154 | 1155 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | intron:exon | 1154 | 1155 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon:exon | 1235 | 1236 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon | 1236 | 1334 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon:exon | 1334 | 1335 | 15 |
| beta | kinase 3 beta; | | | junction | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | exon | 1335 | 1525 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | stop codon | 1400 | 1402 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| GSK3 | glycogen synthase | Rat | X73653.1 | 3′UTR | 1403 | 1525 | 15 |
| beta | kinase 3 beta; | | | | | | |
| | GSK3B; GSK3beta; | | | | | | |
| | TPKI; Tau kinase I; | | | | | | |
| | tau protein kinase I | | | | | | |
| sclerostin | RNF27; SOST; | Rat | AF326741.1 | 5′UTR | 1 | 32 | 18 |
| | sclerosteosis | | | | | | |
| sclerostin | RNF27; SOST; | Rat | AF326741.1 | start codon | 33 | 35 | 18 |
| | sclerosteosis | | | | | | |
| sclerostin | RNF27; SOST; | Rat | AF326741.1 | CDS | 33 | 674 | 18 |
| | sclerosteosis | | | | | | |
| sclerostin | RNF27; SOST; | Rat | AF32674 1.1 | stop codon | 672 | 674 | 18 |
| | sclerosteosis | | | | | | |
| sclerostin | RNF27; SOST; | Rat | NM_030584.1 | 5′UTR | 1 | 32 | 18 |
| | sclerosteosis | | | | | | |
| sclerostin | RNF27; SOST; | Rat | NM_030584.1 | 5′UTR | 1 | 32 | 18 |
| | sclerosteosis | | | | | | |
| sclerostin | RNF27; SOST; | Rat | NM_030584.1 | start codon | 33 | 35 | 18 |
| | sclerosteosis | | | | | | |
| sclerostin | RNF27; SOST; | Rat | NM_030584.1 | CDS | 33 | 674 | 18 |
| | sclerosteosis | | | | | | |
| sclerostin | RNF27; SOST; | Rat | NM_030584.1 | stop codon | 672 | 674 | 18 |
| | sclerosteosis | | | | | | |
| sFRP-1 | secreted frizzled- | Rat | AF167308.1 | CDS | 1 | 475 | 19 |
| | related protein 1; | | | | | | |
| | FRP; FRP-1; FRP1; | | | | | | |
| | FrzA; SARP2; | | | | | | |
| | SFRP1; secreted | | | | | | |
| | apoptosis-related | | | | | | |
| | protein 2 | | | | | | |
| transducer | transducer of | Human | NM_005749.1 | 5′UTR | 1 | 43 | 20 |
| of | ERBB2, 1; APRO6; | | | | | | |
| ERBB2 | MGC34446; TOB; | | | | | | |
| | TOB1; TROB; | | | | | | |
| | TROB1; transducer | | | | | | |
| | of erbB-2 | | | | | | |
| transducer | transducer of | Human | NM_005749.1 | start codon | 44 | 46 | 20 |
| of | ERBB2, 1; APRO6; | | | | | | |
| ERBB2 | MGC34446; TOB; | | | | | | |
| | TOB1; TROB; | | | | | | |
| | TROB1; transducer | | | | | | |
| | of erbB-2 | | | | | | |
| transducer | transducer of | Human | NM_005749.1 | CDS | 44 | 1081 | 20 |
| of | ERBB2, 1; APRO6; | | | | | | |
| ERBB2 | MGC34446; TOB; | | | | | | |
| | TOB1; TROB; | | | | | | |
| | TROB1; transducer | | | | | | |
| | of erbB-2 | | | | | | |
| transducer | transducer of | Human | NM_005749.1 | stop codon | 1079 | 1081 | 20 |
| of | ERBB2, 1; APRO6; | | | | | | |
| ERBB2 | MGC34446; TOB; | | | | | | |
| | TOB1; TROB; | | | | | | |
| | TROB1; transducer | | | | | | |
| | of erbB-2 | | | | | | |
| transducer | transducer of | Human | NM_005749.1 | 3′UTR | 1082 | 1206 | 20 |
| of | ERBB2, 1; APRO6; | | | | | | |
| ERBB2 | MGC34446; TOB; | | | | | | |
| | TOB1; TROB; | | | | | | |
| | TROB1; transducer | | | | | | |
| | of erbB-2 | | | | | | |
| transducer | transducer of | Rat | AF349723.1 | 5′UTR | 1 | 145 | 21 |
| of | ERBB2, 1; APRO6; | | | | | | |
| ERBB2 | MGC34446; TOB; | | | | | | |
| | TOB1; TROB; | | | | | | |
| | TROB1; transducer | | | | | | |
| | of erbB-2 | | | | | | |
| transducer | transducer of | Rat | AF349723.1 | start codon | 146 | 148 | 21 |
| of | ERBB2, 1; APRO6; | | | | | | |
| ERBB2 | MGC34446; TOB; | | | | | | |
| | TOB1; TROB; | | | | | | |
| | TROB1; transducer | | | | | | |
| | of erbB-2 | | | | | | |
| transducer | transducer of | Rat | AF349723.1 | CDS | 146 | 1243 | 21 |
| of | ERBB2, 1; APRO6; | | | | | | |
| ERBB2 | MGC34446; TOB; | | | | | | |
| | TOB1; TROB; | | | | | | |
| | TROB1; transducer | | | | | | |
| | of erbB-2 | | | | | | |
| transducer | transducer of | Rat | AF349723.1 | stop codon | 1241 | 1243 | 21 |
| of | ERBB2, 1; APRO6; | | | | | | |
| ERBB2 | MGC34446; TOB; | | | | | | |
| | TOB1; TROB; | | | | | | |
| | TROB1; transducer | | | | | | |
| | of erbB-2 | | | | | | |
| transducer | transducer of | Rat | AF349723.1 | 3′UTR | 1244 | 2024 | 21 |
| of | ERBB2, 1; APRO6; | | | | | | |
| ERBB2 | MGC34446; TOB; | | | | | | |
| | TOB1; TROB; | | | | | | |
| | TROB1; transducer | | | | | | |
| | of erbB-2 | | | | | | |
| transducer | transducer of | Rat | nucleotides 5191286 | exon | | 2428 | 22 |
| of | ERBB2, 1; APRO6; | | to 5194113 of | | | | |
| ERBB2 | MGC34446; TOB; | | NW_042669.1 | | | | |
| | TOB1; TROB; | | | | | | |
| | TROB1; transducer | | | | | | |
| | of erbB-2 | | | | | | |
| transducer | transducer of | Rat | nucleotides 5191286 | start codon | 545 | 547 | 22 |
| of | ERBB2, 1; APRO6; | | to 5194113 of | | | | |
| ERBB2 | MGC34446; TOB; | | NW_042669.1 | | | | |
| | TOB1; TROB; | | | | | | |
| | TROB1; transducer | | | | | | |
| | of erbB-2 | | | | | | |
| transducer | transducer of | Rat | nucleotides 5191286 | stop codon | 1643 | 1645 | 22 |
| of | ERBB2, 1; APRO6; | | to 5194113 of | | | | |
| ERBB2 | MGC34446; TOB; | | NW_042669.1 | | | | |
| | TOB1; TROB; | | | | | | |
| | TROB1; transducer | | | | | | |
| | of erbB-2 | | | | | | |
| transducer | transducer of | Rat | nucleotides 5191286 | 3′UTR | 1648 | 2428 | 22 |
| of | ERBB2, 1; APRO6, | | to 5194113 of | | | | |
| ERBB2 | MGC34446; TOB; | | NW_042669.1 | | | | |
| | TOB1; TROB; | | | | | | |
| | TROB1; transducer | | | | | | |
| | of erbB-2 | | | | | | |

Modulation of Target Expression

Modulation of expression of a target nucleic acid can be achieved through alteration of any number of nucleic acid (DNA or RNA) functions. “Modulation” means a perturbation of function, for example, either an increase (stimulation or induction) or a decrease (inhibition or reduction) in expression. As another example, modulation of expression can include perturbing splice site selection of pre-mRNA processing. “Expression” includes all the functions by which a gene's coded information is converted into structures present and operating in a cell. These structures include the products of transcription and translation. “Modulation of expression” means the perturbation of such functions. The functions of DNA to be modulated can include replication and transcription. Replication and transcription, for example, can be from an endogenous cellular template, a vector, a plasmid construct or otherwise. The functions of RNA to be modulated can include translocation functions, which include, but are not limited to, translocation of the RNA to a site of protein translation, translocation of the RNA to sites within the cell which are distant from the site of RNA synthesis, and translation of protein from the RNA. RNA processing functions that can be modulated include, but are not limited to, splicing of the RNA to yield one or more RNA species, capping of the RNA, 3′ maturation of the RNA and catalytic activity or complex formation involving the RNA which may be engaged in or facilitated by the RNA. Modulation of expression can result in the increased level of one or more nucleic acid species or the decreased level of one or more nucleic acid species, either temporally or by net steady state level. One result of such interference with target nucleic acid function is modulation of the expression of a bone growth modulator. Thus, in one embodiment modulation of expression can mean increase or decrease in target RNA or protein levels. In another embodiment modulation of expression can mean an increase or decrease of one or more RNA splice products, or a change in the ratio of two or more splice products.

The effect of oligomeric compounds of the present invention on target nucleic acid expression can be tested in any of a variety of cell types provided that the target nucleic acid is present at measurable levels. The effect of oligomeric compounds of the present invention on target nucleic acid expression can be routinely determined using, for example, PCR or Northern blot analysis. Cell lines are derived from both normal tissues and cell types and from cells associated with various disorders (e.g. hyperproliferative disorders). Cell lines derived from muliple tissues and species can be obtained from American Type Culture Collection (ATCC, Manassas, Va.) and include: Caco-2, D1 TNC1, SKBR-3, SK-MEL-28, TRAMP-C1, U937, undifferentiated 3T3-L1, 7F2, 7D4, A375, ARIP, AML-12, A20, A549, A10, A431, BLO-11, BC3H1, B16-F10, BW5147.3, BB88, BHK-21, BT-474, BEAS2B, C6, CMT-93, C3H/10T1/2, CHO-K1, ConA, C2C12, C3A, COS-7, CT26.WT, DDT1-MF2, DU145, D1B, E14, EMT-6, EL4, FAT7, GH1, GH3, G-361, HT-1080, HeLa, HCT116, H-4-II-E, HEK-293, HFN 36.3, HuVEC, HEPA1-6, H2.35, HK-2, Hep3B, HepG2, HuT 78, HL-60, H9c2(2-1), H9c2(2-1), IEC-6, IC21, JAR, JEG-3, Jurkat, K-562, K204, L2, LA4, LC-540, LLC1, LBRM-33, L6, LNcAP, LL2, MLg2908, MMT 060562, MH-S, MCF7, MDA MB231, MRC-5, M-3, Mia Paca, MLE12, MDA MB 468, MDA, NOR-10, NCTC 3749, N1S1, NBT-II, NIH/3T3, NC1-H292, NTERA-2 c1.D1, NIT-1, NCCIT, NR-8383, NRK, NG108-15, P388D1, PC-3, PANC-1, PC-12, P-19, P388D1 (IL-1), RFL-6, R2C, RK3E, Rin-M, Rin-5F, RBL-2H3, RMC, RAW264.7, Raji, Rat-2, SV40 MES 13, SMT/2A LNM, SW480, TCMK-1, THLE-3, TM-3, TM4, T3-3A1, T47D, T-24, THP-1, UMR-106, U-87 MG, U-20S, VERO C1008, WISH, WEHI 231, Y-1, YB2/0, Y13-238, Y13-259, Yac-1, b.END, mIMCD-3, sw872 and 70Z3. Additional cell lines, such as HuH-7 and U373, can be obtained from the Japanese Cancer Research Resources Bank (Tokyo, Japan) and the Centre for Applied Microbiology and Research (Wiltshire, United Kingdom), respectively.

Primary cells, or those cells which are isolated from an animal and not subjected to continuous culture, can be prepared according to methods known in the art or obtained from various commercial suppliers. Additionally, primary cells include those obtained from donor human subjects in a clinical setting (i.e. blood donors, surgical patients). Primary cells prepared by methods known in the art include: mouse or rat bronchoalveolar lavage cells, mouse primary bone marrow-derived osteoclasts, mouse primary keratinocytes, human primary macrophages, mouse peritoneal macrophages, rat peritoneal macrophages, rat primary neurons, mouse primary osteoblasts, rat primary osteoblasts, rat cerebellum tissue cells, rat cerebrum tissue cells, rat hippocampal tissue cells, mouse primary splenocytes, human synoviocytes, mouse synoviocytes and rat synoviocytes. Additional types of primary cells, including human primary melanocytes, human primary monocytes, NHDC, NHDF, adult NHEK, neonatal NHEK, human primary renal proximal tubule epithelial cells, mouse embryonic fibroblasts, differentiated adipocytes, HASMC, HMEC, HMVEC-L, adult HMVEC-D, neonatal HMVEC-D, HPAEC, human primary hepatocytes, monkey primary hepatocytes, mouse primary hepatocytes, hamster primary hepatocytes, rabbit primary hepatocytes and rat primary hepatocytes, can be obtained from commercial suppliers such as Stem Cell Technologies; Zen-Bio, Inc. (Research Triangle Park, N.C.); Cambrex Biosciences (Walkersville, Md.); In Vitro Technologies (Baltimore, Md.); Cascade Biologics (Portland, Oreg.); Advanced Biotechnologies (Columbia, Md.).

Assaying Modulation of Expression

Modulation of bone growth modulator expression can be assayed in a variety of ways known in the art. Bone growth modulator mRNA levels can be quantitated by, e.g., Northern blot analysis, competitive polymerase chain reaction (PCR), or real-time PCR. RNA analysis can be performed on total cellular RNA or poly(A)+ mRNA by methods known in the art. Methods of RNA isolation are taught in, for example, Ausubel, F. M. et al., Current Protocols in Molecular Biology, Volume 1, pp. 4.1.1-4.2.9 and 4.5.1-4.5.3, John Wiley & Sons, Inc., 1993.

Northern blot analysis is routine in the art and is taught in, for example, Ausubel, F. M. et al., Current Protocols in Molecular Biology, Volume 1, pp. 4.2.1-4.2.9, John Wiley & Sons, Inc., 1996. Real-time quantitative (PCR) can be conveniently accomplished using the commercially available ABI PRISM™ 7700 Sequence Detection System, available from PE-Applied Biosystems, Foster City, Calif. and used according to manufacturer's instructions.

Levels of a protein encoded by a bone growth modulator can be quantitated in a variety of ways well known in the art, such as immunoprecipitation, Western blot analysis (immunoblotting), ELISA or fluorescence-activated cell sorting (FACS). Antibodies directed to a protein encoded by a bone growth modulator can be identified and obtained from a variety of sources, such as the MSRS catalog of antibodies (Aerie Corporation, Birmingham, Mich.), or can be prepared via conventional antibody generation methods. Methods for preparation of polyclonal antisera are taught in, for example, Ausubel, F. M. et al., Current Protocols in Molecular Biology, Volume 2, pp. 11.12.1-11.12.9, John Wiley & Sons, Inc., 1997. Preparation of monoclonal antibodies is taught in, for example, Ausubel, F. M. et al., Current Protocols in Molecular Biology, Volume 2, pp. 11.4.1-11.11.5, John Wiley & Sons, Inc., 1997.

Immunoprecipitation methods are standard in the art and can be found at, for example, Ausubel, F. M. et al., Current Protocols in Molecular Biology, Volume 2, pp. 10.16.1-10.16.11, John Wiley & Sons, Inc., 1998. Western blot (immunoblot) analysis is standard in the art and can be found at, for example, Ausubel, F. M. et al., Current Protocols in Molecular Biology, Volume 2, pp. 10.8.1-10.8.21, John Wiley & Sons, Inc., 1997. Enzyme-linked immunosorbent assays (ELISA) are standard in the art and can be found at, for example, Ausubel, F. M. et al., Current Protocols in Molecular Biology, Volume 2, pp. 11.2.1-11.2.22, John Wiley & Sons, Inc., 1991.

Suitable Target Regions

Once one or more target regions, segments or sites have been identified, oligomeric compounds are designed which are sufficiently complementary to the target, i.e., hybridize sufficiently well and with sufficient specificity, to give the desired effect.

The oligomeric compounds of the present invention can be targeted to features of a target nucleobase sequence, such as those described in Table 1. All regions of a nucleobase sequence to which an oligomeric compound can be targeted, wherein the regions are greater than or equal to 8 and less than or equal to 80 nucleobases, are described as follows:

Let R(n, n+m−1) be a region from a target nucleobase sequence, where “n” is the 5′-most nucleobase position of the region, where “n+m−1” is the 3′-most nucleobase position of the region and where “m” is the length of the region. A set “S(m)”, of regions of length “m” is defined as the regions where n ranges from 1 to L−m+1, where L is the length of the target nucleobase sequence and L>m. A set, “A”, of all regions can be constructed as a union of the sets of regions for each length from where m is greater than or equal to 8 and is less than or equal to 80.

This set of regions can be represented using the following mathematical notation:

$$A = {⋃}_{m} S ( m ) where m ∈ N \vert 8 \leq m \leq 80 and$$

$$S ( m ) = \{ {R}_{n , n + m - 1} \vert n ∈ \{ 1 , 2 , 3 , … , L - m + 1 \} \}$$

where the mathematical operator | indicates “such that”,
where the mathematical operator ∈ indicates “a member of a set” (e.g. y∈Z indicates that element y is a member of set Z),
where x is a variable,
where N indicates all natural numbers, defined as positive integers,
and where the mathematical operator ∪ indicates “the union of sets”.

For example, the set of regions for m equal to 8, 20 and 80 can be constructed in the following manner. The set of regions, each 8 nucleobases in length, S(m=8), in a target nucleobase sequence 100 nucleobases in length (L=100), beginning at position 1 (n=1) of the target nucleobase sequence, can be created using the following expression:
S(8)={R_{1,8}|n∈{1,2,3, . . . ,93}}
and describes the set of regions comprising nucleobases 1-8,2-9, 3-10, 4-11, 5-12, 6-13, 7-14, 8-15, 9-16, 10-17, 11-18, 12-19, 13-20, 14-21, 15-22, 16-23, 17-24, 18-25, 19-26, 20-27, 21-28, 22-29, 23-30, 24-31, 25-32, 26-33, 27-34, 28-35, 29-36, 30-37, 31-38, 32-39, 33-40, 34-41, 35-42, 36-43, 37-44, 38-45, 39-46, 40-47, 41-48, 42-49, 43-50, 44-51, 45-52, 46-53, 47-54, 48-55, 49-56, 50-57, 51-58, 52-59, 53-60, 54-61, 55-62, 56-63, 57-64, 58-65, 59-66, 60-67, 61-68, 62-69, 63-70, 64-71, 65-72, 66-73, 67-74, 68-75, 69-76, 70-77, 71-78, 72-79, 73-80, 74-81, 75-82, 76-83, 77-84, 78-85, 79-86, 80-87, 81-88, 82-89, 83-90, 84-91, 85-92, 86-93, 87-94, 88-95, 89-96, 90-97, 91-98, 92-99, 93-100.

An additional set for regions 20 nucleobases in length, in a target sequence 100 nucleobases in length, beginning at position 1 of the target nucleobase sequence, can be described using the following expression:
S(20)={R_{1,20}|n∈{1,2,3, . . . ,81}}
and describes the set of regions comprising nucleobases 1-20, 2-21, 3-22, 4-23, 5-24, 6-25, 7-26, 8-27, 9-28, 10-29, 11-30, 12-31, 13-32, 14-33, 15-34, 16-35, 17-36, 18-37, 19-38, 20-39, 21-40, 22-41, 23-42, 24-43, 25-44, 26-45, 27-46, 28-47, 29-48, 30-49, 31-50, 32-51, 33-52, 34-53, 35-54, 36-55, 37-56, 38-57, 39-58, 40-59, 41-60, 42-61, 43-62, 44-63, 45-64, 46-65, 47-66, 48-67, 49-68, 50-69, 51-70, 52-71, 53-72, 54-73, 55-74, 56-75, 57-76, 58-77, 59-78, 60-79, 61-80, 62-81, 63-82, 64-83, 65-84, 66-85, 67-86, 68-87, 69-88, 70-89, 71-90, 72-91, 73-92, 74-93, 75-94, 76-95, 77-96, 78-97, 79-98, 80-99, 81-100.

An additional set for regions 80 nucleobases in length, in a target sequence 100 nucleobases in length, beginning at position 1 of the target nucleobase sequence, can be described using the following expression:
S(80)={R_{1,80}|n∈{1,2,3, . . . ,21}}
and describes the set of regions comprising nucleobases 1-80, 2-81, 3-82, 4-83, 5-84, 6-85, 7-86, 8-87, 9-88, 10-89, 11-90, 12-91, 13-92, 14-93, 15-94, 16-95, 17-96, 18-97, 19-98, 20-99, 21-100.

Thus, in this example, A would include regions 1-8,2-9, 3-10 . . . 93-100, 1-20, 2-21, 3-22 . . . 81-100, 1-80, 2-81, 3-82 . . . 21-100.

The union of these aforementioned example sets and other sets for lengths from 10 to 19 and 21 to 79 can be described using the mathematical expression

$$A = {⋃}_{m} S ( m )$$

where ∪ represents the union of the sets obtained by combining all members of all sets.

The mathematical expressions described herein defines all possible target regions in a target nucleobase sequence of any length L, where the region is of length m, and where m is greater than or equal to 8 and less than or equal to 80 nucleobases and, and where m is less than L, and where n is less than L−m+1.

Validated Target Segments

The locations on the target nucleic acid to which active oligomeric compounds hybridize are hereinbelow referred to as “validated target segments.” As used herein the term “validated target segment” is defined as at least an 8-nucleobase portion of a target region to which an active oligomeric compound is targeted. While not wishing to be bound by theory, it is presently believed that these target segments represent portions of the target nucleic acid which are accessible for hybridization.

Target segments can include DNA or RNA sequences that comprise at least the 8 consecutive nucleobases from the 5′-terminus of a validated target segment (the remaining nucleobases being a consecutive stretch of the same DNA or RNA beginning immediately upstream of the 5′-terminus of the target segment and continuing until the DNA or RNA contains about 8 to about 80 nucleobases). Similarly validated target segments are represented by DNA or RNA sequences that comprise at least the 8 consecutive nucleobases from the 3′-terminus of a validated target segment (the remaining nucleobases being a consecutive stretch of the same DNA or RNA beginning immediately downstream of the 3′-terminus of the target segment and continuing until the DNA or RNA contains about 8 to about 80 nucleobases). It is also understood that a validated oligomeric target segment can be represented by DNA or RNA sequences that comprise at least 8 consecutive nucleobases from an internal portion of the sequence of a validated target segment, and can extend in either or both directions until the oligonucleotide contains about 8 about 80 nucleobases.

Screening for Modulator Compounds

In another embodiment, the validated target segments identified herein can be employed in a screen for additional compounds that modulate the expression of a bone growth modulator. “Modulators” are those compounds that modulate the expression of a bone growth modulator and which comprise at least an 8-nucleobase portion which is complementary to a validated target segment. The screening method comprises the steps of contacting a validated target segment of a nucleic acid molecule encoding a bone growth modulator with one or more candidate modulators, and selecting for one or more candidate modulators which perturb the expression of a nucleic acid molecule encoding a bone growth modulator. Once it is shown that the candidate modulator or modulators are capable of modulating the expression of a nucleic acid molecule encoding a bone growth modulator, the modulator can then be employed in further investigative studies of the function of a bone growth modulator, or for use as a research, diagnostic, or therapeutic agent. The validated target segments can also be combined with a second strand as disclosed herein to form stabilized double-stranded (duplexed) oligonucleotides for use as a research, diagnostic, or therapeutic agent.

Phenotypic Assays

Once modulator compounds of a bone growth modulator have been identified by the methods disclosed herein, the compounds can be further investigated in one or more phenotypic assays, each having measurable endpoints predictive of efficacy in the treatment of a particular disease state or condition. Phenotypic assays, kits and reagents for their use are well known to those skilled in the art and are herein used to investigate the role and/or association of a bone growth modulator in health and disease. Representative phenotypic assays, which can be purchased from any one of several commercial vendors, include those for determining cell viability, cytotoxicity, proliferation or cell survival (Molecular Probes, Eugene, Oreg.; PerkinElmer, Boston, Mass.), protein-based assays including enzymatic assays (Panvera, LLC, Madison, Wis.; BD Biosciences, Franklin Lakes, N.J.; Oncogene Research Products, San Diego, Calif.), cell regulation, signal transduction, inflammation, oxidative processes and apoptosis (Assay Designs Inc., Ann Arbor, Mich.), triglyceride accumulation (Sigma-Aldrich, St. Louis, Mo.), angiogenesis assays, tube formation assays, cytokine and hormone assays and metabolic assays (Chemicon International Inc., Temecula, Calif.; Amersham Biosciences, Piscataway, N.J.).

Phenotypic endpoints include changes in cell morphology over time or treatment dose as well as changes in levels of cellular components such as proteins, lipids, nucleic acids, hormones, saccharides or metals. Measurements of cellular status which include pH, stage of the cell cycle, intake or excretion of biological indicators by the cell, are also endpoints of interest.

Measurement of the expression of one or more of the genes of the cell after treatment is also used as an indicator of the efficacy or potency of the bone growth modulator modulators. Hallmark genes, or those genes suspected to be associated with a specific disease state, condition, or phenotype, are measured in both treated and untreated cells.

The following phenotypic assays are useful in the study of the compounds and compositions of the present invention.

Cell Proliferation and Survival

Unregulated cell proliferation is a characteristic of cancer cells, thus most current chemotherapy agents target dividing cells, for example, by blocking the synthesis of new DNA required for cell division. However, cells in healthy tissues are also affected by agents that modulate cell proliferation.

In some cases, a cell cycle inhibitor will cause apoptosis in cancer cells, but allow normal cells to undergo growth arrest and therefore remain unaffected (Blagosklonny, Bioessays, 1999, 21, 704-709; Chen et al., Cancer Res., 1997, 57, 2013-2019; Evan and Littlewood, Science, 1998, 281, 1317-1322; Lees and Weinberg, Proc. Natl. Acad. Sci. USA, 1999, 96, 4221-4223). An example of sensitization to anti-cancer agents is observed in cells that have reduced or absent expression of the tumor suppressor genes p 53 (Bunz et al., Science, 1998, 282, 1497-1501; Bunz et al., J. Clin. Invest., 1999, 104, 263-269; Stewart et al., Cancer Res., 1999, 59, 3831-3837; Wahl et al., Nat. Med, 1996, 2, 72-79). However, cancer cells often escape apoptosis (Lowe and Lin, Carcinogenesis, 2000, 21, 485-495; Reed, Cancer J. Sci. Am., 1998, 4 Suppl 1, S8-14). Further disruption of cell cycle checkpoints in cancer cells can increase sensitivity to chemotherapy while allowing normal cells to take refuge in G1 and remain unaffected. Cell cycle assays can be employed to identify genes, such as p53, whose inhibition will sensitize cells to anti-cancer agents.

Caspase Activity

Programmed cell death, or apoptosis, is an important aspect of various biological processes, including normal cell turnover, as well as immune system and embryonic development. Apoptosis involves the activation of caspases, a family of intracellular proteases through which a cascade of events leads to the cleavage of a select set of proteins. The caspase family can be divided into two groups: the initiator caspases, such as caspase-8 and -9, and the executioner caspases, such as caspase-3, -6 and -7, which are activated by the initiator caspases. The caspase family contains at least 14 members, with differing substrate preferences (Thornberry and Lazebnik, Science, 1998, 281, 1312-1316). For example, a caspase assay can be used to identify genes whose inhibition selectively cause apoptosis in breast carcinoma cell lines, without affecting normal cells, and to identify genes whose inhibition results in cell death in p53-deficient T47D cells, and not in MCF7 cells which express p53 (Ross et al., Nat. Genet., 2000, 24, 227-235; Scherf et al., Nat. Genet., 2000, 24, 236-244).

Angiogenesis

Angiogenesis is the growth of new blood vessels (veins and arteries) by endothelial cells. This process is important in the development of a number of human diseases, and is believed to be particularly important in regulating the growth of solid tumors. Without new vessel formation it is believed that tumors will not grow beyond a few millimeters in size. In addition to their use as anti-cancer agents, inhibitors of angiogenesis have potential for the treatment of diabetic retinopathy, cardiovascular disease, rheumatoid arthritis and psoriasis (Carmeliet and Jain, Nature, 2000, 407, 249-257; Freedman and Isner, J. Mol. Cell. Cardiol., 2001, 33, 379-393; Jackson et al., Faseb J, 1997, 11, 457-465; Saaristo et al., Oncogene, 2000, 19, 6122-6129; Weber and De Bandt, Joint Bone Spine, 2000, 67, 366-383; Yoshida et al., Histol. Histopathol., 1999, 14, 1287-1294).

Angiogenesis is stimulated by numerous factors that promote interaction of endothelial cells with each other and with extracellular matrix molecules, resulting in the formation of capillary tubes. This morphogenic process is necessary for the delivery of oxygen to nearby tissues and plays an essential role in embryonic development, wound healing, and tumor growth (Carmeliet and Jain, Nature, 2000, 407, 249-257). Moreover, this process can be reproduced in a tissue culture assay that evaluated the formation of tube-like structures by endothelial cells. There are several different variations of the assay that use different matrices, such as collagen I (Kanayasu et al., Lipids, 1991, 26, 271-276), Matrigel (Yamagishi et al., J. Biol. Chem., 1997, 272, 8723-8730) and fibrin (Bach et al., Exp. Cell Res., 1998, 238, 324-334), as growth substrates for the cells. For example, HUVECs can be plated on a matrix derived from the Engelbreth-Holm-Swarm mouse tumor, which is very similar to Matrigel (Kleinman et al., Biochemistry, 1986, 25, 312-318; Madri and Pratt, J. Histochem. Cytochem., 1986, 34, 85-91). Untreated HUVECs form tube-like structures when grown on this substrate. Loss of tube formation in vitro has been correlated with the inhibition of angiogenesis in vivo (Carmeliet and Jain, Nature, 2000, 407, 249-257; Zhang et al., Cancer Res., 2002, 62, 2034-2042), which supports the use of in vitro tube formation as an endpoint for angiogenesis.

Adipocyte Differentiation

Insulin is an essential signaling molecule throughout the body, but its major target organs are the liver, skeletal muscle and adipose tissue. Insulin is the primary modulator of glucose homeostasis and helps maintain a balance of peripheral glucose utilization and hepatic glucose production. The reduced ability of normal circulating concentrations of insulin to maintain glucose homeostasis manifests in insulin resistance which is often associated with diabetes, central obesity, hypertension, polycystic ovarian syndrome, dyslipidemia and atherosclerosis (Saltiel, Cell, 2001, 104, 517-529; Saltiel and Kahn, Nature, 2001, 414, 799-806).

Insulin promotes the differentiation of preadipocytes into adipocytes. The condition of obesity, which results in increases in fat cell number, occurs even in insulin-resistant states in which glucose transport is impaired due to the anti-lipolytic effect of insulin. Inhibition of triglyceride breakdown requires much lower insulin concentrations than stimulation of glucose transport, resulting in maintenance or expansion of adipose stores (Kitamura et al., Mol. Cell. Biol., 1999, 19, 6286-6296; Kitamura et al., Mol. Cell. Biol., 1998, 18, 3708-3717).

One of the hallmarks of cellular differentiation is the upregulation of gene expression. During adipocyte differentiation, the gene expression patterns in adipocytes change considerably. Some genes known to be upregulated during adipocyte differentiation include hormone-sensitive lipase (HSL), adipocyte lipid binding protein (aP2), glucose transporter 4 (Glut4), and peroxisome proliferator-activated receptor gamma (PPAR-γ). Insulin signaling is improved by compounds that bind and inactivate PPAR-γ, a key regulator of adipocyte differentiation (Olefsky, J. Clin. Invest., 2000, 106, 467-472). Insulin induces the translocation of GLUT4 to the adipocyte cell surface, where it transports glucose into the cell, an activity necessary for triglyceride synthesis. In all forms of obesity and diabetes, a major factor contributing to the impaired insulin-stimulated glucose transport in adipocytes is the downregulation of GLUT4. Insulin also induces hormone sensitive lipase (HSL), which is the predominant lipase in adipocytes that functions to promote fatty acid synthesis and lipogenesis (Fredrikson et al., J. Biol. Chem., 1981, 256, 6311-6320). Adipocyte fatty acid binding protein (aP2) belongs to a multi-gene family of fatty acid and retinoid transport proteins. aP2 is postulated to serve as a lipid shuttle, solubilizing hydrophobic fatty acids and delivering them to the appropriate metabolic system for utilization (Fu et al., J. Lipid Res., 2000, 41, 2017-2023; Pelton et al., Biochem. Biophys. Res. Commun., 1999, 261, 456-458). Together, these genes play important roles in the uptake of glucose and the metabolism and utilization of fats.

Leptin secretion and an increase in triglyceride content are also well-established markers of adipocyte differentiation. While it serves as a marker for differentiated adipocytes, leptin also regulates glucose homeostasis through mechanisms (autocrine, paracrine, endocrine and neural) independent of the adipocyte's role in energy storage and release. As adipocytes differentiate, insulin increases triglyceride accumulation by both promoting triglyceride synthesis and inhibiting triglyceride breakdown (Spiegelman and Flier, Cell, 2001, 104, 531-543). As triglyceride accumulation correlates tightly with cell size and cell number, it is an excellent indicator of differentiated adipocytes.

Inflammation Assays

Inflammation assays are designed to identify genes that regulate the activation and effector phases of the adaptive immune response. During the activation phase, T lymphocytes (also known as T-cells) receiving signals from the appropriate antigens undergo clonal expansion, secrete cytokines, and upregulate their receptors for soluble growth factors, cytokines and co-stimulatory molecules (Cantrell, Annu. Rev. Immunol., 1996, 14, 259-274). These changes drive T-cell differentiation and effector function. In the effector phase, response to cytokines by non-immune effector cells controls the production of inflammatory mediators that can do extensive damage to host tissues. The cells of the adaptive immune systems, their products, as well as their interactions with various enzyme cascades involved in inflammation (e.g., the complement, clotting, fibrinolytic and kinin cascades) all represent potential points for intervention in inflammatory disease. The inflammation assay measures hallmarks of the activation phase of the immune response.

Dendritic cells can be used to identify regulators of dendritic cell-mediated T-cell costimulation. The level of interleukin-2 (IL-2) production by T-cells, a critical consequence of T-cell activation (DeSilva et al., J. Immunol., 1991, 147, 3261-3267; Salomon and Bluestone, Annu. Rev. Immunol., 2001, 19, 225-252), is used as an endpoint for T-cell activation. T lymphocytes are important immunoregulatory cells that mediate pathological inflammatory responses. Optimal activation of T lymphocytes requires both primary antigen recognition events as well as secondary or costimulatory signals from antigen presenting cells (APC). Dendritic cells are the most efficient APCs known and are principally responsible for antigen presentation to T-cells, expression of high levels of costimulatory molecules during infection and disease, and the induction and maintenance of immunological memory (Banchereau and Steinman, Nature, 1998, 392, 245-252). While a number of costimulatory ligand-receptor pairs have been shown to influence T-cell activation, a principal signal is delivered by engagement of CD28 on T-cells by CD80 (B7-1) and CD86 (B7-2) on APCs (Boussiotis et al., Curr. Opin. Immunol., 1994, 6, 797-807; Lenschow et al., Annu. Rev. Immunol., 1996, 14, 233-258). While not adhering to a specific mechanism, inhibition of T-cell co-stimulation by APCs holds promise for novel and more specific strategies of immune suppression. In addition, blocking costimulatory signals may lead to the development of long-term immunological anergy (unresponsiveness or tolerance) that would offer utility for promoting transplantation or dampening autoimmunity. T-cell anergy is the direct consequence of failure of T-cells to produce the growth factor IL-2 (DeSilva et al., J. Immunol., 1991, 147, 3261-3267; Salomon and Bluestone, Annu. Rev. Immunol., 2001, 19, 225-252).

The cytokine signaling assay identifies genes that regulate the responses of non-immune effector cells (initially endothelial cells) to cytokines such as interferon-gamma (IFN-γ). The effects of the oligomeric compounds of the present invention on the regulation of the production of intercellular adhesion molecule-1 (ICAM-1), interferon regulatory factor 1 (IRF1) and small inducible cytokine subfamily B (Cys-X-Cys), member 11 (SCYB11), which regulate other parameters of the inflammatory response, can be monitored in response to cytokine treatment.

Kits, Research Reagents, Diagnostics, and Therapeutics

The oligomeric compounds of the present invention can be utilized for diagnostics, therapeutics, prophylaxis and as research reagents and kits. Furthermore, antisense compounds, which are able to inhibit gene expression with specificity, are often used by those of ordinary skill to elucidate the function of particular genes or to distinguish between functions of various members of a biological pathway.

For use in kits and diagnostics, the oligomeric compounds of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more compounds or compositions of the present invention are compared to control cells or tissues not treated with compounds and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. USA., 2000, 97, 1976-81), protein arrays and proteomics (Celis, et al, FEBS Lett., 2000, 480, 2-16; Jungblut, et al, Electrophoresis, 1999, 20, 2100-10), expressed sequence tag (EST) sequencing (Celis, et al., FEBS Lett., 2000, 480, 2-16; Larsson, et al., J Biotechnol., 2000, 80, 143-57), subtractive RNA fingerprinting (SuRF) (Fuchs, et al, Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), FISH (fluorescent in situ hybridization) techniques (Going and Gusterson, Eur. J. Cancer, 1999, 35, 1895-904) and mass spectrometry methods (To, Comb. Chem. High Throughput Screen, 2000, 3, 235-41).

The specificity and sensitivity of antisense technology is also harnessed by those of skill in the art for therapeutic uses. Antisense compounds have been employed as therapeutic moieties in the treatment of disease states in animals, including humans. Antisense drugs have been safely and effectively administered to humans and numerous clinical trials are presently underway. It is thus established that antisense compounds are useful therapeutic modalities that can be configured to be useful in treatment regimes for the treatment of cells, tissues and animals, especially humans.

For therapeutics, an animal, such as a human, suspected of having or at risk of having a disease or disorder which can be treated by modulating the expression of a bone growth modulator is treated by administering compounds in accordance with this invention. For example, in one non-limiting embodiment, the methods comprise the step of administering to said animal, a therapeutically effective amount of an antisense compound that inhibits expression of a bone growth modulator in order to promote bone growth. Compounds of the invention can be used to modulate the expression of a bone growth modulator in an animal, such as a human. In one non-limiting embodiment, the methods comprise the step of administering to said animal an effective amount of an antisense compound that inhibits expression of a bone growth modulator. In one embodiment, the antisense compounds of the present invention effectively inhibit the levels or function of a bone growth modulator RNA. Because reduction in bone growth modulator mRNA levels can lead to alteration in bone growth modulator protein products of expression as well, such resultant alterations can also be measured. Antisense compounds of the present invention that effectively inhibit the levels or function of a bone growth modulator RNA or protein products of expression is considered an active antisense compound. In one embodiment, the antisense compounds of the invention inhibit the expression of bone growth modulator causing a reduction of RNA by at least 10%, by at least 20%, by at least 25%, by at least 30%, by at least 40%, by at least 50%, by at least 60%, by at least 70%, by at least 75%, by at least 80%, by at least 85%, by at least 90%, by at least 95%, by at least 98%, by at least 99%, or by 100%.

For example, the reduction of the expression of a bone growth modulator can be measured in a bodily fluid, tissue or organ of the animal. Bodily fluids include, but are not limited to, blood (serum or plasma), lymphatic fluid, cerebrospinal fluid, semen, urine, synovial fluid and saliva and can be obtained by methods routine to those skilled in the art. Tissues or organs include, but are not limited to, blood (e.g., hematopoietic cells, such as human hematopoietic progenitor cells, human hematopoietic stem cells, CD34+ cells CD4+ cells), lymphocytes and other blood lineage cells, skin, bone marrow, spleen, thymus, lymph node, brain, spinal cord, heart, skeletal muscle, liver, pancreas, prostate, kidney, lung, oral mucosa, esophagus, stomach, ilium, small intestine, colon, bladder, cervix, ovary, testis, mammary gland, adrenal gland, and adipose (white and brown). Samples of tissues or organs can be routinely obtained by biopsy. In some alternative situations, samples of tissues or organs can be recovered from an animal after death.

The cells contained within said fluids, tissues or organs being analyzed can contain a nucleic acid molecule encoding a bone growth modulator protein and/or the bone growth modulator-encoded protein itself. For example, fluids, tissues or organs procured from an animal can be evaluated for expression levels of the target mRNA or protein. mRNA levels can be measured or evaluated by real-time PCR, Northern blot, in situ hybridization or DNA array analysis. Protein levels can be measured or evaluated by ELISA, immunoblotting, quantitative protein assays, protein activity assays (for example, caspase activity assays) immunohistochemistry or immunocytochemistry. Furthermore, the effects of treatment can be assessed by measuring biomarkers associated with the target gene expression in the aforementioned fluids, tissues or organs, collected from an animal contacted with one or more compounds of the invention, by routine clinical methods known in the art. These biomarkers include but are not limited to: glucose, cholesterol, lipoproteins, triglycerides, free fatty acids and other markers of glucose and lipid metabolism; liver transaminases, bilirubin, albumin, blood urea nitrogen, creatine and other markers of kidney and liver function; interleukins, tumor necrosis factors, intracellular adhesion molecules, C-reactive protein and other markers of inflammation; testosterone, estrogen and other hormones; tumor markers; vitamins, minerals and electrolytes.

The compounds of the present invention can be utilized in pharmaceutical compositions by adding an effective amount of a compound to a suitable pharmaceutically acceptable diluent or carrier. In one aspect, the compounds of the present invention inhibit the expression of a bone growth modulator. The compounds of the invention can also be used in the manufacture of a medicament for the treatment of diseases and disorders related to bone growth modulator expression.

Methods whereby bodily fluids, organs or tissues are contacted with an effective amount of one or more of the antisense compounds or compositions of the invention are also contemplated. Bodily fluids, organs or tissues can be contacted with one or more of the compounds of the invention resulting in modulation of bone growth modulator expression in the cells of bodily fluids, organs or tissues. An effective amount can be determined by monitoring the modulatory effect of the antisense compound or compounds or compositions on target nucleic acids or their products by methods routine to the skilled artisan. Further contemplated are ex vivo methods of treatment whereby cells or tissues are isolated from a subject, contacted with an effective amount of the antisense compound or compounds or compositions and reintroduced into the subject by routine methods known to those skilled in the art.

Further contemplated herein is a method for the treatment of a subject suspected of having or at risk of having a disease or disorder comprising administering to a subject an effective amount of an isolated single stranded RNA or double stranded RNA oligonucleotide directed to a bone growth modulator. The ssRNA or dsRNA oligonucleotide may be modified or unmodified. That is, the present invention provides for the use of an isolated double stranded RNA oligonucleotide targeted to a bone growth modulator, or a pharmaceutical composition thereof, for the treatment of a disease or disorder.

In one embodiment, provided are uses of a compound of an isolated double stranded RNA oligonucleotide in the manufacture of a medicament for inhibiting bone growth modulator expression or overexpression. Thus, provided herein is the use of an isolated double stranded RNA oligonucleotide targeted to a bone growth modulator in the manufacture of a medicament for the treatment of a disease or disorder by means of the method described above.

Salts, Prodrugs and Bioequivalents

The oligomeric compounds of the present invention comprise any pharmaceutically acceptable salts, esters, or salts of such esters, or any other functional chemical equivalent which, upon administration to an animal including a human, is capable of providing (directly or indirectly) the biologically active metabolite or residue thereof. Accordingly, for example, the disclosure is also drawn to prodrugs and pharmaceutically acceptable salts of the oligomeric compounds of the present invention, pharmaceutically acceptable salts of such prodrugs, and other bioequivalents.

The term “prodrug” indicates a therapeutic agent that is prepared in an inactive or less active form that is converted to an active form (i.e., drug) within the body or cells thereof by the action of endogenous enzymes or other chemicals and/or conditions. In particular, prodrug versions of the oligonucleotides of the invention are prepared as SATE ((S-acetyl-2-thioethyl) phosphate) derivatives according to the methods disclosed in WO 93/24510 or WO 94/26764.

The term “pharmaceutically acceptable salts” refers to physiologically and pharmaceutically acceptable salts of the compounds of the invention: i.e., salts that retain the desired biological activity of the parent compound and do not impart undesired toxicological effects thereto.

Pharmaceutically acceptable base addition salts are formed with metals or amines, such as alkali and alkaline earth metals or organic amines. Examples of metals used as cations are sodium, potassium, magnesium, calcium, and the like. Examples of suitable amines are N,N′-dibenzylethylenediamine, chloroprocaine, choline, diethanolamine, dicyclohexylamine, ethylenediamine, N-methylglucamine, and procaine (see, for example, Berge et al., “Pharmaceutical Salts,” J. of Pharma Sci., 1977, 66, 1-19). The base addition salts of said acidic compounds are prepared by contacting the free acid form with a sufficient amount of the desired base to produce the salt in the conventional manner. The free acid form may be regenerated by contacting the salt form with an acid and isolating the free acid in the conventional manner. The free acid forms differ from their respective salt forms somewhat in certain physical properties such as solubility in polar solvents, but otherwise the salts are equivalent to their respective free acid for purposes of the present invention. As used herein, a “pharmaceutical addition salt” includes a pharmaceutically acceptable salt of an acid form of one of the components of the compositions of the invention. These include organic or inorganic acid salts of the amines. Acid salts are the hydrochlorides, acetates, salicylates, nitrates and phosphates. Other suitable pharmaceutically acceptable salts are well known to those skilled in the art and include basic salts of a variety of inorganic and organic acids, such as, for example, with inorganic acids, such as for example hydrochloric acid, hydrobromic acid, sulfuric acid or phosphoric acid; with organic carboxylic, sulfonic, sulfo or phospho acids or N-substituted sulfamic acids, for example acetic acid, propionic acid, glycolic acid, succinic acid, maleic acid, hydroxymaleic acid, methylmaleic acid, fumaric acid, malic acid, tartaric acid, lactic acid, oxalic acid, gluconic acid, glucaric acid, glucuronic acid, citric acid, benzoic acid, cinnamic acid, mandelic acid, salicylic acid, 4-aminosalicylic acid, 2-phenoxybenzoic acid, 2-acetoxybenzoic acid, embonic acid, nicotinic acid or isonicotinic acid; and with amino acids, such as the 22 alpha-amino acids involved in the synthesis of proteins in nature, for example glutamic acid or aspartic acid, and also with phenylacetic acid, methanesulfonic acid, ethanesulfonic acid, 2-hydroxyethanesulfonic acid, ethane-1,2-disulfonic acid, benzenesulfonic acid, 4-methylbenzenesulfoc acid, naphthalene-2-sulfonic acid, naphthalene-1,5-disulfonic acid, 2- or 3-phosphoglycerate, glucose-6-phosphate, N-cyclohexylsulfamic acid (with the formation of cyclamates), or with other acid organic compounds, such as ascorbic acid. Pharmaceutically acceptable salts of compounds may also be prepared with a pharmaceutically acceptable cation. Suitable pharmaceutically acceptable cations are well known to those skilled in the art and include alkaline, alkaline earth, ammonium and quaternary ammonium cations. Carbonates or hydrogen carbonates are also possible.

For oligonucleotides, examples of pharmaceutically acceptable salts include but are not limited to (a) salts formed with cations such as sodium, potassium, ammonium, magnesium, calcium, polyamines such as spermine and spermidine, etc.; (b) acid addition salts formed with inorganic acids, for example hydrochloric acid, hydrobromic acid, sulfuric acid, phosphoric acid, nitric acid and the like; (c) salts formed with organic acids such as, for example, acetic acid, oxalic acid, tartaric acid, succinic acid, maleic acid, fumaric acid, gluconic acid, citric acid, malic acid, ascorbic acid, benzoic acid, tannic acid, palmitic acid, alginic acid, polyglutamic acid, naphthalenesulfonic acid, methanesulfonic acid, p-toluenesulfonic acid, naphthalenedisulfonic acid, polygalacturonic acid, and the like; and (d) salts formed from elemental anions such as chlorine, bromine, and iodine. Sodium salts of antisense oligonucleotides are useful and are well accepted for therapeutic administration to humans. In another embodiment, sodium salts of dsRNA compounds are also provided.

Formulations

The oligomeric compounds of the invention may also be admixed, encapsulated, conjugated or otherwise associated with other molecules, molecule structures or mixtures of compounds, as for example, liposomes, receptor-targeted molecules, oral, rectal, topical or other formulations, for assisting in uptake, distribution and/or absorption. Representative United States patents that teach the preparation of such uptake, distribution and/or absorption-assisting formulations include, but are not limited to, U.S. Pat. Nos. 5,108,921; 5,354,844; 5,416,016; 5,459,127; 5,521,291; 5,543,158; 5,547,932; 5,583,020; 5,591,721; 4,426,330; 4,534,899; 5,013,556; 5,108,921; 5,213,804; 5,227,170; 5,264,221; 5,356,633; 5,395,619; 5,416,016; 5,417,978; 5,462,854; 5,469,854; 5,512,295; 5,527,528; 5,534,259; 5,543,152; 5,556,948; 5,580,575; and 5,595,756.

The present invention also includes pharmaceutical compositions and formulations which include the antisense compounds of the invention. The pharmaceutical compositions of the present invention may be administered in a number of ways depending upon whether local or systemic treatment is desired and upon the area to be treated. Administration may be topical (including but not limited to ophthalmic and to mucous membranes including vaginal and rectal delivery), pulmonary, e.g., by inhalation or insulation of powders or aerosols, including by nebulizer (intratracheal, intranasal, epidermal and transdermal), oral or parenteral. Parenteral administration includes intravenous, intraarterial, subcutaneous, intraperitoneal or intramuscular injection or infusion; or intracranial, e.g., intrathecal or intraventricular, administration. Sites of administration are known to those skilled in the art. Oligonucleotides with at least one 2′-O-methoxyethyl modification are believed to be useful for oral administration.

Pharmaceutical compositions and formulations for topical administration may include transdermal patches, ointments, lotions, creams, gels, drops, suppositories, sprays, liquids and powders. Conventional pharmaceutical carriers, aqueous, powder or oily bases, thickeners and the like may be necessary or desirable. Coated condoms, gloves and the like may also be useful.

Formulations for topical administration include those in which the oligomeric compounds of the invention are in admixture with a topical delivery agent such as lipids, liposomes, fatty acids, fatty acid esters, steroids, chelating agents and surfactants.

For topical or other administration, oligomeric compounds of the invention may be encapsulated within liposomes or may form complexes thereto, such as to cationic liposomes. Alternatively, oligonucleotides may be complexed to lipids, in particular to cationic lipids. Fatty acids and esters, pharmaceutically acceptable salts thereof, and their uses are further described in U.S. Pat. No. 6,287,860. Topical formulations are described in detail in U.S. patent application Ser. No. 09/315,298 filed on May 20, 1999.

The pharmaceutical formulations of the present invention, which may conveniently be presented in unit dosage form, may be prepared according to conventional techniques well known in the pharmaceutical industry. Such techniques include the step of bringing into association the active ingredients with the pharmaceutical carrier(s) or excipient(s). In general, the formulations are prepared by uniformly and intimately bringing into association the active ingredients with liquid carriers or finely divided solid carriers or both, and then, if necessary, shaping the product.

The compositions of the present invention may be formulated into any of many possible dosage forms such as, but not limited to, tablets, capsules, gel capsules, liquid syrups, soft gels, suppositories, and enemas. The compositions of the present invention may also be formulated as suspensions in aqueous, non-aqueous or mixed media. Aqueous suspensions may further contain substances which increase the viscosity of the suspension including, for example, sodium carboxymethylcellulose, sorbitol and/or dextran. The suspension may also contain stabilizers.

Pharmaceutical compositions of the present invention include, but are not limited to, solutions, emulsions, foams and liposome-containing formulations. The pharmaceutical compositions and formulations of the present invention may comprise one or more penetration enhancers, carriers, excipients or other active or inactive ingredients.

The pharmaceutical formulations and compositions of the present invention may also include surfactants. The use of surfactants in drug products, formulations and in emulsions is well known in the art. Surfactants and their uses are further described in U.S. Pat. No. 6,287,860.

In one embodiment, the present invention employs various penetration enhancers to affect the efficient delivery of oligomeric compounds, particularly oligonucleotides. Penetration enhancers may be classified as belonging to one of five broad categories, i.e., surfactants, fatty acids, bile salts, chelating agents, and non-chelating non-surfactants. Penetration enhancers and their uses are further described in U.S. Pat. No. 6,287,860.

In some embodiments, compositions for non-parenteral administration include one or more modifications from naturally-occurring oligonucleotides (i.e. full-phosphodiester deoxyribosyl or full-phosphodiester ribosyl oligonucleotides). Such modifications may increase binding affinity, nuclease stability, cell or tissue permeability, tissue distribution, or other biological or pharmacokinetic property.

Oral compositions for administration of non-parenteral oligomeric compounds can be formulated in various dosage forms such as, but not limited to, tablets, capsules, liquid syrups, soft gels, suppositories, and enemas. The term “alimentary delivery” encompasses e.g. oral, rectal, endoscopic and sublingual/buccal administration. Such oral oligomeric compound compositions can be referred to as “mucosal penetration enhancers.”

Oligomeric compounds, such as oligonucleotides, may be delivered orally, in granular form including sprayed dried particles, or complexed to form micro or nanoparticles. Oligonucleotide complexing agents and their uses are further described in U.S. Pat. No. 6,287,860. Oral formulations for oligonucleotides and their preparation are described in detail in U.S. application Ser. Nos. 09/108,673 (filed Jul. 1, 1998), 09/315,298 (filed May 20, 1999) and 10/071,822, filed Feb. 8, 2002.

In one embodiment, oral oligomeric compound compositions comprise at least one member of the group consisting of surfactants, fatty acids, bile salts, chelating agents, and non-chelating surfactants. Further embodiments comprise oral oligomeric compound comprising at least one fatty acid, e.g. capric or lauric acid, or combinations or salts thereof. One combination is the sodium salt of lauric acid, capric acid and UDCA.

In one embodiment, oligomeric compound compositions for oral delivery comprise at least two discrete phases, which phases may comprise particles, capsules, gel-capsules, microspheres, etc. Each phase may contain one or more oligomeric compounds, penetration enhancers, surfactants, bioadhesives, effervescent agents, or other adjuvant, excipient or diluent

A “pharmaceutical carrier” or “excipient” can be a pharmaceutically acceptable solvent, suspending agent or any other pharmacologically inert vehicle for delivering one or more nucleic acids to an animal and are known in the art. The excipient may be liquid or solid and is selected, with the planned manner of administration in mind, so as to provide for the desired bulk, consistency, etc., when combined with a nucleic acid and the other components of a given pharmaceutical composition.

Oral oligomeric compositions may additionally contain other adjunct components conventionally found in pharmaceutical compositions, at their art-established usage levels. Thus, for example, the compositions may contain additional, compatible, pharmaceutically-active materials such as, for example, antipruritics, astringents, local anesthetics or anti-inflammatory agents, or may contain additional materials useful in physically formulating various dosage forms of the composition of present invention, such as dyes, flavoring agents, preservatives, antioxidants, opacifiers, thickening agents and stabilizers.

One of skill in the art will recognize that formulations are routinely designed according to their intended use, i.e. route of administration.

Combinations

Compositions of the invention can contain two or more oligomeric compounds. In another related embodiment, compositions of the present invention can contain one or more antisense compounds, particularly oligonucleotides, targeted to a first nucleic acid and one or more additional antisense compounds targeted to a second nucleic acid target. Alternatively, compositions of the present invention can contain two or more antisense compounds targeted to different regions of the same nucleic acid target. Two or more combined compounds may be used together or sequentially.

Nonlimiting Disclosure and Incorporation by Reference

While certain compounds, compositions and methods of the present invention have been described with specificity in accordance with certain embodiments, the following examples serve only to illustrate the compounds of the invention and are not intended to limit the same. Each of the references, GenBank accession numbers, and the like recited in the present application is incorporated herein by reference in its entirety.

### EXAMPLE 1

The effect of oligomeric compounds on target nucleic acid expression was tested in one or more of the following cell types.

A10 Cells:

The rat aortic smooth muscle cell line A10 was obtained from the American Type Culture Collection (Manassas, Va.). A10 cells were routinely cultured in DMEM, high glucose (American Type Culture Collection, Manassas, Va.) supplemented with 10% fetal bovine serum (Invitrogen Life Technologies, Carlsbad, Calif.). Cells were routinely passaged by trypsinization and dilution when they reached approximately 80% confluence. Cells were seeded into 96-well plates (Falcon-Primaria #3872) at a density of approximately 2500 cells/well for use in oligomeric compound transfection experiments.

A549 Cells:

The human lung carcinoma cell line A549 was obtained from the American Type Culture Collection (Manassas, Va.). A549 cells were routinely cultured in DMEM, high glucose (Invitrogen Life Technologies, Carlsbad, Calif.) supplemented with 10% fetal bovine serum, 100 units per ml penicillin, and 100 micrograms per ml streptomycin (Invitrogen Life Technologies, Carlsbad, Calif.). Cells were routinely passaged by trypsinization and dilution when they reached approximately 90% confluence. Cells were seeded into 96-well plates (Falcon-Primaria #3872) at a density of approximately 5000 cells/well for use in oligomeric compound transfection experiments.

FAT 7 Cells:

The rat nasal squamous carcinoma cell line FAT 7 was obtained from the American Type Culture Collection (ATCC) (Manassas, Va.). FAT 7 cells were routinely cultured in Ham's F12K medium supplemented with 10% fetal bovine serum, 0.01 mg/mL insulin, 250 ng/mL hydrocortisone and 0.0025 mg/mL transferrin (medium and supplements from Invitrogen Life Technologies, Carlsbad, Calif.). Cells were routinely passaged by trypsinization and dilution when they reached approximately 70% confluence. Cells were seeded into 96-well plates (Falcon-Primaria #353872, BD Biosciences, Bedford, Mass.) at a density of approximately 5000 cells/well for use in oligomeric compound transfection experiments.

ND7/23

Mouse neuroblastoma (N18 tg 2)×rat dorsal root ganglion neurone hybrid cell line ND7/23 was obtained from the American Type Culture Collection (ATCC) (Manassas, Va.). ND7/23 cells were routinely cultured in DMEM, high glucose (Invitrogen Life Technologies, Carlsbad, Calif.) supplemented with 10% fetal bovine serum and 2 mM glutamine (Invitrogen Life Technologies, Carlsbad, Calif.). Cells were routinely passaged by gentle tapping of the flask and dilution when they reached approximately 70-90% confluence. Cells were seeded into 96-well plates (Falcon-Primaria #3872) at a density of approximately 4000 cells/well for use in oligomeric compound transfection experiments.

NRK Cells

Normal rat kidney (NRK) cells were obtained from American Type Culture Collection (Manassus, Va.). NRK cells were routinely cultured in MEM (Invitrogen Life Technolgies, Carlsbad, Calif.) supplemented with 10% fetal boving serum and 0.1 mM non-essential amino acids (Invitrogen Life Technologies, Carlsbad, Calif.) in a humidified atmosphere of 90% air-10% CO2 at 37° C. Cells were routinely passaged by trypsinization and dilution when they reached 85-90% confluencey. Cells were seeded into 96-well plates (Falcon-Primaria #353872, BD Biosciences, Bedford, Mass.) at a density of 6000 cells/well for use in antisense oligonucleotide transfection.

UMR-106 Cells

The rat osteosarcoma cell line were obtained from the American Type Culture Collection (ATCC) (Manassas, Va.). UMR-106 cells were routinely cultured in DMEM/F12 media (Invitrogen Corporation, Carlsbad, Calif.) supplemented with 10% fetal bovine serum (Invitrogen Corporation, Carlsbad, Calif.), 50 μg/mL Gentamicin Sulfate Solution (Irvine Scientific, Santa Ana, Calif.), penicillin 100 units per mL, and streptomycin 100 μg/mL (Invitrogen Corporation, Carlsbad, Calif.). Cells were routinely passaged by trypsinization and dilution when they reach 90% confluence. Cells were seeded onto 24-well plates (Falcon-353047) at a density of ˜5000 cells/cm² for treatment with the oligomeric compounds of the invention.

Treatment with Oligomeric Compounds:

When cells reached approximately 65-75% confluency, they were treated with oligonucleotide. Oligonucleotide was mixed with LIPOFECTIN™ (Invitrogen Life Technologies, Carlsbad, Calif.) to achieve a final concentration of 3 μg/mL LIPOFECTIN™ per 100 nM oligonucleotide in 1 mL OPTI-MEM™-1 or Eagle's MEM (Invitrogen Life Technologies, Carlsbad, Calif.). For cells grown in 96-well plates, wells were washed once with 100 μL OPTI-MEM™-1, Eagle's MEM or serum-free culture medium and then treated with 130 μL of the oligonucleotide/OPTI-MEM™-1 or Eagle's MEM/LIPOFECTIN™ cocktail. Cells were treated and data were obtained in duplicate or triplicate. After 4-7 hours of treatment at 37° C., the medium was replaced with fresh medium. Cells were harvested 16-24 hours after oligonucleotide treatment.

Control oligonucleotides are used to determine the optimal oligomeric compound concentration for a particular cell line. Furthermore, when oligomeric compounds of the invention are tested in oligomeric compound screening experiments or phenotypic assays, control oligonucleotides are tested in parallel with compounds of the invention. In some embodiments, the control oligonucleotides are used as negative control oligonucleotides, i.e., as a means for measuring the absence of an effect on gene expression or phenotype. In alternative embodiments, control oligonucleotides are used as positive control oligonucleotides, i.e., as oligonucleotides known to affect gene expression or phenotype. Control oligonucleotides are shown in Table 2. “Target Name” indicates the gene to which the oligonucleotide is targeted. “Species of Target” indicates species in which the oligonucleotide is perfectly complementary to the target mRNA. “Motif” is indicative of chemically distinct regions comprising the oligonucleotide. Certain compounds in Table 2 are composed of 2′-O-(2-methoxyethyl) nucleotides, also known as 2′-MOE nucleotides, and are designated as “Uniform MOE”. Certain compounds in Table 2 are chimeric oligonucleotides, composed of a central “gap” region consisting of 2′-deoxynucleotides, which is flanked on both sides (5′ and 3′) by “wings”. The wings are composed of 2′-O-(2-methoxyethyl) nucleotides, also known as 2′-MOE nucleotides. The “motif” of each gapmer oligonucleotide is illustrated in Table 2 and indicates the number of nucleotides in each gap region and wing, for example, “5-10-5” indicates a gapmer having a 10-nucleotide gap region flanked by 5-nucleotide wings. Similarly, the motif “5-9-6” indicates a 9-nucleotide gap region flanked by 5-nucleotide wing on the 5′ side and a 6-nucleotide wing on the 3′ side. ISIS 15839 is a “hemimer” composed of two regions of distinct chemistry, wherein the first 12-nucleotides are 2′-deoxynucleotides and the last 8 nucleotides are 2′-MOE nucleotides. ISIS 15344 is a “hemimer” composed of two regions of distinct chemistry, wherein the first 9 nucleotides are 2′-deoxynucleotides and the last 11 are 2′-MOE nucleotides. ISIS 13513 is a chimeric oligonucleotide composed of multiple regions of distinct chemistry, denoted with a motif of “6-8-5-1” and comprised of a 6-nucleotide wing flanking an 8-nucleotide gap region followed by 5 2′-MOE nucleotides and terminating with a 2′-deoxynucleotide at the 3′ end. The internucleoside (backbone) linkages are phosphorothioate throughout the oligonucleotides in Table 2. Unmodified cytosines are indicated by “^{U}C” in the nucleotide sequence; all other cytosines are 5-methylcytosines.

TABLE 2

Control oligonucleotides for cell line testing, oligomeric compound
screening and phenotypic assays

| | | | | | |
|---|---|---|---|---|---|
| | | | | | SEQ |
| | | Species of | | | ID |
| ISIS # | Target Name | Target | Sequence (5′ to 3′) | Motif | NO |

| | | | | | |
|---|---|---|---|---|---|
| 117386 | C/EBP alpha | Human | CCCTACTCAGTAGGCATTGG | 5-10-5 | 23 |
| 15839 | CD54 | Cynomolgus | GCCCAAGCTGGCATCCGTCA | Hemimer | 24 |
| | | monkey; Human; | | | |
| | | Rhesus monkey | | | |
| 113131 | CD86 | Human | CGTGTGTCTGTGCTAGTCCC | 5-10-5 | 25 |
| 289865 | forkhead box | Human | GGCAACGTGAACAGGTCCAA | 5-10-5 | 26 |
| | O1A | | | | |
| | (rhabdomyosar | | | | |
| | coma) | | | | |
| 122291 | Glucose | Mouse; Rat | TATTCCACGAACGTAGGCTG | 5-10-5 | 27 |
| | transporter-4 | | | | |
| 186515 | insulin-like | Human | AGGTAGCTITITGATITATGTAA | 5-10-5 | 28 |
| | growth factor | | | | |
| | binding | | | | |
| | protein 1 | | | | |
| 25237 | integrin beta 3 | Human | GCCCATITGCTGGACATGC | 4-10-4 | 29 |
| 196103 | integrin beta 3 | Human | AGCCCATTGCTGGACATGCA | 5-10-5 | 30 |
| 134062 | Interleukin 8 | Human | GCTTGTGTGCTCTGCTGTCT | 5-10-5 | 31 |
| 148715 | Jagged 2 | Human; Mouse; | TTGTCCCAGTCCCAGGCCTC | 5-10-5 | 32 |
| | | Rat | | | |
| 15346 | Jun N- | Human | CTCTCTGTAGG^{u}C^{u}C^{u}CGCTTGG | 5-9-6 | 33 |
| | Terminal | | | | |
| | Kinase-1 | | | | |
| 18076 | Jun N- | Human | CTTTC^{u}CGTTGGA^{u}C^{u}CCCTGGG | 5-9-6 | 34 |
| | Terminal | | | | |
| | Kinase-1 | | | | |
| 105390 | Jun N- | Human; Mouse; | CTGATCATAGCGAGTAAGTA | 5-10-5 | 35 |
| | Terminal | Rat | | | |
| | Kinase-1 | | | | |
| 18078 | Jun N- | Human | GTGCG^{u}CG^{u}CGAG^{u}C^{u}C^{u}CGAAATC | 5-9-6 | 36 |
| | Terminal | | | | |
| | Kinase-2 | | | | |
| 101759 | Jun N- | Mouse; Rat | GCTCAGTGGACATGGATGAG | 5-10-5 | 37 |
| | Terminal | | | | |
| | Kinase-2 | | | | |
| 183881 | kinesin-like 1 | Human | ATCCAAGTGCTACTGTAGTA | 5-10-5 | 38 |
| 342672 | mir-143 | Human; Mouse; | ATACCGCGATCAGTGCATCTTT | Uniform | 39 |
| | | Rat | MOE | | |
| 342673 | mir-143 | Human; Mouse; | AGACTAGCGGTATCTFITATCCC | Uniform | 40 |
| | | Rat | MOE | | |
| 29848 | none | none | NNNNNNNNNNNNNNNNNNNN | 5-10-5 | 41 |
| 129685 | none | none | AATATTCGCACCCCACTGGT | 5-10-5 | 42 |
| 129686 | none | none | CGTTATTAACCTCCGTTGAA | 5-10-5 | 43 |
| 129687 | none | none | ACAAGCGTCAACCGTATTAT | 5-10-5 | 44 |
| 129688 | none | none | TTCGCGGCTGGACGATTCAG | 5-10-5 | 45 |
| 129689 | none | none | GAGGTCTCGACTTACCCGCT | 5-10-5 | 46 |
| 129690 | none | none | TTAGAATACGTCGCGTTATG | 5-10-5 | 47 |
| 129691 | none | none | ATGCATACTACGAAAGGCCG | 5-10-5 | 48 |
| 129692 | none | none | ACATGGGCGCGCGACTAAGT | 5-10-5 | 49 |
| 129694 | none | none | GTACAGTTATGCGCGGTAGA | 5-10-5 | 50 |
| 129695 | none | none | TTCTACCTCGCGCGATTTAC | 5-10-5 | 51 |
| 129696 | none | none | ATTCGCCAGACAACACTGAC | 5-10-5 | 52 |
| 129697 | none | none | AATAAGTACGTACTATTGTC | 5-10-5 | 53 |
| 129698 | none | none | TTTGATCGAGGTTAGCCGTG | 5-10-5 | 54 |
| 129699 | none | none | GGATAGAACGCGAAAGCTTG | 5-10-5 | 55 |
| 129700 | none | none | TAGTGCGGACCTACCCACGA | 5-10-5 | 56 |
| 226844 | Notch | Human; Mouse | GCCCTCCATGCTGGCACAGG | 5-10-5 | 57 |
| | (Drosophila) | | | | |
| | homolog 1 | | | | |
| 113529 | PARP-2 | Mouse | CTCTTACTGTGCTGTGGACA | 5-10-5 | 58 |
| 105990 | Peroxisome | Human | AGCAAAAGATCAATCCGTTA | 5-10-5 | 59 |
| | proliferator- | | | | |
| | activated | | | | |
| | receptor | | | | |
| | gamma | | | | |
| 13513 | Protein kinase | Human; Mouse | GGACCC^{u}CGAAAGA^{u}CCACCAG | 6-8-5-1 | 60 |
| | C-delta | | | | |
| 116847 | PTEN | Human; Mouse; | CTGCTAGCCTCTGGATTTGA | 5-10-5 | 61 |
| | | Rabbit; Rat | | | |
| 15344 | Raf kinase B | Human | CTGCCTGGATGGGTGTFIT1T | Hemimer | 62 |
| 13650 | Raf kinase C | Human | TCCCGC^{u}CTGTGA^{u}CATGCATT | 6-8-6 | 63 |
| 336806 | Raf kinase C | Human | TACAGAAGGCTGGGCCTTGA | 5-10-5 | 64 |
| 15770 | Raf kinase C | Mouse; Murine | ATGCATT^{u}CTG^{u}C^{u}C^{u}C^{u}C^{u}CAAGGA | 5-10-5 | 65 |
| | | sarcoma virus; | | | |
| | | Rat | | | |
| 30748 | Ship-2 | Human; Mouse, | CCAACCTCAAATGTCCCA | 4-10-4 | 66 |
| | | Rat | | | |
| 153704 | STAT 1 | Human; Rat | AGGCATGGTCT1TGTCAATA | 5-10-5 | 67 |
| 23722 | Survivin | Human | TGTGCTATTCTGTGAATT | 4-10-4 | 68 |
| 114845 | Talin | Human | TACGTCCGGAGGCGTACGCC | 5-10-5 | 69 |

The concentration of oligonucleotide used varies from cell line to cell line. To determine the optimal oligonucleotide concentration for a particular cell line, the cells are treated with a positive control oligonucleotide at a range of concentrations. Positive controls are shown in Table 2. For human and non-human primate cells, the positive control oligonucleotide is selected from ISIS 13650, ISIS 338806 or ISIS 18078. For mouse or rat cells the positive control oligonucleotide is ISIS 15770 or ISIS 15346. The concentration of positive control oligonucleotide that results in 80% inhibition of the target mRNA, for example, human Raf kinase C for ISIS 13650, is then utilized as the screening concentration for new oligonucleotides in subsequent experiments for that cell line. If 80% inhibition is not achieved, the lowest concentration of positive control oligonucleotide that results in 60% inhibition of the target mRNA is then utilized as the oligonucleotide screening concentration in subsequent experiments for that cell line. If 60% inhibition is not achieved, that particular cell line is deemed as unsuitable for oligonucleotide transfection experiments. The concentrations of antisense oligonucleotides used herein are from 50 μM to 300 nM when the antisense oligonucleotide is transfected using a liposome reagent and 10 μM to 20 μM when the antisense oligonucleotide is transfected by electroporation.

### EXAMPLE 2

Real-Time Quantitative PCR Analysis of Bone Growth Modulator mRNA Levels

Quantitation of bone growth modulator mRNA levels was accomplished by real-time quantitative PCR using the ABI PRISM™ 7600, 7700, or 7900 Sequence Detection System (PE-Applied Biosystems, Foster City, Calif.) according to manufacturer's instructions.

Prior to quantitative PCR analysis, primer-probe sets specific to the target gene being measured were evaluated for their ability to be “multiplexed” with a GAPDH amplification reaction. After isolation the RNA is subjected to sequential reverse transcriptase (RT) reaction and real-time PCR, both of which are performed in the same well. RT and PCR reagents were obtained from Invitrogen Life Technologies (Carlsbad, Calif.). RT, real-time PCR was carried out in the same by adding 20 μL PCR cocktail (2.5×PCR buffer minus MgCl₂, 6.6 mM MgCl₂, 375 μM each of dATP, dCTP, dCTP and dGTP, 375 nM each of forward primer and reverse primer, 125 nM of probe, 4 Units RNAse inhibitor, 1.25 Units PLATINUM® Taq, 5 Units MuLV reverse transcriptase, and 2.5×ROX dye) to 96-well plates containing 30 μL total RNA solution (20-200 ng). The RT reaction was carried out by incubation for 30 minutes at 48° C. Following a 10 minute incubation at 95° C. to activate the PLATINUM® Taq, 40 cycles of a two-step PCR protocol were carried out: 95° C. for 15 seconds (denaturation) followed by 60° C. for 1.5 minutes (annealing/extension).

Gene target quantities obtained by RT, real-time PCR were normalized using either the expression level of GAPDH, a gene whose expression is constant, or by quantifying total RNA using RiboGreen™ (Molecular Probes, Inc. Eugene, Oreg.). GAPDH expression was quantified by RT, real-time PCR, by being run simultaneously with the target, multiplexing, or separately. Total RNA was quantified using RiboGreen™ RNA quantification reagent (Molecular Probes, Inc. Eugene, Oreg.).

170 μL of RiboGreen™ working reagent (RiboGreen™ reagent diluted 1:350 in 10 mM Tris-HCl, 1 mM EDTA, pH 7.5) was pipetted into a 96-well plate containing 30 μL purified cellular RNA. The plate was read in a CytoFluor 4000 (PE Applied Biosystems) with excitation at 485 nm and emission at 530 nm.

Presented in Table 3 are primers and probes used to measure GAPDH expression in the cell types described herein. The GAPDH PCR probes have JOE covalently linked to the 5′ end and TAMRA or MGB covalently linked to the 3′ end, where JOE is the fluorescent reporter dye and TAMRA or MGB is the quencher dye. In some cell types, primers and probe designed to a GAPDH sequence from a different species are used to measure GAPDH expression. For example, a human GAPDH primer and probe set is used to measure GAPDH expression in monkey-derived cells and cell lines.

TABLE 3
GAPDH primers and probes for use in real-time PCR

| | | | | |
|---|---|---|---|---|
| Target | | Sequence | | |
| Name | Species | Description | Sequence (5′ to 3′) | SEQ ID NO |

| | | | | |
|---|---|---|---|---|
| GAPDH | Human | Forward Primer | CAACGGATTTGGTCGTATTGG | 70 |
| GAPDH | Human | Reverse Primer | GGCAACAATATCCACTTTACCAGAGT | 71 |
| GAPDH | Human | Probe | CGCCTGGTCACCAGGGCTGCT | 72 |
| GAPDH | Human | Forward Primer | GAAGGTGAAGGTCGGAGTC | 73 |
| GAPDH | Human | Reverse Primer | GAAGATGGTGATGGGATTTC | 74 |
| GAPDH | Human | Probe | CAAGCTTCCCGTTCTCAGCC | 75 |
| GAPDH | Human | Forward Primer | GAAGGTGAAGGTCGGAGTC | 73 |
| GAPDH | Human | Reverse Primer | GAAGATGGTGATGGGATTTC | 74 |
| GAPDH | Human | Probe | TGGAATCATATTGGAACATG | 76 |
| GAPDH | Mouse | Forward Primer | GGCAAATTCAACGGCACAGT | 77 |
| GAPDH | Mouse | Reverse Primer | GGGTCTCGCTCCTGGAAGAT | 78 |
| GAPDH | Mouse | Probe | AAGGCCGAGAATGGGAAGCTTGTCATC | 79 |
| GAPDH | Rat | Forward Primer | TGTTCTAGAGACAGCCGCATCTT | 80 |
| GAPDH | Rat | Reverse Primer | CACCGACCTTCACCATCTGT | 81 |
| GAPDH | Rat | Probe | TTGTGCAGTGCCAGCCTCGTCTCA | 82 |

Probes and primers for use in real-time PCR were designed to hybridize to target-specific sequences. The primers and probes and the target nucleic acid sequences to which they hybridize are presented in Table 4. The target-specific PCR probes have FAM covalently linked to the 5′ end and TAMRA or MGB covalently linked to the 3′ end, where FAM is the fluorescent dye and TAMRA or MGB is the quencher dye.

TABLE 4
Gene target-specific primers and probes for use in real-time PCR

| | | | | | |
|---|---|---|---|---|---|
| | | Target | Seqeunce | | SEQ |
| Target | | SEQ ID | Descrip- | | ID |
| Name | Species | NO | tion | Sequence (5′ to 3′) | NO |

| | | | | | |
|---|---|---|---|---|---|
| c-src | Rat | 4 | Forward | CCGCACGCAATTCAACAG | 83 |
| | | | Primer | | |
| c-src | Rat | 4 | Reverse | GACACAGGCCATCAGCATGT | 84 |
| | | | Primer | | |
| c-src | Rat | 4 | Probe | CTGCAGCAGCTTGTGGCTTACTACTCCA | 85 |
| DKK-1 | Human | 9 | Forward | AAGATCACCATCAAGCCAGTAATTC | 86 |
| | | | Primer | | |
| DKK-1 | Human | 9 | Reverse | AAAAGGAGTTCACTGCATTTGGA | 87 |
| | | | Primer | | |
| DKK-1 | Human | 9 | Probe | CTAGGCTTCACACTTGTCAGAGACACTA | 88 |
| | | | | AACCAGC | |
| DKK-1 | Rat | 12 | Forward | CAAGTACCAGACTCTTGACAACTACCA | 89 |
| | | | Primer | | |
| DKK-1 | Rat | 12 | Reverse | TGCCGCACTCCTCATCCT | 90 |
| | | | Primer | | |
| DKK-1 | Rat | 12 | Probe | CCCTACCCTTGCGCG | 91 |
| GSK3 beta | Rat | 15 | Forward | GGACCCAAATGTCAAACTACCAA | 92 |
| | | | Primer | | |
| GSK3 beta | Rat | 15 | Reverse | TGACAGTTCTTGAGTGGTAAAGTTGAA | 93 |
| | | | Primer | | |
| GSK3 beta | Rat | 15 | Probe | TGGGCGAGACACACCTGCCCT | 94 |
| sclerostin | Rat | 18 | Forward | CTGGTGGCCTCGTGCAA | 95 |
| | | | Primer | | |
| sclerostin | Rat | 18 | Reverse | TCTCAGGTCCGAAGTCCTTGAG | 96 |
| | | | Primer | | |
| sclerostin | Rat | 18 | Probe | CCGCTTCCACAACCAGTCGGA | 97 |
| sFRP-1 | Rat | 19 | Forward | TGCGCTGAGAATGAAAATCAA | 98 |
| | | | Primer | | |
| sFRP-1 | Rat | 19 | Reverse | CCAGCTTCAAGGGTTTCTTCTTC | 99 |
| | | | Primer | | |
| sFRP-1 | Rat | 19 | Probe | AAGTAAAAAAGGAAAACGGTGACAAGA | 100 |
| | | | | AGATTGTCC | |
| transducer of | Human | 20 | Forward | AGAGTGGTTTGGACATTGATGATG | 101 |
| ERBB2 | | | Primer | | |
| transducer of | Human | 20 | Reverse | CAAATGGGTCGATCCAAACAC | 102 |
| ERBB2 | | | Primer | | |
| transducer of | Human | 20 | Probe | TCGTGGCAATCTGCCACAGGATCTT | 103 |
| ERBB2 | | | | | |
| transducer of | Rat | 21 | Forward | GCCTGAATGTCAATGTGAACGA | 104 |
| ERBB2 | | | Primer | | |
| transducer of | Rat | 21 | Reverse | GCCCAGCCCGTACAGAGA | 105 |
| ERBB2 | | | Primer | | |
| transducer of | Rat | 21 | Probe | AAGCAGAAAGCCATCTCTTCCTCAATGCA | 106 |
| ERBB2 | | | | | |

### EXAMPLE 3

Treatment of Cultured Cells with Oligomeric Compounds

Oligomeric compounds targeted to genes presented in Table 1 were tested for their effects on gene target expression in cultured cells. Table 5 shows the experimental conditions, including cell type, transfection method, dose of oligonucleotide and control SEQ ID NO used to evaluate the inhibition of gene expression by the oligomeric compounds of the invention. The control oligonucleotide was chosen from the group presented in Table 2, and in these experiments was used as a negative control. Each cell type was treated with the indicated dose of oligonucleotide as described by other examples herein. The oligomeric compounds and the data describing the degree to which they inhibit gene expression are shown in Table 6.

TABLE 5
Treatment conditions of cultured cells with oligomeric compounds

| | | | Dose of | Control |
|---|---|---|---|---|
| Target | | Transfection | Oligonucleotide | SEQ |
| Name | Cell Type | Method | (nM) | ID NO |
| c-src | A10 | Lipofectin | 100 | 36 |
| DKK-1 | A549 | Lipofectin | 70 | 36 |
| DKK-1 | ND7/23 | Lipofectin | 300 | 36 |
| GSK3 | A10 | Lipofectin | 100 | 36 |
| beta | | | | |
| sclerostin | UMR-106 | Lipofectin | 100 | 36 |
| | (osteosarcoma) | | | |
| sFRP-1 | FAT 7 (epithelial, | Lipofectin | 40 | 36 |
| | nasal squamous | | | |
| | cell carcinoma) | | | |
| transducer | A10 | Lipofectin | 100 | 36 |
| of ERBB2 | | | | |
| transducer | A549 | Lipofectin | 100 | 36 |
| of ERBB2 | | | | |

### EXAMPLE 4

Antisense Inhibition Of Gene Targets by Oligomeric Compounds

A series of oligomeric compounds was designed to target different regions of the each gene target, using published sequences cited in Table 1. The compounds are shown in Table 6. All compounds in Table 6 are chimeric oligonucleotides (“gapmers”) 20 nucleotides in length, composed of a central “gap” region consisting of 10 2′-deoxynucleotides, which is flanked on both sides (5′ and 3′) by five-nucleotide “wings”. The wings are composed of 2′-O-(2-methoxyethyl) nucleotides, also known as 2′-MOE nucleotides. The internucleoside (backbone) linkages are phosphorothioate throughout the oligonucletide. All cytidine residues are 5-methylcytidines. The compounds were analyzed for their effect on gene target mRNA levels by quantitative real-time PCR as described in other examples herein. Data are averages from experiments in which cultured cells, as indicated for each target in Table 5, were treated with the disclosed oligomeric compounds. A reduction in expression is expressed as percent inhibition in Table 6. If the target expression level of oligomeric compound-treated cell was higher than control, percent inhibition is expressed as zero inhibition. If present, “N.D.” indicates “not determined”. The target regions to which these oligomeric compounds are inhibitory are herein referred to as “validated target segments.”

TABLE 6
Inhibition of gene target mRNA levels by chimeric
oligonucleotides having 2′-MOE wings and deoxy gap

| | | | | | |
|---|---|---|---|---|---|
| | Target | | | | SEQ |
| | SEQ | Target | | % | ID |
| ISIS # | ID NO | Site | Sequence (5′ to 3′) | Inhib | NO |

| | | | | | |
|---|---|---|---|---|---|
| 334145 | 1 | 172 | GTCTCCCTCTGTGTTATTGA | 60 | 107 |
| 334156 | 3 | 358 | GTTCCACCCAGAAGCCTCCA | 75 | 108 |
| 143607 | 4 | 1 | TTGCTCTTGTTGCTGCCCAT | 73 | 109 |
| 143609 | 4 | 176 | TCCGAAGAGCTTGGGCTCGG | 68 | 110 |
| 143610 | 4 | 180 | AGCCTCCGAAGAGCTTGGGC | 76 | 111 |
| 143611 | 4 | 185 | GTTGAAGCCTCCGAAGAGCT | 71 | 112 |
| 143612 | 4 | 187 | GAGTTGAAGCCTCCGAAGAG | 52 | 113 |
| 143523 | 4 | 191 | CGAGGAGTTGAAGCCTCCGA | 54 | 114 |
| 143613 | 4 | 193 | TCCGAGGAGTTGAAGCCTCC | 66 | 115 |
| 143524 | 4 | 196 | GTGTCCGAGGAGTTGAAGCC | 65 | 116 |
| 334146 | 4 | 241 | GTCACCCCACCTGCCAGAGG | 75 | 117 |
| 334147 | 4 | 262 | TCATAGAGGGCCACAAAGGT | 74 | 118 |
| 334148 | 4 | 282 | TCTCTGTCCGTGACTCATAG | 75 | 119 |
| 143614 | 4 | 284 | AGTCTCTGTCCGTGACTCAT | 74 | 120 |
| 143615 | 4 | 294 | AGGACAGGTCAGTCTCTGTC | 65 | 121 |
| 143616 | 4 | 295 | AAGGACAGGTCAGTCTCTGT | 53 | 122 |
| 143617 | 4 | 310 | CGCTCCCCTTTCTTGAAGGA | 78 | 123 |
| 143618 | 4 | 314 | CAGCCGCTCCCCTTTCTTGA | 88 | 124 |
| 143619 | 4 | 325 | TTGACAATCTGCAGCCGCTC | 62 | 125 |
| 143620 | 4 | 332 | CGTGTTATTGACAATCTGCA | 69 | 126 |
| 143621 | 4 | 346 | ACATCCACCTTCCTCGTGTT | 49 | 127 |
| 143622 | 4 | 376 | GAGTGTGCCAGCCACCAGTC | 73 | 128 |
| 143623 | 4 | 383 | GCTCAGCGAGTGTGCCAGCC | 82 | 129 |
| 143624 | 4 | 384 | TGCTCAGCGAGTGTGCCAGC | 79 | 130 |
| 143536 | 4 | 445 | TCAGCCTGGATGGAGTCGGA | 62 | 131 |
| 143537 | 4 | 446 | CTCAGCCTGGATGGAGTCGG | 67 | 132 |
| 143630 | 4 | 476 | CCGTGTAGTGATCTTGCCAA | 78 | 133 |
| 143631 | 4 | 485 | CTCTGATTCCCGTCTAGTGA | 86 | 134 |
| 143632 | 4 | 490 | AGCCGCTCTGATTCCCGTCT | 92 | 135 |
| 143633 | 4 | 533 | CCTCACGAGGAAGGTCCCTC | 54 | 136 |
| 143634 | 4 | 545 | GGTCTCACTCTCCGTCACGA | 86 | 137 |
| 143635 | 4 | 573 | ATACAGAGAGGCAGTAGGCA | 70 | 138 |
| 143636 | 4 | 578 | GTCGGATACAGAGAGGCAGT | 77 | 139 |
| 143638 | 4 | 601 | TTTAGGCCCTTGGCATTGTC | 84 | 140 |
| 143639 | 4 | 602 | ATTTAGGCCCTTGGCATTGT | 75 | 141 |
| 143640 | 4 | 611 | GTGTTTCACATTTAGGCCCT | 87 | 142 |
| 143643 | 4 | 707 | ATGTTTGGAGTAGTAAGCCA | 77 | 143 |
| 143644 | 4 | 718 | AGGCCATCAGCATGTTTGGA | 99 | 144 |
| 143645 | 4 | 727 | CGGTGACACAGGCCATCAGC | 82 | 145 |
| 143646 | 4 | 760 | TGAGGCTTGGATGTGGGACA | 45 | 146 |
| 143647 | 4 | 769 | CCCTGGGTCTGAGGCTTGGA | 81 | 147 |
| 143648 | 4 | 820 | ACCTCCAGCCGCAGGGACTC | 71 | 148 |
| 143556 | 4 | 827 | CAGCTTGACCTCCAGCCGCA | 75 | 149 |
| 143557 | 4 | 833 | CTGGCCCAGCTTGACCTCCA | 79 | 150 |
| 143649 | 4 | 839 | GCAACCCTGGCCCAGCTTGA | 72 | 151 |
| 143650 | 4 | 850 | ACCTCTCCGAAGCAACCCTG | 47 | 152 |
| 334149 | 4 | 855 | TCCACACCTCTCCGAAGCAA | 41 | 153 |
| 143651 | 4 | 878 | CGTGGTGCCGTTCCAGGTCC | 72 | 154 |
| 143652 | 4 | 889 | ATGGCAACCCTCGTGGTGCC | 40 | 155 |
| 143653 | 4 | 891 | TGATGGCAACCCTCGTGGTG | 55 | 156 |
| 334150 | 4 | 945 | CTTGGGCCTCCTGCAGGAAG | 61 | 157 |
| 143654 | 4 | 960 | TCAGTTTCTTCATGACTTGG | 60 | 158 |
| 143655 | 4 | 1038 | CCTTGTTCATGTACTCTGTC | 79 | 159 |
| 143656 | 4 | 1047 | GCAGACTCCCCTTGTTCATG | 65 | 160 |
| 143657 | 4 | 1049 | CAGCAGACTCCCCTTGTTCA | 61 | 161 |
| 143658 | 4 | 1070 | CGTTTCCCCCTTGAGAAAGT | 73 | 162 |
| 143659 | 4 | 1078 | TATTTGCCCGTTTCCCCCTT | 74 | 163 |
| 334151 | 4 | 1109 | AGACATGTCCACCAGCTGGG | 79 | 164 |
| 143660 | 4 | 1149 | TCATCCGCTCCACATAGGCC | 85 | 165 |
| 143661 | 4 | 1163 | CCGGTGCACATAGTTCATCC | 77 | 166 |
| 143570 | 4 | 1214 | CACTTTGCACACCAGGTTCT | 48 | 167 |
| 143571 | 4 | 1220 | GTCGGCCACTTTGCACACCA | 79 | 168 |
| 143572 | 4 | 1226 | CCCAAAGTCGGCCACTTTGC | 81 | 169 |
| 334152 | 4 | 1375 | GTGAGCTCAGTCAGCAGGAT | 73 | 170 |
| 143665 | 4 | 1458 | GACAAGGCATCCGGTAGCCC | 74 | 171 |
| 143577 | 4 | 1504 | CAGCACTGGCACATGAGGTC | 74 | 172 |
| 143578 | 4 | 1506 | GCCAGCACTGGCACATGAGG | 73 | 173 |
| 143579 | 4 | 1510 | TTCCGCCAGCACTGGCACAT | 72 | 174 |
| 143667 | 4 | 1513 | TCCTTCCGCCAGCAGTGGCA | 71 | 175 |
| 143580 | 4 | 1516 | GGCTCCTTCCGCCAGCACTG | 79 | 176 |
| 143581 | 4 | 1528 | GGCCGCTCCTCAGGCTCCTT | 79 | 177 |
| 143582 | 4 | 1529 | GGGCCGCTCCTCAGGCTCCT | 81 | 178 |
| 143583 | 4 | 1539 | ACTCGAAGGTGGGCCGCTCC | 71 | 179 |
| 143668 | 4 | 1610 | CTATAGGTTCTCCCCGGGCT | 93 | 180 |
| 334153 | 4 | 1619 | ACACAGTTCCTATAGGTTCT | 76 | 181 |
| 334154 | 5 | 5971 | CACCCCCTGTACAGACGGAA | 60 | 182 |
| 334155 | 5 | 10041 | TCAGCATGTTCTGGAGACGG | 65 | 183 |
| 334144 | 6 | 159 | ACACCAGCCGTAGTGTCCGG | 80 | 184 |
| 353105 | 7 | 69 | GTCCTGACTGCAGGGAGCAC | 36 | 185 |
| 353106 | 8 | 315 | CTACGAAGGAGAAGACAGTA | 8 | 186 |
| 353032 | 9 | 44 | CCGGTTCGGCTGCAGAGTCA | 28 | 187 |
| 353033 | 9 | 71 | GCAAGCCTGGGTCCCCAGGA | 33 | 188 |
| 353034 | 9 | 76 | ACTTTGCAAGCCTGGGTCCC | 33 | 189 |
| 353035 | 9 | 82 | ACCGTCACTTTGCAAGCCTG | 56 | 190 |
| 353036 | 9 | 117 | AGAAGGACTCAAGAGGGAGA | 13 | 191 |
| 353037 | 9 | 132 | CAGAGCCATCATCTCAGAAG | 25 | 192 |
| 353038 | 9 | 157 | AGACCCGGGTAGCTCCCGCT | 60 | 193 |
| 353039 | 9 | 202 | CCAGCAGAGGGTGGCCGCCG | 55 | 194 |
| 353040 | 9 | 237 | GGAATTGAGAACCGAGTTCA | 20 | 195 |
| 353041 | 9 | 291 | GCCTGGGTGCCCCGCAGCGC | 56 | 196 |
| 353042 | 9 | 303 | GCTGACTGCAGAGCCTGGGT | 54 | 197 |
| 353043 | 9 | 329 | CCCGGGTACAGGATTCCCGG | 20 | 198 |
| 353044 | 9 | 342 | GTACTTATTCCCGCCCGGGT | 46 | 199 |
| 353045 | 9 | 356 | TTGTCAATGGTCTGGTACTT | 40 | 200 |
| 353046 | 9 | 373 | ACGGGTACGGCTGGTAGTTG | 9 | 201 |
| 353047 | 9 | 399 | AGTGCCGCACTCCTCGTCCT | 39 | 202 |
| 353048 | 9 | 464 | CTGCAGGCGAGACAGATTTG | 13 | 203 |
| 353049 | 9 | 524 | TTTTTGCAGTAATTCCCGGG | 11 | 204 |
| 353050 | 9 | 533 | CATATTCCATTTTTGCAGTA | 4 | 205 |
| 353051 | 9 | 543 | AGAAGACACACATATTCCAT | 9 | 206 |
| 353052 | 9 | 575 | TCCTCAATTTCTCCTCGGAA | 49 | 207 |
| 353053 | 9 | 589 | TTTCAGTGATGGTTTCCTCA | 15 | 208 |
| 353054 | 9 | 601 | CATTACCAAAGCTTTCAGTG | 34 | 209 |
| 353055 | 9 | 632 | CTTCTGGAATACCCATCCAA | 5 | 210 |
| 353056 | 9 | 657 | ATACATTTTTGAAGACAAGG | 14 | 211 |
| 353057 | 9 | 681 | AGAACCTTCTTGTCCTTTGG | 30 | 212 |
| 353058 | 9 | 692 | CGGAGACAAACAGAACCTTC | 27 | 213 |
| 353059 | 9 | 698 | GATGACCGGAGACAAACAGA | 5 | 214 |
| 353060 | 9 | 719 | CACAATCCTGAGGCACAGTC | 23 | 215 |
| 353061 | 9 | 736 | AGAAGTGTCTAGCACAACAC | 32 | 216 |
| 353062 | 9 | 746 | ATCTTGGACCAGAAGTGTCT | 0 | 217 |
| 353063 | 9 | 751 | TACAGATCTTGGACCAGAAG | 36 | 218 |
| 353064 | 9 | 756 | AGGTTTACAGATCTTGGACC | 29 | 219 |
| 319435 | 9 | 760 | GGACAGGTTTACAGATCTTG | 5 | 220 |
| 353065 | 9 | 790 | TATGCTTGGTACACACTTGA | 31 | 221 |
| 353066 | 9 | 814 | CTAGTCCATGAGAGCCTTTT | 31 | 222 |
| 353067 | 9 | 844 | CTTCTCCACAGTAACAACGC | 9 | 223 |
| 353068 | 9 | 865 | TCTGTATCCGGCAAGACAGA | 37 | 224 |
| 353069 | 9 | 871 | GATCTTTCTGTATCCGGCAA | 55 | 225 |
| 319450 | 9 | 876 | ATGGTGATCTTTCTGTATCC | 46 | 226 |
| 353070 | 9 | 881 | GCTTGATGGTGATCTTTCTG | 57 | 227 |
| 353071 | 9 | 891 | AGAATTACTGGCTTGATGGT | 36 | 228 |
| 353072 | 9 | 904 | TGTGAAGCCTAGAAGAATTA | 23 | 229 |
| 353073 | 9 | 909 | ACAAGTGTGAAGCCTAGAAG | 36 | 230 |
| 353074 | 9 | 929 | TAGCTGGTTTAGTGTCTCTG | 69 | 231 |
| 353075 | 9 | 945 | GTTCACTGCATTTGGATAGC | 67 | 232 |
| 353076 | 9 | 971 | TTCATAGCATCTATTATATA | 15 | 233 |
| 353077 | 9 | 983 | TCATAAAAGGTTTTCATAGC | 18 | 234 |
| 353078 | 9 | 1007 | TCCTTAGGATTGAGTTGATG | 37 | 235 |
| 353079 | 9 | 1035 | GCTTAACTGAAACCACAGAA | 52 | 236 |
| 353080 | 9 | 1050 | AGGTGTTATTGGAATGCTTA | 42 | 237 |
| 353081 | 9 | 1067 | CACTCCAGGTTTTTGGAAGG | 33 | 238 |
| 353082 | 9 | 1079 | ACAAAGCTCTTACACTCCAG | 30 | 239 |
| 353083 | 9 | 1096 | GGGAGTTCCATAAAGAAACA | 28 | 240 |
| 353084 | 9 | 1114 | AATTTACTGCAATCACAGGG | 25 | 241 |
| 353085 | 9 | 1119 | ACAGTAATTTACTGCAATCA | 45 | 242 |
| 353086 | 9 | 1135 | ACTGAGAATTTACAATACAG | 18 | 243 |
| 353087 | 9 | 1149 | CAGGTAAGTGCCACACTGAG | 36 | 244 |
| 353088 | 9 | 1154 | ATTTACAGGTAAGTGCCACA | 27 | 245 |
| 353089 | 9 | 1192 | TGCAGCACCTTTAGAAAAAT | 21 | 246 |
| 353090 | 9 | 1203 | AAAATAGGCAGTGCAGCACC | 28 | 247 |
| 353091 | 9 | 1266 | ATATAGAATATTTGTCAGTC | 18 | 248 |
| 353092 | 9 | 1284 | ATGATTTACTTCAGTTCAAT | 7 | 249 |
| 353093 | 9 | 1304 | TTTAAGAACTATAAGCTGAA | 14 | 250 |
| 353094 | 9 | 1343 | TTCTAGACTCTAGAATTAAA | 27 | 251 |
| 353095 | 9 | 1380 | AGGTACCTATCATTTGTCAT | 46 | 252 |
| 353096 | 9 | 1441 | TAATTTAAGCATTAAGATAG | 9 | 253 |
| 353097 | 9 | 1467 | AAACTATCACAGCCTAAAGG | 8 | 254 |
| 319464 | 10 | 751 | CCCCTCTCACCTGGTAGTTG | 4 | 255 |
| 353098 | 10 | 1350 | TCTCTTCTTCCCGATTAAAC | 12 | 256 |
| 353099 | 10 | 1514 | TATCTGTATGATCCCATCGT | 25 | 257 |
| 353100 | 10 | 1969 | CTAAGTTAAGCAAATGCAAT | 1 | 258 |
| 353101 | 10 | 2153 | TCAGTCATAGGTAATATCCC | 17 | 259 |
| 353102 | 10 | 2368 | ACACATATTCCTAAGGAAGC | 1 | 260 |
| 353103 | 10 | 2509 | ACATCCTTACCTTTGGTGTG | 7 | 261 |
| 353104 | 10 | 2627 | CCTTCTTGTCCTACGAAGGA | 22 | 262 |
| 319464 | 11 | 3573 | CCCCTCTCACCTGGTAGTTG | 41 | 255 |
| 319465 | 11 | 3959 | CAGGACTCACCGTTTTTGCA | 50 | 263 |
| 319466 | 11 | 4037 | TAGCTTTGACAGAACCAGAG | 68 | 264 |
| 319467 | 11 | 4130 | TAAACTTTCAAATGCTCAAA | 31 | 265 |
| 319468 | 11 | 4310 | GAGAGAAGAGAGTTTGGCAA | 6 | 266 |
| 319469 | 11 | 4642 | TCTCACCCGCTAGTTACTIT | 90 | 267 |
| 319470 | 11 | 5009 | ATGCATATTCCTAAGAAAGC | 22 | 268 |
| 319471 | 11 | 5276 | CCTTCTTGCCCTGTAAAGGA | 26 | 269 |
| 319454 | 11 | 5532 | AGGCTGTTGGTTTTAGTGTC | 26 | 270 |
| 319457 | 11 | 5602 | CTTGGGATTGAGCTGACAAA | 71 | 271 |
| 319458 | 11 | 5607 | ACATCCTTGGGATTGAGCTG | 64 | 272 |
| 319459 | 11 | 5617 | GAAGATTCCTACATCCTTGG | 79 | 273 |
| 319460 | 11 | 5625 | TACACACTGAAGATTCCTAC | 82 | 274 |
| 319461 | 11 | 5634 | ATGCTTAATTACACACTGAA | 76 | 275 |
| 319462 | 11 | 5639 | TCGGAATGCTTAATTACACA | 76 | 276 |
| 319463 | 11 | 5676 | CAAAGTCCTTACACTCCAGA | 95 | 277 |
| 319396 | 12 | 12 | GGACAGCTGCCACTGCACGC | 78 | 278 |
| 319397 | 12 | 56 | AGGGAGGCTGCAGAGAGCCA | 73 | 279 |
| 319398 | 12 | 101 | GGAATTGATGAGAACTGAGT | 74 | 280 |
| 319399 | 12 | 106 | GCGTTGGAATTGATGAGAAC | 34 | 281 |
| 319400 | 12 | 109 | ATCGCGTTGGAATTGATGAG | 58 | 282 |
| 319401 | 12 | 111 | TGATCGCGTTGGAATTGATG | 42 | 283 |
| 319402 | 12 | 116 | GTTCTTGATCGCGTTGGAAT | 44 | 284 |
| 319403 | 12 | 121 | GGCAGGTTCTTGATCGCGTT | 42 | 285 |
| 319404 | 12 | 202 | TTGTTCCCGCCCTCATAGAG | 75 | 286 |
| 319405 | 12 | 207 | GGTACTTGTTCCCGCCCTCA | 94 | 287 |
| 319406 | 12 | 214 | AGAGTCTGGTACTTGTTCCC | 52 | 288 |
| 319407 | 12 | 226 | TGGTAGTTGTCAAGAGTCTG | 72 | 289 |
| 319408 | 12 | 232 | TAGGGCTGGTAGTTGTCAAG | 96 | 290 |
| 319409 | 12 | 326 | CAGGCAGATTTGTACACCTC | 84 | 291 |
| 319410 | 12 | 343 | CTGCGCTTTCGGCAAGCCAG | 63 | 292 |
| 319411 | 12 | 368 | CATAGCGTGCCTCATGCAGC | 86 | 293 |
| 319412 | 12 | 405 | TGCATATTCCGTTTTTGCAG | 62 | 294 |
| 319413 | 12 | 424 | TGGCTGTGGTCAGAGGGCAT | 76 | 295 |
| 319414 | 12 | 426 | AATGGCTGTGGTCAGAGGGC | 49 | 296 |
| 319415 | 12 | 429 | GTAAATGGCTGTGGTCAGAG | 40 | 297 |
| 319416 | 12 | 441 | TTTCCCCTCGAGGTAAATGG | 39 | 298 |
| 319417 | 12 | 457 | ATGATGCCTTCCTCGATTTC | 53 | 299 |
| 319418 | 12 | 460 | TCAATGATGCCTTCCTCGAT | 59 | 300 |
| 319419 | 12 | 464 | GTTITCAATGATGCCTTCCT | 77 | 301 |
| 319420 | 12 | 469 | CCAAGGTTTTCAATGATGCC | 56 | 302 |
| 319421 | 12 | 471 | TGCCAAGGTTTTCAATGATG | 52 | 303 |
| 319422 | 12 | 486 | CGGCACCGTGGTCATTGCCA | 30 | 304 |
| 319423 | 12 | 495 | ATCCATCCCCGGCACCGTGG | 56 | 305 |
| 319424 | 12 | 505 | CTTCTGGGATATCCATCCCC | 46 | 306 |
| 319425 | 12 | 510 | TGGTTCTTCTGGGATATCCA | 39 | 307 |
| 319426 | 12 | 515 | CAGTGTGGTTCTTCTGGGAT | 50 | 308 |
| 319427 | 12 | 528 | ATATTTTTGAAGTCAGTGTG | 26 | 309 |
| 319428 | 12 | 533 | GTGATATATTTTTGAAGTCA | 14 | 310 |
| 319429 | 12 | 547 | TCTTGCCCTTTGGTGTGATA | 41 | 311 |
| 319430 | 12 | 553 | GAGCCTTCTTGCCCTTTGGT | 55 | 312 |
| 319431 | 12 | 574 | TCTGATGATCGGAGGCAGAC | 57 | 313 |
| 319432 | 12 | 588 | GCCCTGTGGCGCAGTCTGAT | 53 | 314 |
| 319433 | 12 | 592 | CACAGCCCTGTGGCGCAGTC | 82 | 315 |
| 319434 | 12 | 610 | CAGAAATGTCTTGCACAACA | 70 | 316 |
| 319435 | 12 | 633 | GGACAGGTTTACAGATCTTG | 67 | 220 |
| 319436 | 12 | 644 | ACCTTCTTTAAGGACAGGTT | 40 | 317 |
| 319437 | 12 | 646 | TGACCTTCTTTAAGGACAGG | 53 | 318 |
| 319438 | 12 | 649 | ACCTGACCTTCTTTAAGGAC | 39 | 319 |
| 319439 | 12 | 662 | GTGCTTGGTGCATACGTGAC | 44 | 320 |
| 319442 | 12 | 686 | CAGCCCGTGGGAGCCTTTCC | 78 | 321 |
| 319443 | 12 | 689 | CTCCAGCCCGTGGGAGCCTT | 89 | 322 |
| 319444 | 12 | 705 | AACAGCGCTGGAATATCTCC | 20 | 323 |
| 319445 | 12 | 707 | GTAACAGCGCTGGAATATCT | 28 | 324 |
| 319446 | 12 | 722 | CAGACCTTCCCCACAGTAAC | 79 | 325 |
| 319447 | 12 | 739 | TTCTGTATCCTGCAAGCCAG | 92 | 326 |
| 319448 | 12 | 742 | TCTTTCTGTATCCTGCAAGC | 76 | 327 |
| 319449 | 12 | 744 | GATCTTTCTGTATCCTGCAA | 58 | 328 |
| 319450 | 12 | 749 | ATGGTGATCTTTCTGTATCC | 76 | 226 |
| 319451 | 12 | 776 | GTGGAGCCTGGAAGAATTGC | 37 | 329 |
| 319452 | 12 | 791 | GTGTCTCTGGCAGGTGTGGA | 57 | 330 |
| 319453 | 12 | 793 | TAGTGTCTCTGGCAGGTGTG | 62 | 331 |
| 331926 | 14 | 7 | TGAAATGTCCTGTTCCTGAC | 85 | 332 |
| 331927 | 14 | 31 | GCCGAAAGACCCTTGCTCCT | 27 | 333 |
| 331881 | 15 | 2 | AAAGACTTCGTTCTCTTGGC | 88 | 334 |
| 331882 | 15 | 30 | TTAAGTTCTCCCGCAAGAAA | 70 | 335 |
| 331883 | 15 | 38 | ATGCAGCATTAAGTTCTCCC | 87 | 336 |
| 331884 | 15 | 128 | CCCGACATGATGGCTCTTCT | 74 | 337 |
| 331885 | 15 | 132 | TCGCCCCGACATGATGGCTC | 64 | 338 |
| 117427 | 15 | 155 | TCCGCAAAGGAGGTGGTTCT | 68 | 339 |
| 117428 | 15 | 160 | AGCTCTCCGCAAAGGAGGTG | 74 | 340 |
| 117429 | 15 | 165 | CTTGCAGCTCTCCGCAAAGG | 79 | 341 |
| 117431 | 15 | 188 | AAAGCTGAAGGCTGCTGCAC | 19 | 342 |
| 117433 | 15 | 212 | TCTCTGCTAACTTTCATGCT | 63 | 343 |
| 331886 | 15 | 230 | ACCTTGCTGCCATCTTTATC | 65 | 344 |
| 117435 | 15 | 254 | CCAGGAGTTGCCACCACTGT | 60 | 345 |
| 331887 | 15 | 280 | CTTCCTGTGGCCTGTCAGGA | 29 | 346 |
| 117437 | 15 | 335 | TGATATACCACACCAAATGA | 45 | 347 |
| 117438 | 15 | 340 | TGGCTTGATATACCACACCA | 65 | 348 |
| 117439 | 15 | 345 | AAGTTTGGCTTGATATACCA | 38 | 349 |
| 117440 | 15 | 350 | TCACAAAGTTTGGCTTGATA | 53 | 350 |
| 331888 | 15 | 404 | TTCTTAAATCGCTTGTCCTG | 77 | 351 |
| 331889 | 15 | 425 | CTCATGATCTGGAGCTCTCG | 88 | 352 |
| 331890 | 15 | 430 | GCTTTCTCATGATCTGGAGC | 76 | 353 |
| 331891 | 15 | 435 | ATCTAGCTTTCTCATGATCT | 87 | 354 |
| 117444 | 15 | 441 | ACAGTGATCTAGCTTTCTCA | 81 | 355 |
| 117445 | 15 | 451 | GGACTATGTTACAGTGATCT | 76 | 356 |
| 331892 | 15 | 479 | CCACTCGAGTAGAAGAAATA | 38 | 357 |
| 117448 | 15 | 500 | TAGACCTCATCTTTCTTCTC | 57 | 358 |
| 331893 | 15 | 527 | GGAACATAGTCCAGCACCAG | 64 | 359 |
| 117451 | 15 | 536 | ACTGTTTCCGGAACATAGTC | 64 | 360 |
| 331894 | 15 | 556 | AGTGTCTGGCGACTCTGTAC | 75 | 361 |
| 117455 | 15 | 566 | GCTCGACTATAGTGTCTGGC | 87 | 362 |
| 331895 | 15 | 586 | TCACAGGGAGTGTCTGCTTG | 65 | 363 |
| 331896 | 15 | 608 | TACATATACAACTTGACATA | 51 | 364 |
| 117459 | 15 | 645 | AAAGGAATGGATATAGGCTA | 65 | 365 |
| 331897 | 15 | 668 | TTAATGTGTCGATGGCAGAT | 69 | 366 |
| 331898 | 15 | 682 | AGAGGTTCTGTGGTTTAATG | 78 | 367 |
| 331899 | 15 | 705 | TACAGCTGTATCAGGATCCA | 86 | 368 |
| 331900 | 15 | 737 | TGCTTTGCACTTCCAAAGTC | 70 | 369 |
| 331901 | 15 | 742 | CCAGCTGCTTTGCACTTCCA | 91 | 370 |
| 331902 | 15 | 747 | TCGGACCAGCTGCTTTGCAC | 64 | 371 |
| 331903 | 15 | 752 | TCTCCTCGGACCAGCTGCTT | 76 | 372 |
| 331904 | 15 | 785 | TAGTACCGAGAACAGATATA | 60 | 373 |
| 331905 | 15 | 792 | TGCCCTGTAGTACCGAGAAC | 71 | 374 |
| 331906 | 15 | 878 | CCTAGCAACAATTCAGCCAA | 68 | 375 |
| 117471 | 15 | 893 | GGAAATATTGGTTGTCCTAG | 78 | 376 |
| 331907 | 15 | 903 | ACTGTCCCCAGGAAATATTG | 49 | 377 |
| 117472 | 15 | 920 | ACCAACTGATCCACACCACT | 49 | 378 |
| 331908 | 15 | 941 | CCTAGGACCTTTATTATTTC | 77 | 379 |
| 331909 | 15 | 993 | GAATTCTGTATAATTTGGGT | 25 | 380 |
| 331910 | 15 | 1022 | CAAGGATGTGCCTTGATTTG | 48 | 381 |
| 117477 | 15 | 1124 | CAAGCTTCCAGTGGTGTTAG | 73 | 382 |
| 117478 | 15 | 1129 | GTGCACAAGCTTCCAGTGGT | 85 | 383 |
| 117479 | 15 | 1134 | TGAATGTGCACAAGCTTCCA | 72 | 384 |
| 117480 | 15 | 1139 | AAAAATGAATGTGCACAAGC | 68 | 385 |
| 117481 | 15 | 1149 | TAATTCATCAAAAAATGAAT | 5 | 386 |
| 117482 | 15 | 1159 | TTGGGTCCCGTAATTCATCA | 87 | 387 |
| 331911 | 15 | 1169 | AGTTTGACATTTGGGTCCCG | 90 | 388 |
| 331912 | 15 | 1174 | TTGGTAGTTTGACATTTGGG | 84 | 389 |
| 331913 | 15 | 1179 | CCCATTTGGTAGTTTGACAT | 81 | 390 |
| 331914 | 15 | 1184 | TCTCGCCCATTTGGTAGTTT | 78 | 391 |
| 331915 | 15 | 1189 | GTGTGTCTCGCCCATTTGGT | 99 | 392 |
| 117487 | 15 | 1226 | CTTGACAGTTCTTGAGTGGT | 75 | 393 |
| 331916 | 15 | 1322 | TTAGTATCTGAGGCTGCTGT | 57 | 394 |
| 117491 | 15 | 1344 | GGTCTGTCCACGGTCTCCAG | 78 | 395 |
| 117492 | 15 | 1349 | TTATTGGTCTGTCCACGGTC | 65 | 396 |
| 331917 | 15 | 1436 | TGGCACTCAAGTAAGTGCTG | 28 | 397 |
| 117497 | 15 | 1454 | GTGACCAGTGTTGCTGAGTG | 56 | 398 |
| 117499 | 15 | 1459 | CAAACGTGACCAGTGTTGCT | 68 | 399 |
| 117500 | 15 | 1464 | CTTTCCAAACGTGACCAGTG | 72 | 400 |
| 331918 | 15 | 1471 | TAATTTTCTTTCCAAACGTG | 75 | 401 |
| 331919 | 16 | 618 | CGATACTCACTGCTAACTTT | 37 | 402 |
| 331920 | 16 | 32579 | CCATACTTTTGGAATAACAA | 60 | 403 |
| 331921 | 16 | 77071 | AATTTGCAGACTCAGAACTG | 44 | 404 |
| 331922 | 16 | 97248 | CTGCTTTGCACTGATGAAAA | 48 | 405 |
| 331923 | 16 | 104117 | ACAGTTTTTCACCTACTAGC | 70 | 406 |
| 331924 | 16 | 123838 | AAGTACATACCCGTGCTCCT | 55 | 407 |
| 331925 | 16 | 126520 | AAAATGAATGTGCACAAGCT | 65 | 408 |
| 117469 | 17 | 823 | GCAGACCATACATCTATACT | 71 | 409 |
| 279474 | 18 | 4 | AGGAGGAAGGGCACTCGGTC | 13 | 410 |
| 279475 | 18 | 25 | TGAGAGCTGCATGGTGCCAG | 50 | 411 |
| 279476 | 18 | 62 | CTGCATGTACAAGCAGGCAG | 51 | 412 |
| 279477 | 18 | 67 | GAAGGCTGCATGTACAAGCA | 2 | 413 |
| 279478 | 18 | 72 | GCAACGAAGGCTGCATGTAC | 0 | 414 |
| 279479 | 18 | 84 | TGGCTCTCCACAGCAACGAA | 8 | 415 |
| 279480 | 18 | 100 | GAAGGCTTGCCACCCCTGGC | 53 | 416 |
| 279481 | 18 | 105 | TTCTTGAAGGCTTGCCACCC | 7 | 417 |
| 279482 | 18 | 110 | CATCATTCTTGAAGGCTTGC | 12 | 418 |
| 279483 | 18 | 115 | TGTGGCATCATTCTTGAAGG | 26 | 419 |
| 279484 | 18 | 132 | AGTCCCGGGATGATTTCTGT | 44 | 420 |
| 279485 | 18 | 142 | GTACTCTCTGAGTCCCGGGA | 28 | 421 |
| 279486 | 18 | 148 | CTCTGGGTACTCTCTGAGTC | 6 | 422 |
| 279487 | 18 | 153 | GGAGGCTCTGGGTACTCTCT | 46 | 423 |
| 279488 | 18 | 161 | GTTCCTGAGGAGGCTCTGGG | 10 | 424 |
| 279489 | 18 | 166 | CTCTAGTTCCTGAGGAGGCT | 38 | 425 |
| 279490 | 18 | 171 | TTGTTCTCTAGTTCCTGAGG | 51 | 426 |
| 279491 | 18 | 185 | GGTTCATGGTCTGGTTGTTC | 43 | 427 |
| 279492 | 18 | 186 | CGGTTCATGGTCTGGTTGTT | 36 | 428 |
| 279493 | 18 | 187 | CCGGTTCATGGTCTGGTTGT | 12 | 429 |
| 279494 | 18 | 190 | GGCCCGGTTCATGGTCTGGT | 44 | 430 |
| 279495 | 18 | 192 | TCGGCCCGGTTCATGGTCTG | 52 | 431 |
| 279496 | 18 | 194 | TCTCGGCCCGGTTCATGGTC | 45 | 432 |
| 279497 | 18 | 233 | CTTTGGTGTCATAAGGATGG | 44 | 433 |
| 279498 | 18 | 235 | GTCTTTGGTGTCATAAGGAT | 37 | 434 |
| 279499 | 18 | 236 | CGTCTTTGGTGTCATAAGGA | 20 | 435 |
| 279500 | 18 | 242 | CGGACACGTCTTTGGTGTCA | 30 | 436 |
| 279501 | 18 | 244 | CTCGGACACGTCTTTGGTGT | 0 | 437 |
| 279502 | 18 | 247 | GTACTCGGACACGTCTTTGG | 25 | 438 |
| 279503 | 18 | 250 | GCTGTACTCGGACACGTCTT | 29 | 439 |
| 279504 | 18 | 254 | GGCAGCTGTACTCGGACACG | 44 | 440 |
| 279505 | 18 | 255 | CGGCAGCTGTACTCGGACAC | 63 | 441 |
| 279506 | 18 | 261 | AGCTCGCGGCAGCTGTACTC | 38 | 442 |
| 279507 | 18 | 263 | GCAGCTCGCGGCAGCTGTAC | 25 | 443 |
| 279508 | 18 | 264 | TGCAGCTCGCGGCAGCTGTA | 0 | 444 |
| 279509 | 18 | 265 | GTGCAGCTCGCGGCAGCTGT | 5 | 445 |
| 279510 | 18 | 267 | TAGTGCAGCTCGCGGCAGCT | 45 | 446 |
| 279511 | 18 | 269 | TGTAGTGCAGCTCGCGGCAG | 41 | 447 |
| 279512 | 18 | 271 | GGTGTAGTGCAGCTCGCGGC | 42 | 448 |
| 279513 | 18 | 274 | GCGGGTGTAGTGCAGCTCGC | 36 | 449 |
| 279514 | 18 | 276 | AAGCGGGTGTAGTGCAGCTC | 35 | 450 |
| 279515 | 18 | 280 | CACGAAGCGGGTGTAGTGCA | 0 | 451 |
| 279516 | 18 | 282 | GTCACGAAGCGGGTGTAGTG | 14 | 452 |
| 279517 | 18 | 313 | GACCGGCTTGGCACTGCGGC | 55 | 453 |
| 279518 | 18 | 320 | ACTCGGTGACCGGCTTGGCA | 3 | 454 |
| 279519 | 18 | 321 | AACTCGGTGACCGGCTTGGC | 28 | 455 |
| 279520 | 18 | 322 | CAACTCGGTGACCGGCTTGG | 0 | 456 |
| 279521 | 18 | 323 | CCAACTCGGTGACCGGCTTG | 12 | 457 |
| 279522 | 18 | 326 | ACACCAACTCGGTGACCGGC | 49 | 458 |
| 279523 | 18 | 330 | GAGCACACCAACTCGGTGAC | 21 | 459 |
| 279524 | 18 | 334 | GCCCGAGCACACCAACTCGG | 47 | 460 |
| 279525 | 18 | 338 | ACTGGCCCGAGCACACCAAC | 39 | 461 |
| 279526 | 18 | 418 | GATGCAGCGGAAGTCGGGTC | 15 | 462 |
| 279527 | 18 | 430 | GTAGCGATCCGGGATGCAGC | 38 | 463 |
| 279528 | 18 | 453 | AGCAGCTGCACCCGCTGCGC | 3 | 464 |
| 279529 | 18 | 455 | ACAGCAGCTGCACCCGCTGC | 0 | 465 |
| 279530 | 18 | 458 | GGCACAGCAGCTGCACCCGC | 27 | 466 |
| 279531 | 18 | 501 | GCCACCAGACGCACCTTGCG | 12 | 467 |
| 279532 | 18 | 505 | CGAGGCCACCAGACGCACCT | 49 | 468 |
| 279533 | 18 | 509 | TGCACGAGGCCACCAGACGC | 38 | 469 |
| 279534 | 18 | 510 | TTGCACGAGGCCACCAGACG | 30 | 470 |
| 279535 | 18 | 514 | GCACTTGCACGAGGCCACCA | 39 | 471 |
| 279536 | 18 | 516 | TTGCACTTGCACGAGGCCAC | 61 | 472 |
| 279537 | 18 | 519 | CGCTTGCACTTGCACGAGGC | 55 | 473 |
| 279538 | 18 | 545 | CCGACTGGTTGTGGAAGCGG | 60 | 474 |
| 279539 | 18 | 547 | CTCCGACTGGTTGTGGAAGC | 46 | 475 |
| 279540 | 18 | 550 | GAGCTCCGACTGGTTGTGGA | 41 | 476 |
| 279541 | 18 | 555 | TCCTTGAGCTCCGACTGGTT | 51 | 477 |
| 279542 | 18 | 556 | GTCCTTGAGCTCCGACTGGT | 2 | 478 |
| 279543 | 18 | 560 | CGAAGTCCTTGAGCTCCGAC | 0 | 479 |
| 279544 | 18 | 564 | GGTCCGAAGTCCTTGAGCTC | 64 | 480 |
| 279545 | 18 | 595 | CTTGCGACCCTTCTGCGGCC | 36 | 481 |
| 279546 | 18 | 596 | GCTTGCGACCCTTCTGCGGC | 38 | 482 |
| 279547 | 18 | 599 | GCGGCTTGCGACCCTTCTGC | 42 | 483 |
| 279548 | 18 | 639 | AGCTCCGCCTGGTTGGCTTT | 49 | 484 |
| 279549 | 18 | 640 | CAGCTCCGCCTGGTTGGCTT | 22 | 485 |
| 279550 | 18 | 644 | TCTCCAGCTCCGCCTGGTTG | 39 | 486 |
| 279551 | 18 | 645 | TTCTCCAGCTCCGCCTGGTT | 0 | 487 |
| 299395 | 19 | 17 | AAGAACTGCATGACCGGCTC | 55 | 488 |
| 299396 | 19 | 19 | CGAAGAACTGCATGACCGGC | 81 | 489 |
| 299397 | 19 | 21 | GCCGAAGAACTGCATGACCG | 51 | 490 |
| 279712 | 19 | 24 | GAAGCCGAAGAACTGCATGA | 33 | 491 |
| 299398 | 19 | 27 | GTAGAAGCCGAAGAACTGCA | 51 | 492 |
| 299399 | 19 | 33 | CGGCCAGTAGAAGCCGAAGA | 30 | 493 |
| 299400 | 19 | 37 | TCTCCGGCCAGTAGAAGCCG | 55 | 494 |
| 299401 | 19 | 42 | GAGCATCTCCGGCCAGTAGA | 65 | 495 |
| 299402 | 19 | 49 | CACATTTGAGCATCTCCGGC | 80 | 496 |
| 299403 | 19 | 51 | GTCACATTTGAGCATCTCCG | 80 | 497 |
| 299404 | 19 | 55 | ACTTGTCACATTTGAGCATC | 70 | 498 |
| 299405 | 19 | 59 | GGGAACTTGTCACATTTGAG | 52 | 499 |
| 279722 | 19 | 109 | AGGCTTCGGTGGCATTGGGC | 80 | 500 |
| 299406 | 19 | 113 | TTCGAGGCTTCGGTGGCATT | 66 | 501 |
| 299407 | 19 | 116 | GGCTTCGAGGCTTCGGTGGC | 71 | 502 |
| 299408 | 19 | 137 | GGACACACTGTTGTACCTTG | 39 | 503 |
| 279727 | 19 | 145 | CACACGGAGGACACACTGTT | 18 | 504 |
| 299409 | 19 | 147 | GTCACACGGAGGACACACTG | 71 | 505 |
| 299410 | 19 | 152 | TCGTTGTCACACGGAGGACA | 76 | 506 |
| 299411 | 19 | 158 | TTCAACTCGTTGTCACACGG | 61 | 507 |
| 299412 | 19 | 162 | CGATTTCAACTCGTTGTCAC | 65 | 508 |
| 299413 | 19 | 166 | CCTCCGATTTCAACTCGTTG | 29 | 509 |
| 299414 | 19 | 168 | GGCCTCCGATTTCAACTCGT | 71 | 510 |
| 299415 | 19 | 170 | ATGGCCTCCGATTTCAACTC | 79 | 511 |
| 299416 | 19 | 172 | TGATGGCCTCCGATTTCAAC | 68 | 512 |
| 299417 | 19 | 176 | TCGATGATGGCCTCCGATTT | 70 | 513 |
| 299418 | 19 | 178 | GTTCGATGATGGCCTCCGAT | 82 | 514 |
| 299419 | 19 | 181 | GATGTTCGATGATGGCCTCC | 72 | 515 |
| 299420 | 19 | 186 | ACAGAGATGTTCGATGATGG | 54 | 516 |
| 299421 | 19 | 188 | GCACAGAGATGTTCGATGAT | 73 | 517 |
| 299422 | 19 | 190 | TTGCACAGAGATGTTCGATG | 75 | 518 |
| 299423 | 19 | 196 | ACTCGCTTGCACAGAGATGT | 68 | 519 |
| 299424 | 19 | 197 | AACTCGCTTGCACAGAGATG | 67 | 520 |
| 299425 | 19 | 200 | GCAAACTCGCTTGCACAGAG | 66 | 521 |
| 299426 | 19 | 204 | CAGCGCAAACTCGCTTGCAC | 59 | 522 |
| 299427 | 19 | 207 | TCTCAGCGCAAACTCGCTTG | 55 | 523 |
| 299428 | 19 | 211 | TCATTCTCAGCGCAAACTCG | 45 | 524 |
| 299429 | 19 | 215 | ATTTTCATTCTCAGCGCAAA | 51 | 525 |
| 299430 | 19 | 218 | TTGATTTTCATTCTCAGCGC | 83 | 526 |
| 299431 | 19 | 256 | GGACAATCTTCTTGTCACCG | 69 | 527 |
| 299432 | 19 | 282 | CAGCTTCAAGGGTTTCTTCT | 59 | 528 |
| 299433 | 19 | 284 | CCCAGCTTCAAGGGTTTCTT | 65 | 529 |
| 279749 | 19 | 287 | GGCCCCAGCTTCAAGGGTTT | 61 | 530 |
| 279750 | 19 | 291 | GATGGGCCCCAGCTTCAAGG | 88 | 531 |
| 299434 | 19 | 295 | TCTTGATGGGCCCCAGCTTC | 75 | 532 |
| 299435 | 19 | 303 | CTCCTTCTTCTTGATGGGCC | 76 | 533 |
| 279755 | 19 | 304 | GCTCCTTCTTCTTGATGGGC | 36 | 534 |
| 299436 | 19 | 313 | GCCGCTTCAGCTCCTTCTTC | 80 | 535 |
| 299437 | 19 | 315 | GAGCCGCTTCAGCTCCTTCT | 75 | 536 |
| 299438 | 19 | 319 | GCACGAGCCGCTTCAGCTCC | 75 | 537 |
| 299439 | 19 | 322 | AAAGCACGAGCCGCTTCAGC | 60 | 538 |
| 299440 | 19 | 324 | GAAAAGCACGAGCCGCTTCA | 34 | 539 |
| 299441 | 19 | 329 | TTTAGGAAAAGCACGAGCCG | 71 | 540 |
| 299442 | 19 | 362 | TCCAGCTGGTGGCAGGGACA | 29 | 541 |
| 299443 | 19 | 366 | GTTGTCCAGCTGGTGGCAGG | 80 | 542 |
| 299444 | 19 | 370 | TGAGGTTGTCCAGCTGGTGG | 67 | 543 |
| 299445 | 19 | 376 | TGTGGCTGAGGTTGTCCAGC | 63 | 544 |
| 299446 | 19 | 380 | AAGTTGTGGCTGAGGTTGTC | 61 | 545 |
| 299447 | 19 | 383 | AGGAAGTTGTGGCTGAGGTT | 64 | 546 |
| 299448 | 19 | 388 | TGATGAGGAAGTTGTGGCTG | 62 | 547 |
| 299449 | 19 | 390 | CATGATGAGGAAGTTGTGGC | 57 | 548 |
| 299450 | 19 | 392 | CCCATGATGAGGAAGTTGTG | 55 | 549 |
| 299451 | 19 | 394 | GCCCCATGATGAGGAAGTTG | 47 | 550 |
| 299452 | 19 | 396 | GCGCCCCATGATGAGGAAGT | 78 | 551 |
| 299453 | 19 | 398 | TTGCGCCCCATGATGAGGAA | 51 | 552 |
| 299454 | 19 | 400 | CCTTGCGCCCCATGATGAGG | 70 | 553 |
| 299455 | 19 | 402 | CACCTTGCGCCCCATGATGA | 39 | 554 |
| 299456 | 19 | 405 | CTTCACCTTGCGCCCCATGA | 39 | 555 |
| 299457 | 19 | 410 | TGGCTCTTCACCTTGCGCCC | 77 | 556 |
| 299458 | 19 | 412 | ACTGGCTCTTCACCTTGCGC | 85 | 557 |
| 299459 | 19 | 414 | GTACTGGCTCTTCACCTTGC | 77 | 558 |
| 299460 | 19 | 422 | GTGAGCAAGTACTGGCTCTT | 63 | 559 |
| 299461 | 19 | 428 | ATGGCTGTGAGCAAGTACTG | 74 | 560 |
| 299462 | 19 | 430 | GAATGGCTGTGAGCAAGTAC | 26 | 561 |
| 299463 | 19 | 439 | CCCACTTGTGAATGGCTGTG | 86 | 562 |
| 299464 | 19 | 441 | GTCCCACTTGTGAATGGCTG | 83 | 563 |
| 299465 | 19 | 443 | TTGTCCCACTTGTGAATGGC | 58 | 564 |
| 299466 | 19 | 446 | TTCTTGTCCCACTTGTGAAT | 53 | 565 |
| 180922 | 20 | 22 | CTCTACGCCACAAAATTAGG | 0 | 566 |
| 180938 | 20 | 27 | CATAGCTCTACGCCACAAAA | 8 | 567 |
| 180926 | 20 | 85 | GAAGCTTATTGTACAAATAC | 49 | 568 |
| 180963 | 20 | 95 | CGTCTCCTGGGAAGCTTATT | 59 | 569 |
| 180927 | 20 | 108 | AAAAATGTTGACACGTCTCC | 52 | 570 |
| 180928 | 20 | 185 | CCCGATCCTTTGTATGGCTT | 54 | 571 |
| 180934 | 20 | 197 | ATACATCTAAACCCCGATCC | 25 | 572 |
| 180924 | 20 | 205 | CTATGTGTATACATCTAAAC | 11 | 573 |
| 180930 | 20 | 241 | TGGATGGTTGTTCAATCACT | 46 | 574 |
| 180964 | 20 | 260 | ATGTCCAAACCACTGTGTTT | 0 | 575 |
| 180948 | 20 | 261 | AATGTCCAAACCACTCTCTT | 6 | 576 |
| 180935 | 20 | 408 | GATCTCCTTATCCAACTCAC | 37 | 577 |
| 180940 | 20 | 478 | AGCTGGACACTGATGAGGCT | 21 | 578 |
| 180945 | 20 | 486 | CGATGGAGAGCTGGACACTG | 33 | 579 |
| 180944 | 20 | 487 | GCGATGGAGAGCTGGACACT | 30 | 580 |
| 180961 | 20 | 513 | TACAGCAGCAGAGTGACCAA | 0 | 581 |
| 180960 | 20 | 529 | GCATGAAGGTAGGGCTTACA | 19 | 582 |
| 180941 | 20 | 574 | CAGCAAAAGTGGCAGTGGTA | 22 | 583 |
| 180946 | 20 | 598 | TTTTGGTAGAGCCGAACTTG | 0 | 584 |
| 180925 | 20 | 604 | TCTTCATTTTGGTAGAGCCG | 34 | 585 |
| 180936 | 20 | 638 | GAAGTACGTGCAACCTTGTT | 39 | 586 |
| 180923 | 20 | 663 | CACATTCAAGCCGAGGTTGA | 36 | 587 |
| 180951 | 20 | 694 | AAGAGATGGCTTTCTGCTTC | 11 | 588 |
| 180932 | 20 | 699 | TGAGGAAGAGATGGCTTTCT | 37 | 589 |
| 180962 | 20 | 736 | GCTGGCTACCCAAGCCAAGC | 24 | 590 |
| 180957 | 20 | 738 | CTGCTGGCTACCCAAGCCAA | 4 | 591 |
| 180933 | 20 | 739 | GCTGCTGGCTACCCAAGCCA | 31 | 592 |
| 180942 | 20 | 855 | AGGAAAAATAAATTCCTTGG | 43 | 593 |
| 180955 | 20 | 866 | CCCTGCATATTAGGAAAAAT | 11 | 594 |
| 180943 | 20 | 891 | CATTCCATTGGTACTACTAC | 27 | 595 |
| 180931 | 20 | 905 | CTGTCACCTGGGAACATTCC | 26 | 596 |
| 180939 | 20 | 938 | TTACTGTACTGGAGAGGACT | 41 | 597 |
| 180953 | 20 | 1002 | ATTCAAGCCATCTACAAAAG | 12 | 598 |
| 180937 | 20 | 1024 | ACTGCATGTTATTTAAGCTA | 66 | 599 |
| 180949 | 20 | 1047 | AGGCTGGAATTGCTGGTTAG | 30 | 600 |
| 180947 | 20 | 1064 | TTTTAGTTAGCCATAACAGG | 30 | 601 |
| 180929 | 20 | 1180 | CCCAAGCTTGAATGTATCCT | 58 | 602 |
| 332005 | 21 | 9 | GAAGATTATCCTTCAACCTT | 30 | 603 |
| 332006 | 21 | 33 | TTCTCATTCAAAGTGCTGGT | 44 | 604 |
| 332007 | 21 | 50 | TGTGGCAGAGAAACAAATTC | 28 | 605 |
| 332008 | 21 | 99 | GTTTCAACTCCTCCACCAGA | 64 | 606 |
| 332009 | 21 | 111 | AAAGGTAGGTTTTGTTCAAC | 57 | 607 |
| 332010 | 21 | 137 | TCAAGCTGCATAGCTGCGAT | 67 | 608 |
| 332011 | 21 | 161 | ATAAAATTTAGTGCTACTTG | 16 | 609 |
| 332012 | 21 | 191 | CTGGGAAGCTTATTGTACAA | 37 | 610 |
| 332013 | 21 | 196 | GTCTCCTGGGAAGCTTATTG | 57 | 611 |
| 332014 | 21 | 201 | GACACGTCTCCTGGGAAGCT | 71 | 612 |
| 332015 | 21 | 206 | ATGTTGACACGTCTCCTGGG | 72 | 613 |
| 332016 | 21 | 211 | CAAAAATGTTGACACGTCTC | 50 | 614 |
| 332017 | 21 | 216 | TTCACCAAAAATGTTGACAC | 20 | 615 |
| 332018 | 21 | 223 | CAAGTTGTTCACCAAAAATG | 25 | 616 |
| 332019 | 21 | 228 | TCTTTCAAGTTCTTCACCAA | 33 | 617 |
| 332020 | 21 | 233 | AGAAGTCTTTCAAGTTCTTC | 33 | 618 |
| 332021 | 21 | 281 | CCTTTGTATGGCTTCTCAGG | 50 | 619 |
| 332022 | 21 | 287 | CCTGAGCCTTTGTATGGCTT | 38 | 620 |
| 332023 | 21 | 292 | TAAACCCTGAGCCTTTGTAT | 19 | 621 |
| 332024 | 21 | 358 | CCAGACCACTCTCTTTGGAC | 78 | 622 |
| 332025 | 21 | 375 | ACGAACATCGTCAATGTCCA | 42 | 623 |
| 332026 | 21 | 380 | TTGCCACGAACATCGTCAAT | 29 | 624 |
| 332027 | 21 | 386 | GGCAGATTGCCACGAACATC | 41 | 625 |
| 332028 | 21 | 423 | AACCTCAAACGGGTCGATCC | 48 | 626 |
| 332029 | 21 | 466 | CGTACAGCACCTTCACTGGT | 57 | 627 |
| 332030 | 21 | 482 | TCATTACTGTCATCTACGTA | 55 | 628 |
| 332031 | 21 | 487 | CGTTCTCATTACTGTCATCT | 57 | 629 |
| 332032 | 21 | 495 | CTCACACCCGTTCTCATTAC | 47 | 630 |
| 332033 | 21 | 503 | TTATCCAGCTCACACCCGTT | 59 | 631 |
| 332034 | 21 | 509 | ATCTCCTTATCCAGCTCACA | 38 | 632 |
| 332035 | 21 | 575 | GACACAGAGGAGGCTGGGTC | 46 | 633 |
| 332036 | 21 | 615 | GACGGCAGCAGAATGGCCAA | 10 | 634 |
| 332037 | 21 | 651 | TAAAGGCTGAGTGGACCGGG | 55 | 635 |
| 332038 | 21 | 656 | AAGGTTAAAGGCTGAGTGGA | 31 | 636 |
| 332039 | 21 | 662 | GTGGTAAAGGTTAAAGGCTG | 21 | 637 |
| 332040 | 21 | 667 | TGGCAGTGGTAAAGGTTAAA | 46 | 638 |
| 332041 | 21 | 672 | AAAAGTGGCAGTGGTAAAGG | 49 | 639 |
| 332042 | 21 | 728 | ACCTTGCTGCTACGGCCACT | 70 | 640 |
| 332043 | 21 | 744 | GGGAGAAGTGCGTGCTACCT | 36 | 641 |
| 332044 | 21 | 770 | ACATTGACATTCAGGCCCAG | 69 | 642 |
| 332045 | 21 | 787 | GCTTCAGGAGGTCGTTCACA | 92 | 643 |
| 332046 | 21 | 795 | GGCTTTCTGCTTCAGGAGGT | 94 | 644 |
| 332047 | 21 | 801 | AGAGATGGCTTTCTGCTTCA | 62 | 645 |
| 332048 | 21 | 806 | GAGGAAGAGATGGCTTTCTG | 80 | 646 |
| 332049 | 21 | 811 | GCATTGAGGAAGAGATGGCT | 83 | 647 |
| 332050 | 21 | 816 | AGAGTGCATTGAGGAAGAGA | 42 | 648 |
| 332051 | 21 | 821 | TACAGAGAGTGCATTGAGGA | 62 | 649 |
| 332052 | 21 | 844 | GCTGGCTGCCCAGGCCCAGC | 11 | 650 |
| 332053 | 21 | 849 | CTGCTGCTGGCTGCCCAGGC | 77 | 651 |
| 332054 | 21 | 867 | CTGCGGCTGCGGCTGAGGCT | 38 | 652 |
| 332055 | 21 | 1128 | TCCGTAGGCCGCAAACACAT | 46 | 653 |
| 332056 | 21 | 1133 | AGGCCTCCGTAGGCCGCAAA | 31 | 654 |
| 332057 | 21 | 1138 | CGTTGAGGCCTCCGTAGGCC | 42 | 655 |
| 332058 | 21 | 1227 | TTTTTAGTTAGCCATTACGG | 43 | 656 |
| 332059 | 21 | 1253 | CGACACATGTTCTCTCTTTT | 88 | 657 |
| 332060 | 21 | 1259 | CTTGTACGACACATGTTCTC | 71 | 658 |
| 332061 | 21 | 1271 | GATGCATTTTAACTTGTACG | 65 | 659 |
| 332062 | 21 | 1279 | CTTGGGCCGATGCATTTTAA | 75 | 660 |
| 332063 | 21 | 1284 | TCCCCCTTGGGCCGATGCAT | 77 | 661 |
| 332064 | 21 | 1336 | CCTTACTATAAGCTTAAAAA | 32 | 662 |
| 332065 | 21 | 1341 | TGTATCCTTACTATAAGCTT | 69 | 663 |
| 332066 | 21 | 1346 | TTGAATGTATCCTTACTATA | 60 | 664 |
| 332067 | 21 | 1351 | CAAGCTTGAATGTATCCTTA | 72 | 665 |
| 332068 | 21 | 1394 | CTTGGTTGGCAAATGAAAAA | 33 | 666 |
| 332069 | 21 | 1409 | TAAAATAACATTGTGCTTGG | 34 | 667 |
| 332070 | 21 | 1435 | GTATACTTTAAAATATACAG | 0 | 668 |
| 332071 | 21 | 1445 | ATATCTGAAAGTATACTTTA | 0 | 669 |
| 332072 | 21 | 1478 | GTCCTTGCTATATCTTAAAT | 77 | 670 |
| 332073 | 21 | 1531 | CCCACTTATTAGTGCCAATT | 75 | 671 |
| 332074 | 21 | 1574 | CTTTGTACTAAATTAAATTA | 0 | 672 |
| 332075 | 21 | 1581 | TTACAAACTTTGTACTAAAT | 21 | 673 |
| 332076 | 21 | 1640 | GCAATACCGTGTCGTAGAAA | 77 | 674 |
| 332077 | 21 | 1675 | GCTGCCACAGATCACTGTAG | 64 | 675 |
| 332078 | 21 | 1684 | CATGAAGCCGCTGCCACAGA | 55 | 676 |
| 332079 | 21 | 1726 | CTTTAAGATGGATATTTTAC | 19 | 677 |
| 332080 | 21 | 1731 | GATGTCTTTAAGATGGATAT | 45 | 678 |
| 332081 | 21 | 1754 | TGTACACAATTTTCAGAATA | 39 | 679 |
| 332082 | 21 | 1771 | CCACTAAAGGAATATCCTGT | 43 | 680 |

### EXAMPLE 5

Design and Screening of Duplexed Oligomeric Compounds Targeting a Bone Growth Modulator

In accordance with the invention, a series of duplexes, including dsRNA and mimetics thereof, comprising oligomeric compounds of the invention and their complements can be designed to target a bone growth modulator. The nucleobase sequence of the antisense strand of the duplex comprises at least a portion of an oligonucleotide targeted to a bone growth modulator as disclosed herein. The ends of the strands may be modified by the addition of one or more natural or modified nucleobases to form an overhang. The sense strand of the nucleic acid duplex is then designed and synthesized as the complement of the antisense strand and may also contain modifications or additions to either terminus. The antisense and sense strands of the duplex comprise from about 17 to 25 nucleotides, or from about 19 to 23 nucleotides. Alternatively, the antisense and sense strands comprise 20, 21 or 22 nucleotides.

For example, in one embodiment, both strands of the dsRNA duplex would be complementary over the central nucleobases, each having overhangs at one or both termini.

For example, a duplex comprising an antisense strand having the sequence CGAGAGGCGGACGGGACCG and having a two-nucleobase overhang of deoxythymidine(dT) would have the following structure:

| | |
|---|---|
| cgagaggcggacgggaccgTT | Antisense Strand |
| \|\|\|\|\|\|\|\|\|\|\|\|\|\|\|\|\|\|\| | |
| TTgctctccgcctgccctggc | Complement |

Overhangs can range from 2 to 6 nucleobases and these nucleobases may or may not be complementary to the target nucleic acid. In another embodiment, the duplexes can have an overhang on only one terminus.

In another embodiment, a duplex comprising an antisense strand having the same sequence, for example CGAGAGGCGGACGGGACCG, can be prepared with blunt ends (no single stranded overhang) as shown:

| | |
|---|---|
| cgagaggcggacgggaccg | Antisense Strand |
| \|\|\|\|\|\|\|\|\|\|\|\|\|\|\|\|\|\|\| | |
| gctctccgcctgccctggc | Complement |

The RNA duplex can be unimolecular or bimolecular; i.e, the two strands can be part of a single molecule or may be separate molecules.

RNA strands of the duplex can be synthesized by methods routine to the skilled artisan or purchased from Dharmacon Research Inc. (Lafayette, Colo.). Once synthesized, the complementary strands are annealed. The single strands are aliquoted and diluted to a concentration of 50 uM. Once diluted, 30 uL of each strand is combined with 15 uL of a 5× solution of annealing buffer. The final concentration of said buffer is 100 mM potassium acetate, 30 mM HEPES-KOH pH 7.4, and 2 mM magnesium acetate. The final volume is 75 uL. This solution is incubated for 1 minute at 90° C. and then centrifuged for 15 seconds. The tube is allowed to sit for 1 hour at 37° C. at which time the dsRNA duplexes are used in experimentation. The final concentration of the dsRNA duplex is 20 uM.

Once prepared, the duplexed compounds are evaluated for their ability to modulate a bone growth modulator. When cells reached 80% confluency, they are treated with duplexed compounds of the invention. For cells grown in 96-well plates, wells are washed once with 200 μL OPTI-MEM-1™ reduced-serum medium (Gibco BRL) and then treated with 130 μL of OPTI-MEM-1™ containing 12 μg/mL LIPOFECTIN™ (Gibco BRL) and the desired duplex antisense compound at a final concentration of 200 nM (a ratio of 6 μg/mL LIPOFECTIN™ per 100 nM duplex antisense compound). After 5 hours of treatment, the medium is replaced with fresh medium. Cells are harvested 16 hours after treatment, at which time RNA is isolated and target reduction measured by RT-PCR.

### EXAMPLE 6

Phenotypic Assays

Selected oligomeric compounds were evaluated in functional assays, for the purpose of identifying compounds which modulate angiogenesis, cell proliferation and survival, metabolic signaling or the inflammatory response. The effects of the compounds on each of these processes are assessed by measuring changes in biological markers specific to each of these processes following oligonucleotide treatment.

Cell Culture

HMECs

Normal human mammary epithelial cells (HMECs) were obtained from American Type Culture Collection (Manassus, Va.). ECs were routinely cultured in DMEM high glucose (Invitrogen Life Technologies, Carlsbad, Calif.) supplemented with 10% fetal bovine serum (Invitrogen Life Technologies, Carlsbad, Calif.). Cells were routinely passaged by trypsinization and dilution when they reached approximately 90% confluence. HMECs were plated in 24-well plates (Falcon-Primaria # 353047, BD Biosciences, Bedford, Mass.) at a density of 50,000-60,000 cells per well, and allowed to attach overnight prior to treatment with oligomeric compounds. HMECs were plated in 96-well plates (Falcon-Primaria #353872, BD Biosciences, Bedford, Mass.) at a density of approximately 10,000 cells per well and allowed to attach overnight prior to treatment with oligomeric compounds.

MCF7 Cells

The breast carcinoma cell line MCF7 was obtained from American Type Culture Collection (Manassus, Va.). MCF7 cells were routinely cultured in DMEM high glucose (Invitrogen Life Technologies, Carlsbad, Calif.) supplemented with 10% fetal bovine serum (Invitrogen Life Technologies, Carlsbad, Calif.). Cells were routinely passaged by trypsinization and dilution when they reached approximately 90% confluence. MCF7 cells were plated in 24-well plates (Falcon-Primaria # 353047, BD Biosciences, Bedford, Mass.) at a density of approximately 140,000 cells per well, and allowed to attach overnight prior to treatment with oligomeric compounds. MCF7 cells were plated in 96-well plates (Falcon-Primaria #353872, BD Biosciences, Bedford, Mass.) at a density of approximately 20,000 cells per well and allowed to attach overnight prior to treatment with oligomeric compounds.

T47D Cells

The breast carcinoma cell line T47D was obtained from American Type Culture Collection (Manassus, Va.). T47D cells do not express the tumor suppressor gene p53. T47D cells were cultured in DMEM high glucose medium (Invitrogen Life Technologies, Carlsbad, Calif.) supplemented with 10% fetal bovine serum (Invitrogen Life Technologies, Carlsbad, Calif.). Cells were routinely passaged by trypsinization and dilution when they reached approximately 90% confluence. T47D cells were plated in 24-well plates (Falcon-Primaria # 353047, BD Biosciences, Bedford, Mass.) at a density of approximately 170,000 cells per well, and allowed to attach overnight prior to treatment with oligomeric compounds. T47D cells were plated in 96-well plates (Falcon-Primaria #353872, BD Biosciences, Bedford, Mass.) at a density of approximately 20,000 cells per well and allowed to attach overnight prior to treatment with oligomeric compounds.

HUVECs

Human vascular endothelial cells (HUVECs) were obtained from American Type Culture Collection (Manassus, Va.). HUVECs were routinely cultured in EBM (Clonetics Corporation, Walkersville, Md.) supplemented with SingleQuots supplements (Clonetics Corporation, Walkersville, Md.). Cells were routinely passaged by trypsinization and dilution when they reached approximately 90% confluence and were maintained for up to 15 passages. HUVECs were plated at approximately 3000 cells/well in 96-well plates (Falcon-Primaria #353872, BD Biosciences, Bedford, Mass.) and treated with oligomeric compounds one day later.

Human Preadipocytes

Human preadipocytes were obtained from Zen-Bio, Inc. (Research Triangle Park, N.C.). Preadipocytes were routinely maintained in Preadipocyte Medium (ZenBio, Inc., Research Triangle Park, N.C.) supplemented with antibiotics as recommended by the supplier. Cells were routinely passaged by trypsinization and dilution when they reached 90% confluence. Cells were routinely maintained for up to 5 passages as recommended by the supplier. One day prior to transfection, 96-well plates (Falcon-Primaria #353872, BD Biosciences, Bedford, Mass.) were seeded with approximately 3000 cells/well. On the day of transfection, preadipocytes were transfected with oligomeric compounds of the invention. After 4 hours, the transfection mixture was removed from the cells, replaced with Preadipocyte medium and cell culture was continued for 3 days, to allow the cells to reach confluency. To induce differentiation of preadipocytes, cells were cultured in differentiation medium (Zen-Bio, Inc., Research Triangle Park, N.C.) consisting of Preadipocyte Medium further supplemented with 2% fetal bovine serum (final of 12%), amino acids, 100 nM insulin, 0.5 mM IBMX, 1 mM dexamethasone and 1 mM BRL49653. Cells were cultured in differentiation medium for 3 days, after which they were cultured in adipocyte medium (Zen-Bio., Inc, Research Triangle Park, N.C.) consisting of Preadipocyte Medium supplemented with 33 mM biotin, 17 mM pantothenate, 100 nM insulin and 1 mM dexamethasone.

Dendritic Cells

Dendritic cells (DCs, Clonetics Corp., San Diego, Calif.) were plated at a density of approximately 6500 cells/well on anti-CD3 coated 96-well plates (UCHT1, Pharmingen-BD, San Diego, Calif.) in 500 U/mL granulocyte macrophase-colony stimulation factor (GM-CSF) and interleukin-4 (IL-4). Dendritic cells were treated with oligomeric compounds approximately 24 hours after plating.

Treatment with Oligomeric Compounds

Oligomeric compounds were introduced into cells using the cationic lipid transfection reagent LIPOFECTIN™ (Invitrogen Life Technologies, Carlsbad, Calif.). Oligomeric compounds were mixed with LIPOFECTIN™ in Opti-MEM or Eagle's MEM (Invitrogen Life Technologies, Carlsbad, Calif.) to achieve the desired final concentration of oligomeric compound and LIPOFECTIN™. Before adding to cells, the oligomeric compound, LIPOFECTIN™ and Opti-MEM/Eagle's MEM were mixed thoroughly and incubated for approximately 0.5 hrs. The medium was removed from the plates and the plates were tapped on sterile gauze. Each well of a 96-well plate was washed with 150 μl of phosphate-buffered saline, Hank's balanced salt solution or serum-free culture medium. Each well of a 24-well plate was washed with 250 μL of phosphate-buffered saline, Hank's balanced salt solution or serum-free culture medium. The wash buffer in each well was replaced with 100 μL or 250 μL of the oligomeric compound/Opti-MEM/LIPOFECTIN™ cocktail or oligomeric compound/Eagle's MEM/LIPOFECTIN™ for 96-well or 24-well plates, respectively. Untreated control cells received LIPOFECTIN™ in Opti-MEM or Eagle's MEM, without oligomeric compounds. The plates were incubated for approximately 4 hours at 37° C., after which the medium was removed and the plates were tapped on sterile gauze. 100 μl or 1 mL of full growth medium was added to each well of a 96-well plate or a 24-well plate, respectively.

Cell Proliferation and Survival Assays

Cell Cycle Assay

A cell cycle assay was employed to identify genes whose modulation affects cell cycle progression. In addition to normal cells, cells lacking functional p53 were utilized to identify genes whose modulation will sensitize p53-deficient cells to anti-cancer agents. Oligomeric compounds were tested for their effects on the cell cycle in normal human mammary epithelial cells (HMECs) as well as the breast carcinoma cell lines MCF7 and T47D. The latter two cell lines express similar genes but MCF7 cells express the tumor suppressor p53, while T47D cells are deficient in p53. A 20-nucleotide oligomeric compound with a randomized sequence was used a negative control (ISIS 29848) a compound that does not target modulators of cell cycle progression. An oligomeric compound targeting kinesin-like 1 (ISIS 183881) is known to inhibit cell cycle progression and was used as a positive control.

Cells were transfected as described herein. Oligomeric compounds were mixed with LIPOFECTIN™ in Opti-MEM to achieve a final concentration of 200 nM of oligomeric compound and 6 μg/mL LIPOFECTIN™. Selected oligomeric compounds and the positive control were tested in triplicate. The negative control was tested in up to six replicate wells. Untreated control cells received LIPOFECTIN™ in Opti-MEM only. Approximately 48 hours following transfection, routine procedures were used to prepare cells for flow cytometry analysis and cells were stained with propidium iodide to generate a cell cycle profile using a flow cytometer. The cell cycle profile was analyzed with the ModFit program (Verity Software House, Inc., Topsham Me.).

In further studies, T47D cells into which the p53 gene has been stably introduced (T47Dp53) are used to assess the effects of oligomeric compounds on cell cycling. T47Dp53 cells are T47D cells that have been transfected with and selected for maintenance of a plasmid that expresses a wildtype copy of the p53 gene (for example, pCMV-p53; Clontech, Palo Alto, Calif.), using standard laboratory procedures. Transfection and flow cytometry analyses of T47Dp53 cells are performed as described for T47D cells.

Fragmentation of nuclear DNA is a hallmark of apoptosis and produces an increase in cells with a hypodiploid DNA content, which are categorized as “subG1”. An increase in cells in G1 phase is indicative of a cell cycle arrest prior to entry into S phase; an increase in cells in S phase is indicative of cell cycle arrest during DNA synthesis; and an increase in cells in the G2/M phase is indicative of cell cycle arrest just prior to or during mitosis. Cell cycle profiles of cells treated with oligomeric compounds were normalized to those of untreated control cells and are shown in Table 7. Indicated in the “Marker” column of Table 7 are the cell type tested and cell cycle phase, for example, “HMEC, G1” indicates HMEC cells in the G1 phase of the cell cycle. Values above or below 100% were considered to indicate an increase or decrease, respectively, in the proportion of cells in a particular phase of the cell cycle.

Oligomeric compounds that prevent cell cycle progression are candidate therapeutic agents for the treatment of hyperproliferative disorders, such as cancer or inflammation.

Apoptosis Assay

Select oligomeric compounds of the invention were assayed for their affects on apoptosis in normal human mammary epithelial cells (HMECs) as well as the breast carcinoma cell lines MCF7 and T47D. HMECs and MCF7 cells express p53, whereas T47D cells do not express this tumor suppressor gene. Cells were cultured in 96-well plates with black sides and flat, transparent bottoms (Corning Incorporated, Corning, N.Y.). DMEM medium, with and without phenol red, was obtained from Invitrogen Life Technologies (Carlsbad, Calif.). MEGM medium, with and without phenol red, was obtained from Cambrex Bioscience (Walkersville, Md.). A 20-nucleotide oligomeric compound with a randomized sequence was used a negative control (ISIS 29848), a compound that does not target modulators of caspase activity. An oligomeric compound targeted to human Jagged2 (ISIS 148715) or human Notch1 (ISIS 226844), both of which are known to induce caspase activity, was used as a positive control for caspase activation.

Cells were transfected as described herein. Oligomeric compounds were mixed with LIPOFECTIN™ in Opti-MEM to achieve a final concentration of 200 nM of oligomeric compound and 6 μg/mL LIPOFECTN™. Oligomeric compounds of the invention and the positive controls were tested in triplicate, and the negative control was tested in up to six replicate wells. Untreated control cells received LIPOFECTIN™ in Opti-MEM only.

In further studies, T47D cells into which p53 has been stably introduced are used to assess the effects of oligomeric compounds on apoptosis. T47Dp53 cells are T47D cells that have been transfected with and selected for maintenance of a plasmid that expresses a wildtype copy of the p53 gene (for example, pCMV-p53; Clontech, Palo Alto, Calif.), using standard laboratory procedures. The caspase-3 activity is measured as described herein.

Caspase-3 activity was evaluated with a fluorometric HTS Caspase-3 assay (Catalog # HTS02; EMD Biosciences, San Diego, Calif.) that detects cleavage after aspartate residues in the peptide sequence DEVD. The DEVD substrate is labeled with a fluorescent molecule, which exhibits a blue to green shift in fluorescence upon cleavage by caspase-3. Active caspase-3 in the oligomeric compound-treated cells was measured by this assay according to the manufacturer's instructions. Approximately 48 hours following treatment in HMEC, MCF7 or T47D cells, or 24 and 48 hours following treatment in T47Dp53 cells, 50 μL of assay buffer containing 10 μM dithiothreitol was added to each well, followed by addition 20 μL of the caspase-3 fluorescent substrate conjugate. Fluorescence in wells was immediately detected (excitation/emission 400/505 nm) using a fluorescent plate reader (SpectraMAX GeminiXS, Molecular Devices, Sunnyvale, Calif.). The plate was covered and incubated at 37° C. for an additional three hours, after which the fluorescence was again measured (excitation/emission 400/505 nm). The value at time zero was subtracted from the measurement obtained at 3 hours. The measurement obtained from the untreated control cells was designated as 100% activity. Caspase-3 activity in cells treated with oligomeric compounds was normalized to that in untreated control cells and the data are shown in Table 7. The cell type in which data were obtained in the “Marker” column of Table 7, for example, “HMEC, Caspase-3” indicates caspase-3 activity in HMEC cells 48 hours following treatment with the oligomeric compounds of the invention. Caspase-3 activity in T47Dp53 cells was measured after 24 and 48 hours, for example, “T47dp53, 24 hr, Caspase-3” indicates caspase-3 activity in T47Dp53 cells 24 hours following treatment with oligomeric compounds of the invention. Values for caspase activity above or below 100% were considered to indicate that the compound has the ability to stimulate or inhibit caspase activity, respectively.

Compounds that cause a significant induction in apoptosis are candidate therapeutic agents with applications in the treatment of conditions in which the induction of apoptosis is desirable, for example, in hyperproliferative disorders. Compounds that inhibit apoptosis are candidate therapeutic agents with applications in the treatment of conditions where the reduction of apoptosis is useful, for example, in neurodegenerative disorders.

Angiogenesis Assays

Endothelial Tube Formation Assay

HUVECs were used to measure the effects of oligomeric compounds of the invention on endothelial tube formation activity. The tube formation assay was performed using an in vitro Angiogenesis Assay Kit (Chemicon International, Temecula, Calif.). A 20-nucleotide oligomeric compound with a randomized sequence (ISIS 29848) served as a negative control, a compound that does not target modulators of endothelial tube formation.

Oligomeric compounds were mixed with LIPOFECTIN™ in Opti-MEM to achieve a final concentration of 75 nM of oligomeric compound and 2.25 μg/mL LIPOFECTIN™. Untreated control cells received LIPOFECTN™ in Opti-MEM only. Compounds of the invention were tested in triplicate, and the negative control was tested in up to six replicates.

Approximately fifty hours after transfection, cells were transferred to 96-well plates coated with ECMatrix™ (Chemicon International). Under these conditions, untreated HUVECs form tube-like structures. After an overnight incubation at 37° C., treated and untreated cells were inspected by light microscopy. Individual wells were assigned discrete scores from 1 to 5 depending on the extent of tube formation. A score of 1 refers to a well with no tube formation while a score of 5 was given to wells where all cells were forming an extensive tubular network. Tube formation in cells treated with oligomeric compounds was normalized to that in untreated control cells. The data are shown and Table 7 and are identified by the designation “Tube Formation” in the “Marker” column.

Compounds resulting in a decrease in tube formation are candidate therapeutic agents for the inhibition of angiogenesis where such activity is desired, for example, in the treatment of cancer, diabetic retinopathy, cardiovascular disease, rheumatoid arthritis and psoriasis.

Compounds that promote endothelial tube formation are candidate therapeutic agents with applications where the stimulation of angiogenesis is desired, for example, in wound healing.

Adipocyte and Insuling Signaling Assays

Adipocyte Differentiation Assay

Select oligomeric compounds of the invention were tested for their effects on preadipocyte differentiation. A 20-nucleotide oligomeric compound with a randomized sequence was used a negative control (ISIS 29848), a compound that does not target modulators of adipocyte differentiation. Tumor necrosis factor alpha (TNF-α) is known to inhibit adipocyte differentiation and was used as a positive control for the inhibition of adipocyte differentiation as evaluated by leptin secretion. For all other markers assayed, an oligomeric compound targeted to PPAR-γ (ISIS 105990), also known to inhibit adipocyte differentiation, served as a positive control.

Cells were transfected as described herein. Oligomeric compounds were mixed with LIPOFECTIN™ in Opti-MEM to achieve a final concentration of 250 nM of oligomeric compound and 10 μg/mL LIPOFECTIN™. Untreated control cells received LIPOFECTIN™ in Opti-MEM only. Oligomeric compounds of the invention and the positive control were tested in triplicate, and the negative control was tested in up to six replicate wells.

After the cells reached confluence (approximately three days), they were exposed for an additional three days to differentiation medium (Zen-Bio, Inc., Research Triangle Park, N.C.) containing a PPAR-γ agonist, IBMX, dexamethasone, and insulin. Cells were then fed adipocyte medium (Zen-Bio, Inc.), which was replaced at 2 or 3 day intervals.

Leptin secretion into the medium in which adipocytes were cultured was measured by protein ELISA. On day nine post-transfection, 96-well plates were coated with a monoclonal antibody to human leptin (R&D Systems, Minneapolis, Minn.) and left at 4° C. overnight. The plates were blocked with bovine serum albumin (BSA), and a dilution of the treated adipoctye medium was incubated in the plate at room temperature for approximately 2 hours. After washing to remove unbound components, a second monoclonal antibody to human leptin (conjugated with biotin) was added. The plate was then incubated with strepavidin-conjugated horseradish peroxidase (HRP) and enzyme levels were determined by incubation with 3, 3′, 5, 5′-tetramethylbenzidine, which turns blue when cleaved by HRP. The OD₄₅₀ was read for each well, where the dye absorbance is proportional to the leptin concentration in the cell lysate. Leptin secretion from cells treated with oligomeric compounds or TNF-α was normalized to that from untreated control cells. With respect to leptin secretion, values above or below 100% were considered to indicate that the compound has the ability to stimulate or inhibit leptin secretion, respectively. The data are presented in Table 7, indicated by “Leptin Secretion” in the “Marker” column.

The triglyceride accumulation assay measures the synthesis of triglyceride by adipocytes. Triglyceride accumulation was measured using the Infinity™ Triglyceride reagent kit (Sigma-Aldrich, St. Louis, Mo.). On day nine post-transfection, cells were washed and lysed at room temperature, and the triglyceride assay reagent was added. Triglyceride accumulation was measured based on the amount of glycerol liberated from triglycerides by the enzyme lipoprotein lipase. Liberated glycerol is phosphorylated by glycerol kinase, and hydrogen peroxide is generated during the oxidation of glycerol-1-phosphate to dihydroxyacetone phosphate by glycerol phosphate oxidase. Horseradish peroxidase (HRP) uses H₂O₂ to oxidize 4-aminoantipyrine and 3,5 dichloro-2-hydroxybenzene sulfonate to produce a red-colored dye. Dye absorbance, which is proportional to the concentration of glycerol, was measured at 515 nm using an UV spectrophotometer. Glycerol concentration was calculated from a standard curve for each assay, and data were normalized to total cellular protein as determined by a Bradford assay (Bio-Rad Laboratories, Hercules, Calif.). Triglyceride accumulation in cells treated with oligomeric compounds was normalized to that in untreated control. Values for triglyceride accumulation above or below 100% were considered to indicate that the compound has the ability to stimulate or inhibit triglyceride accumulation, respectively. The data are presented in Table 7, indicated by “Triglyceride Accumulation” in the “Marker” column.

Expression of the four hallmark genes, HSL, aP2, Glut4, and PPARγ, was also measured in adipocytes transfected with compounds of the invention. Cells were lysed on day nine post-transfection and total RNA was harvested. The amount of total RNA in each sample was determined using a Ribogreen Assay (Invitrogen Life Technologies, Carlsbad, Calif.). Real-time PCR was performed on the total RNA using primer/probe sets for the adipocyte differentiation hallmark genes Glut4, HSL, aP2, and PPAR-γ. Gene expression in cells treated with oligomeric compounds was compared to that in untreated control cells. With respect to the four adipocyte differentiation hallmark genes, values above or below 100% were considered to indicate that the compound has the ability to stimulate or inhibit adipocyte differentiation, respectively. The data are illustrated in Table 7, where the adipocytes differentiation hallmark gene expression measured is indicated by the presence of the gene name in the “Marker” column, for example, “GLUT4” indicates the expression of Glut4 relative to untreated control cells. The apoptosis assay is also performed to measure caspase-3 activity in differentiation adipocytes.

Compounds that reduce the expression levels of markers of adipocyte differentiation are candidate therapeutic agents with applications in the treatment, attenuation or prevention of obesity, hyperlipidemia, atherosclerosis, atherogenesis, diabetes, hypertension, or other metabolic diseases as well as having potential applications in the maintenance of the pluripotent phenotype of stem or precursor cells. Compounds of the invention resulting in a significant increase in leptin secretion are potentially useful for the treatment of obesity.

Inflammation Assays

Cytokine Production Assay

The effects of oligomeric compounds of the invention were examined on the dendritic cell-mediated costimulation of T-cells. A 20-nucleotide oligomeric compound with a randomized sequence served as a negative control (ISIS 29848), a compound that does not target modulators of dendritic cell-mediated T-cell costimulation. An oligomeric compound targeted to human CD86 (ISIS 113131) is known to inhibit dendritic cell-mediated T-cell stimulation and was used as a positive control.

Cells were transfected as described herein. Oligomeric compounds were mixed with LIPOFECTIN™ in Opti-MEM to achieve a final concentration of 200 nM of oligomeric compound and 6 μg/mL LIPOFECTIN™. Untreated control cells received LIPOFECTIN™ in Opti-MEM only. Compounds of the invention and the positive control were tested in triplicate, and the negative control was tested in up to six replicates. Following incubation with the oligomeric compounds and LIPOFECTIN™, fresh growth medium with cytokines was added and DC culture was continued for an additional 48 hours. DCs were then co-cultured with Jurkat T-cells (American Type Culture Collection, Manassus, Va.) in RPMI medium (Invitrogen Life Technologies, Carlsbad, Calif.) supplemented with 10% heat-inactivated fetal bovine serum (Sigma Chemical Company, St. Louis, Mo.). Culture supernatants were collected 24 hours later and assayed for IL-2 levels (IL-2 DuoSet, R&D Systems, Minneapolis, Minn.). IL-2 levels in cells treated with oligomeric compounds were normalized to those untreated cells. A value greater than 100% indicates an induction of the inflammatory response, whereas a value less than 100% demonstrates a reduction in the inflammatory response. The data are illustrated in Table 7, where “IL-2” in the “Marker” column indicates the IL-2 levels in cells treated with oligomeric compounds of the invention.

Compounds that inhibit T-cell co-stimulation are candidate therapeutic compounds with applications in the prevention, treatment or attenuation of conditions associated with hyperstimulation of the immune system, including rheumatoid arthritis, irritable bowel disease, asthma, lupus and multiple sclerosis. Compounds that induce T-cell co-stimulation are candidate therapeutic agents for the treatment of immunodeficient conditions.

TABLE 7
Analysis of phenotypes following treatment of cells with oligomeric compounds
targeted to a bone growth modulator

| | | | | % | SEQ |
|---|---|---|---|---|---|
| Target | Treatment | | | Untreated | ID |
| Name | with ISIS # | Assay | Marker | Control | NO |
| transducer of | 180937 | Adipocyte | GLUT4 | 65 | 599 |
| ERBB2 | | Differentiation | | | |
| transducer of | 180937 | Adipocyte | HSL | 93 | 599 |
| ERBB2 | | Differentiation | | | |
| transducer of | 180937 | Adipocyte | Leptin Secretion | 111 | 599 |
| ERBB2 | | Differentiation | | | |
| transducer of | 180937 | Adipocyte | Triglyceride | 76 | 599 |
| ERBB2 | | Differentiation | Accumulation | | |
| transducer of | 180937 | Adipocyte | aP2 | 94 | 599 |
| ERBB2 | | Differentiation | | | |
| transducer of | 180937 | Angiogenesis | Tube Formation | 66 | 599 |
| ERBB2 | | | | | |
| transducer of | 180937 | Angiogenesis | Tube Formation | 100 | 599 |
| ERBB2 | | | | | |
| transducer of | 180937 | Apoptosis | HMEC cells, | 249 | 599 |
| ERBB2 | | | Caspase-3 | | |
| transducer of | 180937 | Apoptosis | MCF7 cells, | 124 | 599 |
| ERBB2 | | | Caspase-3 | | |
| transducer of | 180937 | Apoptosis | T47D cells, 48 hr, | 123 | 599 |
| ERBB2 | | | Caspase-3 | | |
| transducer of | 180937 | Cell Cycle | T47D cells, SubG1 | 23 | 599 |
| ERBB2 | | | | | |
| transducer of | 180937 | Cell Cycle | T47D cells, G1 | 95 | 599 |
| ERBB2 | | | | | |
| transducer of | 180937 | Cell Cycle | T47D cells, S | 111 | 599 |
| ERBB2 | | | | | |
| transducer of | 180937 | Cell Cycle | T47D cells, G2/M | 104 | 599 |
| ERBB2 | | | | | |
| transducer of | 180937 | Cell Cycle | HMEC cells, | 73 | 599 |
| ERBB2 | | | SubG1 | | |
| transducer of | 180937 | Cell Cycle | HMEC cells, G1 | 98 | 599 |
| ERBB2 | | | | | |
| transducer of | 180937 | Cell Cycle | HMEC cells, S | 105 | 599 |
| ERBB2 | | | | | |
| transducer of | 180937 | Cell Cycle | HMEC cells, G2/M | 102 | 599 |
| ERBB2 | | | | | |
| transducer of | 180937 | Cell Cycle | MCF7 cells, | 11 | 599 |
| ERBB2 | | | SubG1 | | |
| transducer of | 180937 | Cell Cycle | MCF7 cells, G1 | 104 | 599 |
| ERBB2 | | | | | |
| transducer of | 180937 | Cell Cycle | MCF7 cells, S | 89 | 599 |
| ERBB2 | | | | | |
| transducer of | 180937 | Cell Cycle | MCF7 cells, G2/M | 115 | 599 |
| ERBB2 | | | | | |
| transducer of | 180937 | Inflammation | Interleukin-2 | 99 | 599 |
| ERBB2 | | | | | |

### EXAMPLE 7

Antisense Inhibition of Bone Growth Modulator mRNA Expression in Cultured Cells: Dose Response

In a further embodiment of this invention, the effect of oligomeric compounds of the invention on the expression of bone growth modulator was determined in cultured cells.

The indicated cells were cultured as described herein and treated at the concentrations indicated in the respective table. The RNA was then harvested and the expression levels of the bone growth modulator mRNA were measured by the methods described herein. The results are expressed as percent inhibition relative to untreated control and represent the average from replicate experiments.

TABLE 8
Antisense inhibition of sFRP-1 mRNA expression
in FAT7 cells: dose response

Oligomeric Compound
Concentration (nM)

| Oligomeric Compound | 12.5 | 25 | 50 | 100 |
|---|---|---|---|---|
| ISIS 129689 | 94 | 88 | 92 | 92 |
| ISIS 129694 | 96 | 94 | 79 | 81 |
| ISIS 129695 | 95 | 80 | 76 | 71 |
| ISIS 279750 | 54 | 59 | 38 | 31 |
| ISIS 299463 | 56 | 50 | 35 | 22 |
| ISIS 299458 | 92 | 61 | 36 | 31 |

Oligomeric compounds of the invention directed to sFRP-1 significantly reduced sFRP-1 expression in FAT7 cells relative to control oligonucleotides in a dose-dependent manner.

TABLE 9
Antisense inhibition of DKK-1 mRNA expression
in ND7/23 cells: dose response

Oligomeric Compound
Concentration (nM)

| Oligomeric Compound | 12.5 | 25 | 50 | 100 |
|---|---|---|---|---|
| ISIS 129689 | 106 | 91 | 97 | 100 |
| ISIS 129695 | 92 | 89 | 98 | 89 |
| ISIS 129700 | 96 | 93 | 76 | 84 |
| ISIS 319395 | 92 | 86 | 70 | 49 |
| ISIS 319411 | 92 | 87 | 94 | 49 |
| ISIS 319443 | 91 | 80 | 50 | 23 |

Oligomeric compounds of the invention directed to DKK-1 significantly reduced DKK-1 expression in ND7/23 cells relative to control oligonucleotides in a dose-dependent manner.

TABLE 10
Antisense inhibition of sclerostin mRNA expression
in UMR106 cells: dose response

Oligomeric Compound
Concentration (nM)

| Oligomeric Compound | 3.125 | 12.5 | 50 | 200 |
|---|---|---|---|---|
| ISIS 279490 | 145 | 110 | 96 | 66 |
| ISIS 279475 | 116 | 104 | 76 | 56 |
| ISIS 279476 | 105 | 108 | 70 | 48 |
| ISIS 279517 | 99 | 105 | 64 | 40 |

Oligomeric compounds of the invention directed to sclerostin reduced sclerostin expression in U106 cells in a dose-dependent manner.

### EXAMPLE 8

Treatment of Ovariectomized Rats with Oligomeric Compound in a Delayed Dosing Model

The ovariectomized rat is a rodent model for osteoporosis. Oligomeric compounds of the invention were tested for their ability to enable regrowth of bone in bone-eroded ovariectomized rats. Six month old ovariectimized Sprague-Dawley rats and sham surgical controls (Harlan, Indianapolis, Ind.) were dosed subcutaneously with 10, 25 or 50 mg/kg oligomeric compound or saline three times a week for eight weeks starting 30 days post-ovariectomy. The 30 day waiting period prior to dosing was to allow the progression of bone erosion to simulate osteoporosis. At experiment termination, long bones were recovered and bone marrow mRNA and bone mineral density was measured.

### EXAMPLE 9

Antisense Inhibition of Bone Growth Modulator mRNA Expression in Bone Marrow after Ovariectomy in Rat: In Vivo Dose Response

In accordance with the present invention, the bone growth modulator inhibition by antisense oligonucleotide was demonstrated in the post-ovariectomized rat.

ISIS 279480 (GAAGGCTTGCCACCCCTGGC, SEQ ID NO: 416) and ISIS 279505 (CGGCAGCTGTACTCGGACAC, SEQ ID NO: 441) are oligomeric compounds targeted to rat sclerostin. ISIS 279480 and ISIS 279505 are chimeric oligonucleotides (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Ovariectomized rats were treated as described in Example 8 with the above oligomeric compounds and sclerostin mRNA expression was measured in the long bones of the rat subjects. The results shown in Table 11 are the average from eight rats per group.

TABLE 11
Antisense inhibition of sclerostin mRNA expression in proximal
tibia post-ovariectomy in the rat: dose response

| | Proximal tibia sclerostin |
|---|---|
| | mRNA |
| Subcutaneous Dose | % Sham control |
| Saline | 52 |
| PTH | 116 |

ISIS 279480

| | |
|---|---|
| 10 mg/kg | 59 |
| 25 mg/kg | 50 |
| 50 mg/kg | 86 |

ISIS 279505

| | |
|---|---|
| 10 mg/kg | 39 |
| 25 mg/kg | 50 |
| 50 mg/kg | ND* |

*ND Not Determined

ISIS 279750 (GATGGGCCCCAGCTTCAAGG, SEQ ID NO: 531) and ISIS 299463 (CCCACTTGTGAATGGCTGTG, SEQ ID NO: 562) are oligomeric compounds targeted to rat sFRP-1. ISIS 279750 and ISIS 299463 are chimeric oligonucleotides (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Ovariectomized rats were treated as described in Example 8 with the above oligomeric compounds and sFRP-1 mRNA expression was measured in the long bones of the rat subjects. The results shown in Table 12 are the average from eight rats per group. The data demonstrate that the oligonucleotide of the present invention inhibits expression of sFRP-1 mRNA in vivo.

TABLE 12
Antisense inhibition of sFRP-1 mRNA expression in bone marrow
and proximal tibia post-ovariectomy in the rat: dose response

| | Bone Marrow sFRP-1 | Proximal tibia |
|---|---|---|
| | mRNA | sFRP-1 mRNA |
| Subcutaneous Dose | % OVX control | % OVX control |
| Sham | 47 | 63 |

ISIS 279750

| | | |
|---|---|---|
| 10 mg/kg | 58 | 89 |
| 25 mg/kg | 23 | 84 |
| 50 mg/kg | 8 | 44 |

ISIS 299463

| | | |
|---|---|---|
| 10 mg/kg | 56 | 65 |
| 25 mg/kg | 39 | 38 |
| 50 mg/kg | 11 | 36 |

ISIS 143631 (CTCTGATTCCCGTCTAGTGA, SEQ ID NO: 134) and ISIS 143640 (GTGTTTCACATTTAGGCCCT, SEQ ID NO: 142) are oligomeric compounds targeted to rat src-c. ISIS 143631 and ISIS 143640 are chimeric oligonucleotides (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Ovariectomized rats were treated as described in Example 8 with the above oligomeric compounds and src-c mRNA expression was measured in the long bones of the rat subjects. The results shown in Table 13 are the average from eight rats per group. The data demonstrate that the oligomeric compounds of the present invention inhibit expression of src-c mRNA in vivo.

TABLE 13
Antisense inhibition of src-c mRNA expression in bone marrow
and proximal tibia post-ovariectomy in the rat: dose response

| | Bone Marrow Src-c mRNA | Proximal Tibia Src-c mRNA |
|---|---|---|
| | % control (normalized to | % control (normalized to |
| Treatment | saline treated OVX rats) | saline treated OVX rats) |
| Sham | 100 | 112 |
| PTH | 116 | 180 |

ISIS 143631

| | | |
|---|---|---|
| 10 mg/kg | 90 | 140 |
| 25 mg/kg | 72 | 250 |
| 50 mg/kg | 32 | 330 |

ISIS 143640

| | | |
|---|---|---|
| 10 mg/kg | 97 | 110 |
| 25 mg/kg | 74 | 370 |
| 50 mg/kg | 63 | 440 |

Oligomeric compounds of the invention inhibited expression in bone marrow. Paradoxically, said compounds increased expression of src-c in proximal tibia.

### EXAMPLE 10

Increased Bone Mineral Density by Treatment with Oligomeric Compounds of the Invention in a Delayed Dosing Ovariectomized Rat Model.

Bone growth increase by oligomeric compound targeted to a bone growth modulator was demonstrated in a delayed dosing ovariectomized rat by measuring the bone mineral density (BMD).

ISIS 279480 (GAAGGCTTGCCACCCCTGGC, SEQ ID NO: 416) and ISIS 279505 (CGGCAGCTGTACTCGGACAC, SEQ ID NO: 441) are oligomeric compounds targeted to rat sclerostin. ISIS 279480 and ISIS 279505 are chimeric oligonucleotides (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-O-methoxyethyl (2′-MOE) nucleotides. The internucleoside (backbone) linkages are phosphorothioate (P═S) throughout the oligonucleotide. All cytidine residues are 5-methylcytidines.

Ovariectomized rats were treated as described in Example 8 with the above oligomeric compounds and BMD was measured at experiment termination in vivo and ex vivo (surgically removed) in the femur and in vivo in the L4/L5 vertebra by dual-energy-x-ray absorptiometry (DEXA) using a PIXImus mouse densitometer (Faxitron X-Ray Corporation, Wheeling, Ill.) according to manufacturer's instructions. Table 14 shows the results of such measurements.

TABLE 14
Increased BMD by treatment with oligomeric compounds targeted
to sclerostin in a delayed dosing ovariectomized rat model.

| | |
|---|---|
| L4/L5 Vertebra BMD | Distal Femur BMD |
| (gm/cm²) | (gm/cm²) |

| Treatment | In Vivo | In Vivo | Ex Vivo |
|---|---|---|---|
| Sham | 0.24 | 0.22 | 0.21 |
| OVX | 0.18 | 0.17 | 0.16 |
| OVX + PTH | 0.26 | 0.23 | 0.21 |

ISIS 279480

| | | | |
|---|---|---|---|
| 10 mg/kg | 0.21 | 0.18 | 0.17 |
| 25 mg/kg | 0.21 | 0.19 | 0.18 |
| 50 mg/kg | 0.22 | 0.20 | 0.18 |

ISIS 279505

| | | | |
|---|---|---|---|
| 10 mg/kg | 0.21 | 0.18 | 0.17 |
| 25 mg/kg | ND* | ND | 0.16** |
| 50 mg/kg | ND | ND | ND |

*ND Not Determined
**Experiment terminated at 7 weeks.

Generally, there was a dose-dependent increase in BMD upon treatment with oligomeric compounds of the invention directed to src-c.

ISIS 143631 (CTCTGATTCCCGTCTAGTGA, SEQ ID NO: 134) and ISIS 143640 (GTGTTTCACATTTAGGCCCT, SEQ ID NO: 142) are oligomeric compounds targeted to rat src-c. ISIS 143631 and ISIS 143640 are chimeric oligonucleotides (“gapmer”) 20 nucleotides in length, composed of a central “gap” region consisting of ten 2′-deoxynucleotides, which is flanked on both sides (5′ and 3′ directions) by five-nucleotide “wings”. The wings are composed of 2′-methoxyethyl (2′-MOE) nucleotides. The internucleoside (backbone) linkages are phosphorothioate (P═S) throughout the oligonucleotide. All cytidine residues are 5-methylcytidines.

Ovariectomized rats were treated as described above. Table 15 shows the results of BMD measurements.

TABLE 15
Increased BMD by treatment with oligomeric compounds targeted
to src-c in a delayed dosing ovariectomized rat model.

| | |
|---|---|
| L4/L5 Vertebra BMD | Distal Femur BMD |
| (gm/cm²) | (gm/cm²) |

| Treatment | In Vivo | In Vivo | Ex Vivo |
|---|---|---|---|
| Sham | 0.224 | 0.215 | 0.215 |
| OVX | 0.171 | 0.171 | 0.167 |
| OVX + PTH | 0.238 | 0.215 | 0.219 |

ISIS 143631

| | | | |
|---|---|---|---|
| 10 mg/kg | 0.171 | 0.168 | 0.169 |
| 25 mg/kg | 0.203 | 0.181 | 0.163 |
| 50 mg/kg | ND | ND | ND |

ISIS 143640

| | | | |
|---|---|---|---|
| 10 mg/kg | 0.188 | 0.173 | 0.162 |
| 25 mg/kg | 0.196 | 0.190 | 0.175 |
| 50 mg/kg | ND | ND | ND |

Generally, there was a dose-dependent increase in BMD upon treatment with oligomeric compounds of the invention directed to src-c.

What is claimed is:

1. An oligomeric compound of 13 to 80 nucleobases targeted to a nucleic acid molecule encoding a bone growth modulator, wherein said bone growth modulator is DKK-1, GSK3 beta, sFRP-1, sclerostin, transducer of ERRB1, or src-c and wherein said oligomeric compound inhibits the expression of said bone growth modulator.

2. The compound of claim 1 having at least 70% complementarity, at least 80% complementarity, at least 90% complementarity, at least 95% complementarity, or at least 99% complementarity with said nucleic acid molecule encoding a bone growth modulator.

3. The compound of claim 1 comprising a single stranded compound.

4. The compound of claim 1 which is a chemically modified compound.

5. The chemically modified compound of claim 4 having at least one modified internucleoside linkage, sugar moiety, or nucleobase.

6. The compound of claim 5 comprising a chimeric oligonucleotide.

7. A pharmaceutical composition comprising a compound of claim 1 and a pharmaceutically acceptable penetration enhancer, carrier, or diluent.

8. A method of inhibiting the expression of a bone growth modulator in a bodily fluid, cell or tissue comprising contacting said bodily fluid, cell or tissue with the compound of claim 1 so that expression of said bone growth modulator is inhibited.


SEQUENCE LISTING (680 sequences)

SEQ ID NO: 1 - DNA, 574 bp, Rattus norvegicus
cggccgccgagcccaagctcttcggaggcttcaactcctcggacactgtcacctccccacaaagggcggggcctctggcaggtggggtgactacctttgtggccctctatgactatgagtcacggacagagactgacctgtccttcaagaaaggggagcggctgcagattgtcaataacacagagggagactggtggctggcacactcgctgagcaccggacagaccggttacatccccagtaactatgtggcgccctccgactccatccaggctgaggagtggtactttggcaagatcactagacgggaatcagagcggctacttctcaacgccgagaaccccagagggaccttcctcgtgagggagagtgagaccacaaaaggtgcctactgcctctctgtatccgagccagccaggccctgcttgcagggaaggatgccaagcagggcctgccctgacatttccacttcactcccatggtctcctgcccaagtctccccatgcacctggcgagtctaagcttgccacccaattctccctacccaactccaactgcccaaggacctttgtgt

SEQ ID NO: 2 - DNA, 1611 bp, Rattus norvegicus
atgggcagcaacaagagcaagcccaaggacgccagccagaggcgccgcagcctggagcccgcggagaacgtgcatggggcaggtggcgccttccctgcctcacagacaccaagcaagcctgcctccgccgatggccaccgcgggcccaatgccgcctttgtgccccccgcggccgccgagcccaagctcttcggaggcttcaactcctcggacactgtcacctccccacaaagggcggggcctctggcaggtggggtgactacctttgtggccctctatgactatgagtcacggacagagactgacctgtccttcaagaaaggggagcggctgcagattgtcaataacacagagggagactggtggctggcacactcgctgagcaccggacagaccggttacatccccagtaactatgtggcgcccttcgactccatccaggctgaggagtggtactttggcaagatcactagacgggaatcagagcggctacttctcaacgccgagaaccccagagggaccttcctcgtgagggagagtgagaccacaaaaggtgcctactgcctctctgtatccgactttgacaatgccaagggcctaaatgtgaaacactacaagatccggaaactggacagtggcggattctacatcacctcccgcacgcaattcaacagcctgcagcagcttgtggcttactactccaaacatgctgatggcctgtgtcaccgtctcactaccgtgtgtcccacatccaagcctcagacccagggcctggccaaggatgcgtgggagatcccccgggagtccctgcggctggaggtcaagctgggccagggttgcttcggagaggtgtggatggggacctggaacggcaccacgagggttgccatcaaaaccctgaagccaggcaccatgtctccagaggccttcctgcaggaggcccaagtcatgaagaaactgaggcatgagaagctggtgcagctgtatgccgtggtgtcagaagaacccatttacattgtgacagagtacatgaacaaggggagtctgctggactttctcaagggggaaacgggcaaatacttgcggctaccccagctggtggacatgtctgctcagattgcttcaggcatggcctatgtggaccggatgaactatgtgcaccgggacctccgagccgccaatatcctggttggcgagaacctggtgtgcaaagtggccgactttgggctggcccggctcatagaggacaacgaatacacagctcggcaaggtgccaaattccccatcaagtggaccgcccctgaagcggctctgtatggcaggttcaccatcaagtcggacgtgtggtcctttggaatcctgctgactgagctcaccactaagggcagagtgccctatcctgggatggtgaaccgagaggtactggaccaggtagagcggggctaccggatgccttgtccccccgagtgccctgagtccctgcatgacctcatgtgccagtgctggcggaaggagcctgaggagcggcccaccttcgagtacctgcaggccttcctggaggactactttacgtccacggagcggcagtaccagcccggggagaacctatag

SEQ ID NO: 3 - DNA, 523 bp, Rattus norvegicus
gtcgcctcccttcatcctctctctggggctgagttcctctgcaatgccccttttggagcatggaagggcttcagggacggggcagcctggaaggacaggttgaggctggtacagcgactgtcccacgcctgccatggccacaggctctctgtgcattctctctggagtcggcctgcttcatactgggtgacgagccgagctgggggcggttttccaacctcaacccactctacccacctggctcctccccactttctaaaccctgggtctgtttcaagctggccaaagagcctttccaaagaggaataaggcccagcccttggcctatatgtactaactgcccctggtcatccttgttggaggcttctgggtggaacattgggtctagaaccctctcccatggctctcaggctagacagcccaagacccaacaccagttgggataagtgtcagagtgagcactccctcagccctgtcctcaccaaggacccttgagatcaccaacacattaccctcatttctc

SEQ ID NO: 4 - DNA, 2001 bp, Rattus norvegicus
atgggcagcaacaagagcaagcccaaggacgccagccagaggcgccgcagcctggagcccgcggagaacgtgcatggggcaggtggcgccttccctgcctcacagacaccaagcaagcctgcctccgccgatggccaccgcgggcccaatgccgcctttgtgccccccgcggccgccgagcccaagctcttcggaggcttcaactcctcggacactgtcacctccccacaaagggcggggcctctggcaggtggggtgactacctttgtggccctctatgactatgagtcacggacagagactgacctgtccttcaagaaaggggagcggctgcagattgtcaataacacgaggaaggtggatgtcagagagggagactggtggctggcacactcgctgagcaccggacagaccggttacatccccagtaactatgtggcgccctccgactccatccaggctgaggagtggtactttggcaagatcactagacgggaatcagagcggctacttctcaacgccgagaaccccagagggaccttcctcgtgagggagagtgagaccacaaaaggtgcctactgcctctctgtatccgactttgacaatgccaagggcctaaatgtgaaacactacaagatccggaaactggacagtggcggattctacatcacctcccgcacgcaattcaacagcctgcagcagcttgtggcttactactccaaacatgctgatggcctgtgtcaccgtctcactaccgtgtgtcccacatccaagcctcagacccagggcctggccaaggatgcgtgggagatcccccgggagtccctgcggctggaggtcaagctgggccagggttgcttcggagaggtgtggatggggacctggaacggcaccacgagggttgccatcaaaaccctgaagccaggcaccatgtctccagaggccttcctgcaggaggcccaagtcatgaagaaactgaggcatgagaagctggtgcagctgtatgccgtggtgtcagaagaacccatttacattgtgacagagtacatgaacaaggggagtctgctggactttctcaagggggaaacgggcaaatacttgcggctaccccagctggtggacatgtctgctcagattgcttcaggcatggcctatgtggagcggatgaactatgtgcaccgggacctccgagccgccaatatcctggttggcgagaacctggtgtgcaaagtggccgactttgggctggcccggctcatagaggacaacgaatacacagctcggcaaggtgccaaattccccatcaagtggaccgcccctgaagcggctctgtatggcaggttcaccatcaagtcggacgtgtggtcctttggaatcctgctgactgagctcaccactaagggcagagtgccctatcctgggatggtgaaccgagaggtgctggaccaggtagagcggggctaccggatgccttgtccccccgagtgccctgagtccctgcatgacctcatgtgccagtgctggcggaaggagcctgaggagcggcccaccttcgagtacctgcaggccttcctggaggactactttacgtccacggagccgcagtaccagcccggggagaacctataggaactgtgtgcacagaactgtcacctctaggcttttgggtgtcgcctcccttcatcctctctctggggctgagttcctctgcaatgccccttttggagcatggaagggcttcagggacggggcagcctggaaggacaggttgaggctggtacagcgactgtcccacgcctgccatggccacaggctctctgtgcattctctctggagtcggcctgcttcatactgggtgacgagccgagctgggggcggttttccaacctcaacccactctacccacctggctcctccccactttctaaaccctgggtctgtttcaagctggccaaagagcctttccaaagaggaataaggcccagcccttggcctatatgt

SEQ ID NO: 5 - DNA, 16001 bp, Rattus norvegicus
aggatttggagacccccttctaaaggatatgacttgaaacatttttgttgcttgctcttccttccctccctccctccctccttccctccctccctccctccctctctcccttcttcctttcttctgtgcgcttatgggaaggcatgtatgtgaaccacgaatgtgtgaaggtcagacgtcagaggacagcttgcaggaccctttctcccatctctctctccaccatgtgggttctggggactgaactgaggtcagcagggtttatgataggtgcctttacctgctgagccatctccccaacccaggaagtgactttgagctgagagttgaagtaaactgattggggagatgtcttagtaagggttatactgctgtgaacagacaccacaaccaaagcaactctcataaggacaacatttaatcatttaatcggagccggcttacatattcacaggttcagtccattatcatcaaggtgggaacatggcagcatctagataggcatggtgcaggagaaactgagagttctacatcttcatctgaaggctgctggcggaatactggcttccaggcacctaggatgagggtcttatagcccacacccacagtaacacacctactccaacagggccacactttctaatagtgctgatccctgggccaagcatatacaaaccatcacaggaggggaagttgtcagggaatttgcaaaggtcttgtggctggaaggcctggccatctcctcttcaagccactatactgctgcttcaattaacacttttaacacctttcccccagtgttagacaacttgtcagacatgtattcattcagcaaaacttaaatgagtccctcctatgtgctggggacacagatatgactcgtgtccctgcctatgaaagactaaagctgactttgggtcctgctggacccagggctttggactggtatatatgtcttctatctgtagctgtgtcctaaggcgccaacatctcattttacaggtgaaactgtgagacgaactctcccagggtggcagggtccccttctcaggaccttagtctgcctatctgtttagtgggtagggtgtcttgtctgttcccagatctctcctctacctgggggatttctgtcgcccccagccaggaccatgggcagcaacaagagcaagcccaaggacgccagccagaggcgccgcagcctggagcccgcggagaacgtgcatggggcaggtggcgccttccctgcctcacagacaccaagcaagcctgcctccgccgatggccaccgcgggcccaatgccgcctttgtgccccccgcggccgccgagcccaagctcttcggaggcttcaactcctcggacactgtcacctccccacaaagggcggggcctctggcaggtaggccctcctccttgctgagtgtcccagagctgtggtgcaccctttctgggttctggggacttggttttccagaggctgcgaatggagaagggctcgtaaactggaaagcggggctcctgccgctttggggatgccagattcagagtataacgggttccgaaggagagggttgtaggggaccccgagacccggaggagaaggaccaccatggactagctgccacctcccgcccagcctttgttcctggctctgttgtgtggtggcccgagtgcgcccctgcgccctctgctggcgctgcgtggtgcggcaaccctgccgcccaggggctgagcagggtgactcatgctcctgcatacatggctacacccacacgagcaaatcccacgctctgacggagccatactcacggctgcacacccgtgcgtgcaaatcccgcactcggactcagccacacgcacggctgcacacccactcgttcagatcccacccagactcagccatgctatgtgcccacattgggcatgtcctcgcactgagtggagacgacaacaacaacaaaaggcccccagtgttcctagagacacacctaggctagacgcctgcagtgcggagcacacacagacacaggtgtgcatacaacctcgccccctcaggtgggcctgtgcgcctgcttgcacgcagaaagcagacatgggctaaggttcccagccccacatcccaccacagacccttggacaggctgcccactccctgccatgcagccacacacccattcgaccatttgagaggcgtttgtggagccttgggcaacggagggtgggctgcaggagtccaaggagcagccgtgggtgaggggagaatcaggagaagagtcctgaagctttgcaccaaagccttaagttgccaggcacacatataggcggggcttagggcacctattgtgtgctgggtctgagtgcagagcaagggaacctgttctctggggcttctgcttgtgtcagtagacatatcagataaggagcacctagtctgcagcccggcagggaggcacaggctacttagctccctctcagggacaggctggtgagtgagaaaggcagcctcccacgcccaccccctgcagggttctgacacctcccatcccactctgcaggtggggtgactacctttgtggccctctatgactatgagtcacggacagagactgacctgtccttcaagaaaggggagcggctgcagattgtcaataacacgtgagtggatggacggggactgcactgggagcgtgggtccatctatacccagagctgccggcagagggcggtgcagagcctacagggctgcttctgcagttggtgggtggtggtcctccctagaccccgactagtctcctcccttgtcctgggtcctgtcttcgcacctcagcctctccttctctctgcttctctctcgctggcccttaggaggaaggtggatgtcaggtgtgtaccgaggccaggtagagggggatgcttcgctgaggctggggactgctctctgcatgtgcttcctccactacccctgtgtgtctccagctctctccctgtccttttagcttatcctgcatcccacctgtttgatccgaccctccattttctccccacaccttcccgttagctctcttcctccctcttctggtgtgtgattccatgactgggtgccagctacttgcaggacccagtgtattctggaggccagggagggaggcagtgtggtggggaatgggttaccccagactaggccaggggtgggtaggagcagtctggggtccctgcaagatgaccctgagagagggggctgtctctatcggactgcccatccaaggaaggacagtaagctactgcaggtaagggtgggcagtctccctgggaggggggcagctgatggtctgtgctgggctgaggacctctccctggagcaggtaaggcttggggactttgatctgagctgttctggagggtagagaggatccgtcagagagtgctcccaagtcctggctaaagaggtgggttttccatttccctatgcgtgtctcggtgctcatacctcctgatccttgtgcatgctactccgtttgcctgaaatatttcttctcctgtatccacctggtgaactcctattcacccctcaagatccagggtagggctgccctccctgcaagcaggtgccatgtgctgttctggccactctcattcatacccctccatcttgttatcactggtcttgggggcagcagtgacgggggaggggctgctctagtttgtgaacgaggaaactgaggctcagagtcatgaaggcccttgtcagaggtcacgtgactgccaagtagtaagcccagaatggagcctgctctcctttctctggactggcacctggggaggggcctcctcagtacctccttcagtggccagcgctccttccatcctcatgggaagcccctgagtccccatgtccccagctgttgatagaattggtggccaggaggagaagcggtgtgaccctggtgctgagaggatagttggctcgcctggagtcacacggccgacagagacagagctgagggaaggaatacccacagaccttgaagggtgtgccgggtgcttgacctacccaccctgtggggacactctgcacaagtgacaggaggcaggaaaggcttggggacatggactctgtgtcttactctggggtcacagcagcctccactcctttggggtacagtttctttctccctctttgttgccttttagaatggtggggacctgtgcagccaggcagttagagtcagtcacaaaggagtcttatccactggaggccccggggtggtggattgggccacagggctgctgctgggacttcctgaccaacccctgctctacccaggctcagaaaagttcaggtgtcaaacagttctgcctggcggtaaatgtcattactctcaggtcatcttggcctggcacctcttcctagctgagatgctggggcccctatcatgggagagtgccagacccctggccagccagtccctggctacccgaggcataggtccactgaagcctaggtggtcatccctaccccagcctgaagctatgtccacactctacttgctagagactaaatattgcctcctttataaatgtgagcacatgcccagagagggcatgctatttgttcaaggtcacacagtgtttctccaggaactactcagctctggggggccggggcagtacaggcaggcacactaggagctgtggttttagtgcttccttcctgtttagctcttgtgatagcaacagttttatgcgtattagcacctgctccatgttggtctgtacactgacctttgaccttctgggagccagcgagggagctcaggcttcagatgggggcgggagcagtgggatacttctctgttcattttcttggagaggtatcccccaaacaggccagtgaacccctagggacccgtgggctgatgtcaggctgttttgctagctctggctatagtttggccacctcattctgtggtctctgatgtcaaacctgggtcccatcctggtacaagcctgtcttctgtcccagtcctcctgtctccctgtctcttgtcccctggcttctggagatttggagaacttgcatttggctgtggacattagggacccctgatttcagcctctctctgccattgactatgcgtgtggctttgggcaaatcatctcctcctctgtgcctcagtttcctctactctataaagaaaacactggtctctcttagtagagtgactgggttttccacctctctcaaatatagtttcctcagctagggtgggggccttgccttctttttttttttttttttttggttctttttttcggagctggggaccgaacccagggccttgcgcttcctaggtaagcgctctaccactgagctaaatccccagccccgggggggggggggggggcttgccttcttccttgttggatggtgtctgaggtcaagccggacggaaatgtgtgctaagcaagggtggacctttgggtgctgggtgagcttgcaccttgcctgtgtctgtgtccctcatgactccactcacaccctcttcactgcgcatgtccatttagaagggacccctctggcccagcattggccaagacagagtctgatgctctccccgcccgcctccagcctttccttcaagctcctccagctcagcttgctcctggcctcaacttctccatctgtgaagctgggcaaacacgtgctcagacctccagtgccgtcaagttgggactgtgtgactcaaggccaggacctttctgtctctggaccagggtgggatgctgtgtaatcttctcaggatcaaggcagggccaaggccaccagctgaataatttgaccctcatcttgtctactccccaagccttgcatgaagagctgcagagtcattgagcctcatggtacccacagggaggcacctggcctggctgtgtgaccttgcacagagccctttgcttctccaccctgtctttccgtctgtacagggggtgtgggaagggctggaccctagctcccaggccttgccctctgatcttccctcaaacatccaaccttctccccgtcccctttggttgctttggatgtgaggtctctggctgccccctacaccccattcccagtgtccccagcactgcctggctctctcgctctctctcccttgcccccccccccccatcacggcctctcaccacccccatctctgcctcttctctcctgtcgggctctcgcttgctcctccctcctctgtctctggacccctgctccagccagacctggttcacattcagatggctgcaaaggtacttctgcccttccccctcctgcccaggcccagagcccgagccctgctgggatccctcctccttcttgccctcctcccacctccctcccccctcttcctggggcagttagtgtagagggtcctgagcactgggctcagccacctctgttcccgtgacttgctgtgtgactttgggcatggcccagttctctctggcttcactttccccacagacaggggtaggctgcaggaggaggcctgtagttctgttaaggacttttaggagtgagctgtgatttgggacaggggcaagcattgcttgtttgaagggatgggactgaattattgctgggccagacgggctagcccttgtctccattgggcgttagggctctcagggcttggggagactttgaaggtctaatctggcatctgtctcatgtaaaccttgcttctcccttctcctccccgcaacagggagcttcctctctcctaaggcagcccttggcacctgcaggccgctgtgcacttctggacagttcagtcttgggtgggatggggctcagttctgctgaggttttaggcgccatccctctgggtcagtgtgtctgggaagtagaggttccaggccagttcctgctggcttgctgtgtacacaggctgcccctgcttcccttgctacagaacctggccatgcacctggggaagtcagggtcaccgaccctgctcccccggtgacatcattttttgctccctcccttctgctcagagagggagactggtggctggcacactcgctgagcaccggacagaccggttacatccccagtaactatgtggcgccctccgactccatccaggctgaggagtgagtatgttgttcggctatggtctgctgccactgccacccctagtacagggtcctttgtgggcctgagtgggcagcaggttggctaccctgaaaccccagtacccagggccagcattgtgggcagcgaaccctggtcaacagacacacttgtccctcccactccaggtggtactttggcaagatcactagacgggaatcagagcggctacttctcaacgccgagaaccccagagggaccttcctcgtgagggagagtgagaccacaaaaggtatgcttgagcacccctgtggctggcagaactcagcttcgagggtattatttgagctgggtattggagagtgaatctgagtttggtgcacgggatggggaggggagagcctttcagtggaggagatagcaggaattaaagttaccgtggtacttgaggaagactgagtgatccagaggtggcttccctctggcctcggttttcacacagggagtgggtacaggttgccagggatgtgacacagttgtagatgtgttctcaggatgggaacagaagcagtgtgcccctctgtttcccttttccttttcccaggggccttttctggccttgcccttcccccacagactgtctccgctctgattctgtcctttctcccagcccatcaccatggcaaccaagccaggctgcttcttgtgggcctggggctcagcaggagatggaaaagtgctcgtgctgtgccccctgtgtgctgtgcacagtacacgatgatgggtgtgtgtgtgtgtgtgtttgtgtgtgtgtgtgtgtgtgtgcgcgcgcgcgagcatgctcctgcactgctgggcttggagggtgctgctggagtgaaatgtggtgtggcctggctcctggggcttggggatatgtggccacagggcataggtatggccaccagcctgtgccttctgtgtgcacatgaagtgggcagactgtaggaaacatgacttcccagtattgtttgagacaggttgagtgttaccagaagatcatcatatttacctgcagaggttcactggggctccagggacctcctcagcgggtggtgggcagtccttccgctcagcactccctactacccagcaggtcgctcgaccctgtgccccacctctatctgagtcgatccaagctttatttaacaaaggtttctgttcagaacattaccagctgcagttcttctaggctccagagaggccaggcaggggcacagcctgcaggccacactcagaatgtacctcctgcccagggctaattactcccatgtccacataaccaacccagtggctactcctgtctgctcagctggtgttcagtaagcagatgaggacagagtgggcatacagagagcgcagtagccagcatttatggagtgcttgcggagtatcctatgaatcttcacaatagtgtcattgtgcaggggaggaaacagagaggacaagctacggctagttagggtcacacagttgtgagcacatttgggactcagacgtcttaccagggctgcgagtttgggggagggggtctctgccattgggggttttgtgatttgcctggctgtgcccggagcccgctcctctaggcacattaggggctgaaagagagtggaaccaactcacttcctcaagcaagaagcacactagcagggtgcctcggggtctcctccctgtctacccacaggtgcctactgcctctctgtatccgactttgacaatgccaagggcctaaatgtgaaacactacaagatccggaaactggacagtggcggattctacatcacctcccgcacgcaattcaacagcctgcagcagcttgtggcttactactccagtgagtgttcccggccgcgtggggactcacccaggggagcagggtgccacagggtgtgcctcctcacccctggaatgcctctaccctccctgcctctcctccatcttctcctttctcccctctctctccgcccagcctctccttcctccgcagctggctggtgaggagggggtctgccttgagcaagctagaggggaggggccagaggctggtgctgattgtttctgcagccctaagactggtctgaaccggttcctgggagagaaggagggggaggcttctttgacagagagcagcagagccggcgtgcgggtgcgggcgcgggcgcgggcgcgggccgtgtgcaaagatgggcttggtgtgcacccttctgtgcgggggctgcctcctcggtctgggcctccagcctctctaggactagctgccctctgtccgtctgcttctactctggggaccatttgattcccagaccaaatcctccaggaaccttgtgatacagttccaaggctccaagccccgggggcctttacctgccggttcctccaacgtcacctgctgcttcccacctcccggcctctcttccaacccctcagttgatcttgccttgtgccctggcataggtgatgtgctgtatggggtgccacctagttacccccccaaatcaaggccaggaacctgccttccagggctcccaaaggattactaaagcctgcttgactccccactcccaaactatcctccgtggcatctgtgtgctgagatcttgctaggtgactggggtgtgggtgtggcctccctagagcaggctctcacacctgttcctcactggctctatactcccaaatatggtgggtaactgaacgaatgagagatttgagcaggtgacttttgtgccacaggcccttggggaatagaggtctacaggagggtgtctgcctgtccccagagctaacgtgcacacgatgcctagggccatacacacaggacagggaggccagcttgctgtgggatcctgcctgagccccatttcctacttccgtctccagaacatgctgatggcctgtgtcaccgtctcactaccgtgtgtcccacatccaagcctcagacccagggcctggccaaggatgcgtgggagatcccccgggagtccctgcggctggaggtcaagctgggccagggttgcttcggagaggtgtggatgggtaaggaacacagccctatcctcatcccagctagacagctcaaagacctgagtccctgcctttaattgtctctgctgaacaactgggctctggttttgttttgttttattgccttacattaactatgcaaattattacgttttccctgtgacattttcaaactcacctgtaatatactttgctcctcaccctcacccccatacacactatgactcaactagggtcagcactgagggtgggcacctgactccttgtagcccatgccgctggagactagggcatctgggtaccaagagctgctggggactgggcaccatggtcctcaagggcctgagtgagtgtggccaggtctcacaccacgcctcagcctcctgatgtgaacacggggttgatgttaagcttcaggaggcatctgcatgttagttcagcacagagagagacagtgctgtgcggagggagtaatgggaagtaggtgtccggtctcccgtctaattgtgcagccgttgggaggcccttgggacagtcactgagctgtcctagccaaacccggctcccctgctgtcagctgctttctgcccacccaggaggagtaaggggactgttgggacttagtggccgtttccacttcactgtcttcttttcagagggcattagaggggttgggccaggtaggcaagggacgtcctgggttgtgaggagaaagggcagatggagaggccagctttaaaacagaccaaaaggaaggtttgtacagagcctgggtgagccacagtcttggtccttctcaaccttaactgtgattcttggtatgtggtggggatctgaggacttgccactttttcctggagttctgcacagaccagcaacctccaggaatgctggccctaccctcagaccccgcccctgtggcaaggaggtctcccaggctggcctgtctgttccctgtctaatgcagcccaggtgctgagctgacctcttgtccactgtgacctcagacacagccctccctctccactgagccatgttctggttgctggtgcctggagcactgaccgcccacccatcaggctgccttcttcctggcagcattcctaatcaatcaacatcctagtaacagttactgttcatgagcagactccagccccgggaggggtcgccattcctaagtgggaatagagtctcagtgtagtgtctggccttgcccgggcccatgccatgagttcagaggcagggattgagacagacacccatatgggctagcctgggtggtcccagtgtgacataatggtggcctgtgctgatgttgctatgcccctcccccacagggacctggaacggcaccacgagggttgccatcaaaaccctgaagccaggcaccatgtctccagaggccttcctgcaggaggcccaagtcatgaagaaactgaggcatgagaagctggtgcagctgtatgccgtggtgtcagaagaacccatttacattgtgacagagtacatgaacaagggtaaggtctgagcccaggctgacctctgctcagatgcccgcacggcagactgagggtaacttattccttaccccaaaccaaccagatgagtggacagctgtgtcagggactgcctctctcctttcgtaaagaataacaaatcaactattttttcccctaaaagacgtttcttttgtcccttaccactgacaggatccctgttgttgctggtgtgatggcacacacctataatctcagtgcccatggctctgaggcaggaggatcttgagtttgaggccaggccgggtacatagcaagactgtctctgtcttggcaaacagcggtgtggggctgggggtgtagctcggttgggagagtgcttgcctggcatgcacaaggccctgggtttgaccttaggtgtgatctccagtaccacaaggctaggcataagacacagacctgtaatcccagcccaggggacgtagagacaggaggatccagagttcgaggtcggcctccgttgcaccttgagttggtttatgtgagaccctgtctcagaacaaaaccatgtatagcgtccacattaccccagagttctctgctggacttagtgatgtgactcagccccagccccaggtagccactggctctccggtgcggccctgataaagcagtttctctcgggctttacacataatctcttgcatcttacaacccctgttgtgttgtctgcctgaccgcctccctacccgccttaccacaagtccttgctctaactccctgcagtgttggcctttgagcagggggccaggttctgggagccacagtttcctgcacagggagacttagacaaggctagagctgggtctctctctccagggagtctgctggactttctcaagggggaaacgggcaaatacttgcggctaccccagctggtggacatgtctgctcaggtgagtctcctcctccaccttcctccacccttagctctcgagaccagagcgcccccttcaggagatctgccttcattacaccagagtctcaccttgcaggtgcctggggtggtacctgggagtcagaggggcacaaacacgtaatgaagaataaaaataaaacattaaaagtttaaaatgtactacttccatttgctagatgaggaaaccgaggctcagagaggggacttaggacaacaggcaggatctagcacgcccctgtgtctgtggggcctccttttttatatactcacctgcccccctggctgcgtcttattgtgacctcccattgtcctttgctctcccctcccccactgagctccggtctacttttcaagccagcgctgtttattttaagctgaatgaagttccttgcttgaacagacacaggctaggcagggtgtttgagtgaccagcccgaggcacctgctcacagcacagggatgggtgtggttagcccctgagccaggctctctccgtctttacagattgcttcaggcatggcctatgtggagcggatgaactatgtgcaccgggacctccgagccgccaatatcctggttggcgagaacctggtgtgcaaagtggccgactttgggctggcccggctcatagaggacaacgaatacacagctcggcaaggtgggagtgcctggcggttatctgatggtatcggtctgtcaggtgggagtgagagcaatgcccctgcagcagactggtatcgatagcctgtgctttcttgcttgtaggtgccaaattccccatcaagtggaccgcccctgaagcggctctgtatggcaggttcaccatcaagtcggacgtgtggtcctttggaatcctgctgactgagctcaccactaagggcagagtgccctatcctggtgaggagcctgtacctgtgcccctgaccttggccctagggcaagcctcaccaccaccaccccaatgctcccccacatcacccacccctgggcttcctctttctaggctggatgtgtctgtcaaagaggctgatctctgatcacaggctgcctctggtcaggactcttagcctccaaggcagtgttctgtacagcatagggtaaaagtggtctaggctgtgccagtctctggagccatctcctcatagccctctctctgtttgttcatagggatggtgaaccgagaggtgctggaccaggtagagcggggctaccggatgccttgtccccccgagtgccctgagtccctgcatgacctcatgtgccagtgctggcggaaggagcctgaggagcggcccaccttcgagtacctgcaggccttcctggaggactactttacgtccacggagccgcagtaccagcccggggagaacctataggaactgtgtgcacagaactgtcacctctaggcttttgggtgtcgcctcccttcatcctctctctggggctgagttcctctgcaatgccccttttggagcatggaagggcttcagggacggggcagcctggaaggacaggttgaggctggtacagcgactgtcccacgcctgccatggccacaggctctctgtgcattctctctggagtcggcctgcttcatactgggtgacgagccgagctgggggcggttttccaacctcaacccactctacccacctggctcctccccactttctaaaccctgggtctgtttcaagctggccaaagagcctttccaaagaggaataaggcccagcccttggcctatatgtactaactgcccctggtcatccttgttggaggcttctgggtggaacattgggtctagaaccctctcccatggctctcaggctagacagcccaagacccaacaccagttgggataagtgtcagagtgagcactccctcagccctgtcctcaccaaggacccttgagatcaccaacacattaccctcatttctctgttggaaaaactgaatccaggaggggctgggacctgtccaagaccacgggcagtttcgggttgaggtgggcttggcacctggtgctccctctgtcttcctcaggaactaacgagagctgtcctccaagggagtgggcaggctgaggcccgagagtggcttcaagttgtcagggttctgctctcactgatgctcatgggtctatcatgggggaagaggcaagcaggaggctgagcccggcgtgaggggtaggaaatgacggtgtggaagaggtgcgtcacaatgcagctgggctttgaccttaagagctggccgtgaagtggagtgagcgctcaggctgcagaggcagtcaggtagaagaggaagaactaagaagtgaggagccccgctcctcaatttgtcctgatcatttcaacaccctgcgcatgctcttctgcctatgctgggattccctaactcacagggagggaggggagctccccggctggggaaaggagggagacgatccttactgctatgcaccaacactgaccctgccctctctaagctaaactctctgcccctgtgactctggcttctgccattgctcagcccctttatagatagatccacagctgggtgaccaaggggagcacggccctggaacagcagacagctgactgcttggtccctggctcttctgcctagccttaggatatgtggtagcttctctctgcctcagtgtccctatctgtaaggtgggcagttggtgatttgtggcatttcaaccaaggacccctgtgtgtgtgtgtctgtgtgtgtctgtgtgtgtgtgtacacatgtgcctgtgtcttcccttgtgtccatatttaacgtgtaaaaacatccctactctgtccccagacatggccatttcacaattaccccatcacagcaataatgtccccagcgccggggatccgagccagccaggccctgcttgcagggaaggatgccaagcagggcctgccctgacatttccacttcactcccatggtctcctgcccaagtctccccatgcacctggcgagtctaagcttgccacccaattctccctacccaactccaactgcccaaggacctttgtgtaaggtggcctcagcggtccggttttctccttttctttctttttaacagtgttttgtagatttcggatgactatgcagaggccttggggacccccagctctcggcagggcctggggatcccacattccatggcccaggtgtgggggagggttacccagactggttgtacatactctgcatactgtctgattaaacacgaacagacctcagtttggccagcactttattgaacatggattgtgtgtgggaataagaggagtcctggctgtgagggaagatggagtcct

SEQ ID NO: 6 - DNA, 420 bp, Rattus norvegicus
  misc_feature 224: n is a, c, g, or t
tgctgagtgacctgtgtaaccatgacaggccgagccgggatctgaacaaaggcagagtctctgcagatgcagatgcagatgttgagacccagagaaggggagctgctcgcctagggatacgtagcaggagaggaggagcagggcctttaagccaagacccggacactacggctggtgtctgtgccaagcccccagactgctcccctgatccactggggtagccntgctacctaccagccagggccatgggcagcaacaagagcaagcccaaggacgccagccagaggcgccgcagcctggagcccgcggagaacgtgcatggggcaggtggcgccttccctgcctcacagacaccaagcaagcctgcctccgccgatggccaccgcgggcccaatgccgcctttgtgccccccgcggccg

SEQ ID NO: 7 - skipped

SEQ ID NO: 8 - DNA, 899 bp, Homo sapiens
  misc_feature 671: n is a, c, g, or t
  misc_feature 680: n is a, c, g, or t
  misc_feature 723: n is a, c, g, or t
agactgccactgtcacagctgttagcagtaatgcattacaaccctgaagttaatcactatttcctgcttccttaggaatatgtgtgtcttctgatcaaaatcatttccgaggagaaattgaggaaaccatcactgaaagctttggtaatgatcatagcaccttggatgggtattccagaagaaccaccttgtcttcaaaaatgtatcacaccaaaggtaaggatgttaagactcattcttagcacatcagaagtgtcttttgaattattttagtgaaacgatgcaggtttagcagtaactatgtacttttttcctactgtcttctccttcgtaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagcgttgttactgtggagaaggtctgtcttgccggatacagaaagatcaccatcaagccagtaattcttctaggcttcacacttgtcagagacactaaaccagctatccaaatgcagtgaactccttttatataatagatgctatgaaaaccttttatgaccttcatcaactcaatcctanggatatacnagttctgggggtttcagttaagcattccaataacaccttccanaaacctggagtgtaagagctttgtttctttatggaactcccctgtgattgcagtaaattactgtattgtaaattctcagtgtggcacttacctgtaaatggcatgaaacttttaattatttttctaaaggtgctgcactgcctattttttcctcttggtaatggaaaatttttgaa

SEQ ID NO: 9 - DNA, 1554 bp, Homo sapiens
ggccattaccaatcgcgaaaccccgcagggggctcccggaccctgactctgcagccgaaccggcacggtttcctggggacccaggcttgcaaagtgacggtcattttctccttctttctccctcttgagtccttctgagatgatggctctgggcgcagcgggagctacccgggtctttgtcgcgatggtagcggcggctctcggcggccaccctctgctgggagtgagcgccaccttgaactcggttctcaattccaacgctatcaagaacctgcccccaccgctgggcggcgctgcggggcacccaggctctgcagtcagcgccgcgccgggaatcctgtacccgggcgggaataagtaccagaccattgacaactaccagccgtacccgtgcgcagaggacgaggagtgcggcactgatgagtactgcgctagtcccacccgcggaggggacgcgggcgtgcaaatctgtctcgcctgcaggaagcgccgaaaacgctgcatgcgtcacgctatgtgctgccccgggaattactgcaaaaatggaatatgtgtgtcttctgatcaaaatcatttccgaggagaaattgaggaaaccatcactgaaagctttggtaatgatcatagcaccttggatgggtattccagaagaaccaccttgtcttcaaaaatgtatcacaccaaaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagcgttgttactgtggagaaggtctgtcttgccggatacagaaagatcaccatcaagccagtaattcttctaggcttcacacttgtcagagacactaaaccagctatccaaatgcagtgaactccttttatataatagatgctatgaaaaccttttatgaccttcatcaactcaatcctaaggatatacaagttctgtggtttcagttaagcattccaataacaccttccaaaaacctggagtgtaagagctttgtttctttatggaactcccctgtgattgcagtaaattactgtattgtaaattctcagtgtggcacttacctgtaaatgcaatgaaacttttaattatttttctaaaggtgctgcactgcctatttttcctcttgttatgtaaatttttgtacacattgattgttatcttgactgacaaatattctatattgaactgaagtaaatcatttcagcttatagttcttaaaagcataaccctttaccccatttaattctagagtctagaacgcaaggatctcttggaatgacaaatgataggtacctaaaatgtaacatgaaaatactagcttattttctgaaatgtactatcttaatgcttaaattatatttccctttaggctgtgatagtttttgaaataaaatttaacatttaatatcatgaaaaaaaaaaaaaaaaaaaaaaaaaaaaaaaaaaaaaa

SEQ ID NO: 10 - DNA, 3869 bp, Homo sapiens
tttgcagagcctatcacccctcggctctgtaaagtatttcagatccggataattcaacccttactgccaggcaagggcacccaagttcccagagttcctgctgccgcctgcatttattaaagtcgtctgctataacgctcgctggtagccttcaccccgaaggtgagccgggccagccgagcgactaagcaagggaggggcggggtgaagagtgtcaaaggccccccttcatgtacacaaacacaccccctcccagcccctcccagcgctttgaaatcccatcccggctttgttgtctccctcccaaggggccggaatgctccgggcccgcggtataaaggcagccgcggtggcggtggcggcgcagagctctgtgctccctgcagtcaggactctgggaccgcagggggctcccggaccctgactctgcagccgaaccggcacggtttcgtggggacccaggcttgcaaagtgacggtcattttctctttctttctccctcttgagtccttctgagatgatggctctgggcgcagcgggagctacccgggtctttgtcgcgatggtagcggcggctctcggcggccaccctctgctgggagtgagcgccaccttgaactcggttctcaattccaacgctatcaagaacctgcccccaccgctgggcggcgctgcggggcacccaggctctgcagtcagcgccgcgccgggaatcctgtacccgggcgggaataagtaccagaccattgacaactaccaggtgagaggggtcgggcactcagaggatgctctgaccttgaaagggtcctatctggagacgagggagtagaacgtgctgaatgtgtgcggttcagggagcatttggtaaccctgcatttgggagcagtgggcagtaacaggttttggagaggtggacagataaggactgtgatcagcgcccgggtccaagagggcgggtacctggacgtctgggtgcctcaccctctccccgaacccttcccacagccgtacccgtgcgcagaggacgaggagtgcggcactgatgagtactgcgctagtcccacccgcggaggggacgcaggcgtgcaaatctgtctcgcctgcaggaagcgccgaaaacgctgcatgcgtcacgctatgtgctgccccgggaattactgcaaaaatggtgagtcctgaaagctccctttcacactaaaactgtccagcctttgagcgtctatgaattgggcgggggcggggggttggggggggtggggggagaaatctccgccctgagaacactgcggcgccacctgcaaatgggtgttcagcatgcaggattccgctgaagtatcttcattgcaagtgtttaatcgggaagaagagagaagttgggaggtctctgaggtccttatcttcctccgtgtccctcaatgattcaatacaacggttcacctcgtagtggactcttccataaaaatttgtctggggagaaccttgaggtcaattgagaagggggatgatgaggaggacgatgggatcatacagatatcaaagaaaccagctttggcgttcagctgtccttttcatttttcttattgtgattctcacagttttgccagactggcctcgccttctgatttttgctgaaactgttgtcagccagaggactgatttatctgcattggccatttttaagagagatcttggtaccctcacaacttgacttctccatccttccccctccctctaccctctgctgccttaatataagtactcctccccccaaccacagatccactaacttttaatttttcaaaaggtggatttagacagctaaagaggtctccagaaataagctaacagggcgacgaccctccttaggacccagcagtactccaggatgaactaagtaaatctctgtaatatagaaattacacagtaaagtaactggccggtgagattgcaatgttccaggcagctggaattgcatttgcttaacttagtctggtaggacccatatagcaccaggggcagagaagttatttttaagaacatttttaaagtgactgagtttctttttcttaccccaaaggtaatattaaaatgggcagaaatctcacttgcccctaccacagttggtgggaaagtattttaaatggctatactggggatattacctatgactgactgtatacagtatacacattggtgcttctgtccatctggagagttttgcattaacagtaaagaggaagtttggcttgtgtttaaaatgatgtcactgcaatgaagtatctcaagttgatgctggcataacagactgccactgtcacagctgttagcagtaatgcattacaaccctgaagttaatcactatttcctgcttccttaggaatatgtgtgtcttctgatcaaaatcatttccgaggagaaattgaggaaaccatcactgaaagctttggtaatgatcatagcaccttggatgggtattccagaagaaccaccttgtcttcaaaaatgtatcacaccaaaggtaaggatgttaagactcattcttagcacatcagaagtgtcttttgaattattttagtgaaacgatgcaggtttaacagtaactatgtacttttttcctactgtcttctccttcgtaggacaagaaggttctgtttgtctccggtcatcagactgtgcctcaggattgtgttgtgctagacacttctggtccaagatctgtaaacctgtcctgaaagaaggtcaagtgtgtaccaagcataggagaaaaggctctcatggactagaaatattccagcgttgttactgtggagaaggtctgtcttgccggatacagaaagatcaccatcaagccagtaattcttctaggcttcacacttgtcagagacactaaaccagctatccaaatgcagtgaactccttttatataatagatgctatgaaaaccttttatgaccttcatcaactcaatcctaaggatatacaagttctgtggtttcagttaagcattccaataacaccttccaaaaacctggagtgtaagagctttgtttctttatggaactcccctgtgattgcagtaaattactgtattgtaaattctcagtgtggcacttacctgtaaatgcaatgaaacttttaattatttttctaaaggtgctgcactgcctatttttcctcttgttatgtaaatttttgtacacattgattgttatcttgactgacaaatattctatattgaactgaagtaaatcatttcagcttatagttcttaaaagcataaccctttaccccatttaattctagagtctagaacgcaaggatctcttggaatgacaaatgataggtacctaaaatgtaacatgaaaatactagcttattttctgaaatgtactatcttaatgcttaaattatatttccctttaggctgtgatagtttttgaaataaaatttaacatttaatatcatgaaatgttataagtagacatacattttgggattgtgatcttagaggtttgtgtgtgtgtacgtatgtgtgtgttctacaagaacggaagtgtgatatgtttaaagatgatcagagaaaagacagtgtctaaatataagacaatattgatcagctctagaataactttaaagaaagacgtgttctgcattgataaactcaaatgatcatggcagaatgagagtgaatcttacattactactttcaaaaatagtttccaataaattaataatacctacctaaatggtcaatatttttcggacaaggaagaaaatcatccacaaaaataatactccaaagtacttggtgattggcaggaacaggatgtgtgcccataaatacagttaacaaatacatgcagattttg

SEQ ID NO: 11 - DNA, 9001 bp, Rattus norvegicus
atgctatcctcgagtgacacacacagttcaattccttctacttttaaatctaacataaaacattaactgaattgcagtaactgtggtcatatttagggcatattcgacaagagacaaagttccttctagaacaacgttgactaagtttaatagatatacccggcactccaacacctagctacatgaggctcagagtcatgagacctctgacttcctgagactggggtgtggttctagtgcctggacctctcctcttttctcccctgacaaatatataccaagtaaaaatctacgtaaatagcaagactgagccagatgtagtgatgcacgcccataaccctcacaatggaggaaacagtgatagaaaatctgacctgcagactacatagtgagacaccagtggtctaaaggcccagagacacacacccccatgccaccccaaggcgacaacaaaaaccaaaccaaacaacaacaaaactccctctgtggcctgatatcatgtttcccaggctactgagagactggaaggagcctgggagactggaaggagcctgccttcagcatactagagaactgaactgttcttaaggagctcctctgggtgtgctctgcactgaagaagcagcgagcatctctttctcgggagaaagctaagtcaaagtgagcgtccacagacgcagaaaaggctgcaccgaaaagggcaagtttctcccgctgcatcatttgtgagcctgcatcctcttttcctgaataacctggaggcagctgttcctcttttatttaatttttttttattgaagcaacgagtacagggcttagcctcaggtgtgtgagcctgttcacaccttcagaaggaactgaaaaagcagggagtgccaaaactagcccgtggcttcaaccgtgggcctgagttctaggcgggttaagagctgctcctttagagtagatgcattaatcaagcatcattccctcaggatacagggaaaggagtgcaacatttgtgtctttctagaattattgtaacaggagaacagggccagggaaatctctctttactcttccaagtgggcaggggcctcattccctcctcttcaatgtcctatgaatttatattaagagatactcaacacattgatcactggggttgctagcaatatgctatatgtgatatgagccccaccaccaccaccactttatccttctatcccaccgtctcccagcatctatgcaaatttcagacccactgaccagtaggtctgcatgaagtcaggaaaatctgatttattgttatctctttgctcccccttttattatttctaatattcacttatttaaaaaatgttttagcaaggccctgggttcggtccccagctccgaaaaaaagaaccaaaaaaaaaatgttttaaaaccttttctttgttttcaaattttctaagcatctataggtaactagccttgctacacaattgcacacattcctaataagagccagataaatatggaggatttaattcatgttggactttcaaccaaccaccggaaatctccccgggatgaatggagttttggggtgagtgggaacagcattgggtatcattaaagaactgagacaacattaaaagttacttggaagacacttaagttctgaaatttaccaaacagatatttaaatattttcatgatatccctcctagctcaaggtcaggattatatttgtatgagactatcattgggggtgggggtggtcccaagaatgactattatagataaggcagtgttgctaatcaagaagctaacaatgattttcaatcgtttcttccttttcaatctcacacaacacagttgcttatgatctcacacttatcaatctgttcttaaacattattaatgcacatttcttatcttttgctatatcatgacaaccgtctcttttagcaccacaatggttggataaaggacttgacttttattctcttgatacattacctctaatttataagcacagcactcttaggtccacgttgtttcttattttacacactggttgaaaccacttcaaattcattctctgatttaggttcagtgctccttttccaggaggtcgtctgttatctccctagagggttattcctcctgtcctgtcctgtcctggacgaggtttgatttgggatcaaaagcaagccctctcctgctcggcgatatgaaacactaatgaggaagccgggagctaagccttcggggtttcttatcttcttctattaacccaccgctgggaaccgggaaccagggaaagaggactgaccaggctccgggaccctgtccgctctggctcagagccactttgatctcacgtgtctgcttaatcaagttcatctactgctgggattgaccagattcaaagaaccacattaaatagtttgattatcgggtgcccatttccctcttccctccatccctgatttaatttccaaactgcagtctctcgcctccacccccaacccccccaaactcaccatcaaaagcgaattagttcttcccttggcatcctcatttgatgtgcaaatttttttgtctttgcacatttttctcctggtgtgtgtggggttcgggaaggggggcggaatgggagggggaagcaactagacaaacttaaaagacaagaacctcatttgcaaatcgcaacgctaaagggttaatcagcaactatgggagccacctgggctttccacaaagcctttgaatgactgctcacgttgtatttaatactgaatatttgcgtgcacagaaaacaagggtaggcgtaaggggaagagaagaaacgtggttttgtatattctatcacttttcactctgacaagtatttcagaccgaactaacccaatcccgtaacagagcacttcagagcacttcgcagagagctccctcccgccttttttttttttttttttaattattaaagttcaggctaggctctaaagctctagtaacccacactctaaggtgagctggacagggggactatagtgccagggaggggcgaggggagagtgtcaaagtcctcccagcttccgatacacaaacactccccctcccagctcctcccaaagctttgaaatcccatccctgctttgttgtcttccccgaggagctgggaaggctgggagcctgagtataaaggcagccagaggtggcggtggcggtgcagagctgtgcgcctggcagccgagactccacccgccgaggttccgaagttgaggttccgcagcccagactctgcagccgctgtgcaaagtgatctctctcttcctgactttttcctgtttgcgaccttctgagatgacggttgtgcgtgcagtggcagctgtccggttcttggtcgtgctttcaacgatggctctctgcagcctccctccgctcggagtcagcgccaccttgaactcagttctcatcaattccaacgcgatcaagaacctgcccccaccgctgggtggtgctggggggcagccgggctctgctgtcagcgtggcgcccggagtcctctatgagggcgggaacaagtaccagactcttgacaactaccaggtgagaggggtctctgggatccgaggacgctgtgatcccgaagcgggggtctcagagaagagggagtggagtatcctaggtgtgtgagatttggggtgtgcttggaatcactgcactgagagcgcgcttagagagcttttggagaggcagatggggactgtaaaggtgtgggtaccgggatgcttgagttgctcaccagttcgctcccttcccacagccctacccttgcgcggaggatgaggagtgcggcactgacgagtactgctccagtcccagccgcggggcagccggcgtgggaggtgtacaaatctgcctggcttgccgaaagcgcaggaaacgctgcatgaggcacgctatgtgctgccccgggaattactgcaaaaacggtgagtcctggactcccttttaaattagaaccaccttgcctttgagccgccaggaatcaaatgaaatgctctggttctgtcaaagctacggcggcggcgccacctgcaaaagggtgctcgtcaggctagggttcatcctgacatagcttcagaggcttgtgtttgagcatttgaaagtttatcttcccttgcctccctctgtgatctaacaacaggacctgaaagtgggacccttccatcaaacaattaccttcagggagccttggggcacttgagaaagagggagaggtctttttgaaagggaagccagacgtttttcttaaggtggactctgcgtggctttgccaaactctcttctctctcaaggtctccggcagttaaaactgttgtcaaccggggaggggtggggggctagattgattaatctctgctggccaacttttaaaagagatctctgtactccttacatccctggctctccaccctctcaccctttccctccacccttaactaccttcaaagaagtactctctggggcaaccacagatccacaaaatgttaatgtctcaaagaagtctccagaaataaacttacagggtggctgacttcctaggggacagcagcattccaagagtgaactcacaaaatctctgtaatatcaaaattacacagtaaagtaactagcgggtgagagcgtagtcccctaggcagctagtacccagagcagagaagttatttttaagattaattttttaagtgactgaatttctttttcctacccccaaagggtaatattaaaatagacagaaatatctctgctgcctccaccacaattagtggggaagcatttaaaatggctaccctgaggtgttccataggatccaacagtacactcattgacactgctttctctggggagttttggtcaacagaaaggcagtgaaatagtgaggtatctcactctagtgtctgcataactcactgctcctgtgttgactattagcaacacattatggcctgaggattaattactatttctggctttcttaggaatatgcatgccctctgaccacagccatttacctcgaggggaaatcgaggaaggcatcattgaaaaccttggcaatgaccacggtgccggggatggatatcccagaagaaccacactgacttcaaaaatatatcacaccaaaggtaggaaaaaggcttccccactcagcccctccatttagaatatcagaagtgcatttcaaattgtcttaacggaacggtacaggcctagctgtaactgtcattttccttctgtctcctttacagggcaagaaggctctgtctgcctccgatcatcagactgcgccacagggctgtgttgtgcaagacatttctggtccaagatctgtaaacctgtccttaaagaaggtcaggtatgcaccaagcacaggaggaaaggctcccacgggctggagatattccagcgctgttactgtggggaaggtctggcttgcaggatacagaaagatcaccatcaaaccagcaattcttccaggctccacacctgccagagacactaaaaccaacagcctaaatgcgatggactctcttatctaatatatgctatgaaaatccttaatgatttgtcagctcaatcccaaggatgtaggaatcttcagtgtgtaattaagcattccgataataccttccgaaaactctggagtgtaaggactttgtttcttgatggaactcccctgtgattgcagtaaattactgtgttgtaaatcctcagtgtggcacttacctgtaaatgcaacaaaactttttaattatttttctagaggtgctacattgtctcgtttctcttgcatgtaaattttttgtacacggttgattgtcttgactcataaatattctttattgaagtaatttcagtttacagttcttaaatgtggaaccctttccacttttgtctctagggtgcagaacgcaaggagtttttggaatagtcaatgataggcatctaaaatttaacctgaaaatgctcatttatttcctgagatgtactctctttgcttagatattctctctctctctctctctctctctctctctctctctctctctctctctctctctttctctctctcctctcctctcctctctcctttctctgtctctctcagctgtgatagtccttaaaataaaatttaacatttagtaatcatgaagttacaagcatagatacatttgaggctgtgtgtgtgtgtgtgtgtgtgtgtgtgtgtgagagagagagagagagaccaagacagagtcagagacagagagagacagagagagagctttttaaatgataagatatattgtctatgtgtgagttactttaaagagaatgtattctacattgataaactcaaatgactttggtaatatgggtttgaatttgatattactactttcaaaactactattttactatgtgtggttggtgcttacgcctgtaatcccagggttctgggggatgaggcaggaggaatgtccatgaattcaggtccatcctggccaacttatgctatagggtaaggttcgatttcaaaaatgcatcaacaaaagatagttaacagcaaattaatatttccctaagtgaccgacagtaattggaagggtgggaatgtgacttcaaataatgggcagagttcagaagagattatgtggaaggaaacccaccaacttaagccaaataattaattctgaatattttagatactctgacatgctgaagaatggtcccaattactttgatttaaaaataattactaaacaattataaattctagaatgtaataccttttcttttcttgccaagaaattattccattttttgcttcgcctgtactgcattgattggaatgagtatggctttatataaaaataatccccaaattaaattctctctgcatatttcctcactaaacttcatgtttctttttggggggtaggacaaaacaaaccttcctaatttatttattctctagtattctggaagtctgcatcaaaaccacaaaatgctgggttcactattcacaggatagtaggggctgaccgattgtcccagcattctccttaaatagcataggtggcctagaatattcatctgtttttgtgttcccggaagtggttaattttccagggagacatacaacagcacatgcactctactggctcactgagaggagcccttctgttcataggtctctgccttttcatccagagaaactctctccctaagtttccttcctcattgcctgttgtgctaggaaggccaggcctgcaatcttcagtaatacacaacaagaggagaaaggtaatggggaaatgtccatcaaacaggagaaagaagggctttctaggagaggagcgggacagatgtttggtgcattctggttactggatcctttatgcaggggcctgctggggtggggatgctggggtggggatgccttttcctgcacatccctttcagcacattcagttctggcctttcctgttgattctcaggatggggccagagagctaacctccatccacctgggatcttaggaggacaatatcaagaaaatgtatgtgtgtgtgtgtgtgtgtgtgtgtgtgtgtgtgtgtgtgtgtgtaatatacatatactatatataatatattgtatatattttgtagaatatattatatagtacattgtatatactatgtattcatatattatacatatatatgaagatgaaaaaaatcataactgtaggtactttttaattcaaaaactagaacagagacattacataccacgcaatcactaaccaggatcgtagggaatctagaatccatttgccgtagctgggtcaaatgctgtgtggtggttgagtcctaacaacgtatttgactggctgcccgtgagaacagctcaccagctcgttctactgtttcatacttccataggttaccctctgcctcgctcctggctctctctgctttaggtgccttgactactgtcctatacagcaaagagtaggtgactcagcctttgagtgggtctcattgtttataggctctcctctgacttatccaaccgggtgccaaaatacaatcacgtgatttacagagtaactttaataaatactgtgccacaggcataaaatcctcctgttttatcatggtatagctgtaagacaatgtttgagttttgaaacaaaatttaaagtcaaacaccatgctttttctacctactcaaggtctggcatggtcacaatggcaagattcctatttgtgtggtgataaactcttatgaaaatatgttaaaggagaaatggggtgggggagaagctttacttaaaatgtttacagtcagcatcgatctcttatactagaaggttaacttactctaacttcctttatatcaaagtagttatagaaggccatcacctatttcttatattcagttggaattcttgaaaatttttcctctctttaaaattatttcatttatttttaatagtctattatagttacattgttccattcttccctttcctccctccaatctctcctatatacccctccttgctctctttcaaattcatgggctcttggtttttatgtaatttttattacaacatatatgtatctacatatatatatacacatatgtatatatgtacctgtgtatatgtatatacactaattgtaaccatcagagtctgtatagtgttagttgtgtatgtgttctcagggctgtaaacttgatttaatctgacgccaagacaaggataggatccattatctaaaagtgaaaatgggcagcaggaatggctgatgggttcagagcacccactctgcaggcctgagaacctgagtttaaaacccagcgactgcatggatgtctttcatgggtgcatgttcctatcaccccagaactgctgggcagtgggcagcagcggcagtggtgaggaaagacttgtaaatactaagcatgttcctcaccactgagctat

SEQ ID NO: 12 - DNA, 813 bp, Rattus norvegicus
atgacggttgtgcgtgcagtggcagctgtccggttcttggtcgtgctttcaacgatggctctctgcagcctccctccgctcggagtcagcgccaccttgaactcagttctcatcaattccaacgcgatcaagaacctgcccccaccgctgggtggtgctggggggcagccgggctctgctgtcagcgtggcgcccggagtcctctatgagggcgggaacaagtaccagactcttgacaactaccagccctacccttgcgcggaggatgaggagtgcggcactgacgagtactgctccagtcccagccgcggggcagccggcgtgggaggtgtacaaatctgcctggcttgccgaaagcgcaggaaacgctgcatgaggcacgctatgtgctgccccgggaattactgcaaaaacggaatatgcatgccctctgaccacagccatttacctcgaggggaaatcgaggaaggcatcattgaaaaccttggcaatgaccacggtgccggggatggatatcccagaagaaccacactgacttcaaaaatatatcacaccaaagggcaagaaggctctgtctgcctccgatcatcagactgcgccacagggctgtgttgtgcaagacatttctggtccaagatctgtaaacctgtccttaaagaaggtcaggtatgcaccaagcacaggaggaaaggctcccacgggctggagatattccagcgctgttactgtggggaaggtctggcttgcaggatacagaaagatcaccatcaaaccagcaattcttccaggctccacacctgccagagacactaa

SEQ ID NO: 13 - DNA, 712 bp, Rattus norvegicus
gatttcttctactcgagtggcgagaagaaagatgaggtctaccttaacctggtgctggactatgttccggaaacagtgtacagagtcgccagacactatagtcgagccaagcagacactccctgtgatctatgtcaagttgtatatgtaccagctgttcagaagtctagcctatatccattcctttgggatctgccatcgagacattaaaccacagaacctcttgctggatcctgatacagctgtattaaaactctgcgactttggaagtgcaaagcagctggtccgaggagagcccaatgtttcatatatctgttctcggtactacagggcaccagagctgatctttggagccactggaagcttgtgcacattcattttttgatgaattacgggacccaaatgtcaaactaccaaatgggcgagacacacctgccctcttcaactttaccactcaagaactgtcaagtaacccacctctggccaccatccttatccctcctcacgctcggattcaggcagctgcttcacctcctgcaaacgccacagcagcctcagatactaatgctggagaccgtggacagaccaataacgccgcttctgcatcagcctccaactctacctgaacagccccaagtagccagctgcgcagggaagaccaacacttacttgagtgccactcagcaacactggtcacgtttggaaagaaaatt

SEQ ID NO: 14 - DNA, 528 bp, Rattus norvegicus
cacgaggtcaggaacaggacatttcaccccaggagcaagggtctttcggccccgaactccaccagaggcaatcgcactgtgtagccgtctcctggagtacacgccgaccgcccggctaacaccactggaagcttgtgcacattcattttttgatgaattacgggacccaaatgtcaaactaccaaatgggcgagacacacctgccctcttcaactttaccactcaagaactgtcaagtaacccacctctggccaccatccttatccctcctcacgctcggattcaggcagctgcttcacctcctgcaaacgccacagcagcctcagatactaatgctggagaccgtggacagaccaataacgccgcttctgcatcagcctccaactctacctgaacagccccaagtagccagctgcgcagggaagaccagcacttacttgagtgccactcagcaacactggtcacgtttggaaagaaaattaaaaaaaaaaaaaaaaaacctttgcggccgcaagcttattcccttta

SEQ ID NO: 15 - DNA, 1525 bp, Rattus norvegicus
ggccaagagaacgaagtcttttttttttttttcttgcgggagaacttaatgctgcatttattattaacctagtaccctaacataaaacaaaaggaagaaaaggattaaggaaggaaaaggtgaatcgagaagagccatcatgtcggggcgaccgagaaccacctcctttgcggagagctgcaagccagtgcagcagccttcagcttttggtagcatgaaagttagcagagataaagatggcagcaaggtaaccacagtggtggcaactcctggacagggtcctgacaggccacaggaagtcagttacacagacactaaagtcattggaaatgggtcatttggtgtggtatatcaagccaaactttgtgactcaggagaactggtggccatcaagaaagttcttcaggacaagcgatttaagaaccgagagctccagatcatgagaaagctagatcactgtaacatagtccgattgcggtatttcttctactcgagtggcgagaagaaagatgaggtctaccttaacctggtgctggactatgttccggaaacagtgtacagagtcgccagacactatagtcgagccaagcagacactccctgtgatctatgtcaagttgtatatgtaccagctgttcagaagtctagcctatatccattcctttgggatctgccatcgagacattaaaccacagaacctcttgctggatcctgatacagctgtattaaaactctgcgactttggaagtgcaaagcagctggtccgaggagagcccaatgtttcatatatctgttctcggtactacagggcaccagagctgatctttggagccaccgattacacgtctagtatagatgtatggtctgcaggctgtgtgttggctgaattgttgctaggacaaccaatatttcctggggacagtggtgtggatcagttggtggaaataataaaggtcctaggaacaccaacaagggagcaaattagagaaatgaacccaaattatacagaattcaaattcccccaaatcaaggcacatccttggacgaaggtctttcggccccgaactccaccagaggcaatcgcactgtgtagccgtctcctggagtacacgccgaccgcccggctaacaccactggaagcttgtgcacattcattttttgatgaattacgggacccaaatgtcaaactaccaaatgggcgagacacacctgccctcttcaactttaccactcaagaactgtcaagtaacccacctctggccaccatccttatccctcctcacgctcggattcaggcagctgcttcaccgcctgcaaacgccacagcagcctcagatactaatgctggagaccgtggacagaccaataacgccgcttctgcatcagcctccaactctacctgaacagccccaagtagccagctgcgcagggaagaccagcacttacttgagtgccactcagcaacactggtcacgtttggaaagaaaattaaaaagaggaaaacaaaaacaaaaacaaaaaacccc

SEQ ID NO: 16 - DNA, 144524 bp, Rattus norvegicus
  misc_feature 44465-44514: n is a, c, g, or t
  misc_feature 56258-56345: n is a, c, g, or t
  misc_feature 61126-61175: n is a, c, g, or t
  misc_feature 61362: n is a, c, g, or t
  misc_feature 92768: n is a, c, g, or t
  misc_feature 92772: n is a, c, g, or t
  misc_feature 92774: n is a, c, g, or t
  misc_feature 98987-99113: n is a, c, g, or t
  misc_feature 110087-110136: n is a, c, g, or t
  misc_feature 114039-114088: n is a, c, g, or t
accgccggatccctctgctgccggagccgcagcgtttgccgtcgcatctccgagccatctcctccctccttcccatcccccttctcttcaagcgtgaggctcgtgatccttccgccgcttcccttcttcattgactcggaaaaaaatccccgaggaaaatataatactcagagtacttattttcaatcaagtatttgccctcgttcacgtgatatatatatattttttaaggattccactccaccctttttctcctctcccaggaaagggaggtgaaagaattatattttccccccagctctaaatcatctatgtgttaaatatccgtaccgatctgtcttgaagaaatacatagctccttttttttttcttcttaaatagctatacaaaaggagtgaaaggccaagagaacgaagtcttttttttttttttcttgcgggagaacttaatgctgcatttattattaacctagtaccctaacataaaacaaaaggaagaaaaggattaaggaaggaaaaggtgaatcgagaagagccatcatgtcggggcgaccgagaaccacctcctttgcggagagctgcaagccagtgcagcagccttcagcttttggtagcatgaaagttagcagtgagtatcgattttattttacaccctttctccacctcaccattaagataagaaacctcgaaataccaggatctaaactaacagacctgatagaacagtcagtcagccagtggtagtggaaaagggcaaaacctgtcttgactcaagatgaggcagctcctttttctcctcccatacccatacccccctcccccagcctcattaaccttgaattgagagaatattagttttatttggctgattggattcgaaacttattttcttacgttctccgtgctgttggtagtcagtcttttcaagtgccccagtgcctccgggttaagcggtggaagctttcgcttgcccatccctttgaataaagaggacaacgcttgattaataaaccatgatgtacatttgccgagtccgtgtcttctggaatgagggggtttattttagttagctcaggttatcacgggtctgcattctaaatagagatactaaatgccccagcctcctaatatttcctttttgcctgtgtgactacacaaagtatttttgaatctggggaggaacttgcttggtaacccacattcccgttggaggcgagctagtatccatccctcactcttctccgggctggtgggagctgagaaatatcccgcagcagatgcatctctgagtgcattgtttttaatttttaagtggtttaatgtgttatctcttaaccttcctgttttgacattaccaatctgccaccatataatttgtgtaatcaaaaggtaacttggagattcaacttttgattgaggttgtattgccatattgccaaatactaagttaagtgttgtcactgactgtcatttaagtttaattttgaagtggtgttgcatttattacttgttaattttttttttaaagagtacaagtggatacctttttaggctgatttaaaatgtatctatgagtatctgtcagtcttttgctggggaatctgtgctgaaaaaaaaaaaaagtctgtgttcttgaagtgtttggtataccgagtatccagtgttcaaatgtatctatttgtggagatgtttatgtggtatttcatgacagcttattagaacatagttgcagattctggccaaaatatctatggtattttccttattccttgagaaagaagagggaggagatagataaaactgttttgtggctgagaggagctgctgtaactgaagaactaagatttttaaaaagttaaaatgcatgtgctgttatgtgcaattgattaagcaactgggagtgaggaatcctccagttgcacgcaggattgttatgtgcaattgattaagcaactgggagtgaggaatcctccagctgcatgcaggattgcagttctgtaggcttttggaccttgtctggaacagagaagtccaaagttgtatccagagcgatagctgagtgggagagatttttgcaagttaacttttttctttccttcagtgagtgactagccaatttagaacgcagggcatgttaagagggaagctgatttcacatctttcagggaagggtcatgctttctaagcactttatggagtcatttgaaaggaattttaaaaaatgctagggtcattttcagtgatgatgaagtagacttcatggtaactactgaagagcctttcatggtacttcagtatttgtgctttgactagtttaaaaaaaaaaaaaacccagtcaattttagaccatttatgaggtttatttcagagatctgtaatgccagactaaaataccaaagttgggaatttctcttcttgtttccaggattttctttgaaaacaacttagccttgatagagtttctttagggtggaggaaatcccagaggaatatagtttaaagtgttttaattttatatttaccatatcagtcaacattaaaaagatgctaattattactattttttttttaaaaataaccttgatattaaggacacatagatgaaagaggaatttgttagggtgctggtgttcaaaaaggcatgtatgacattaagtccttgttcccagagcctctgatgtaaaggcatacacattcagaaaagggcgaaggcgataagcctgataaccgcagtattataaaacgccaagtgaaccatttgttcagagggacacaggaaaagggtgatgaggaaggagcagggaggtttccgcagaggtgcttcttaaaacaggtgctggctaagtccatgagcctgtggagacacaagcagtgcactgggtagaaggcaggcccagccagtctgttctgtatgggaggtaaggtggtatatctcaattaccccagcctacacgggagaaccgggacgtgggagctgaggcttagaggaaagtaagttaataaagggcagaaaaggcctgtaggttcgggtggggatctaggagctatctgtctgcactaactgtgcattgctgaacaagacgttctgaagggatttggatagctgaagggagagacattagaagccttttgggagatagtagactatgttagagatcacagtgaaatgtgaacatgggatgaaaggaagagatttgaggactcatgagggaaaagctggcagggctttggattggactgaaggagaatatttgtggagtcaggagttaaaaataaagtctgaggttattggtctggaagacttgaccgctgggttacattgttcattgtaatgggaaattgagaaaatgctgagacgagagggcggatgagttcagtttttaaaaatggcatacaataagtgtctggcggtcggttactagcagagcttaactttctagtctgttaatttctaatcctcacctagaaatgccaggttgtatttgaggacagtaactagaggttatacttaaacacatattttgttttgttttgagacagggtttttctctgtgtagccctggctgttctggtacttacttgctatgtagacctgcctagccttgaattaacagatacccacttgcttctgcctcccaaatgctgggatgaaagtgccttccaccactagtcattttatatagtcttaatctttgggtcattcttaaatgtatttttcataagtaatgcttcttaggtaaaatttgcctgtaaaataatacctttaagatttaattaaaagttgattgttaacatttttttctgttgaggcattattaaataagtatatttcaaatattttttattttaatttatttttattttgtgtgcattaatgtgttacctgtatttctctctgtgtgggtgctggatcttggagttacagacaactgtgaactgccatgtggttgctgggaattgtagtccatgctgttaaccactgagccatctctctagcctctcaaacactttttccttgctgtaatgaaatcaatttttattcacatccagatttatggagattaaatgaatattaacatatacacagattctactgttgaataacaccttttactcaaaatagagtgagtgcacggtgattttggttaacattttgtcagatgcttatggtgaggctgggcagtggtggcacacggcttttgatcagcactctggaggcagagggacttggatctctgtaagtttgaggccagcttggccaaggcttatggcgaaccattggaagacaacttggctggttgctcagatatagtgcttgtatataaggtatggaaagagggaaatactatcacttaggttgtttcaacacataacattttcattagcagctgtggcacacatctttaatcccagttctgaggaggcaggccggtcactggttgccctgtcgacatggcaagtgaagtagttccaggtcagccaggactgcatagaaagacaacaacaacaaaaaaaaaatcatcagtgaaaatagaccaatttataagttgcatagtattctgcaatatcaaattatactgtcttataatcaaagactagttagcaacttggaggcgccctaatttccaaggaaggttcatttccttgtgatatggaagaaataggttattaggaactattcctcacccccccccctttttttttttttttgagacagggtttccctgtgtagccctggctgtcgtggaactagctttggagagacagagatctgccttcctctgcctcccaaatgctaggatttaagtatgccccaccacacccatctcactagctaatttcttgattttcctatttaatggaataaaaacaaccctgtttgtgagtgagattgaaaaccaggaccttgcttttcactcctattagctagactgactcacagctgttaagacttaggatatttcggtcagcgtcgcgtggacctctggtgcatacaccttgaggatgctaagtatcaagtggcaagcactattccaaaactttagggacctagagattgagcagtttatcatggctgactagaaactgcatttagaaaatgaggtgtcttctcatattaccaccccttcagatggttcttgtgcccagtttcatcagaatcttactctggacctgaattgcagttgttttgggcagacaagtctgggaaaggccaagttatcaagtacttagcagtatactgtgatgagaaactatttatttattcagtatttatgagtgctttgtcctcacacacaccagaagagggtttcagatcccattatagatggttgtgagccaccatgtagaggctggaattgaactcaggatctctggaagaacagtcagtgctcttcaccactgagccatgtctccaggcccaggaaactttgtgcgagctttgtgtatagagcaggctaagaaacatgctgaccctggtgccacagttcctttttttctgtactttccattaaagtttttacatttgttaatgtctgtatgtgcatgtggaggttggaggaatcggctctctccttccattatgtgggtgctgaggtttgaactcagatcgtctggtttggtggccagggcttttagcagctttttagcactgagctgtcttttttagtattttaaaataaggtttaggggcttgaggtacagggatgagggcaggcagggtgagattagtgccctacagtgtgagtggggagcagaggtgcatgggcggggcagcagtaaagcctgctcccaagacaggaccaacctgattccccaggcactgttccaccgagcacatagtcactctgaggcggtgaagttgttgaaacggttaacatcgttctctgtctgatctttccggggtgttttagagctggtaaaaggaaaaccatgcgtaattttcagaagaaaaagtttcatggtgcaggaacctcggcctggttgttgcagtcttggtacctaactttggttatctaaatgcttttttaattatcacaaaaattgcctttggaacttttgaatatgatgtggaaatgtgtgtgggtcaaagcttctgtgcacagaagacaaagtgaccattgatcgagaaacagttttctcgagatgaccttacagttaagtttacagctgagggcggttggcgtggctcagcgggtaaaggctctcagtgatggtctcagtttaatcccagatcaggagttgggaagtcaagctggctaccatgtccaacaactcttaaaggatgtccaagccctttaaaggataatttagcaatgactctgtgacttttcaccatagtggttttgaagttagtttcaggtatgcctattagtaaatacagtatagttccttgaaaaaatattaggatttatgccagcacaaaagggaaaataacatgtgactttcctttgttgttgtgtaatgttaagttagcactggttgctttgggagagcattggcaatctgtgaataaatcttctgctacatatttttatccctgtataatatcaggaagtgcttctccaaattagtcattaataagagctctcgctcactctctctcccccacatcctcccctctctctccagggtttttccgtgtagctttggctgaactcactctgtagaccagactgtccccaaactcagatctgccagtctctgcctccctagtgttgaaattaaaggcttgtgccatacatgtctgatgtccctttcaatcgttcatgaaaagatacttatccctttttacagaatttgatgctaacttcaaacagtagttatttttccggaagtactttttaaattgtgttttcaaccagttattcaggtatgtattatgtattcgttagtgcctgtaattggagcctccttccttctggtcctcgtctctcccaagaatagaagttaatcaatatgcagtagattctttggaatctgacccttttagaattggttagtgattgacaaaataaaccaaaaaaatcaattaaaaaactgaagcattagctatgctgctgttagacctggcatcaggttgacagattcgtagctttgtgtcctggtttgtaggtgtctcaaagctgtttttaagagttattatttaacactttctagtctgctttttcaactttttatccactgatccattttgctggtcctcatttaagtgttttttttcttttcatttttttgttctgttttttaaaatgttatgactagctgtgtagagagtaattgtatgaagccatggtctatgtcgtgagaattgagaaggtgggtatctcaattgtgccaaagaactctttattttgaatgtctgagaagtttgttggagcttcacaaagaccaagcctctctttgcatgtgacattctctacttcccctcttctataagatcaggtaatgtctttcaatgaattcaccgtctccttcttggcacgctgaagagactgagttgagtcaaatacatacacttacttggtcatctaccttccagacccttacttctcaggtacagccttaacactgactgctgttctctccagtagtctttgtcctttcattgattagtataattttagtttatctttaaaaataacacgagagcaagtcctgtgatcccagctgctcagagactgaggcaggaaacaagtccaagggctgcttgggcagctgacattctgtcaggatacgcagagaaagcgctggggataacactgagccgtgggtgtttgtctaacattgtgaggccatggttcattctccagcactaggagaaataaattcaaggcaaatctaaagcagaatgaaattaaccttagatattgtttctccctgcttgtagtatatgtcattgtcatgtccatccacctcctgttatggtataattaaactactatttttagtcaaattattgaatcattcccactatctagacattgaacagtagtcagagccaaaacaggttgtgaaaacgcccacacttccttttgagaagagctatattggaatataattcacatatcataatgtttatccactgaacatttagtggttattagtattttcatagaattgtgtagccatcaccatgactcaattttagaatgtttttctcaacgcccgctctacacacacacacacacacacacacacacacacacacacacacacaaaccggttcattactggtctctttctatcatctattggctagctataggcaagcattaatctgtgtatatctagtctagatttcatataaatggatctatagtaagatctccgccctccctccttatttctttgtttttgtttttgagccagggtctcactgtctcttccttgctggcctggaacttgttgtgtagactaggttggttgccctcaatctggagatctaccgttctctgtcctcttgagtgggatgacaggatcttcatgcccttgcttccccagggtctggtagcctgaaattgcagacgtgatgtttaccacatctgtccctgcctttcctttaggaccagcttcttttccttgacacagcttacagtctttcatgctaaagaatgcacgattaaaaaggcctccttcctttttatcgctgggtaataattgcattcaatggaagtgtcatgtgcatcagtccattagcatgtgggttttctgggctttggctgcactgctttggacattcatgagcaagttttgatgtggatatattttaagtcttctgaatatgtacctagaaatagaactgctggatcatatggtaacttgctgatcattttaaagaactgtgaagcctgttctaaatgcacagccaatatattttcgttattttgtttttaaatatcttttttttttgcagttttttttttaaatatctttattgttaccatttcttgtattgttcgtggtgatacaggcttgattttcctctacagagtaccgttttttcaggctctaccttttggatttctacctgctgcgactccttttctctccccgcagcatccttgttcatagagccctaatgtgactgctggttccggagctgctgggtctccaggaccttttgagcggctgacttcattctcctgttacttggatcatgtgtttgtatatcgttgcagggttattatctcatattatcttgtgacatcagccactagtgctcaaagttatgatttggtaaatatatctggaaatgttcttagtttgtatgtatttatataatgcctttatttatttttgaggcagaatttcactatgtagcatgggatggccttaaatttgctattttcctggtcagcttcctgagtaggggttgggtgtgtgccactatacttctgtcagtgatttttctgtatttgttccaaagggaaataggacagggttttggcaaatatctttacctcttctgcattagacacagtgattggatgtttctttttaagattttatgatcagttgatgactaagaattctgccaacttctaagtgtatgtatatcaattatacgttctaggactagggaaataactataggatgaattcagagacccactggacctaatataagttggatttcagccaaaaatattaatgacctgacatccctaagtccctgctttaactggagggttgcggtgtttcttggggtctcctggaatcagtccaattcagaagcagtatctagtagatcctggaaagtttgattaccaccccccccccccaaaaaaaaaaggtacagttacccttgtaaagaggtatatatctctctggggaaggagtggggaaaggctaacagtaagacttgtagatatttattttagtcagtgttctgttgctgtgaagagacacatagctcatagaagagggggcacttagttgggcttagcttatagttgcagaggtttagtcagttagcatcctggggtggagtaagggagcatgcaggcagactcgatgctagagaagacaggagctaagagtgctacatctggatccacaggcaggaggaagaagagagctattgggcctggcccggcttgggtttttgaaatctctaatcccactaccagtgacacacttccactgagaaggccatatctacaccaatgaggtcacatctcctaatccttctcaagtaatgccactaccgggtgactaagcattcagatacatgagccactagggggccattttttctcaaactaccacagtattttatcaaggtctttcctcaggagagcctggccatctcttcttcattcaaggggttctggttctgcagactggctcaagtctggaaataggttcatgggccaagattctcatcgtaagatacaatgtagccattctttaatttgtttgagaaattttttgagtagacagatcaggagttcagtagtcatctttatctatttcatgcctggaaacagcatgattgattaaccagtactaaaggtccaaatgagttattcccccattaaattcattttgcctgtactgacttttacaggccaggccatttgacattgctttgtctatcctacccactgcaatagctttgatcacttggccgttggtgatttagttcttccactggcccctgcttctttggagcccagttaaaccattgcatttcattttgtccaactaagcaggagtatctccagctctaaggtctggcacaagggaaaggacaacagcaaagctcttcaaatgtgctggtgcccctttcaccattttgcatcttataggatggcttagggtttctattcctgcacaaacatcatgacaaattagcaagttggggaggaaagggtttattcagcttacacttccacattgctgttcatcaccaaaggaagtcaggactggaactcaagcaggtcaggaagcaggagctgatgcagaggccatggaggaatgttgtttactggcttgcttcctctggcttgctcagcttgctttcttatagaacccaggagcaccagcccagggatggcaccacccacaatgggccctcccacccttaatcactaattgagaaaatgtcttaaagctggatctcatggaggcatttccttaagagaggctcctttctctgtgataactccagcttgtgtcaagttgacacacagaaccagccagtacagaggattagtaaaaggcatgtcttatggatcttcctattgtggaggattaggttttacacagcatactcactctagcattgccccctttccttgccttaaaatcccttcatcagctaagcaaagggataccaggcatctcagctccttttcagtaggctcccttttgacaaatacttaagccaattcaaacaaacttcagtcacctttttaaattgtgtaagcttccgtattaaacctagaatctccactcagtgggcccctatcaataaacttagcatgatccagttttaatgttcttaccattattatcccacttaaaatccattccacacagattccccagacttttggctgaatgaattagaaaactcattaaggtagttagtagtgtagtacctttcctcatggactacacttcctgcctcccctctaggagcctgctttattaactttgaatataggtgtagaggcaactattggtagaccctgagggacatcggtattatggtcttgcctggcatcaccttcagagaaagtcacttctggtttagcagacagtggaggattaatttacttagaggtgaagaagacaggatttcaggcagtgcggctgggcgcatatctgctgagggcagagagactgctttctcaggtgagacactcttgagaacccgaggttcagagttcctggctccagtagggtcttccctcaatccccatcccaagtttcaggatcttactctttgccaattaatgcccttattagccaccgacaccccctaattgacacgaagcaaaggggtatcagccatctccagctccttttcagtaggccctcttttgacaaatgcttcagccaacccgtcaaacaattttggtttggttcaaacacatttttgctataattcagccaaccttactcagtttgattttccacaacttgagctctgggtgcaggagagaagattctcttccacggtgcacgtagaaagctctagactttatgggcatctggagcccatcatttttatcactcgagttcatagaagttgctgagatgctgcaagttgcttgtattttccttggtcagtttatccagatgtgctaggaacagcctgccagcagcgtcgttttccttatgttcaaccagttgtcaagcattttacatgaagaattgccaaagccatagcctttcataattcgtgagtcagggtgaccaaatgcatttgtctccttatgttcgtaaaatggctcacgccatgggctttcagtacttactccccaaaggcttcagtaactgttagggttggcaagttgggaagactgtttcagatacttaaaagaccccttcttacacttaggttgctcgagaaccacttctgtttccaaaatctgtattggcagagttctctagagggacagaacttagagaatgcatctttctctatatatagaaaagtgatttttaaaaagtgacttacaggctgcggttcagctaatccagatgactgcctatgaatgaaaggtacaagatcaaagtcattgagagacacgtaaaccccaggtgcaagaccctgcatcttaacatttttggtcacataaagtaaaagtgacaggttaaatgaaatttatgtatttacatatttttaaccctgtatattaaactatttcaacatgtattatgaaaattattgtttcctactattttttcatattaaatcttcattgcctagtttttcccacttaatttcggattctgagtggccacagtggccatggtactgacagtgcaggttagaattttattcaagaattagttcaatttgctatttatgtattttagttattttcaagtagtaggaaaaatgtatatgggttaagaacacttcattgattcatgcttttctcagttgtattttccacttcttttaaaattttatattggcaaaggttaagagaattttatgttcttcaaagggtgttgactgctagggtaatatttccttctgtattaacataactgccaatatggtttttctcccctccccctcccctccccctcccctttctcctttttttttttggagagggtttctctgtgtagccctggctgtcctggaatttactctgtagaccaagctggccttgaactcacagagatcttcctgcctctgcctgccaagtacaccaccttgcctggcaatatgctaatgtttattgctttgcttgttagatgtatccttgctatgattgccctgatctcaatgaaattgtactggtctggacggtctttgttatgtattctctgccttgatgttataaagactttctcagcgtttagtcccagatacccttgtccttaaccacttagtcatccatttcttacccatggttgagaggttcacttgtgtgtgatttttttgacactcctagtgaccttttatacatgcttccttacattctagtctgcttgtcttgtttctttctagaaggtgataagctacagcgtgacagaaaagcatcttatttaccttggtttcctgaagtacacagagcagctttctgtatgtgtcatttatacatactgagttgaaggcatgtgctgtgtcatggtgtgatgatacatggcactggtatatttagaaaccgaactcaggtcttttggaaagagccacaagggctcataaatactaagctacctcttcacttctttgttacttaaaggagaaagggaaagaagggaagctagacagagaccagttatactgtgagcttagtgtttatggtctttgaactcttgggatgacggctgcgtgaatgaagtgatccaacgactagaacagagctgctaggctgtacaaagcgagcatggaacatacttcgaaacattgagcatgtgcagtgccacagtggcttcaagccttcaaggacttcatttggagcgggtaaggatggaagagagattcagaacagggaatcctgacataagtttccttttaccaagaagttggttctctaaaagaatagagagtaagcgaacagacgaagctcagatatgaatagttctttcatctttgacacacacctgtagtcttttgagggtgtcatgatagccatgggtctttaaggcttttgataaactagtgtatattggtctatcacttgagtttcccagagatagagtggagaatatcattaataatttctgctaaatagtgtggtttcaaaatatgtgtaagcagtgactgtttatatttgagtatgcaaagtagttttgactaaccagcacggtctatttctgtatttgtgtgcatagtgaggtagctgccctcaggtacagttctggaacatgggagagatttgtttcctcagtgaataggaagtaagcttttcttgaaccaaggggtaaagaactagatgacatgtgttcaattatgcattagggccactttactcagacagttttgagaactttaaaataatctctgagaattcccgagttttcaaaaaacaaggagtggccgtgcaccttggaagaagatgtcctagttttaaattttttttctctatgtcgaggatgataagaacttattacaaaactaaatgggattgagtaggtcagcttcctcagtccttagaagaatggtttccagtagaaccgtcagggactgcgctgcgtttgagatggtctcagggcaaatggcacacctggttccctttcaggattagtttagaagtgagagaatatttgaaaatgtagttgtgtgttatgtgtaatgaagccacagcgttttgttgttatatgcttgtctcatattttagaaagcccgtgcatgtcctaaagtttaggagaagagttgcagctgctgatgtggacaaaagagtgtgggtgggatagaaactaaaatggttgggtggtaatggcagtgagtctccctggatctaggtgacggccaatcttagcagtctaatccagacttcacttgtggagtgcctactgtttaacaaactctgtcatttttaagtccaaaacacttgagacattattgtcagtatttactagatgaggaaggaaggtaactttattgatgtattcagaaatgctttatttgctgttttaattgatatattcagaatcttagagctggtagagaaaagtccaggtgtggtgttgatctagcctctccaggttcggagtatttattgcatggctatacaacaccaaggaaggtaattttatctttaccacagtcctagaagacacatgatttataaaagaggaaactgaggcttaggggacctgacatttagcaagatccacagccatacactgaacctgtcacctcacagcgaagcaggggttgggaaactggaaacaaggcagagcgggcagcgagtcacagtggctgtggctcctggagttcactttcccaccttactgtgtataccacagcttgattgctgccaactgatggttcttgggtttgccccttaagacagcagcagctcagcagcagtataaggaagagcactccttcatttaataggaggtacagatgaaccgcgttaccatgggaagtttgcggggcatatgataagttacgctgcttttttgaataaggcttgctatactcttcaggtatctttattagttctttagaatcagtttacctgtgtaagatcattctgggctccactcggaagagatggagtagacctctgttatttacctgagaaaaagtggccacatttccttctgcaagtggtggaatccacctctgaggtgttctagaggactttaaggaaggtgcacttcttaggatctagtgttttaagtatcagttgtaagcagatgccttcgtattttgtgtagatcagatatttgtatagacattcttttagactgtattgtttttgaaaactctttgtagtttgtgtgtaatatgatagcaacatcagatacaatggagtataaaatccgttccctctcctgagctatgtaatgtgttagtaggagccatcactctgagatgcccttcagaagtatggatagctattggctcattgagaactactgaggttgttagttaagcattaagcggaatgttttatttcccccactcagttttagtcccggtttgcagcaatgctgcccagaagccttttctccatgctaaaggctgtcgactctgaggtagctgacgtcatccagagctctaggaacatcagccagcgttgactacttgtggctgagaggtttttataggaggcctttgagtaagctactttgtccttgccaggacatttttaaagagtcagccagcagatataagtagagagagcagtcggaagaaggaatcatgagctcctttctgtttccacctttgtctcccctctccccacttggctctttctgggtgctgaattcagctttgcatagaattattttcttcagacttttgttaacttggtacttgtaaatagatgctggatatccccacttatatcaaatagtagactagaatatttttatgcgccaactgcctttctaaaatttgttacatacgcttaagacagtgaacaaacacagtggcagcaccgtggttcgtgcctcttaaatattaaaggaacatccttccaaaactggagtggtgccgtggtgaccttgtggaggtcagatgaccatttgaggggattggttctctttttccagcttgtggttccctgggattgagttcaggttgtctggcttagaggcaagcaccttttcctgctggcccacaatggttcttcatttggcctgccttagtgctgcgtgcctttaatccctgcactcgggcaggggcaggggcaggggcaggggcaggggcagggcagggacaggggcggggcaggggcaggcgcaggggcaggggcggttcattgagtggttgtgccatttctcctcaccatagattctggcatgaacctgcaaattctatgaatttccttgttcttgatgtgtttttttttttttttttttttttggttctttttttccggagctggggaccgaacccagggccttgcgcttcctaggtaagcgctctaccactgagctaaatccccagcccccttgttcttgatgttttatataaaggtaattgtacactgtgatcatggggccttttgtgtttggcctatttcattcagtatatttttagggttcatttttgtttgtgccatgttttattttaatggctagatattgtgtgtcgtgcatgtgtgtgtgtgtgtgtgtgtgcatgtgtgtatgtgtgtgtgtgtgtatgtgtgtatgcgcgtgcgcgagcgtttccatttccctcatgttatttattcagttatttctaccttttttgggtctgttctgaacattctgtatagagtttctctaaagccctgtgtagaagattttgtttgagcaacaggtatttctttaggctatatccccagtgatattgcttggttatgtggcttttcagtatttaatatactatgggagagtcaatatgtttttcataggggttgtaccatttggcattaccaccaatttttccaatttttgggttccaatttttctgcaccttctagtacttattttctattataaaggatttattttcttttatttatgtgatgtgtgcctggaagtatgggtgcctgtggagatcagaaaagaggattggtggatttcagccactcgagtgggtgttgagaacaaaactggtttattgcaacagcagcaaacacttcattgctgagctgtctctccagctctgtcttttctgctctttgattgtggctgtctattctgtagtgaaatgacataacacttccatccatttgttcctggttgtggcttttctcctttggagaagtgtgtatgaaaatcttttgcctttaaaagtgtactgtttaaatttgttattgaattgtataggaactttggaatagtttaccttcatttttattatgaaggtattttacctttattgtaggtacatttaaaaattcactttttgtggttttccttttcaatcccttgatagtgccctttgattcagagaagttaagttttgattaaggtgaatgtattcaatattttttcttctcttgcttgtgcttttataaaaatgcagggccaacttgaagtgtttgtttttctgtagtgctagcattgaacggaggacctcctatctgctctgtaaccagctctatctagcccttacttaaaaaacaaaacaaaccttcttttatgtatatggtgttttgcctgtctatatgcttgagcaataaatgcatgcctggtggaggccagaagagagaggtgggttccctgtaaccggagttacagatggttgtgagcctctgtgtgggagctgggaattaaaccctggttctgtggaagagcagccagtggtcttaactgctgagccatctacagctccatcgagatctgtttatttaaggtcagggtttcactgtataatcccagttggtcgggaacttgaaattcttctgcctcagcatcttgagtgtatgccaccatgtctgactcgtactttttaagtcagaaatacatcatgacttcatgtttcacgaactatttcagggtgacaagtttggattataaaaattccctttcagactttaagttttaaacttagcgttctgttcttctatgaggaaatcttaagtagtaaatgactatatttgtacacatataatctaattactattatttcaaggttatgaaaagtaatagtaatgtaagactcatttgtaattaataattgctaagaatgtatagggggattactgttttaaaatcatttgaaaagatctcgtggattaagctgcatatttgataaatatatagtgttattagttagggaaatatagagaaacaacttacagaatgaatgtatatatatatatatatatacacacatacatgcatacatatacatacatatgcacacacatacatatatatgggctttattagagtgaattacagggtgtggtccagctagtctagtctaacaatggctgttaagaatctagttgttcagtccacgaggctggtcttcagtatacactggaatcctgaagtagactctaatgccagtgaaggaatgttcttgcgaaccagggagagcgcaggcaggcaaagaaagccaccttgctttttctgtgtactttatatgtctgccagcaggtgtaggccaggttaaggtggattttcccaccttagaagattcaggttaaaagtggatcttcccacttcaaatgatttaattgagggaaaaaaaaaatcctcacaagtatacccagccacttgagttttagttaattccagatttagtcaagcagctagcaagaatagccatcacatgtggataggctttttttttttttttctttaaatcttgctgtcaatgttttttttttggacacggtctgactgtgtaggctctagctggcctgaactcactatatacaccttgctggccttgaactcacagagatccacctgcttctgcctgctgagttctgggatgtgtgtgctaccacaccagattgtcagtgttttcaaaataatattttaaatttttaagatttgttttattttctgtacatgagagttttgcgtgcatacatgtatgtgtactgtgcgcatgccttcacctgaggaggttgggtgtcaggccctctggaatgaagtcatgggtgatgtgctttctaggggtctaactcagggcctctgtaagaactcttacctgctgagccatcacttcagcccattaaatttatcctttaaatatccatacgtttagacaatgcgttttgactatacctatcccccccctccctgcacctcccttaatgcctctgccctctctcctccccgcttcatccctactttgttgttgttcatccctgagttcagttagttcctccctggggcactggggcacacgcacatgcactggggcatgggcaaactgccaaggccaggcagcaggagagcgactgcacccctcgtgtcctttggttgctaagcagcttctctgctggtgttgctgtcaggttttaggggtgcttcctcactcccgttttgagttttgtttttataattgtaaagtatctacacaattccaaaccccaacctgaatgtgtgagggaaaggaaaatcttacagccttccactgagtaaagaaccatgtttactaggctttggttaaaaataagcataggtacacatagtcatgttttcttagaagaaagctagctcactgtttgtatctcacagttttattttacttgaaaaattgttcaaggtcattcatagcagtacttggcacacttttctttaaaatttatttttcattcactcactcatgtgtgcacggccgcacgccctcgggcacgccctcatgtgtgggcacatactgtgttgcagtgtgaggtcatcaacttcctgaagtcactctctcctaccttgtggctgtgggagtgtgtagctggtcattaggtgtggtggcaaatgcctttaccgtctcttgatgaccatctttttgtgtctgtaccaactcttgatttttttttcagcacatagttttatatttattgatattgggatttttgtgtaaagatattttagaaacgttggagtatttttgtgttttagcagatggacttagaggtgtacacagtaaaatgtttaaattgtcttgactctggtttgtgtccctggacatgcacctttgaaacttgacacagttagttcaagacatcttcttaactcaaagtgattttaaacaaaatatcatattgtaattattctgtaactccagccaggagccattatccttggttgaatttgtttcaggtgacatccgttactattaggttgttgaatacaatgaggcttttaatactggttttgatatcttttttgtcattaatattatatatactgcctaaatttcctatacttgttccaaaatccataaaattgtatatattgctgcattcctctttatttttctatagatttaaacttaattttcaaaataaattggcacaaatttcactgattaataacaaaagtagatttgataaagcgggatgcgtttctaccatgaaaattgacaaaaatttctgaaaatagagaaaagtctttgcagtacttttgcttggactttatttaccttttgcctctggcattggtatgtttaatttgttttcatgtgttagtggaagatagtaaacttaagagttgatgaaccgagagtggttctatgtgatttttgaaggaaatatgtttttgtagaatgttactatgtaatatatttttaaatttatgtacttaaggctgaatattagaacaactaaatactttctgaaggcagtttatacaaggaggttaaccagtcagtgtgtgaaacagtatttagtatagaaacagaataaagataaagtgaaataaaatgaacagtttatgttcctcaaattgtcactttctgacagaggctttctagaaatatgttgctaaagtagaataaattcttatttttttcccctggaggacagttttgcagtttcagaaatgaatgtgtagccagtctcattttaaggagaaaagttaaatgtgttctcagacttagatgcccactaaaactgtagtcctagcacagatacttgagtatctctgttgcatatgttatggtcattccgtcctttcaacaatcctatgacacattttcacatgagaaagacgatagtgtaggagagaggttcctaatggtgagtgaaaacagtattactaaggaatatgtatcaagggtttcactgactgcaacacaatctacaagagaacccattagaccatcgtctagctttatttataggtcatgtcctagttagttctaattgtcaacttgcctagagtaatcaaggaaaagcttggttaaggaactgtcccgagcatgttggccagtgggcctgtctgtgggggattgtcttgattggtaactggacaggagctgtagagtactagcccactccgggagggtgggatcttagggcaggtggccttggcagtgtgagaaagtggaacgcacagtactgttcctccatggcttcctaaagttcgtgccctgacttccctcacagatggactgggacctggaagtaagtaagccacataaaccattcttccacaattttttttttgttcattttgtttttttaaagcagcagaaaggaaactagaacaagtagtcttacttttttctctttgctatctttattttcctttttttttttttttttggtacagtgaacatttattgtcttaagaagcctgctaaaaagtgtcttcacttcccttttttgtagaaatgattcacttaaggattcctgtaaatctcaaagaacccttaaaatagaactccaaaacgatttaaatctataaggaaagttggcgagtatataaaataggaattaaagctgatttgtatattccttgctctctgctgtgtccgtcttttgtttatatttgagaccacaatctcatcacatttgtccattccctttcctccctccaaaccctccccgtacacttctgctctccttcaaatccatagcttcttttttcactgaattgttattatataatatatgtttgtatatacataaaagctcctagtgtatagcctatgagttcatgtgatattccttgtatgtatgttttcagggctgatcttttggcccaggaaagctaactggtgtgctcctccctgggaggaccacctcgcctgcttccagctttatcagctgcctaggactggagacccagagaactactcagtgctagcgagtgatgtcatgccgttggaagagactataactactaattgctaaaccagcgtaatccccagcaatactccgtaaacgcttgtccttacacccacggaaaagtgtagtcttcactcctcatcagggaccctttctgcaacagacagactactgcaggggaccacgactactcgagatgcagagctgtggggcccaggcccagtagatgcatcagcaacacccccacatctgaggctcactgtgaaggggggcagaaagactataagagccagagggacaggggctgtgtcgtgagactgggtctcctggtaccgtcagaagccacaccctcaattctcaccagcattctggctttctgtaatgagaatgcggagcatctgaagctggaagccagcagtagcattgccgttgctgtgagtaaagcctttcaaaatggatgagtgggtagcctcgctgccttttcatgacagtgcagtgcagtgcagtcggctttcaccccagtaggaactcagttagctgttgcaattagtgttcccttatggcagtctcctgtgaagcctagcttgccttagcgtccctcctacgggattatagggcagcttcccttttcctctctcctctttcagccctggctgtcctggaactcagtctgtagaccaggctaacctcaaactcagaggtctgcccgcctctgcttcccgagcgttgggaataaaagtgtgtgccaccaccacccggctaaatctggtttttcataactcttctgaatcttacacttttatcaagaagtaaaaaaggtagagcttgcaagatgggtcagcatgtcagagcacagcttcaaggccagtgacatgaattcagtcccctggccccgtgtgatgaggagaaaacaggctcccctaagctgttgtcttgtatgctctgcctccaacacacattactgtactcatttgggtgtttgcctgaatgcgtatctgtgcaccgtgtgcagcagtgcccgaagaggccataagagggattggacttacagacggttactagccgccatttaggtgaacctggatccttggtaggaacagtgagtactcttaacgactgagccatttctccaaccccagaaaataaaataaaagcctttttattttaaagtgaaaaattagcccaacagtttgctggttgtgaatctgttctatccatggttaaaatctgcgctcattttaggtatgattatctttaaaatctggttatgtcattttaaaggtcatccttgaagttcttaaaaacctgcatttcctggccctgtttttctactttcttgctaacacttaacattttacctgtataattagtgtgtctactgtctgtctttgccccttttattttttcaagatagtctttccccgtgtagccctggctgtcctggaactcactctgtagaccaggctggcctcaaactcagagatccacctgcctctgtctccctagtgctgggattaaaggcctgcaccaccacctggtttttaaatgcagaattttgattttgattgtattttcccaaagtttagaatgatgcttgtgggcacaggagatttcaacctgtttgcttttttaagtgaatagtaggtaggcctttggtattggtagattggtgctctgtttggtggtagagatgacgtttcagtgtgtacaggtaaggatcatttgtgttttagggtggctgttttttctaagttagcgatggaatgagataattgtaggctagttagagtttcagaaccgtgctctttgacacatggtagcttattaactagtcttagataaattatagaaagggaaaaaacaggtagctagaaagctaaagaaagagtatattgcagagaatatcctcctagcttgctgggagttctgtgttaaccggtacagcattcttgtgaggtaggcactgcagccaccatttgaaagatggagggttttcattgttctacacaacaggtggctttttagtcaggggatgcaattgcaatttcaggctttcttcttctcagtaaaatttacagaagcttcctcggagaaatagaatgtagtggggtgaagtgggcagagccactcaggatgtgccgatctgcagtcgttcgttgagcttcactcagcactgaagcgttgtgggagggttttcgggttaagggtatggttggactgtgcactttaaggcaccccacatttatcattcagcattcagggtcagctgcagcaggtgcactgtcgaccctgaaaggtcaagtaaggcacccagcagccaggtgaggagtttgaggatgaagaaaggtaaaagtggtttgtgccaccgcactacttccagcttgctgcttcccaaacagcagaagaattttattttcttggaccttttttgaagttggtttaatttttttttttaataaatttatttattatatatactatagctgtcttcagatacaccagaagagggcatcagatctccttacagatggttgtgagccaccatgtggttgctgggaattgaactcatgacctctggaagagcagtcgggtgctcttaaccgctgagccatctctccagctccgaagttggtttaatttaaaaaaaagattatttgtttgtttgtttgtctagtgtatgtgtgtacctaaatgtatgcatgtgcctgcattagccccaagggttcagcagagggtgtcagattccttggaattgaagtgtgataggcagttaggagccatctgatatgggtactgggaactggaaccaggtcctttgcaagagcaatacatattcttaaccactgagacattttctagtccctggtttaattaaaaacattttaagaatatgtttattacttgtgtgtggattttagaggacaacttgcgggaataggtttgttctgtccatggtgtgggttctggcatcactcgggttgtctgggttggtggtttgtaccttacacagctaagcccatttcctggcccagaggtcgatttgatttagtagaaacagtgagagctaaacgatatatacaacttgtataattttattgcttattttccagtgagtaattgtttctgggtatgggtctttttgaaggaattagcatcttctgggtgtttactatatcttttgctacctgtagtttgcaaaaacagaattgagtttagaaaagttaagtaatttgtttaaagttcacgccattaagttgtgccttttaaaccgttttcttcaatgcagggttctatccgtagattggtttagaattctagccctaaacacttagctctaactttgaaccaaatttctacgaaaacgtcctttatctttaaaataataaaaaatatcgcataccccatgcaaatgctgtagaggttaaatatgtaatgcacagaaaacagtttggtattagcatcgagaatatactcagtgttgttagttaataattatttttccatcttcaaatttttggcttttcaagtgcactgtttctggaagtcttgataagaagggtgttgcagatctgaactgtaatttcgggtgtgatgcgtttgacaaaaggaagagtgaagcagtcaggtgacagacttaaggtctaggcgggcagggagagtctgcagcaagtaggttaagttggttgttacggtttctcagactcattggaagcagtgtttcctgcagggatcctgtaacccgtttctttctaattgaacagtataaagtatttaagaacaagggggaggtgaagcaatgatgtaggaggtgtttgcttcctgggggtttgtgagctggctattttaagacaggaactcatgttacctgatggggttcatgaatattgcaggttcagtagaagaaccactgggtgacctgtaattcagagctggagtactacttagaagacaggaagttaggatttgtgccacggtctgagaaagcatgtgtgtacttttatatataagacatgtgctctcaagagagccagagagggaaccaagagcttggtcattcctcctcccatctgtaggatgttgtgctttgtttaaagagtctggagaaaatgtactttttgataagaaaacattagcggatggaaactgtttagtgggtcgcctcctctttttgaagttctgtaacacctgcccctctatagctatataaaggcaaggaacacttggcttgtttaaaaatagtaaacttctttctgagaatttcttgggatgcattaatggtgcgttaaacatcaatattactaaacctacatgagaattttatatctgttttatatggtacagtaccacccccacccccactccccatccatttttttaaaaaaattgacctagaattcactttgtgaaccaggctggccttgaactcagaggttcatcttcctcagcctcccaagtgctggggttaaaggcgtgggccagcactgcctggccaggttttacttgtatggataactggaagaaaagaaaaagatgatgttgttaaattcagacagtaagagttttatttcctttcataaaaatgtttcctatttacactcccgtgtctgaagtgacaccaggtgactcagagatgccctggttcctgccctacaaacaaagtcattgtcagcagtaggcacaactcagtgacgtgtttcacagtgcagggtcagcacctggaccattgcaggtgctgtctccccagaagggaacagagagataacctctgcagatcgttcaggtagcattgtgtccttatcagtgggggaactgttgtactgttagagaagtgctgaaccaatatcatttagtatttgtgacttgataagctttgattgtctcatgttaagaagttgatgtcagggctggagcgatgactgcagttagccatagcaaatagcattggctgcttttccgaggacctgggtttgattctcagcaccctggcagctaacatttatctgtagctcccgtctcaggggatttgatgttgtcatctgcttcctcaggcacccgtcacttcacatggtacatagatatacatgtgggcaaaaacacccatacatataaaaacaaataaaaaatgtacaattatgttattatggtttagtgcttttaaaaaccagtagttggggctggagagatggctcagaggttaagagcactgtctgctcttccagaggtcatgagttcaattcccagcaaccacatggtggctcacaaccatctgtaatggcctgcagatgtacatgcaggtggaacgctgtatacataataaataaatctttaaaaaaaaaaccagtagtcacagtaagatatggaaactttagtaactctactgtagactcctgttatttaaactagaggaataaacaaaccaccccaaacttgtatttggtgagtggtaaaaataattatctataatgctggagtagttaggagtgcagaaccatgtccaggttaggtgactctccactgctgtccctctggctacagagggatccagggcctctggcctgtgaggactcttccgcactcattcgtgtatgcacacttccttgtagatacatagttacaaaagaaagcaagtcctaaaacattcactgttaaaattgattttgaaagaaaccatgctatagaaatttgcttttacatttttaacggttttaagcttttataattttaggaaaccagagagaattctaatgtttgtcctttattgtttaaatattaagttacaaactgttaaatgaaaatatcagtctgaaacttggcttaaaacattcactagttgtaagtgtttgtataggtaggctggactctgttgctgtggaccacacagagctagctgatccccctgtggcgtggtcacttaactggtaaactgagggtagctgagctggaggaccgcacttaaatacctagtggttggctatgtgttgcccagatggcagaacccatgaattcttatctgtttgttctgtttggcttggccttgtgttctgcatagctggacagattcccaggagagaagaattcgagcgtgcaaggcgtggttctgatggtctagcttcagatcgagtgcagcacacatgtctgctgtcttaaccaaacccagtctgtaaagacacatggacttacatgcggaaggcagcagcaattgggaaagttttagaattaatctcctgcatttaatgtagcgcttttataattactcatttatgtaacatatatatatgtgtgtgtgtatatatatatatatatatatatatatatatatatacttaaaaacccttgttaatgataaaacacatctactgagtagctcttttaaaacttcatagtacacactagggtccctcattaaatatctctaattagtagaggtgataaaaagagataattgagtgcttttggacccatgacatcggggaaagtgatgcctaaagtcagtcagttggataaatcctgtttcgtcatagttctttggcttgccttatctctccccccaccccctgcctttgtaaatttgaggtgaaaggtcataaaactttatcctgattctgtgttatttagtacattcaccgtgtacatccactacctttatgacgttctgaaacattttcagtaccccacaaagaagccacccattacgcagccagtctccattgcctcttccctctagcctggtaaccatcaatctttcagtgccatcagttagtctggtgtgtctgtgtgtgcatgcctttgttcaagtttgtggaagtgtgtggaggctagaggttgatttcagctgcattactcattgttcttcaccctagtttttgaggaaaggcctctcactgaacctggaaatcaccaattggcctagactggctgactatcaagccccagggatccttgtgtctctttctccttgggtctgggattataggcgcacttccatgcctggctttttggtgggagcggggtgtcaaaacagccaaactcaggttctcgttgcttgtgtgccagactctctgctgagtccttgtcagccctcatttgcattttactcactgggccaattggtttatgttccttacaaccagaggattgtagacttgatcagtgtggctcactcaaagtcatccctggaagattctgtcagttagtttccacttggctgacgatatcctcaccgtttccccagtcttccctgcagactcttcctcccctttccttttcacttgagggcttagatttttatgttagcctttgctgttcccacattcctccttggctggcctttgctacttctgtggccaaatttagacttaagactctgtcccttagactttcagacctgttatatgtattcctaaatgtaaccacctaaaacccatccagttgaaacagacaccatcacagaacctaaactattttcttgctctctgtcctttaaccaaggcaaaacacctctgcttgtctgtcgccctcttcttgagtaaatagcactgtacttcctaggcttccttgcctttggtcccaggagtcagtgttgtcaccatcaccattctgcaggcctactcttgaatcaagagctatttcccaatagtaacttttaagttttggatgctaataccctttgcttagaccagtgttatgcaaactctagcttttaaggctaaatatgggtagttaataattgtgagaatcaagctgtaattaaatactgccttgctcattaatttgtatgttcattacattacccggtggtctccagaacctaaactattttcttcttggccctttaacgatcaactttccctactcttgaactgttaactagttttctgtttctagctcttgtctacttttcttttctttccaggtaaagatccagaagatccagatacatcctcttgtgcgcacctttatgtaaaaagtcattccttacagagtaaaatggaagacctttatcgtggcataagatccccatctgcctgcctgtctttgtgtttttttgttcagaggtgtcctgggaatttttttttcttgacctttcctgagctgttctttcctgatatctgtctgtctgtctgtctgttcccccctctctctctctctctctctctctctctttttctctctctctctctttctctctctctctctctctcacacacacacacacacacacacacacacacacacacacacacacacacacacacacacacaccgccatgtattgaatgcttggtccccagaaggtagtgctcctggggaagtagcttcatgagaatgcagagtaaactgttatgaggtgagtcttagttgaagctgggctctgaagtatccatctttgtctccagaacttcctgtttctcagtctttgccttctatctgccacaagtaagttgtgcagcttttcatcacgttctggttgcctggatgttctgctccaccatgacatggagacactgtagagtattacatgagaaacaatgagtgagcccaaacattaccagcattttgtcacagtgacaacagctggtggatcttctcacattctcgattgtgtccccttccttgctttctcacacagattacagctttcgttgtatttattaacccattgtgtagttttcctcattagatagtattctcctagtaaactaaatgccacatggggcatgcactaaagggagatgttaaatccatgttactagttgatactagttactagatgttaaatccatgatactagttgaattgttgatttcaatagtcatatagataagaaccatggttccattttggagggtaagttagttacttttctcattgctatgattagatcccttaccactcccccactctccacctccttagagtttctctgtatgcagccttggctgtcttggaaccaggctggccaagaactcaaaagatctacctacctctgccttctgagtgctgggactaaaggtgtgcatctccactcttgactataaaaaagcaatttaagggttgagagaaaacagtccgtcatggtggagggggtatagcagccagtgtgtgaggtgactggtcacactgtgtctgaagtcaggaagcagaattttgaattttgatgctgtccatattcaggaaggttaaacctttttggaaatgcccccatagatgggacccagacaggatcttaggttgcttctaaatcttgtcagattgacatcaagatgatgaaccagcacagaagaagtggacatccttaactgtttgcccactaagtaccctacttgtccatatagacattttcctcctcttctattttagaattcattgacttccattaaatactgtgtggttttagctaggcattcaaagatctattctgcgtagtgcatatgtaaggaacagttaagatggactgtacaaaattgatacattactgtagcattgttattccaaaagtatggattgcttttgttaaatctgaagcagaaatagactgctgctgctgtcacagctatctatgctatttgtataggtattatataggtaatagctaatattgcctggtttttgttttgttttgtggcatttaggtacttactacctctggtagtaagatacatagtagcagcaacagcagtggtagcaggtaccttatctcctgctcctcatgtgccaacttctcttctaagcacagtgcaccttctcttctaagcacagtgcacaagttcacttatttaatctgtaacaatcctctgagataagagcctatcatccttgtacagatgagggacttgaggcaccgtgaattgaaatacgtaaatttggccatggtatagctagtaggagttccgggcagaatttaaatcagactgactgtagaagggatgctttagtgactgattatgttagattctgaatggaccgtgcaacatttgcaagtgtggtgtgtggtgctgggaattgaacccagatcctctggaagaacaactagtgcttctaaccaccaagccatctttccagtccaccattgtgttctcattgttgagtattaagaatcctttttacaatttgcgtacaagtttcttatcagataattagttttacaagtattttcccctcagtcttttttttttctttttttggtaaaacaggtatttttaattttaataaagcacagttttacctagataacctatttaagtgtgtgtgttaaataaattttttcaaggtcatttagattcttgttctctttagaagttctatagatttacatttttaggtaatgaatcctgaattaatttttgtgagttttgtgcttttgacttaatttttgcatagttattttaagttcatttttttacatggttatctagttcaaatcatttttcaaaatgattattatgccttttaacttttctttttaaagattcatcctaatttacatgtgtctttgtgggacatgcatgcaggcgcatagtaggtgaatgcagagatgagatgccgtgggcctggagttgcaggagctgtgagccacttgacatagatgttgggaattgagcatgggtctttggaaaacagcacacactcttaaccactgagccatctttttggccctttatttttccattttgcaatactggggaccaagtctagggccttgcaaatgctgggtatatccatatcccttgccatccctttttaatgttctctcagtagaataccctttaagcatgcattatgataattaaaatcaaatggaaaactgaagttaccacaattaatattttgactgtggcatagtataccctaaagttgcttagtaggggattgagagatggctcggcagttaagaccactgctgcagaagacctgagtttgattcctagcaaccatatggtagcccagaactccagatccagtgaataagctccccactttgcttgtcatacacaccagcactcacatgcactgtatacatgcagtcaaaacacatacacatataaggtgagctaactaaataagtcttaaaaactaaaaagaagggaggaggggaataaatttaaagaatataaagtgagaaactggggttactgtattcaacatgtttggcactttctgtaaggaaccactgacagttgtgtgtaatatattccctactgttttatcactgttaccactcgatagctctttaatccctgagtcaatggaggagcacttcctagtacctgtatttcaggttaggactggacagatgaagtgtttggcgtgataccccgagtaaaagctgtttggttcatggagttgattagtacctaaagcagatccataggaatcaggattttgaggtgctcattgttcagtgccctcatagaaaccacttgctgggtcagcaaaatttttgagttttcattttagcaagtttggtttttgggttgattgtatttctgaatttaagaacttgggtgtctgatagtgactttggggtattcattgtagtatgctgtggttttggtctcttggatacttggtcctttctgaaacctacttcatggcatagatttattttaacacaaatcattttcctcagaattatgtaatgattaaataaatcaagcacctacagcaacagcaattagtgatcaggcacaaacaggttttcctaatgtggctgtcttcttagttattacagtaagctggcatttctttttctgctaaataatgccatgaggtttaagaattactgcctgtgtatgtgtgggcacacgagcgcatgcgtgcacacacagggaagtcaggaggtaacaggagaagctctttcacatgggtgcaagggattgggtttaggtggtcatgtttgcagcaagtgccttacctccaagctgtcttatgcccctttactcctcatcagacctttggtaggctgtagccactgctcttttcagatctttaactcgttaaggtgcatactgtatagattaatccctcatgaaaaaacaaaaaacaagacaaaagcaattcagcaggagcagaagataattttgggtttgagtcctctccttccaccagttcacaggttccagggaaggaaccaggtttactgggtttacacaggagagccttacctgaggaaaggaagggttcattatagcttcctgcaggttgtagcgtacagtcaaggggaaccagagcaggaacctggaggcaggaactggagcagagactgtgcaggagtgctgctgcttgacttgcttctctgccttccttacctacttgcttatgcagctcaggtccacctgcccatggtccacagggggctgtgccttctgttcagtcagcaaataagaaaatgcccacagtgtctgagggagacaactcaattaagatcctcctctttctgggcatgtctaggtttatgtcaagttgacagaatttaactagtgtatcgactctttctttactgactacaaacacccaatgtttaaataataaaccttgactctgagtgtgccgtttaatccagctccttgtaatgctctataatattttagaactaagactgcattagttaagaagtagctctttgtagttgtgcatggatttaatcccagatagattgcatgagtatgaggccagcctggtccatgacatagtagttccaggacagccagagctacatagtaaaaccctgtcccccaaatggaagacagacagacagactgactggggttggggaagggaaatagctactgtaatgttgtagttaaaatagaagttaggtaatatcaagactacctctggttggcatgacgatgcacacctttaatccagtacttagtaggccaacctgatctacatagtaagttcaaagacagactatgtagagggaccctgtttccttgtgtcccgttgtgtcctgttcctcatcctgttctgtggtgtctgtccctgttctcccatgcccccaacctccatcccctgtcttcctgcaaacaagaagcaagcaaaaaaaatgtttggaggatggagagatggctcagtgcttagtacacgtgctgctcttgcagaggacctgagtttcattctaaggatccacatggtgtctcacaaccatctaactcctgttgtaggggatccaactacctcttctaaccttggtgggcaccagacacacatgcagtgcacatacatacatgtatgttggcaaagcattcatacacataaataaatctaaaaagcaaaaccaaacaaaacaaaactgtataagaaatgtccttatagaacttgaaacaaatttaatttttaaaatgtacctgctcagttataggagatatagaaattataaaaagagaaactgcctatattttcatgattcagagccaatttctgtttgctttttgatggagaccacatagtaaatagcactttataacctactttttcatgtgatcttgtattgcaaaaaacaatcttttataccaaatagcatttgtagttttttcccattaatttaccttatatctcgatccctgccccccactgtttgtaattttaatggctggttagtattctgttcactgtgagcatatcatagtttacctaatgatttttaaagtattgttctagattgcctctagtttactgctattcgtatatatatatacatatatatgcacaaattaaaattacacacacacacatacacacaattttcttttgctacatgcctagaaatgaaatattggctcaaagaatgtgacttggctgttactagttcttctaccctgtgaaagagaagctggaatttaaagttaatgtgaacatatgtactgggatatgacatttattcatctattctaacagtggttaatttgtatgaggattttcactcttgtaactgcagatccttgtaaggtagtcctgaaagataggcagactctactggagaggatcatctgactttagtttgtctttgggtagttttcaaaaagtttctctttcgtttagtttcactaaaatggcaactttagcccttgccattaactcgtttcttccctcttaatttagtgatttggccatgaccaaaataacttgtgagtgtgagagatttatttggcttacacttccacaccatggttcttcatagaagaaagttaggacaggaggttgaaggcaggagctgatgtagaggccatggaggagtactaagtactggcctcctcctcatggcttgttcactctgtgttcttactaaacccaggaccaccggctcagggatggccccacccacagtgtgctgtaccctccctcatcagtcactgagctaaagctctacagctggacctagtgaggcattttcttaattgagcttccctcatttcagattaccctaacttgtgtcatgttgacataaaactagccaagacaattggaaactaagtgcatttgccaatttgcaagatttaatatcgtaggcaatttggggatgagaggaatgactgaaactatttattttctcttttgacctgtaacttattaacaactggaaagtgaaagaaactgaaaaatcgagtgtttgaaattgtcattgtggtggtttgagtatgaagtgtccctgtgacttgattgtagtttgtggtgctggttgcaggggtttagaggatgcagtctgtgctggagaaagtttgtcactgaaggtcccctgtgagattacgtagccttatcctgccccaggtagctctctctaaatcatgcttgtggtaaaagatgtgctccctcagcttattgcccctctgccatgcttgccattgctgccacaatccaactgccacaaacacgagtgcaccctggcttctgtaagttgccgtggtcatggcgttaattcacagcagagagaaaactaatacagcccccatcgtgtagcttctgtggagtcacagagtatttaaagttcacgctgctcgtctcttctgcttaaactttctaaagcccagatcctttgcccctaataaaacaaagcaaaaccatacagtgtattgggctatgcttagaaagctggactttactgcctggcctccaggtatcgcttagtgatggtttgtgtatttgggtgttgtctatccggtagattaccttttggtctaggacaccactttatatctctctctctctctctctctctctctctctctctctctctctctctctctctctctctctctgagctgaggaccgaacccagggctttgcgcttgctaggcaagcgctctaccactgagctaaatccccaaccaccccccctctctctttttaagttttaattcattcattaaaacaaacaaaaccgactcttcccttctgtgattccctttctcccgtccattctgcctccattgatcatgtctcttctgttctttgctgtttcccgcatattagctctgtgagtgttttgttaggtaaagaatagctgagaactgggcgtgtgaaaatggccctttggaaaggaagctctgtcaagtgcttaagcaaagcactgagggttggatggctggcgaagggtggcgtttctctgaagggtggcgtttctcctgtcctgaggctgggaagtcttactcctagatttggttctttgactttcttgacagcttctcactgtattctcacatggcagagagagagagagggagagggagagggagagggggagagagggggggggggagagagagagagagagagagagagagagagagatctcatgacttcatctaaatttaattacccaccagtggccccacagcctgacatcaacttgtcatgacttagagcttcaacctgtgcaccctaaggggagctcagatatttagtcaagaatggtattcatatcgagttctgcggcttagggtggtactttgcctgtgagatgagtttttgtagattgcagttaatttaagaatcttgaaataaggccattgtaaatgatccattgggacagatatccttaggaaagagagagacaggagagatttaaccaaggaggcagagtagactggcagagctgtcagggtgcagggcaggttttgttcctaactaggagaaacaggagggattctcagggcctctgagaggcacagccttgctggtgttgcgagttttaatttctggtttccacccatgagagagaattatttttgtaacaaaaaactctaatgtagagactagaaatgatttcctgtttttacttattattttatcacatttatcttggattcttgctttacttcatgctcttatgttctcctgtagcttctaaatctataggattttttttctacttgtatgatcttgtaggtttttgtttgtttgtttgtttgtttaagacagagtctcaccaagtaatccaagctagcattgaactgccaaggagtcagaaaagaacaggtgtgccccggccatcccctgaggtcaatgacggtttttagtttgctgttcacatcttttttcctatagtatgtttgtgtcttaaacaacccccagaccttccacatggtttacactgagctgtttcccccatgtctttagttgtctgtgacttcttcctggttccttatttccttattccgggtctttgtcctttatgaatgtggcttgttttagttcactctgtgcacctccaggtgtagtagtgaggtggagaacaggcttcagggaggcttcagtgttgggtactgggtaggtcctttgttgatttcgttccttccctcagcctggttcactaacctggaaacctggtgatgttacagatgatgagtagggtgctggggaccggaggtgagaaatgaagtgtaggaagctcttccacttccctttccttcctcacgagtagctgcggattgaggactgtcacagacaggaacacggtgctgcgggaggaaacagagccatgaaacaaatgggctttactttctcagcctggatggtttttaaaactaaaacagcatatgtgttttgatttcactcactatcattgttttctaaatgtagcttggtaaactgtctgattttttacaaatgatttgtttaattaggagcttttcacttctcatcgacttgaagtccaattgaaatatctgactggagatgacaaaaatatctgaaaagggaagaattagagacattcaggccatccaaggatgaaattgtttcctacctttgtcctcagtttctgacatttcacaatatcagcaatgtgggaggtgctctgaaggctgtcctaaccagtgtgggaggtgctctgaaggctgtcctaaccagtgtgggaggtgctctgaaggctgtcctacccaatgtgggaggtgctctgaaggctgtcctaaccagtgtgggaggtgctctgaaggctggcctacccaatgtgggaggtgctctaaaggctggcctacccaatgtgggaggtgctctgaaggctgtcctaaccagtgtgggaggtgctctgaaggctgtcctaaccagtgtgggaggtgctctgaaggctgtcctacccaatgtgggaggtgctctggaaggctggcctaaccaatgtgggaggtgctctggaaggctgacccccagcaaggtcagccgcatttctgaccagtgtgtgtagggcggtgtcattcagggctgtgtcttttttctttacccagggagcattgtttctaatgcagctttgtgaggagagacacaaagtggcttagctctgtgatcggacgtagttggctatactcttccttcctggatcaatggactttggtagacctaagcataatttaggcatttaatatgcattagaaagatttggatatttggggaaatggttagaaaggtaattttatttatgtatgatgtctcttgcagacctgacgtaaatgaccatgctcttatctggttacttttcatagttacatgggttcctatgaagctattaagtagtattctttgttgaaattttaaaatttcagggtttgtttcttaggtgtattttcctaagttgtcctataaaggactgcgtaatggttaatataaatatgtgtgtattcacagtgacaaataagccaaataagccaagtcctgtgccttcgcttactacctgtggttaagcttggttttagatcttggggtttagaatgaaatctgtagggagaagaaaggaagactccccctgccccagtaacaactaagatgcgagatgtgggataaatttgtctgggattgattttactgacgtaaaactgttgcaagcttacgggaaagggacggttttgtgtgactgattgcagatgtaggaacgtggcaatgttaagagtcaggctggccgtcccggtgctgctcagtaactgagtgtctggagtctggtcagatgggaagtggggacagtgcatctctttgccatttttatttgaagagtctcacatcaacggtagtagtttataaactaaaggcacttttatttaatacctattttaaagcaagtttgtgcttttggatttataaaaattatagtattaaatttacatcaaaactgttgggtgatctagtgagatctgttcttcacacttctctttgcttgtagaaaatcatgctcccctgttttccatttctgttccttctggtcttgcaagagcaatagctggtagtaagcagttgattattcctttgtatgatgatacattttcttaaatgtgtgctctcaagtctccttttactcattttagatgagttatgaatctaaaaagttggtttttatcaggcaaaattaaactgttgtattttaaaaggtgttcttaacactttatattaaaaggtgtgaagactttactcaaagtgtgagcttctgttggactgagttttcttggatttatttggaatatgcatgactcagtttacttccacttttaggtgcattgaacagtgtacagaacttgtggatcggatgaccagggtttagtttctgatcttactatataatccagttagtttctgagttttcacccccttctccctggtttcaaatcatttttctctcccttttatagtggttggcttatgatggatacagataacacaccttgtaaactctgaagtgctccgtcagcgttgtgtggtgatcttggctgggcgcagggaagctcgtcagacagtctggttgtgtgtttgccacctcttttgaagtctgctgtgtttatttcagtcactctcatctctgtagtttctaaaggatactggcagctgcagatttaaaaaaaaaaaaaactcaaacgagcagttactttacgttctgctgtgtgtgggtgtgaaatggggtcgtttctagaaagactggtgaacatggttgatgccttcaattaatggagtttggttgttaagtagtgattagtaaaacatgggtccacctgtagtaaacagtggttctagagaacacagactctgtgtcagatacttctctgcagtttttagattaataattttacactttaaagtatgtaaagatgaacttgtgtgatgtgctatgggaaagaagcaaggtttttgttttttgtttttgtttttgtttttttttttttttttttttggtgcctatttcagtggaaagcaaaattgaacgagctgctacaatgtgtactgggttgtcagaaggcaggccatattttccatgaaagggttgagtggtaagtcctaacagacctaaaatgtgtagagtgttagttctgatgctgcactcaggatgggctagaactcgattcaccagttttgtaagatgatttttctggctttcttttggatttgtggctgggcaggaagccctcattcagaatcatagatcttaaagtaattatttgcttttttactgtaagtacttttaatgaattctttccttctgggtatggtgtattactacattggtaatctggtaaattagctttttaaagggaaaatgcctgtctatctccctttgtttctcaggtgtgcagtttggtaggcacatgaaaattttaaatgtcaaagttctaaagtcaaagcagcttgggcatactgcatatcctttagatatcagatacttgtttctaaaacctgtggtaattatttcaagtgtggtattagatcaagtcaaatatttattcatttttggtttggtttgagtctcttctagttcatggaagctaaaaaaataggaaatgtgcttgattctatgaattttagtaaaataatatcagttcagagttaaattagtgcagttcatttttattttgacatttttgcttggtttaaagtacagttatagttaagagaagatagaaatatgttcattaaagctagttcaaaacaagtatagctatttccaagtaaaattttgggtagctactagggtctgttaccagacttttctgtgaagtatttgaagtttagttttgctgaacacagttaggttttggacctggacagcaatgtgtcctcttaatagccaagcccatagtatattgttggagagcgagcaacaattctggactctggtaaacccacgtgtggcaaacaccaccctattttcagtaagttctgaatcatattttgctgttctaaaggcgtttatgtgctttcaccccgggatttggatccgatcagcggcaagcatagaaagaagtggaagcagaggtgcagaatcagtcagccctcagcggagcagtgctgctgcgagagcagtctccgctaatcccgagacagcatttgctgagcgctttgattgtgttaaatcctgtgctaataataagcaggagggaaatggtcaagataacctctgtttttctaagagttcttagctaagtgaggggccagtgcttttactaagtatagaacattgcgataagcaccacagtaggccacagtcagaggtacgagagccctggagacagggaggatgttgttgtttctgctcagtaagtcagaattccaagagtgtaatttattgagcacccagtatatagtgcatcattaaataagtcaaactacctagtgagtggttagcattcctcagagaatcctccaaagtgactggcactcctatattacagatacacagcagtaggctggacgcttcaaaacatggggtccgttacccagcagcggggtggagtcctttggttttatttggaaagtgggcaggcaaggagaaactcttcaaggactggtcttgactgactttctctttctttctttctttctttctttctttctttctttcttcctccctccctccctcccacacacacacacacacacacacacacacacacacacnnnnnnnnnnnnnnnnnnnnnnnnnnnnnnnnnnnnnnnnnnnnnnnnnntccctccctccctccctttctccctccctccctccccccttctctcttttgaattttttctgtttctagtgtgagtgagtggtaacagtggcagcaggcaagcataacaagtgatgaactggctggttttgtgtgtcaacttgacacaagctggagttatcacggagaaaggagcctcccttgaggaaattcctccatgagacccagttgtaaggcattttctcaattagtgatccagtggggcctgttgtgggtggtgccatccctgggctggtggtcttgggttctataagagagcaagctgagcaagccagggaagcaagccagtaagtaatatctccccacggcctctgcatcagctcctgcttcctgacctgcttgagttccagtcctgacttcctttggtgatgaacagcagtgtggaagtgtaaggtgaataaaccctttcctccccaacttgcttcttggtcatgatgtttgtgcaggaattcaaaccctgactaagacaagtgggtaacaccttttgttttgtagctcttgtgtaggcccgtttaccagaacagttaacagatcataggcagttggccttgcagcttatgtacctcagttttgggtttctcagctcttcacagcagctactgttacagctttgtaacagtgttatggctctcccatgctcgctctcccacatgtgcgctctctctctctctctctctctctctctctctctctctctctctctctctccctccctccctccctcgcacacacacacacacacacacacacacacacacacacacccgccccattctccactccccacccccagttgttgcttctgttggccttaacctgtttctgtaactaagggcacctggccgcttcagtgcactggaagctctgcttgtctcctgcttagctcgccctctactgtctgatcactctgggcagttgtgcaactccatgcctctgctgcaagtgcagaattcccttggttttgcagttcagttagaagatccatgtcctctgcagctttcaaaacagctggaaactccacacctctgtagtgtgttgaactgcggcatccatcgttgccccactctatggccactgtcctgcgacgctgcctgtgcaatctgctccactagaagcaggcaagaaaaacccacctgggaaaatctttttattaatttaatgctacacctaatgttgtagcattaggggaggcaacacaggagtgttaattagttggctcaaaaagtggtcctccttcatacacatgagggtttatgccagtcatgctattgtcctttctgggtctgtcacagcggtcctgctaggccaccagtgaaagtgcactcccagtctgacctgggggtaatgactgaagtgtctcagggcttcgtaaactttttcttcttgtgacccctctttttgtttgaaacaaatctaggcaacaccaggtgcatcgatatctaaaataggtgcacaacgctagcaagatcgctgagtgggtagggacacccactgtcaaagccttatgatgcgagttcatcccggtgtgagacaaactacacacacacacacacacacacacacacacacgtgcgcacacacacagaacatagtgtcattttaaaattctaaaacaaatatatataatacagtgtacatttactgctaataagtcaaatatatttcagaaaattcattataggtataataatcccacttactaaagacaaaatcaagtttgcatactaataatgtagatgtttatttttacataaataattaaatcggtaaatatttgatactgcatgatgaagagtatcttcagaattctatgtgaggtaaacacactgccgttgatccctgcttcagaggtttcatagttgatgaatatcttcaagtagactgatcaatgctgagaataccactactcagggtagcatggtagaagtgtgttgacacggtgttaacaacgtgacagatgttagtactgtctgagaggtgagcctttggataagtctgtggggattgttgagtgaggtgatctggcccacccactgcagatagcctcgttccccaaacgtggaatcctagaacacatacctgggcacaaacacttgtgcatgtctctgctcaagctcccatgcagggactcttctgttgcagtagactgtagcctccgctgggagctttgcatactgttgtggggttctgagtcgtgtctttctaaaaccacctagttttaagatatttacttcattgtgtgtgtgtgtgtgtgtgtgtgtgtgtgtgtgtgtgtgtgtgtgttttcctgcatgcatgtatgtcatgacatatgtagagtgtgggtgtggagcccttagaactggagttaaagttgataactgtaagtctaccgtctgggttctgggacttaaactctggtcctctgaaagagcaacaattgctctaaactgctgagccatctctccagctccaacagcaattttaaaaataaaacattttaatataatacaatttagagatatgtaatttgttatacatccaggaatgacggtagaaaatatgaaaagtctttgctttcgctaaaccattatctgtaagagccaaaaactttgtatgtatttatatgcatagtaaaaccctgtagttcatagtgtacagtagtcttcaacctgaatgaaggcatttcaggatcggcagtctgtgccgtagcagtgcagtcatcagcacagctcacggtgtgtttagagcaacgctgcctgcagcaaatgcctgcagcgcggtgcggccggccgagccctttcagaaccagattcagctcacccttcttgcttggttagtttaccctaatgctttacataggattaggtgggattgacatttttttactaaaaatttttttacctagacattgccctggaatgagctgctgcccattaggagtgactgctttactgatttcccggtattccttttctttcttcctcagcttcattgccccagcatctgtgagcagtgacactgtggtgtagaaaatccccaaagaagagttgaattttagaatttgtccattctgaagctgtagctcttgattttcctttttgacttttatgttggttcatgtatctaagcaaagttgttttctttgggagagcatctgctttaatagttgtcctttaggcagttaacatagagctgggtttttttaaacctgaaaattgacagcagccagtgtttccttttccgacaaccataatcttagttgaaaattactgttatttccctttgtgattaagaaatgataggtagtgttactctccacacagtgccgctcttaggtaggaagcatggaagcttggcagccccttggcagccccagttggcatgttgtactccggtcagtgtgtgtcatacaaggaggccagggtcttgaggcagagggcgagttttacaggtacagttgttacaactctactgcccccctcaggtcacggagcagcacgtggacggtttcagggccattctctgggctgtgggacttagtaacattccctaaaaccctgttggttagatgtttcatttgttgctttttctcagctctgaatcttgagaactggcctctttaagaggaagaaggaaaaaaagggaaagggctggggagatggctcagaggttaggagtacgggctgctcttccagaggacccaggttcaatccatagcactcaaatgggagctcacaactgtgtgttccagggaatctgacaccctcacacatacatacatgcaggcagaacaccaatgcagaaatttaaaaagttacttaaaaaaggaaaaaaatcaaattagtggttttgctaaataagtaaagtttgtgttaagatttattaattcatgtaattttgtgtaaaataaacgaccgtgctagaatctcttaaattattagtctttttttaattcttactattgaggtgggcttttgagtttttagatgctcattccacatttgtattctagatttaacagccttcagcgaaacagtctttgttttgtatgtatcattgtatgtgtgatgtgtgatgggtgtgagcagcgtgtgttccgttgtacacgtgtggtggtcagaggtcctgagcatcgtgtgttccatggcgcatgcgtggaggtcagaggacctgagcagtgtgtgttccatggcgcatgcgtggaggtcagagcacctctccagtttccagcctgggttttccctgcacagcagcactttacttgccaagccatcttgctgtctggaatgtggttgttacattgcataaggtcttcccattattttcatgagggatttattgtgattgatatatttcgggtctgctccactgctctgtgttctacccgagagatggtgacagttactaaagaccgattttattttaatggtttgtgtcatataactggagtgagaactgttatgccatatattctaggaaattttagtgctaggcagagtagtttatttctttttctaatgtgacagacatattataatttttagtttttcatgaggattttgatttttgtatgccagtggtgttctaatgtagtacaaggtaatctaacttttagtggtgagtaatctccagttgtttggatgtcaaggagtgtaatctttgaaatttttgtttgtcattttatttttttaatattttattttactttttatttttcaagacagggtttctctgtataatagtcttggtttcgctggactcagtttgtagaccaggctgaccttaaactcacagagatccacctgcccctgcctcctgagttttgggattaagtgtttgtaccaccatgcctggctcattcctgaagacagatccacactcttttgaagttaaactttaaataaagccgtcagaaataaaagttctaggattcttgagctgtgtatagcctgaaagccattgcttctcggttactgcaggcgcggcatgatacaagagctctctgcggtctctctaagtgcttaagagaaaatgaaataaatctagtttctactaaaggattggtcattgtctgcagcagtcaccactcagaggagagggcatcaagaatcttggtgtggtggggtagggtagcacttggggtagaggcaggtggtctttgagttcaaggatccctggtttatatgaggagttcgaggccagccagatctatacagtgagaccctgctccccacatccacacttcacctcccccatcgaccccaaatcttcactaggcacagataaaggctatttagtttttctactctattcagtactttagcagttactttatttccacctcttcccatttgccagtcaggatgctggggtaactaggcattcccacgccgaggacttggcaatggaaaaagggttgttggttgtctgcagtagtgtgcactctgtcctgaaacatgaggaggttctgggagtcctccctgcagaggctcacgggggagatcatcctagatgctgctgtgctagcatgtcttacaggaaccaagtcaaaactctggttcccctttctgtgttgaattgtcttcgtacagtagaaaggcatacttttttttttttttttttggttcttttttttggacctggggatcgaacccagggccttgtgcttcctaggcaagcgctctaccactgagctaaatccccaaccccgaaaggcatactttttctttgtactgtggagcccagcgaccattcatacaagttatctgctccaaagtaggaatgctttgttctgtgaattagacctgagatttggaaagcagacttgtttaatagacttagtaattcagagttcttgagaatgtggcttatctttcatattatgaaaaaaacactttaggaaatcatttcagctttttatttttctaataaaatgtattcagtactttgtgttatagtttttctacatcttactgccctgcatctcttgatttagagtttaattttaaaagactgcgaacctatctttaacaaataaggtgggtattgtactgaggcaggtgggtctctgaattccaggctgactttgtatatagtgagttccaagacagtcagggcgatagacagagaccttgtcttaaaaaccaagagccaaaacaaagcaaaagaaaaaagaaaagaaaaaggtaagaaaactaagattttttttccttaaggaagagcgagaaggtagaattgacagccaattaaaaaattgtagttaagataagcagtcagaattttgaagatagaacttggtaggttttagaaaacatctgggttatgactaagcacatatttaagagctctgaggcgtgcaatatgtgtgttggcttctgctgctgtttgtatgcattcaaaacatacataggagtgcttaaataaaaactggtgttgatgtgtcattcacaaatgatgctatgctaatttgaaatttgcgttaattaagcaatatatttttgagtgttttgccttgaaactaaaagcataatgcagactacccgcagttatgcagtaaatcacctggccatctttaggagtcactcataactttgtcactgtttcctacctgatatgaaggacatcttaaggaaaggctgatgtcagtcattgcatatcggcttttgtctgtgttaaatggacatgcatattgtcattgtttgccacaacattttaagtgtccatctatcaagaacctgctgtttgggaaatgcttaagagacttagtgtgagtttacttagcatcttaaaatcccgggctggacagatggctcagcgggcaagggcgctgtctgctctcccaaagatcttgagttcaaatcccagcaaccacatggtggctcacaaccctgtgtaatgacatctgatgcgctcttctggtgtgtctgggtttatatataataaatgaataaatctttaaaaaagagtaaaagacacagtggaagagaaatagagacaaagatcgttcagtgaggccaaagagcttacattatatacatatagttttatattttgtattggtttggtgttaacatttgttaggaaagagagccgagggggttggggaggcatgattgtcatttctaggtgattgtagtttctggaacttgggtgtattgtgaagtcactaaaagaacagtttaaagcccttgagaaccatctgtggcctaggaaagtttggagttcagagacgattgtggactgcctagtcagatgggaagttggtcctgagcttcaagggctcattttggattgtcgccaaggatgaggcaacggaaaaaggtaaggaaaggtctgtaaatgagatgaaggtgtggcggtaagccacacatgagtgcaactcagactggggtttattgtgctttgttttatgctatccctaaagtaaagctctagtcctcgtgtgactatgttctcacaggtattactatacacacgcacttgaggttaaaagagtaagatcaaggagagcagaaactcaaggccttattttccagaagtcacaatggtcaggtcggagttggtctactcatacatttttagttatatttatttgtttttaagtaaatgggcttaaagatgagttagacacaagtcatgagggagtccactttgtcttggggtagctttaatttcattgcatctatttatgtatatagcctctcagtgtgtgcccagagtggcattagctgtgacagctggcttcccattagtagtgttcaaaattacatattctccacagtttcattcacacacattttaatcctatctctcctccttgttgtcttctgttattcacccttcctccagacctcttcttacaactccggtatctgtgccatgacaataatacattgcaggcacttgttttcagcaaaagatattatgtatttaaatgaagcagaataggcataattagaatgactaaataaatggtgggttaaattgcttataattgaaaactatcttacatgctaaattgataagtatttaagtaaagaatgcctttactttgtatgtgttggtaggtttgggtttttgcttgtttgtttgtaaacttctatatagttctcatttttgttttacatctctcttcttgtaggagataaagatggcagcaaggtaaccacagtggtggcaactcctggacagggtcctgacaggccacaggaagtcagttacacagacactaaagtcattggaaatgggtcatttggtgtggtatatcaagccaaactttgtgactcaggagaactggtggccatcaagaaagttcttcaggacaagcgatttaaggtaggacgccgtgctttgtgtatcttcattcttaaactttttaaaaatttttattactcttaactattggactgtgtcccttcccttccccttctcccctctcatcccctcccctactccttctcctcccaccccttccttcctgtgtttctttatagcccagacttgctctgaaatcatgatcctcctgtgtctcagcctctaaaggccgatttattggcttagatttgttttccacagatttgtctgtcgtttactatgcgtcctaaatgatgagaactagcagtgggatttgtgactgcttcttgttaaatctctcatcttcatcagacaactgctggattgcaggggaaattatataaaagttatcagttgggttggagaaaagtatacataaccatttagcttgcttctcccctcttctctcccacagcattgacttaaattttttttcctacacttatttatttcggttgtgtgtgtagggttgagggagacgttgacgttggcgagtgtggagttggagggtgcgttgtatgaggcctgggcttgtatgaggcgggttgtatgaggcctgggctgtcaggcctggcagcaggcacctctgcctgctgagctgtcctatttgtcagctccaggggcacccaaggtttctctgagtaacccttgctgttctggaacttgctttgtagaccatgctggccttgaactcatagagacctgtttgactctgcctccccattgctgggattaaaactactacctggcgctccattttaatttttaaagatgaaattttataattttttttaaaaatgtggattgtaataaactttctaaactttgctggagatgtttaataaatttatattaaccttatgagatttaaaaaaaaactagcaattttatttagtatttaaaggaagaattttgggtgagctgtaaattttaattacatttatttattgtgggcccccacatgtgctgtggtgcatgaccgtgtgggtcctggatggagtttagattatcacatttcctcagaagcttctgtagccacggagcccccgccccagtcccgggtaccagtttatattccaatttactagacttattatggattcctttttattttaataaaccatagcaactacaaataatgtattcacatttttccttttaaataaatattcgtattgtcccaatgaccaagactcccacagtaatgtgtaaatgttgacaacagatttaattcttgtccttttttaaaatttgtgggtgtgaggtctaaataaagatgcaagtatattagaagaaatctttcttcatggtttcttactactgcttactaattgagtcttgacagtttatgggtatctaaggtgtacttttgttacttggcgatctggaagagacatgtcaatcgcattgtgcagatctcctgtattagaggaggttttatatctggatagataggctgctccttcaaaatatatatggttatgctaaattctacctcacaagggtttgctaatgagcattcagctaagtaatgagtgcttgtttttccatggctgtgctggcaaacttgctttacttttgagttttgctaatctgataggtgagaaatgatacctcgttctacagattagtaaagtcctaagtagaccacagtttagagagctaatactattgcaccaccctgccgtgcaatgctccatgctaggagtttagcaactttaggaaggtcctagatatttcataaagcctggctggtccctgtgcagtagcattctagcctttcacttgtataaaagaaaagaaatgactttgtagagccaggtcggttggaggataatgacaccagaggatttaggggtgcctacttgagttcttggtacttcttgatgaactgggcacattgtatgggttgtggctaaactcttccctctgttggaagtttaattaggatttcagtaaatgtataaattaggaaggatacgtttatgactgcatatttcattggttttcatacatacttctgtcccttgcatagctgcattgctcagcctgggatatttccactaacgtatcctttcctcatggaatttacgccgttctccttaactacacaggtagttgagaccctgataacatttgtaatttcacttataatgtaagttatttaacatgagggatttacataattgctgctttggactgtattaagtaattaggtttgaccagaggaaaaactagctagctagacagacagacagacagaaaaagaaaccaattggcctatgctactagtgatactaagaagcagtttgttttattctaaaaataagatgagagataataagactgagaaaaagaaaagcaagaattgtccccattgtagtaatttttacttttagaaatacttattagatcttaatctatagatattttaaaactactgaatgataatcaaatcaaagaataaatcaagaaagaaattttaattctgaggattgaatgaaaatgagaccataacacactaaaatctatggaacataatgaagacagtctggaggaacatttacagctctcagtgcctacatcaaaaaacatctcaaattaataacattagtaacattaataacaaaggctgaaatgagatgaaaacataggagatactgaggaaatatggagaattataaggacacatttaaaactgtatccttctaaactgaaggaatgtaaaagaaatgcacacatatctagatgacctacaaagttaagtcaaagtgaagtatgattcaaacagccgcaaacaatcagtgccatagaagcaacaaaaaaaaatatcttcagtagaccaggccagatagttttagtgaagaattctaccagacctgtaatgaagacagactaatgccagtgttactcaaattacatagaaaaggaaggaatagttccagattcttttttcatagtcagtattattttgataccagatgaagatccatagaaaaagaaaactataagccaatttctcatatgtagtatggatatgaatgcaaaaaattcccagtaaaacacatacaaactgctttagtcacagttctgttgctgcgaagatacacagtgaccaaaggaactcttagaaagcttttaactggggggcttacttaagatttcagaggcatagctcattatctccacggtggggaacccggcagcccatgggcagacattgcactggagaagtaagtgagagttatacattaggatctgtgggcgggagggtaggaatataaaatatgtgagatacaatatttgtcttgttaatttgttaaaacctttcatgtggctgtgcatgtatgtgatctgtccttcacagagtnnnnnnnnnnnnnnnnnnnnnnnnnnnnnnnnnnnnnnnnnnnnnnnnnnnnnnnnnnnnnnnnnnnnnnnnnnnnnnnnnnnnnnnnaaatcaaaataaaaaagcagaccacatacttccaatatgcaacagcacagactatacattagcatcccaaaagggaggaaagtgagtacgtactggaccaaagcaagaccaaaagacagccaggcaaactctaaatcttaactttacatgtctgatgtcaaagtgctcttcagatctccaaccccttccaggtttgttgactgcaacagactttttcccttgggttggttccacattctgttagcaagtgtccttggccagtatcccacgactctagcatctctacaatcttggggtcttcaacaaaaatctaggcctcttcctcacagcttcatgcagtggcctctccgagatactcctggcacagtcctggtctcggtggctttccttagattccttccacaagccttctcttgtatccttgactctaaaggcagaatcctgaggctaaggctgctaaggctggctgctgcttggtggggctggaacttggccctctcattcagaaacatttgcctcagctttctgttggtgatggcttccttcactgctaagcctttcccttcacaagttggaagcttagctgggtgagatggggtcaccacctctctcttcattgagcatcaggcttttctcttttctttaagcttcagcttcacctttcctgatgctctctgcccctcacactgcatattttgtatctctttgtccagcttcatccttttctgttataatatctcacagccacaaaacagaattaattaatactaggctgtctcttcactttagccttagacagaattttaggaccaaaaaaagcaaccacaaaaatgttctctaggccatttcctagtatttttcccctcctgaactgtcctgagccaggtcctccacagttcagtttgccctcagcattgtgctccaactagagcccccattaaggtctgcttaaagcgtccagcagcattctagtacaaaagtctcaaacaaagacttccacatttcctcaatgaacagcatggtcagacctgtcacagccataccccatccccagtaccaacctccgtttcagccactgttctgtggttgtgaagagacaccataaccaaggcatctcttctgaaaaaagcatttagttggagttttcagaggttggtcaaagatccttgtgttagagagcatggtgtcacatatgcagacataatgctgaagatgtagctgagagttcaacaagcctgacccatagggcaacgggggttgggggggagctaactcgatactaggtctggcatggacttttgaaaccttaaagcccacccccagcgacacacttcttccagcaaggccacatctactccaacaaggctatatctccttactccttgcttactaaacattaaaatatatgagcttatggggcccattcttactcagtccagcacataaactaaattcagtaacataagataggccctaatcaagtctgctccatcccagaagtacagagagggttcagcaacaaatcaataaatgtaactcattacatagattccaaaagagctttgacaaaacctggtcaatccttgggaatacagagagagaggacatatatctacgtaataaagaaaatatacaataagcccatagccaatatcatactgcataaagtagagctcagagcatttccaccaaaatcagggataatacaagtatgtctgccttctctatttctgtttaatgcagtgtttgaagtcttagccagagctataagaagtgaaggagataaaggaaatagaaaaggaagaattcacagttttctctacagccgaggggaaggaagtctttactagctctgcatccaacagagggcttgtgtctagaatatacagagaacgaaaacaaaagctacacatcaaaattcagttaaaaattggggaatggaagctgggcttgggggtaaatgcctttagtctcagtaggagagacagaggcagggagatctctctgagttctcggatagccaggactaggtaaagagcccatttctaaaggtgttgggagttggcatggaaatcagcagaatgttcaagagaagaaagacaactgactgagaaaaacttaaacaaaaatgtttactatccttacaccaccaggcaaaaataagctatgacatttcatccccattagattgcctaagacaccaaacacagataaggacgtagacaaagggagctacagggccttgtggtgggagtgcagacgtgtgtagccactgttggagtcagtgtgagactgcctcacatgtgctgggaatacatgatctagctatgctgttcctggttatagatccaaaggaaccgcttttaccacggcaatacttgctctcccatgatcagaatgccgggaaacggaaagagcctaatgtctatcagctgattcataaataatgaaaatctggtctatattcacacaatggaatattggaaaagcaaaatcatgaagttttcaggcaaacgtaattctgtgtgagggaacggagacctagagagataaatgttgtgtgttcttttatcttaatttagctgtgaacactgggagggagcggcagtgactggcctggcgagacacgcccactgctgtggtagtggcaggactggagtgggactaaccaaccactttttaaattagattgaatccagctgtgcaagttgaaacccatgtccggccaagaacttgtggctggggagagcctactacttactatttacttctaagtggagtagcattaaaatgactcctaaggatctctcgtggtacccagagacagtagctgtctaccgttcactagagagacctttgtggtagattgtggaatgcttccctctgaacgggatacctcatacctctgctcttaaggctcgggagttaagataaaagtaagtctgagtaagccagagtcggaggatgactgaaaaggaactgtcttctggacacgggatgactgaaaaggaactgtcttctggacacggagggcagttacacatgaatttctagtagttcagaccgagtgcagaggaccatcccaagcccaccctagacaaaatcccagcagggggcgggggagtgtgcacgagtcacacccatgagctattggccactgaaaactgttgggagagtcagtttactttaagggtgtagccccggacaagttgaccacatccaggagtagatggaaacctcagctgtatttgtggaggggagagaagggagttggtgagaaaggggacagagacaagcacaaaagtggggttggtatgggagacatggagggagggccagataccaagatacgttgaaggaaattctcaaataaccgattttaaaataaaggcggatgtggtggcaggtccttaattccagtactggggaggcagaggcaggcggatctctgagtttgaagccaggctcatctacagagtgagttccaggctacacagagaaaccctgtctgaaaaatccaaacagtaagagagggggaagtggagtcagtccttcatgtagtgctgagttcttagtagtggctagcggcctacaaaagtcacacagtgaaatctgaaagtacagattaaaaactattacagctttgccagacaaagtcagtccgctgttctcccagggtctaaaacgcattgttccatgcctccttacccttagggcgtttactaagagtctggtgttagctggacagtttgcttctgtagattctttgttagagcttcgagtgatattccttggttctctggtcttagcattttcatagcaatgtgaagaaattatttgttcaggattcttctgtcttttttgctcagatgtctttttatgtcttatattttggaaacttatgctatgatcgcattgtgcatccgggttttttctcagcccttctgtccttggatttacttattacgtgttaacataacagctctttttttttttttttttttttttttttctcggagctggggaccgaacccagggccttgcgcttgctaggcaagcgctctaccactgagctaaatccccagcccctaacataacagctcttagatgttagagcacccttatcatttttctttgctaatgtctgaatgttttttaactttatttcagtctttgatattctgcttggtctagtgtattggtagtaactttccattttaatgtttaattcctctattgggtaatccatatttgacattgcttgggggagctcccccaatgttaatttctttgcttattctctctcttttgttgctggatactctcattcaaggcttgaatgaattcccttatttcttcaatctgcctttttatattttttgaattagttgatcacttttaaattcatttgtcagcatttcaaccattaccctgtcttttttgtgtggtcattaagttataatcttttgaaagcgtcagtttcttgctttattcatctgttttttgttgctctttgtgcatctttgggatgattatcccatcgggttatatctgggaatttacttcataggcgctccctcctccttcccttcctcctttttatttgtttaaggcaggttttctgtgtagccctggctgtcctggatctcattctgtagaccaagccagcctggaactcagagagatctgcctgcgtctacatcccgagtgctgggactaagggcatgtgctaccaggatgcattctggaaggaagggagggagggagggagggagggaggnnnnnnnnnnnnnnnnnnnnnnnnnnnnnnnnnnnnnnnnnnnnnnnnnngaattcaatatgtaaatcagtctgtccttgaactcacagaaatctacatgccttttcatgtgccatgaggccttacgacgatatacccccttataatcccagtactcgggaggcagaggcagacagatctcagcagtacctgtttataccaacttcttcagaatttgaaaacatttttttctctagngtgttctgtcttgtggtaaggaaggaaggaaggaaggaaggatgggttttacatgcacgtatgtctctgtaccacatgcattggtgcctttgaggccagacggcgccatcagagcctctggaaacacaagctgtaggtgaccatgaaccacccccagtctctgtctatttctgtctgcagacttttcagctgaccttttatctcctgtctttggatgagtctgtctttgttatgtggctttacttactgcacgtcacggaggcaactttcgtccatcttctctgctcctctttgtctgtcccaagtagtgcttgtctggtctacacgtcagctgccccagtgcaggacacagcacagcccagaagtctgtgcaggtcagagctcgtttgctgctgttcaggaggctgcatcttggccttttggagcactaggttcctgatactgaattacgtgctatggccaaatgccgtcaaagggtggtgcgtttggttgatgctcagattgtatctttgttggttatatttttgtcttttggaaataatgtcactatgactgtttatagttattgttcagacatttacacacacacacacacacacacacacacacacatctgggagtggaatttctgggtcgcatggtgattctgtgattaaaatcttaagggaatggtgacctgtttttctaaatagctgtaccactttaaagtcccacaacagtgttgagctgtaatttctgcctgcacttaacgtgaaaacacttgctgttgtccactgggttttttcagttctaaattaaacttactgtagtgagtgtgaggcctcactgtctttcctccttgtcatatgggctttgctcacacgaaacccagagtttctatatttcatatagacatatttcatgtatctctaactttctttgcttttacttcattatagctataaaatatgtgagatacaatatttgtcttgttaatttgttaaaacctttcatgtggctttgcatgtatgtgatctgtccttcacagagttctgtgggtgcttgaaaatattacataatatcaatgaggggtttgtatgtttggtaggactattcaattaagttttactattatttgtgtgtctgtgtgtgcatatgcaggggtgccatggtgcctatgtggaggtcagagggaaatcctcggagatggttttctccttccattctcttaagttctggaggtgcactcagctcgccaggtctccatggcgagtttcttgtctactgagccatcttactgccccccccccaatgtttttgtctttctttattgacacagctgactggattatctttcattcctgatactaaattaccatattaagatttgtcagtttatatatgtagacgatgtaggggtggatttcagttggattcagccttttatcactattcttttttcttttgttggtttttgacttagaatttattttagctaatatgtcatcctacctgtcttctggtgaccatgggaagtatcctttctgtctattcactctgagctgctgcatttgcttcagcttatggtgagtctcgtagacagtgtccatgagtaatcgagtcttgctttactcattcgttcattggccctgtgctttaggctgctgagtttattctaaaggacatttgttgttttctgcttttgtgtttgcaatttgataattctttatcattttgtggtttgcgaatatgataactgcattcacatagaaccatttaagctgcaaacttattgcatatgaaaatttcatatagttttatattattaatatcacattttacttgatatattgtatatccattcatattttgtggttataattattagtactttcatcttgatttttgtaccagaattaaaagttaatttacatcctgcccctagaggcactgcaggtgtggctgctgtgtcacatgtgcgtcagtaggtggcagagaggaaagaggctatgtctacgctcagtgttctgacctgtgaacatttgaatgattagtagcctaatattccctgaaaaaaggtgaccctgagaccactacaagtctggggccagagggcccagacgggagctagcctgaggcagccactgtctttgccaggcagacctggaagggctcccaggacaagcaagccggagactagaagagctatcttggaccataggccccaggtgttagtgggccccagacagatggccacttgggtaggccaggagaggagtgagtctcagaggaacatcagactggtgccatgggagcctgacaggtggctaatctgagatgaaccccagttttgtgtcatgttagactggaggtacagaacatgcttgagatgacccttgcctggtgccataatgcataagcagggcctagcactcctgagaccacttctgacatgaccccaggcactcagagaacccaggcaccactaagaggagcaggtgagtggttgagaaatgaaagagaggggttggggatttggctcagtggtagagcgcttgcctaggaagcgcaaggccctgggttcggtccccagctccgaaaaaaaaaaaaaaaagaaaaaaaaaaagagaaatggaagagaaagagaaataaggatgtgcactggacacttaagggtagtaaggagtagccttatataagtagtcagtgatgccacctgagatcatggtggggtcctggcctgtgctggcactggagaccacatctgggagttgcggtctgttgccatgaaaagccagaaaaacatccctggtctgagctgccaacagtggctttgtttatgtttgaggactatgcagaactggcccctcgcctcacctgggagagctggctctagctgtgcaggatccttgacctgaccctagccagctgtagtactcaggagagcagccctgaccctagccagctgtagtactcaggagagcagccctgaccctgaccctgaccctagccagctgtagtactcaggagagcagccctgaccctgaccctgaccctagccagctgtagtactcaggagagcagccctgaccctgaccctgaccctctgtagtactcaggagagcagccctgaccctgaccctagccagctgcagtactcaggagagcagccctgaccctgaccctgagcctagccagctgtagtactcaggagagcagccctgaccctgaccctacccagctgcagtactcaggagagcagccctgaccctgaccctgaccctagccagctgcagtactcaggagagcagcccctcacattgcaggagcagcaggtgagctgggctgaggatgtgtgcatgggagagctggccctaccactcgtctccctgtggtggcttggatgagggaaagataccctccttccctgtacctcctcaccatcgacagcaggtgggagacctgtgcctaggctcattagagcagaagagctggccctgtccttcaccagctgtagcactcagaagagcagcctctgtacctcacttgggcagcacagtagacggatgagccagtccagagggctcactgggacacactgcagcttccacagcaagatttgtttttctctgttgtaggggagcttgcaagagtgaggggctggtttgggagtgttgaggggagatgagtgggattggggtgcatcatgtgaaattcacaaagaatcaataaaaggttaaaaaatgaaacttaaagtagtttacatagcacaattatggtttttcagttattgcctatcagtcttgaattttcaatatccttttgtatagctgtttagaatatactttttgctaaagtaaaattttagtatttattactagtgatgataaataccctcattttttaattagctgggaaactgatacatctatctggagaaggaaacctgggtggggtgtgtgtgtgtgtgtgtgtgtgtgtgtgtgtgtgtggttggttggttggttggttggttggattttggtagggatctggttttgaaagggctggtagattcctgtactgttaaacgtagtcagcccctgtcattgtctgttgagtcctttcattgagtgtcctctatagtcttgcctcagcacgtgcatctgtttcggttgacctcactctaccaatcacccctcgtctgtggaagtggcaagaaactgcagcacaccaccagaagtgttttggttcatttctctctatggagccccaacaaatgcagctcaactacacagtgtaagacagaccagtacatgtgtgccgttagcgaagagtccttcatcacgtgtccttttagaaccatcctctctcctgtgtgtctgcttcagcgaaacattcttccacgagtctgccttagcctttcacgcttctgtgcagtttaacaaaacattccttctcgtgtttgccccagcacgtctaaccaataccatctaaccaacgttccaaagaaccctcaagtttccactttggattcctccaaagctaagatactaaaagtaaacttaagagcgtatcagtaaacgaattcagagatagagagtcatgcattttatcacttgccacttgagggtctgtgattgtcacttcagtgttgtctgtaattatgtaggtaacagacctggacatgctggtgaaggattctagattggtttaatgatgggagggaggaggagaccagcatttatcactgtttcctgaagtgggggagtcacccagcctttcccactgtgatggactgtgaaacttctctccagctcctcttttattatttccatactggcagcaacatttcctacaatacaatgtgttgtactttctaaaagtagagctatctatcattcatctaaatacatagtgctgacttgtattaatatgacttggatattgttatcagaacaaggttctataacactaaaatattcctaaaatatggctcagagtttattggggagagttggacactaatgaataaacgcaagttaggtataatgtgttaggtataatgttataattttggtattctgcagaagaatgaagccaagacttggataaagagaggctggagctgttgaggttgtgatttaagataggataagaatgtagcatttgaggaaaacctgaagtaagtgaggggacctagattagttacttatgcagttagaagaaatttattgtgcctgtgtgtgggcatgcacaagtcatggcatgtgtataactcaggagaactttcagaagtaggttctttcctctctgtgagttcaggaatcaaacttaggttctccagctttgttgcaagtgacccttgaaggtctttagtctaagcgttggtttatatcgttactacaggcgagattggaagcactacaaggactgtattattctttgataaccacgatatctttctttccctgtcattcctccgcccttaacactcaacatacccctggtcacaaaaagtttatcctaccatctgaagagatggcaggacctgggttctgttcctgccacccacatggtagttcacaactgctcatcattctttcagtctatcagagtatctgacaccctcttctggcttctgcagctgctaggttatacgtggtacacagacacacacagaggcaaaacactcattcacatacaatgaataaaactttggggattgtctttttttaatgtaatatttttctggagatttagatatgtttttgagaatcatagtatgatatagatgtactataacttgtttatacatttcctcccatgaaggacatttgatggctgttcacattaagcttatagtttatgttcctgtgtagcttttcgtataaattccattgctagggaacaaatacacaagattgtcactcctggagtcctgtgtgaattttagagctagagccaactgtggaatgtgaggccagagagagaaccgaatcaatactgcaaggattacttaggtacctggaggcgtgcaagtgccagccattgaaatgggaaggctgtgggcggagcaggtttaaaggcagtcaggaatttgctaatgacatgacacaatctaaaaaggaaattgttaagtagatagttcactggagagattggcttggtgatactcagttgggagtcaccagctgtcaagctgcagactggacaggattacaggcagttgaagagagctgcgagaacaggagagagagagagccttgaaggaggaggagaatggttggtcacttgctgtctgtggttagtctgcagcagtctgagattgtaattcagttctattctagagggatatcgaaaccttctcattttcttctgagaagctgtagggtttttgtgtacttagcaagtcagtggtcccagagtaggattaagacttacctaaagcttaattttatatcctttcatgtaaaatgactcacattttctccaggtgcatgtttctaaaagatagttttagaactctcattattttaataaaatcgttttcattgcacacaccatgttctgttgtacggcatctgtgtgtatatatgtctatttgccttgttttgaatgataaattctgagcactgaagtctgctttattgttgtgtctctccgtcaatagtaatttgaagaccaaaacaggttacattccattgttagagtcagagcatgagttctgacgcacacacacccgtttaagtaaattaaaaaaaaatcaccgctggtgtttgctagcatcttaagtaattttttaaataaatgtgcattggtgtttggcctgcatgtaagtaagtctgtgtgaggggtcagatccatgggaactggaattacagttattagccgccacgtggtgctgggaactgaaccctctgaaagagcagctggtactctcagctgctgagccgtctctagccctactagcatcttcctgaacccttctgtacttacagcctctgtaaaatgagaataatggtgttgccaacgtgaaaggtttaaatcataatggtaaagctgctggcatagtatatatgctaaatatgtatatacatatatacatatcggtcatatattttcagtattctgtggtcattggtatcatcactgttgctaaacacagggtttaaggaatgcattcaagatagcatttatctatatgctaggacggcctctgaaataggatttcaagagcctcactcaggccttctttaacccaccacagctgatgggaagtatgagtcctctacttctctttttgccacacagttggatcctttataactataatttacaattcgatattctatggactggttatagtagcactataggaagatattataaattcctagagcacgagtggttaaattaatctgcaaaatttattctgtgtgtgtatgagagacgtaggggtaatagataaaaaatgagtatcaggaatttttctctcttgttttgtagaacagtgataaactgggctttcccttttttttaaataaagttttattagaacatggtcatgtttggtctttttaattttgtcaatggctatttttacagtgtagctgtacacttgagtagttacagcaaagtccaggtggtttgccagactttactatctggtccttcatagaaaatatgtgtccactcttactttagggtattagttttgtggcttttgtcttataacttcttatcacttttttcttcttgtgtgattactttgatctacatataaataagcctcaaaactcttcttttgaataccttaaaattttatgggactagggtcaataatgtgacaccaatgcttaggttctccagtttatttttgttattgtttattgtcgccgactctaattaggagtccaggcaagtctgggcgtcttttcttgctttatacacatttttatgctgctgtgacttccagagccctcctactgaggagggagtgaatgtattcaggttccattctgcacctttcaccctcagtgtctgttccctcaggtgaactaacctccctgcttcccaaagaaccttctaggccagtggctcctgctgctgtgttaatagccatgtgagtaagacacctgagagatccccaaattccacttaccagctgttaatcagcctctcagaccttggttcccattctcttttgagcatttccttctctatagtcttggttttgtttctttgcctcatcccttaataacttctttcatagtcaataatattccccaaatcaaagctcttactcagaacacaatcagtggttttttgttcatggcttgcaaaattttatctttgtgtataagacttttttccccctctctgataggtttgcagacctttacaaaataacaagcaggggttttttccccctccttgtcttctattgctgtgataagacaccatgaccaagacaactcagaagaaagaatttattgggagtttacaggttcagagggttagagtccatccatgatcatcatggcggggaacatggcagtaggcaggcatgatgttgaaacagtaacgaagtgcccacatcttgatccacaaacacagggtgcagagaaaactaactaggagtagtatgggattttgaaacctcagggacaccttcagtgacacacttccttcaacaaggccacaccacctaaatcttctcagagagttcctgtaggggtggttctgatgctaaggtcccaattgattcttgattgatcagtaaagatgcagtgagccaattgcttatgccccgccctcccaccccccatgggcaagctagcagatgagggaagaagagaggtgttgaccatgcttcggagggagaaaagccgaccagccacgtgagatgctggcgagatgttgggtggagcacccatgagctgcttctccaggcgggaggttgggatgctgagctgggactgaaggtggctgagcaactgaaattgagggcagatttaggatgctgagctgggagtactgggaggggcacgccagacgccgtggatcggaggtgttgagccataaggcaggttgaaattgagcaatgtgtatatctgtggttttcacccacagatccaatattctctgggcggtggctggaagcacagtccgcccaggacttaaggcagggtagcaaaagtgacaacaatttctccaactgcggaccaagtagacaaccataggagcccctgaggccattctcatttagaccacccttctgtcacacacatggtgatgaaatccctggcactgtgctggaaatttggtgagaccattcactagcatattattttgcagaccaagacaccaagttcttatgtcagctaagtattaaaaatatttgttatatacctgaagctattttgagattttctttttagaagccatttgtagagtacttgggggacatcaattttactatggaataaaaaatattagggattaaagcgattcatttctttggataacattctctttggataccattaatttttttacagaagttatatttagggaagggagtcttgaaaacaaagtctttgaggtgaatcctgtttttcagtagcagcagagttctctgtgatgagggataagatcttccagtgggacagactttggggaagagggaagatgatcgacagatgtgtgctgcctactttacacccacagctttaagcttgctggtgtttctcacctgaaagttaactttctccaggttacataatggctcctgaagtacctttctgcttatctgtggctctctggcttagctttcagatgcaagccagccaacgtgctccctccctccctggtataacatcagttatggtaaaagtgagtcaaaccctttatgtaaacacctggcagaaattacttaagggaaggagagactttctggttaggatttattttagcaggttcagtcctgggtgagatgaccctacagttacagtcccatggctcgatactacagtgcaagattgtactggagcagagatgctcactgggaagccgatacgctcccagagaactcctttctctcgctaggctcgtcccaaacatactatgaatccctaaaggccttaggattcagtcactcagtgattggactcactagccatggatcaagactttaacatatgcgtcttacagggaacgtatatctaaaccataacagttgttacatgtattttatatgtattttatatgcattttaatgtttcttttataagaagaagaaaagcaagttttgattgacatgtccatgtcaggctttctcatttgtgtggcttttgttttatatggagtagctagctttttgcgtgtctgttacaattacaagtcttttgtatgtaagagtccacttgccttcccttcctgttctcttcccaatgatagctggccaatgaagacagcaagttcaggttttgctcttaaggagtttgtagtaagttgattagacatggggtatatgttgtctttgtcattgcagtataatacatgagttctgtaaggtctgggattcatttatataatagtgttggcctgtgcttgtagtaatacactgtaaaataaaacttcagtagacttaaatatttttaagtcctataaggggagtttaatgtgtgttgggacagccgaggagggcctgaagatgaagggatgggaatgaagtaagtcgcagtctgagaacaggtaggtgtgtaggtggggaaaggtcatggcactcctgcgacagtgtgatcagtgagcaggtagcattgacactctggacgaggcagcaaggtttagggatagtgtggagagcattatttctgtgtgcatggaatccggatacggactgtcagagagggagttaatatcagcactccagtgttccttagggtatagtttctgtggttacttcttttactttgaaataatctgtgtgtttctatctagattgtatcttctgtatacagggatgtatttagagctaaaaatagaatccagtttgagttgggtggtggtggtggtacacacttttaatccctggtctacagagcaagtcccaggacagccaggacaaggctacacagagagaccctgtctagaaaaaatataaaagcccagttaggcaatctctgcctttggattgttaacatcattcataatttatgcagtttcatagaaggaattactggtgccattttacttagtctctttcttatgttggttttgccctcccttctagaatgtactgacttctttgtctttaacaaataccttttccttatttccgacttctttgtctttaacaaataccttttccttatttccgacagtttcataagtgaatgtaataaattctagtgagttttatagccctatccccttcctccacctcaccctgccccttcccacaaaccccctttctttccggtaagccccactcttattttcatgccgtgtgtgctcccctctgctccatgagcatgtgtggaagatggtactacagtttctgtgcttgagtgccagcccttccttagccaccattaactgtcagttgttccttgacgaggagaacactgcccctttcataaagaggaacggatgatcccactcggagcagaaactttttggtacactctactggctttctcatcgctgtgggaagatatggtggccagagcacattgtgcccacagtcaggaagcagaaagagttggacggtggtgctttttacattgcatttgttctgtggtctaggatgctagcccaggtaatggtgcttgctgtcagcgttcagtctgggccttcccacctgctgaacaactctgtctcctcacagtcacgcctggagacgtgtgtcttaggtgactctttagtgtggttgtggggattacacttggcacctcactttatgacaggctttcagactaatattgcattagttgtaataaaactgtagctcaaatacagctctatctcccttcctttatgccattattgtctgatgcagtgtatttgtgttctgtggcttgtattttatcttttgatggaattaaaggaaatcactacgtttataacattgcacattatgattacagtagctgttttatagacactgctcagtgcactagctttatctatcaagaacagggtttgctatgggaaatgtcaacctttttaaaattcgtttattttattttctgtatataagtgttttgtctgaattagtatgtgcatatttggtgctcatggaggtaagagggcactggatcctgtggaactggagtttcagactgttgggagcaaccatacaggtgctgggagccaaacctgggtcctgtgcgagtgcatcaagattccctaactactgtgctatctctttaaaatcctcgtggattatttagaatattgtgttttattttattaatctcttacttgactgtagtttataggctttccccccatagtaattagcctttcaattctaccttaagattaatttcttggttcagctaatcagttatgttattcatgagtgattatgtttaacattgtagttttaaaggtttcttatgttttggtacttatgtatgtgtgatttggtaaaaacacccggaagcgtctcagggtgcctttgctttgcctgaggtgccctcggctctcctgggcatgactgcacaggtgtctggggagagtgggtttcctgacctgtcatcctgctgtagctggaacttgctgagcctgcctcatctctcctgcgcctcccacgcccacttgattcaccctcattagtctgtgttgtcactgatgttctgaaatcggcttcttacattagccagattgtcagctttatggttttaatcttaatctcttggcaacttttgataaaaatgaccacatttcaaagttctttcttggctctgagataacagtctattctttcttcatttgcaactccattatcctttctgtttcatggttcctcttaccccctattattcaaacttattctgttcaatttttaaaactctctttcagtatggtaagtcagatcataacttcacctgtcaaaatctccgttgacttccgtttacatttaaagtgagattgagtttcttaccctggttgatcaggcagttttcctttttacccctattgtctttctctctggacttatcttctacctatctctccctcactcttgatgcagctgcagtctctagcttactggtattttagaagtaccttgatcattaagatgagctgttatccttaaatgtttcatgtgcttcctttgcagaaccgagagctccagatcatgagaaagctagatcactgtaacatagtccgattgcggtatttcttctactcgagtggcgagaaggtaagcgtgagaaagtatgaactggctgttcttgtggaagtacccccccccacccccaatcaactagctttgctaattgattgggaacattcaaagcacaaaccagggattctgaacccaagatatccagttgatgttagcgtgaaaatgtcagtgagaagagtgaattttaagggatctgcccggggctgctggaggtgcagcttcacagctcttggtacaccaactggaggaccttagttcaagtcttcagagcccacagttaaagctcaacatggctgcttacacctatagcccagctctgtggggccatgggacagatgggtccctggggattgctctcagccagtcttgctgcaaattcaggagagagagcacgtctcagggaactaatagagctggacactgaatgtcctcttctggtctctcacatggacatgggcttgtggtcttgcacgtgtgggttccttctgccccacatctccaatacacaatttagaatgaaagcaaattttctgagcttgctgtgctggtgtctgtctagaacccgagtgcttgaaaggctgagacattcgctgtcaagtgagaggttatccaaacacagccctgtcttaaatgttaagcccttggaaaatggagggaaaggggttttctactttaaagtaaacatgtcttcagttctcaagcacatttcatttttattcatatttctgtattccttgtgtgcttatggaaccctctgatagttcatctgtttattaggggaagaaaaattaatgtcattatgttcacttggagctcgattagtagagaaagggaagtagggggttttgtttagccaaggaaaaacactgaaaaatctgtttctggtcgctttgaattctgtgaagtaagggcaggcctgtgatgcgacggcacccacctcctcctcccattgtctgaggagatagatgtcagggcacttctcagtctcctccttgctgcctctcactttcctcctcatctcttcagcccttttacccacgttccccccttccatccccccttactggtttgaagaagtgagacaataacaagggaacaacggaagcacctgatttgttttttcaaattactcctaatgaggtcacgtgatacttggacttgagatttcatgcttgtaatgtctagtgttaggcccacatttccttttgactttacagaaaagttctagtataaacatttttaacaattttcactaaactttatgtgtgtgtccttaactattagtcttacctcagacctataatccaagaagatacttaattcgtgtttgtggctcatgtctgcagatctgtatcgtcataaccaatgttttaaccaccacagttagcaagttgatctgatagtaacacttgtttctgttctttttacgtttaaggtctttctgcttctgttaggaatggtagactctcagtatttggcgctttattgtagagcgactggtcagtaggcctattttcagaccgtctctcaggctagcaagctgcccattagtgtccattctggcgtgcctaccttctcctgtcccttccaaacaaggtcacagagcatctccttggcccacagcattgttagaatgggatttcgtatcctcatgacttctttattattagatgccagacattgtagtaccccggagaggaaagtaaaggtcagaattatgtctttcctgatctactagataacagacagattcatttgcaaatgtctcctgaaaaattccctaaacagtgtgttgtatacgttaataaatatagcccctacataccagggcttattttaatgctttgtaaatgttaacacatttaacttggaatgaaagggcagagagggaggcttttcaggttttggttttgtaggaaggctctgtcgttctgactgaaaactgactggctattctgtgagtgccacagatggggatttttccttgctcctgcttttagttgtctgtttatggctgctcctgcagatgctggaagacctctaaaggaaggttatcagaagagcaataaatcagtctctactaaaagtaaaatatcttaacttttaagcaaagttaaaagatacttactatttggagtactttccctccccaaatctttttctgtttcatttctcctagactggtttctgtcactctgcaactttattcctcacttcccacccttgaatctatgtgaattcaatcataggtctctgcacctttcttttgttaagttttgccctatcttgcctaccccctcccccacccccccatccccacccttttccagttctgagtctgcaaattccctgtctgtcttgcatatttcttactttctcagtacctttatcactcttggcttagattttttttttggggggggggaacctttgtattttaatgtgggtgggtgttttgcatgtgtgagcctgtgcactatggtgtgctgtacttaccaaggccagaagagggtgtcaggtcatgtcaggttatgtcaggtctggagctgaaattacagacggttgtgagccaccacgtaagtgctgggaaccagactgggttactccggaagagcagccagtgctcttaaccactgtgtcctctttccagtgcccactctacccctaaagacttttcctttaggcagtcagctctctgcttataactagggaaatgctaccagctattgtaactagaaggggttattcagagcccaggctgagcattcctgctgggattattgcttgctgtggtgacagaagcttgttgcaatcagcatattgtaggttgctctgtaagttttaggtgctctaatttagaatgaatagtgcctttccccttgattagataacaacgagaatttatagagtgttttgaaactttagatttaataattttgtgagtttcaattctacagaatagtgatttattttcccccaccactttagtatttagctgtacgaaacatcctcatgaaatttcatgtagaattgtgatagaataaatgaaccacaaaatagctcttctgtggaaacagatgtttgcctgtccttaacaataactcactccacagaactttatgagtcaggatatattataaagccacattctgcctgtaggaatcccaagctctatttaaattgttttctcaagttatcttgcatgtgaaaatacttcacatcataggttgttttgagtgaagggaaaaactttcccttctgcttacataacagacttttaaagtataatgatgtcactttttgtaacttcatgtggtgttgagattgggcagagttatgctaagctaagcttaactatgactgtgtaccctggctttggggagggaacgggtaaagatgatgcagcaataacccattcagaaaatgaatgacacaacccgtctggaaatgaagtggatggcctgctttgtagctgggaggtgggttagcgctgcagttcaggcatgcagtagaaccatgactcgggcaagttattcatttttaagtaacctccgatctcttaacatttggagttttgtaaggaacttatttagtgtttacccattactgatagcattgggctgagaaccccacacgttaatttaccatttaataaactactctatttagtaatagtatgaattcagagtacttgctgtcattagatggtatcatgaccctttcacttgttcataactcatattgtatggtacccctgcgaagaatactatttttagcttcatgttaccgatgcggactacactgagctaatagtatataagtatttacctcagatttatacagctactaaatggggtttgggagtctgaatccagagtataaaagatgccatattattcaccatagtatgtgcaatagaaacgtaacatttaaagtagcgtactgatgatggttgagcaacagagttgtagtggcaatgatggttgtaagcgtagtaaagataacaatagctaaaatgcgttagtacttaccggacaccaccatggttggttttattagacaggttacaagtagcatctgaaaacttgttgatttctcttcttttattatatatgacgtctctgtccctaacctaaacagtcacttaaagcagatccatagttaccttatttttgctattatcaactgcaaagggttgtatgggtcattgtgttgggaactgagtatctgctcttagcgagatgttgataattcatggccactagaagtggaagttgagctgttctgtgtaaatcagcagtgggctgctttctgatggcatcgctactcacttaaaaggcaaattccaggggttggggatttagcttagtggccctgggttcggtccccagctccaaaaaaaagaaaaaggaaaaaaaaaaaaggcaaattccagatttcttagcataacttagaaggtctcctcatttttttatatttgttactttttatgagttttttctacatgtatgtatgagaaccatgtacatgccaggtgcctacagaggtcagaagggatgttggagtccccgggaactggagcagcaagcgttcttaaccatcgagccagctctccagcccagcacctttgagtatctaggttccagtctggcccaagcacagttctctctgtgcgcatcttctctgtatcctgaggctttcggaatgtctgctctgctctgctgttctgtagtgctctctggactctacaccactgctctgctcatacagattcctactcactgtttacctcagttcagcagataactcaccactccctttcctctgcctttgtaactcctcagagtttatctaaaatatcttgcatatacttttaaatattttcttaagttttaccacaattttaaaaagctatgtttttttttttttactatattgtccttctcaaaagcattcttagatcttactcagttttgtttcttcaatacttagtataattgccaagtcagtttagttagttttgttgatgatgtattgaaagccttctagatattcttagaaaacagagtgctatgctggggccatgactcagtgggtaagggttcttgctgtgcttctgaaagcttgagaatcttgagttcagatccacagtgtccccataaaaaccaggctttgcatgcccttaatcccagcatcgtgggctagagacagtctctggcctacaaatgcttacacacatatacatgcatatgcacatgcatatgcacacaattaaaaagtccaagtgccaaaatagagctcagtatgataacatttactatttgaattaggatttattttatactattttttaaaatgtatttaattttatgtgtatgagcaccagtgaaagaccctgtgaatggcccctaaggaataatacccaaggctgtcctggtttccatacactgcacatgtatgtacatacacccccccgccccaaactcaacttctccttttaattcggtagttttctattctctgtcgtctctgcttaaatgtttgaaattattttcttctcagctttatatttagttttaccccccaaagtctcagattgttgatttgagatctttatgcaagggtgctggtgtgtgtgtgtgtgtgtgtgtgtgtgtgtgtgtgtgtgtgtgtctgctctccttttgcatatacctgtgtctcctgctgaactccttattagatctgatctcttctcctctctctctctctctccccacttcccttcctccctctccctccctccctcttcccctcctactcccttttccttctttccaattttagttttttcttttttgagatgttttattctatagctcaggctaacctggaactgacagcagtctcctgcctcagcgtcccaaatgtttggttatacggataagtaactattcccatgaggtcttcttttatgtgtgtgtggtcagcatgtgtgtgctttcctgtgtgtgcaggtgtggggctgcaattgtttttacaccttttttgaggcagggttgctcactgaacccaggacttgcctgttgggctagactgactgagcagcaggtccccgggatcctcatgaccccttttccaacagtgttgctaagttcatctagtcttgccttgcttttatgtgagtgctagggacctgaactcagttcctcaagcttgcttggtgagcactctatccactgagccggcagtctagccagaaatctttttttaagatttattcatttattacatatgagtacactgtagctgtcttcagacacaccagaagaaggcattagatctcattacagatggttgtgagccaccatgtggttgctgggaattgaactcaggacctctggaagagcagtcagtgctcttaaccgctgagccatctctccagcccccagaaatcttttttttaaaaatacttttaattgtgtgtgtctgcatactgcatttggtgcatcccataaagttttagtatgtggtatttttgtttacagttaatctcaagtattttctcatttttataatttctgatgcttaatttcctgtctcattcactccttgtctggattggattcatttccacacatttgtaggttctccagttttcctcatatttttgctttgcgtgcgtgcgtgcgtgcgtgcgtgcgtgcgtgcgtgcgtgtgagcgtgtgtgtgtgtgtgtgtgtgtgtgtgtgtgtgtgtgtgtgtgtgtgtttatgcgcttgtgcacgcatgcaccgaagcatgagcatagaggtcagaggataacttgtgggagccagttctttctttccaccgtgtgtttcaggtcatcagacttagtagcaagtgccttccttagcagccaagccatctccacagcgtcctcccgtctgtggtgctggtgtccagtgtggctggagaaggtgttccacatgctttcactctttttaaacctaccgaaattgtcatgtgacttcctgtggtttggtcatggataatctcctgcttgagaagagtgtgcagttgctattagggtgaagggttggacatggcgtgcaagttgtttgctggttttgaagacagttccgtatggttcccagcagttcctgaggaactcatcttccttactttacatcagtgttggccttcatatgtttgtggctctgacgcttgctgggatgtgtgtagaacggtaacactctggtgaatcgatgtgcttggcagtgtggagctctgtagccagtttccactgttgtggatttactgcgttgtatgtagcccccagtgcactgtggctctggcaccacatcctaggtgtcttacccaggctagcagaatctcacaactggtgttttcatttgctgtatgtccttagaagagtttccaacaccagattacttcacaggttgagtgttcgttgtttgagatcctaagttagcctcctttctttccttgtctctacctcctcatgactttctacactttgtttttgaactgctttgagaaattgctgttaggtctaaaagctgagtgaaattgaagcttccagttgtcatggctgcaggctgtagtacaggggcacagactaaacactaatgggtatatcatattaaaaaaagcaagcacagcaaaggaataacacttaatcacacattatagaacataccaaaatatttcaaattagaaccaaggaaaataacatgaacacttactcttttaaagtataatgatctttaaagttttctgagaattttattttttcttttaaaaatagttaattataactgccacaaggaatgtgacaatgttataagctttgttattgagaaaaatcactctagtgagattaaaatttactgtttttcatagtttgcaaccatatgtgttggttaattttaggtcagcttgacacaactagaatcatttgggaagaggaactaactctccattgagaaaatgcctcagtaagattggcctataggcaaggctgtggggcattttcttaattaataatgattgatgtgagagggcccggcccactgtgggtggtttcagtcctgggcaagtggttctttgttcggtaagagagaaggcagaacaggctgtaaaagccagcaagcaacattcttcatggtctctgcatccggttcctaccttgagttcctggcctggctgccttcggtgataggagttgctggtcttcatggcagcgatggaaatcccaagacttcatccatgggagtctcagatacagaatcatctctatggtccttgttctttccaattcttgctttttaagtttttggttttttattgtttaaatatatcgaggtataagtacaaaagtacatgctaatacacacctacaatctcttaacactcaagcggctgaggcagaaagatcaggagttcagggacatcctgggctgtgagagactaagttttaacacagaaagggggagggaaagaggggcaaagaggagagatagaaaaaccacaaaaatgttggaaacttggattcctgtctggggtgtgtcagaaaacaaactgcacaagacgataagtagtgcttctccatggcttctgcttcagcctcggcctccaggttcctcccttgagttctgtcctgacttccctcagtgatgggatcaacctgagagttgtaagctgaccttttccctccccaagttgcctttgatggtagactttatatcctagccatagagagcagtgaggacataggcatgtagattatgtttagttcctcactctccatgaatatgtcttcttgtccatacatacgggcaccatacacataggcaggcatacatagttttaatggacagttccagcaaatagcattgctaagtcatgatgggctctgaggagcttcactttcatgagggtattaactcacagacttctcaaggttccaggagcttagagctaagacttaatttcctcacattggattttcctgtctcattgagaagagaaaatgtagcttgtttatcttcatttcattgctgtaattatgagtgaagttgcacttcttccagctttcttcagcttcagtattcgtgttttcctattgttcatacatcaatccttaaaaacaaaacaaatcattcagtccttcgttagtgcagtagaaatccctttgcccaaaagcattttttgggcatggtctgtgtttgattttgttttatttcgcctttgaatagcatgagttttatatttttatgtcatcaaactgatttgtttttcttaatatcttggggttttttgttttgatttttggatttttttttttgtttctgtttgttgtttcggttgttttttgttttgttttgtttttgagacagaatttctctgtatagtcctgccatcctgcctgtcctggagcaagctaggctggccttgaactccctcacagagactagtgcctttggctcctgggtgctagaattaaaggtgtcctttcctttccccttcttaatatcttgcagctgctccatcctccaaggtttactttgcgagtccatttctttccgcacaaaagtgttggtgatttagaagttagttttagatgtttgttccattttctgttaggtgttggtgtaccctctgcgcacaactcttttctgttgtagttgtagtgtgccaggtgccctggcaaaaaacgttgcagggagaaaccgtttatttggtggtagagttaaagtgggagcaatccatgatggcctgtaaggtgttggcagcagcacagcctggcacccaggatgcaaaagttttacgtttcatatcccacacggaacagaaagagaacagagactggggtcaggctgtcaaacctcaaaccctgccctggtggtgtacctgctccagggctccacctcctaaaggttccataactaccctgagagtgccgacagctgggcaccaagtgttcagacatgcaagcctatagggacgttggttatttaaaccacagcgccttccttccttccctatcttctccacagcttctcaaggaaaagatttcccattaatcctcagcccattgtatgactgctgcaatgtctgtcctttggtagcacagtgcacccatctgaggaaggacaactgattacgacattttgtccataaattctgagaatgcagggttggctatcttattactaaactctctgaatcactgcttctcctccatgaacattttagagtgatatttgtgtctctaatgctttgacctcaagccattttatcccttggtcttacaagtcatgtgaggtgggggatgatggaccatcttacattttatagtgataaggtggagtctccctgcccctgcagctactgttaggcggtgttcaaccatgctattaaacgccgagttggatagcttagaccagcctcatggaggacccattgagcctctgtggagcagtgctccatagttacacactgtgtgagtttgggcagttcctcagtaaggtccactctgtctgagagaggaggctctcacagacgctgtaggagcaaacctaagacagatctgggggctcagcagtggctctgagatgctggcgaacttcctttaaggccgtgttctttcttgtgtatttctgtcagtcactttaatttacatccagttgctgaattgctgaattttctggcaactcacaggaaattgttttgagattacagcaaaccagagttttagaaaattagaaatgccagaagtggagcatcctgggtcttctttctttttaaaatactgtcataaatatgagtcccctgaataatctttataggaacctccaaataagaaaattaaaaactactgtgagaacatgaaatagcacttttttctacatgttttctgtagttgaatgttctcaggagttcaggaattaatatcatggttatttacattacaacatttaaacttacaaaagcccttacttaatcttcctttattctcatactgtaataagttggactactggccttgtagcagacatgattttatcacctctctggtgcattctgcgctgttaaaactgctgcctactggacttttgtcagtaacgaggcctgaactgtgcagagcagagttggccgtgatggtccgaacacagcacgtatgctacgtctcacgtgtccccttaggagtgcctcagtgacccatccttcttgagttctgcgtagttacacctggtgcatactttccagatgactgttgcatttgctcttgattttgtctggttgatctttgtatattgatgttctgaattttcattataaggctttctgctttgttagatatcacattaaggcttcgctcagtctggcctattaggcttgacagtccgtatttgctcttccatttaagccattgttagtggtgtcagctttagatggctggttaaaacgggagattgcagtctgactgtcagacttgtctcctggtgcttttacttggttggtacgctctgtgaattgtttatggatgtgtctagttaaatcgtgtactgatgtaccacccgctgtccctgtttgtatactttacatttgttacttaactgttaaagtcacttgtttcttcaaacctttaatctataagcttatagagtgtcagccacctcccgaaacactggcgtgacaaaaatatacatggaaaacatttaaattatttcaactcacaatttaagacgtcaacttctgttttcaggttgtcagttgtgatgagtgagacctttagtaacctgaaaataccatcatgcagacatacggtgggaggagtgcaaatggacatccgagggctaggatttaatcccatccttgtttggttttggttttctactctgcttcccttttgcatcatgctaacttgcctgtccactgttgtgtgttagagcatgtgacttactgtcacaggctcagagctcacggtcaggtctgaggctcaggagactggtttcgggctgctgagcagtgctacaagtgctgctgtagtggagactgttggatggagtgcactgtggtgtgtgcactggcgtctagagcagggacaaggttctgtccttcaaggcatgcccagtgacctgtgtcctccatcatggtctacttcttaaagcgtccaccacctcccagtaccattcagtgtatgagtcagtaggggacatttgcgatttcaagagtgtaatataaagtaagcggtcaaagccccagaccaaatgctaacacttactgacagacagacccggagccttagggccggaagctcctgaacatgcaacagtcatccttaattgttgcatcggtccttctcggtgtgctccgttagcaaatgctgaaaccgtaatctagcattgcccacacttctgggtttctcattagtgtgtcaatcatgataaactctgtcaaatacgttttagaatcaggagcccttgagtctagcaatcttttaaataaaagccttctaactcctgaggttcaccatacttggatcctataacacaagttgaatttctcttatttgagcttgtgaacccaggagtggttttttggaaagactgtagaatgttacttagtgagatgttttgagaacggcctctgaatctcaacaggaaatgcatttgtgtttcacgtatacctgaaaaatgtagcctgacaatattaagttgacacaaaatttcagcacacctatgtctcctgtgctcttttttgctgatgttgtttatttttagcttagttttctgtactttttaatttaaattttattttggtatttgggtgttttcctacatgtatgcctgatggtggtgaaggccagaggagggcagtgagctcacaggagggcacctgtgagcctccatgtgggaactggagtaagtgaccttaaccaccaagcgcccctgcccgtcctctctcccctttgtgttccccttaccagtcctggattcgaatagtctttctgtttcaccctcctaagcagctggagaacacataccatgcatcccacttatctactgcctgtttggactgtgaccagcctttcctatggtatgatggagaacttacccatttgtgatgccgtgttcattcccagaatgtttcagacctcagagcattttgaagttttactgtattgttcacctctctgtgttcttgtttttgtttcacagtattgtaaagcagtggttctcaaacttcctagtgctacacctctttaacacagttcctcatgttgtagtgaccccaaccataaaaatattgttgttgccacttcataagccgtaatgtaagcattatgatttagggcgcattttcattgttatgttgacccgaagagatcacaccccccagactgagagctactgctctagagtcgtgtttcaccagtctttccttagacttttgtcctagcttcttaacatacatatgcatttgccagtaaaaatccagaacaaggagcacagtagttttgtattggctatgccttgctttgatcactgcctgccaactctcctcttctaccaacacaagttatgtaagaacaaacttgggggctatctggttgaaattaatcaactggtttttatttctttcttgctgtttactcacttgtgaacctagaaacccccatcccccatccaccctttctctttttatctccttttcttcttccaaggcagaggttcactgtttatttctgactggctttgcctcacagagatccctgtgctgaaattaaaggcatgtaccaccacccacagcctgaacccaaatttcacattcattcattcattcattcagtgtgccttggtgttttgcctacatgcctatgtgagggtgtcaagatccctctggaactagagttccagacagttatggcctgcacaccgggattaaaggcatgaaccatcactgctccaacccaaaattcttacagggactttagatagctaaaccaagatagaatctatctctccaacttaatttatcgatgatgatttaaatatgagagcagtcaacacaatggaggtttccttgtagccatgctgcttttgagctcaggctgtgcagaaaactaaatatcaccagggttatgaaaaacacccattttttctttgatactttgagttttgcaacctacccagtcactgccacaagtacttgattcttgtcattttagccccaaacagcaacaggcagtgtttaattggtgggtgtcgctgagctgcagttagactttgtttataaagataatcgcaggctgttgtcatcatttgcttgcctctgtgctgatgagagtgatagaccagttagctatggaagaaggaagaccatcaaaaaagatatattcatatcttgaataaagttttctctctgttgtttcagtcttcatttgaaagataagaagctatacttgttcctagtcacagagtgggagagcagagttacacgtgggtaacttgacacctgcacccacccttccctcctgtagcagctctgacctgtagatgcgcccaaaacactgcctcagacttgagtttgataggtggaattatgaacagtcacctgcagtttcactactcagtaaccaagcgtaatccttaagttcacttttcgtcctcagtttacccaactggtgctgttcagagaacagggtgtcactagtgctttctcctctttcctgtttggctctagctcccttctttctcttgactgacattgaactcttgcctttatgctgctacaccggagaccagtggacaagcttgtgggcatgatctcatccatatgtttgagatatatatatatatatatatatgtgtgtgtgtgtgtgtgtgtgtgtgtgtgtgtgtgtgtgtgtatgtatgtatgtatgtatgtatgtatgtatgtatgtattagtgatctagaccgtcagacaaacaaagcatttgtgtggaggctacagtgtttccaagaatacttagttcattcttcctcaggagctgaagaaactgaagccaagacatatatatcagtcagacatacacatcagttgacagcagagccagggcgagcagcctgctttcttggcttttgactcagtgctgtcttcaagcaatctttccacagaggaggaaagatattgattttttcagcctagaaaataataaacattatgataaacgaaaagctatcataacacatgtgtaagcaatgtgatggattataactatacccggattataaagtctgctagatgcatttgtacagttctgctcatgcccagaagtaagtttgtgtaattaaaattgaccatatgaagcttttctttaaacatctgaataagaggtatatagagatttactctaacttgaatacttaagatgagcctctaatctggttttgtccttttcttcttttaaaaccgtacagaaagatgaggtctaccttaacctggtgctggactatgttccggaaacagtgtacagagtcgccagacactatagtcgagccaagcagacactccctgtgatctatgtcaaggtatgtgacaatgggaccccctccagttcacagaagggttgggatgtgtcaaccccactgacactggtcactccttgttaaaacagcaagctctggatggtgctaaattgttacagttttaaagcatggagttgttttaaagctttcattactcagcagtgggtaaaggtgcttgctgtgggagactgataactggagttttattcctggacctacgtgaggtcgaagagaaccagcctcatgaagttgtcctctaatcctccacgtgcatatcccatgtgtgtgcacacacatgcccatgagataaaggaattgaataaatattctgataaaatatggtaaagctttcaaagttaaggattttaactgtgatagccatacacctttggtgtggtgtggggaggggagcacaggtgaattcaagccagagtcagactcctgctttcacctttaccttgggctgacctcttcttgggccctgtataagcttttagttgggttttgtcttggtctctcttcccaccagtggtgggatggcagccaagtgcgcctcgtcccaacttcatgcatggtgctggggagtcagggtgaggtttccgtgtttgtacagcaaactcttttacagcgaggcggctcccagccatagcatcactttcgctctcagtgttttcgtgtgccacacaccacaaatacgtaagtaaactcagtctccacagttcccgtgtagcattctattactgtttattgcattcctcatagacatttaaactgcccgttttaagcaaacagattatattatttaggtgttggaatctcatattaatccctccccccccttttttttttcatgtaactgtagttaggaatttatctagtatttttctaattgcagttgtggtagatgatgggaaactttttttttctttttaggtgagagatagggaaggggtggatagtgtattgccctgtagttcagaccaccctgaaacttttgatacttgtagcaatcctcctgcttcagcttcctgagtgctgagattataagtgtgagttaccaaatctagttaggaacacatttttaatatgtgtgctgtcaactaggtgctgattttttttttctatttctcagtgacagcaatataattacctactttaccccacccctgacatagagctgatgtggtgactgtcataatgctgccactttccactgaaagtcatttaaagcaggggttaggagggagcatagctgaactctccccgggatcccgagtaaatcatcgtatggccaaactctaatggagaggaagattactcatgtgtaaaactgtgtggggtgtacttcctgaatgggtccagatttttcccagcacaggatttagtccctctttttgtccttcacgggttttctagcaggttctagcatcaggttgtggagcaggcaacaatatagagatgccattctttttttaattgaaaaaaattagttaaaggacaatgtataagtgcatagggagttggatttaatttaattccaaaatgctgacattaaagatgttatgcctacatttttgtcatggttcattttgaagaaggctttaacttgggtagtgagatttaaatcaaaattgaacctgtctagccttagcagttttctcatacctctgaaggaagtatttcctgttaacattagtaatgccagtccctagcagtacagtcggaaaagatggtgtctttatcagtacttctcctgtttttttaatcagacagtagtagttttgatttccatggtattaagtagggagtagagctcagagagtgagcgtgtgcaacagattggaggtgatgatgctggagaaggcagcacatgtctataatctcagcactttatagactagaggatgacaggaccagggccagactgtattccatgggagtctgtcttaggagagaggaaaaatgaaggaagaaggaaggaaggaaggaaaagaagcttggcctccagataagtagaagattaaaggcgttacattggtacatttttcaaagctttaaaacaatctcttaagatattatttctctgcttgctctgatgaaattccagacaaaggcgactcaaggacagaaaggtttattttggcttaatagttctaggggatactgtgcatcgtggtagggaaggcatggtgggcgagcatggggcagctagccacattcagagaggatacacggtgcgtgagctccttttcttctcttactcagttcaggtagctagcccatcgaatagtgcagatgcccttacagacatgtccaaaattgttttcatcgattccaagtactctcaagctggcagttaagatttacctctgcaatgatgtcaagtcagctagacatctgtttgtgtgtggtttttttttttttttttttttttttttttttttttttttttttttttttnttcngngtaaccctggctctcctggaactcactctatagactaagtaggccttaaaactcagatctgtctgggcatctgcctttaaattacattggaagtctacattattctaaggatgttaaaggctaatttaccatccgagttactggtgtctcagtaggaattgataagattacttactcatgagctttcctatgtctctcttaatgatttgaggctaacttttcagaaaaacatttcagaaaaatggttctaaaagatgacactgactacatttttacagcctgagattctaggaagaatgatggatctgcctcctaatctaaaggctgctgctgtctatcacccatatggcttctgatctctgtctctgttaagttaaaacccagctggcctgagtgcagggctgtgggcatgctaggcaagtgctctagatccctgactctctgaatcttagtgtgagacctggtccgacttgcccaggtagttcttaaacttagaattccctgcctcaggtgtgtgcccccacacctggatgcttttctaaattactgaaggatagcacaggtatatatttaatgatagtttaatttcctgatttatgtagaaatgccatgaaactttgattagggaaaactgtattgataatcaacattttgcatgttctgctgagaacgttatatactgcataatgactcagatgtgtttgaaagcaggggtatcgagtgaatgtctcttctttttccagttgtatatgtaccagctgttcagaagtctagcctatatccattcctttgggatctgccatcgagacattaaaccacagaacctcttgctggatcctgatacagctgtattaaaactctgcgactttggaaggtgagctagcctcctcttcccttcctttcttcctcctccctccctcacactttcttaaatagactcctgtaacagtactagtctgaaccgcactcattactcggtactcggggaatacaaactaatgaaggtccatgtcagaatggttttgataattttatgggaacaataaatgtgtgtgccctttatcctacagttaacttctgaggattatttctttatgcatatttagggatctttaatacagtgttgcttgcaaatatctagtagcgagaaaatgtccaaataaattaatatatacagatacagtgttacacatgctgctgagggaaaagtgagggagctgtctaatgtgggagtgatgtctaggaagtaccaagtgaggaagtcagtcggcaaaggcacttgctttcaaggctggtaacttgagtttgatcccagggacttaagttgtcccgcctgccacaggagtgctggaatgctaacattcaggctcaaaaaggccgtggcggcacttgctgataattccagtcactcagtagatgtgggaggtcattagtttgaggctggcctaagccaggtagtgagaccctgtgcgaaaggaaagcctctgtcaagtcagtactgtatgcatggtagcttttgttttaaaaacccttttgtgactagtgtgcaaacacacataacaaacatggcttattctgagttgatatgtaatacctcatacacgattgtctgggacgggatgtttgaggattatcgtgtacttgggaaacttacattcccaatgactgctctttagtataaactccttgagcttatggaatacatatatctgctccatctttattactaataaatctcttaagttatacatttgcatatattcctttaacttcacactcaacctcaatacaggagtgcagtaaaatacagagaggcaatatttttctctattaatatttactattaatatttcctctattatttccccatttttgcagattctaacaagtatgatttttttttaaaccgccgatttcttggttatatactgttgctagtgtaaactcctcatgctttctctttgtgtagcatttagctataaagcctcccaacatgcagccaagcctataccccacaggacagatcaccatatcccttcttcattctctatctgtcgcctccgggggtagccctactctctgtgctgagacttctgtccttataacttgggtgctttttcatccttgttagaagttcttctttttcagtttagcaccacagatgatacattgactcctgctgtctatagtccctcctctttatatagacgtctataccaaaccgtagattagattgtgggtttaacatcagtgcaggtgaagggaggtatttggaaaaaccgagccaacctgctgtcagtaggttcatgcttagtcttgttctgcagtttctttcaagccattccacttaacaaaccctatcccagggtctcagcactgcaggctgcggctcaggtgaaaatacacactaaggcgtttttttatttgatggttgaatggtgggctgctgcttgtttcacctatgaaaaggcacccaaatcacatggggtttgttactggggagagtaactgccgagtcctgcaagagccacctagtgcaagttcctctgctcaccaccatgtcagagcattgttcgcgcccgcgcccagcggattctacgggtgtaaagtagctgtgcctggatacatttgaaagagggctttcacaggtgtgaaatagctcattctttagcaactctgaggtaggattttggagacacttaaggtgaaaacttaacaaaactgccttcttaataatttttaattaggtgtaacaggtctggcagtagctgaaatggggtgtgaaaaaaacccattttatagagtctaactggggtgtctaagggccattaggtaaagggaaaagttagtttcgttggtgatgagggaaaaggatacaggctgtcaaagctctcagttggatgactgagttgttgtgcgtttctcttctaagcatctgctttacccattaaaaatgtgcctcgtggggctggggatttggctcagtggtagagcgcttacctaggaagcgcaaggccctgggttcggtccccagctccgaaaaaaagaaccaaaaaaaaaaaaaatgtgcctcgtgtgtttggtttctgagacagggtctctgtggccctggctgtcgcagcagcagctgcctctgcctctgtagagcgctgagactgaaggtgtgggccactgtgtcctgcttgctggtcattcgttcgggagctggatttaaactccgcatcctagagctgctttttcaaggggccattccaagtccccaaggcgcacttctgaagatagaaacgtctgttatcgagggcactaacactgaatatcttgacttttttgacagtctgaaaaccttagtgttgttaacatagcgaacactgttttatctgggcctaagaaaaagattaaaatttgaaacagataatgaaggaaagaacaaatcaagtttagagccatcattgagtagaagtaaactgaaagggaggaaacctagacccccaagactgaatgaccattttcacatgtctgtgtcctgcactgttacctcagattttccacctgtttttggcatttatttatttttttctatttgttcattcatttgattttatcgcatgtatttcttttctgtatctcatgctactgtcagaacagctcctagagcagcctgcttatatgagatggagttacccagagagacatttcaagagacactgtgactcactttaacatttaaaatatgtactgtaaactactttaggaggaaagttgtaataatatgttcaacgtagcactgcagcgatggagcagatcgggcctgagtcacttaaatcaaatgccttttacagtttgtttagcagtaggtgtttaaatcgactgcggtgtggaagcatggattggctgtgtaaggccgagctggtcatcccgggtctgaagaccaagtttaacagagtagccaggtcccccccacactcccccttttaaaagcatataggtggggaatactgatacattcacttctatgatttgctaacagtcttttgttgcagaaggttgctctttaatttcctgcccttcatctttatattttatttagttacttattattttgaggcagtgtctcactgtataccattggctgacctggaactctcactacagagtggcctccatctcgcagaggtccgcctgtctctgcctcccgagtgctgggagggaaggtcgcaccaccttgcccgtcagccattttatatttcacaagttgaatgtaggaaatggggaaaccagaagaaattgaacatgtgtgtccttttgtaattttttaaatctctttgttccttagctaaggcttgacaaatgctcactttaaaaattcctctttaaagtatcaatttgctgaacttgtgtatttctcttgttggaaacatttcaagtgtgttgcttacaaggtttagacgattaaagattgttttttatttttatttttcatcagtgcaaagcagctggtccgaggagagcccaatgtttcatatatctgttctcggtactacagggcaccagagctgatctttggagccaccgattacacgtctagtataggtaagtgctggctctgggtgcggtgggtgtgactgttaactgtcctgacccttctccaacaaggcagggcacgacagagcagggctgcagcagtacacttttgcagaaatgagtagaattaattttttttacattttagaatttatgcttttctactattagaattataaaaatattattttgtattgtagttgcttaatgcatttgtttggctcttaagtttcaaacaaatgttgataagcttttaaaaacattactgaggcagagatggatggcaaaaagacgtgtgtggagctcagtcccagccgcgctggagaaactcagctgctgagttaactaatggggtctgcactggttttctagttctgaaatgtttacttctgagcctaaactacttagaaatgtgttaataaataatgttacttttaaaatgttaatattgtaatcggtagggcaatgatattcctgaatccctacaatgatgcatcgcttaatttttgagcaacttaattcattagcttttcagctaagcccactggttgttcagatctcgctggagatgtctattgatagaagggttagagatggttgaggtctgcattgaccctatcttcttaacctgttctagtgccattgctatgttagcagacaaaaacctgtttatttcatgtatttatcagggcagtggcgggggggggggggaggagggggagggtgacgtcaggtgtcttccttcattgctctgcactatatttttgagtcagggtgtcccactgatgtggctagaatagctggcccatgagtctgaggagactccatcctgtcttcaccataagccacctccactgccccttatgctttgtttctgataggaaccccttagtcctaagaacgaaagcttacatactacactcctcccccagggaaaatcccatagtcatgggggtgaggcagaatacaaaataaaacacaaatataaaacccagctaatttagtacagttttttctctgtctttctgatatgaacaccagcacgtgttttttattttcagaaagggcaatatgccaacgcttcttttcactcatgccaagaggctggtgctttttacatactaggtcatttgtgctgttggtgtaatgttgtatggaaatagctttacactctaggatctgaatatgtccaaaacacttagggctctatggtccacacttgatgaaacctgccacaggagaggaggggtgttgagtgtgtggtaccccgtgtggtggtagtgctgtcttactgtggctgtagggacatggcgtatgtaactgagaagtgagacgtgctacagagagattgttttctccagtcatcttccagactcataactgagtatctgagctgccaagtggagatcctgattctgcttcctcttcatggttgctgtgaggagtcaataaaatgttactcaagtactgtaggtgacattctttagtctccttattaattcagctgtctccaagagcaaatcttagtccgttgtcctcagtaatgccattagatacagttactgattggtgttctgttcaaatgtttgataactagttatctattgtgtgtgtgtatatgtatatgtatgnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnnncacacacacacacacacacacacacacacacacaaagataatagattgagttgtgccagggtctgcaaaaatatgggcagtccttctaaaataaccacaaccaaatcacctgacacattttagaaaacatgggtttggatctaggatgcttgccttttacatttgttttatgtgtatgagtgtttggcctgcatgcatgtatgcataccacatgcatacttagtgcctgtggaggtcagaagagggtatcaagtctcttagaactggagatctagatggttgtgagccattattatatgggtgctgggaattgaactaggttctctgcaagagcagccagtagtctttctcagtggataaaccatatgtatagccatggcttgcttcttaagactgtaggatttatagaagacgtgggtgatggcgtacacgcctctagtctcagtgcataggaagcagaggcaggtggctctcttgtgaatccagccagcccagggctacatgtactgagccagtctctgtctctgtgtctctgtctttctgtcttcacgctttctcaggcatgcagaagagatttgtgaaagttgaatacttataaaaatacttattaaaaaataaaaagggttcaagtatatgaatactttttggaaaaaaattaacaatgttgtaaatgaaaccaccactgattttgactaaattattgtcatatttgaatataatcaatactatgcaatctaagttaaatatacatttcacttcaaatttaatgtgataagcctttcttttattttgttttttttttcttcagatgtatggtctgcaggctgtgtgttggctgaattgttgctaggacaaccaatatttcctggggacagtggtgtggatcagttggtggaaataataaaggtgagatggttttacacttttgaaccaaaaaatgttacacacacacacacacacacacacacacacacacacacacacagaaaaaccacacaataacacttttaaacaatgggaaagcaggctggggacatgatgcggaagttcaagcactcactgtacaaacatgagaagtagagcttggctccccgaaacctgcatgggtgtgcctgggcctagcatttcccatccagagagccccagagcgagctgcctaggaagagtagccctatctacaagctctcggctagatggagacgctgcttaaatgaatggagtagaatagtgctcaggggtaactgctgatacctcccaaccttgggccttcacgtacaggcatacagagggtaaagaggagaaaggattataggtcatttagccagctgtgctatagagtagagcaggttgggggaagccactggtttcctttttacagatctatttttatcgtatggcgttttttcgcctgtttgtacatttgtgtatcacgtgtgttcacagtgctcttagaggctagcagagggtttttgatcatttggattcttgaactgggagggttacagaccattgtaagctaccacgtggattctgagaatcaaacctggattctctggaagaagagcctgtgctattaattgctaagacacctttccagctccatgatcacaatgttttaagatctgtgtcataattcaaaattgctttttcttttactataaggaaaaatagggactcaatgttactgggtccaaatgtagttgaagaggctggtgattggtcatcttccttaactctgtggagttgggtttcatagctgagaccactgtggtaacttctttttgttgtgtgatgtcgttttgtgttattttttgagacaaggtttttccttgtagccctggtaatcttgtaaacaaggctggccttgcactttgacatccttggtagccatttcttatgtcagactgaatgaacccacaaccttgttaagagtactgcttcctccttactcactagaaacacagagtagaaaccatggaatcgctgaagcagtgttaaagaagggctttttcaggtgtacagagtaccgttcagcatttatgaaacagtctgcactttttaaatatgtgtatatgatacatacaggaaaaacatgaaagaaatcctagctctggtattacactagtgttagtgaactggtgttcagttcttaaaagaaacacaatctaggcgttagatagagacacctcctgtgtgtctcaaagggagtagtactgtttagtattctctcatacattacatcccaattgcagcttccccttcctcctcttgccagtccttccctctcccctcccatcaattcctcagaagttcagaacaaagcaggcctcctgtggatgtcaaccaaactctgtgtatcagcttgtaataaaactaagcacatcccctcacattaagctggatgaggcaaccgggtaggaggaagaaggtcccaagaccagtcagaagagtcagggacagtccctgctcccactactaagagtcccacaggaagaccaaggtacacagccatgtttatgcagaggctctagctcagacatggccatgcaggctccctctccgtgagccctgtgagtccacgttagttgattccatggggtttgttttgtcatgtctttttgggtgtccttaacccctctggttcctactattaaagttgcagaaacagaagtattgttcatttaggggtttatttaacctaagattttcagacagcccctttcttgaatattcttaggctgagattcacctagattttttgtttgtttgtttttgtttttctacaaatatattcactttaatgctcagtgttttttatataatcacagaatagttaccttttttacaggaggatagaacttgtttctaagtatttatgactcaaggctgatagctaaaacagttctgataaagaataggttagaaggcttgcattcctagtgtcagaattaatcgtacagttatagtaattaagagaatgtagaacttgcatgaatgtagaaatggagtataattgagggctaaggaataaatctttacatttatggccagttgatttttttcaacatgtattctaagacaattcgttggtagaaaaccgtaattacagacaataatactaaactactgggtatgtatgttcagaagagtaaagttagaaccttccttagtcacatttctgtggaggaaagagcatttatacaatccatggaactgtgaattagtatagacagtatggagaacgtattttgaggttccttagaagacaacaaaatgtttaactctagcaggaccaagcccattcctgtatgagtatctatcccaaatatgagtgtgtgagaactgtactcacacggtcatcgcagcagcatttgcagcagccaagatatgaatgacactgacctgtgtcactgagtaaacggatggtgatgtcatcactgagtaaacagatggtgatgtcatcactgagtaaacggatggtgatgtgctttatttcggcttatagctcaggccacactccatcactgagggaagtcaggatggagtgtctaggaaactcaaggcagtgtcctagaggcagggcctgaagcagaggccatggaggggtgctgcttactggcttgttcctcgtggcttgctcagcctgctttcttatagcacacaggaccaccagcccagtggtagcaccacccacaatggcatgtgtcctcccgtctcagtcttcagccatggaaaatgcactgtaaacttctcttcaggccagtcttgtggaggtatttttaagttgagagtccctctttccaagtgatactgtcttatgtcatgttgacataaaactagccaacatgccccttatccttttaaagtctttcagtgcatacacccttttaactgcatgaattgagttagtcaaagatgttgtcttgggattgggcctggaatgtagatgttcttgaactgaaggagctgaaaggtaggttgagaggagacaggggagaccaaggagtgtgtggtgtcatctcagatcactgagcagtatgagcagtgtgcagactggatttctgctggaggtggtgtatgtggaaaggcttcttcttaagaaactaacccttgagctgagtatcttcaccatggaaacgcagccactatttaaagaggaaggtaatgcttagctatttacctgaagtcaggtccgtggtgtgttttaaacacagcttgtggttgtgaatagcagggttgatagactgagtgcatcaggagaagtctagagctgtgagcagcaggcagatgctatattacagggacttagaaagccactgcagggttttaagcagaggaataacatttctaatatagcttctttagtacacagtagttttaaacagaactggcttcctttgttgaagtttggtgccctctggtgtggagccataaaaaggcacccagtgcctcctgcgtgttgtaactgcattgcattgcggttgctttgtctgttgttctttaaggtagcgtagtccttgttcaggtcagtcttagacagccatggagggaagctcaggagaccccaaccctagattaagagctacaggcagccaaggaatgcagagagcaggaaaagtgattttctctagcaaatagctcacttaattgattatacagtccaaatggttatccctgaaaatgtatacaagaaagtaacattattcagactgagcgctccctctttctctctcctccgctccccctccctctgtgtgtgtgtgtgcacctgcacgtgtgagctctgcatacacgtatataacaatgattaatgaaaaaagccgtgaaatttgagtggggtgggtgagtggtaaggtttgggaaaagggaagggaaaatgatataattacctcaaaaattaagaaaattattttttaaaaaacctatgtaagaaacacccttttcagagctttttacagatattttctgtgatgtttgtgttttccatatgcggtcagtttgtagctagtaggtgaaaaactgttgtataaaatgtagacaataccagatctagcggaaatattatttaagggttgtcaaagggatgtagcaggtagagcctcgattctttgaatctttctgctcatctctacatgatacaacgatgggcaaaagaaaaagtactttttgcataggtggacaggtcataaactgttacaatagaagggtgattctacctctatttgggttataatttattatttttccttcctctttgactatgtgaatattctagtttcctttctgttgctatgattttaaaacaaaacaaaaaatactctgaccaaaagctccttaagggaaaagaatttattcttacaagtatggcttacacttccaggtcacattctatcattgaggaaagtcaggacaggaatttggtgacaagcctgcttgctacatctgcacacaacattacttctgactgggacgctcgttcgtagctgaagaagtgcagcagaaacttgtcagggtgctgcttggtggctctcctacacagccggggactcacctgcccagggtggtgtctcccacgatggcctgggctcgcctgtagcagttagcagtccatagaaaccaataagcatacccacaggccagcctcctccgggctgttcttcagttgagactcccttctcaggtgacttgaggctgtgtccagttggcagttaaaagctcactaggacattgaaatgccgtgcttagagaaaagtgcataaagccaaggggactagttcaagtcattgagggactcattagtagggactcataagtaagcattcttgtttgtgaatgggtatctttcatcaagttttttggagattaaaataataattgtatttcttaaaatccaactgaactttgttctgtgtctgaagtgatacggtccttcttaaacatgctatccataaaatgaaaaaattaagctgtccaatttcaaacattagctatgatcaatcagataaaatgctctctaaacccattaaaaactaatgtgactactggaccttccaaagttgagctgtaatagttattaaaatgtcttccacttgtggtttggctttactctgctgtcacagtgtctccctaggttttagattttgatctaagcagtccttatttcaagccacataattgtttttaccttagctgccagtgggaatagaatttgcttctgtcccatctgcctcctggattttcctacataaaccctggctcctcgctctccatcgaggtgatttctgttgtctgaattatataggccttggtgttcttcttatgttggtggtccagggactcctggctttgcttcttttgcgttgagatagggaattgctgcacagccctgcctgcccttacacttgtgtaaccctgtctgttgtcagactccgtgtcttgccttagcctcccaagtgcagggattatagctgggagccgccaggccttatcccgttggcctgtttattagttattttcatactgccgtgacaaaattcctcacagtaattgtctcacagtcaggggacacagtcatggtggcggggaaggcatagccgcaggagcctgaggccgcccttcatactcattcatgaccgggaagcagagcgcatggatgctgttgtttagcccttctcctttctattcatccagaaccccagctcgtggaagcccctcatagacgcctagaggtgcttccttggttacagtaaatcctgtccaggtgacagtgaggactaactcatgactttttaagttatattttcttgtgttgtttagaggttcttctaggattaaaaattcagctctttaaaatgttggtgtttctaaacaagaacaacaacaacagtaataataataacacaacaatgtgccacctcacacacaacccagtgttggctgtatctcctcatcctgtgcagtataactgtaacatctcttctgcccatgttgcagtattttgcctgcatgcatgtctgtgcaccgtgtgcatgcctgttgttcgccaagggtagaagagggtcttagatcccctaggactggagctaaaaatggttgtgagctatcatgtgggtgcagggaattgaaccccagtcctcttaagaacagcaagtgttcctactcactgaaccaactccctgctactcccctttcatttgatttttaaagtaacttttgttagcatcctgacgttatttaatgcctttgccctcctttcctgtattatcctccctgcctattaaaagcccagcatattcctctgatatacctgtaaattttccctttaatgattttatctactgttactcgacatgatgccacagttatgatagttgggtttccttgactctcttagatttgtgaactctgcatggtatttatcaaattaaaaaaattttgtagtttttaaaattttggttttgtatcggtttcttttttatcctcaatccttcactcttttctcagctaaaatgaaatacacatacatacatacatacatacatacatacatacatgcatgcatgcatacatacatacgtgtgtatatattagttatatggttgagcatccttaatccaaaaatagaaaatatgaaatactcaaaatctgaagtggttttttttgcataatgcacaaatgaataccacatctgacttcatacaatgggttgcagtaaaaatcaatggattgcagtaaaaatgcagacacattgaaaatcttgtataaaattatttttagcttatataaggtatatatgagatatgagatatttttgtttaaacttatatcctacctgcaggatatctcatgtatatatgtatgtatggatggatgatggatggatgggtggatggatgggtccaagtgtgttgagtaaggagtgagaggagtgcttttgttcttcctgtggattcagctgcagtcgttcctctgctagttttcgttcactgagtccttctgtgtgtagctccctctcgtcaggtctacgttttacaccctgtagtaaatgcacgttccccgtgcttctccattctcttttgttccctggtcacgatggctaacccagtgtccgtatgtaccgatgctgtcattttccagctctgagtgtttgatcctcccacccacttcttgctggtattttctgacttcagtgctgttcatttttagttctttggctttctgtggcttttgtttgtttgtctttaacttgtttattcctggaagccagttactactactccaaaaatagaagcagaattctgtggtgtacagtgtctttccccaaccccccccccccaattactcaggttattttccatcgcgcccttctcctgaactgagttgcacagcagttacacctgtatagctttggctggtattcggccgctttaaaggggccattccctgttggagttttcacaggaaccatactttgtatggttggaccttttggttacgaaaactctttgaacattacatttactagtgtccttctatggttctttgacatcacactgtcatattgtgtatacctaaacgtgaatgagtcagacatttctagtgtaagtctacagctacagaaaccttacaagtgatttcccttgtttgtttaattttatacagtatatttatgatctcaatagccagttagataactgtgattccgttgccgaacacatattataaacagtttatgatactctgttttctaagttgaatttccatcacgagtcaattttatttaaatgagctcttttcaggttgttgagaagtctgtttaacagtttgttactttacagtttgctgttttttgttaattttattactgttacaggtcatggccaggcagtggttgcatgcctttagtcccagcactttctaggcagaggcaggcagatccgtggtctacagaggccagcctggtctacagagctagttcttggacagccagggctatatacaaagaaatcctgcttgaaaaacccagaagaaaaaaaatatattcaggctggagaggtggctcagctgtaaagagcactgttcttccacaggtcctgagctcaagtcccagcaaccacatgtcagctcacaaccgtcaaccccggggtccaatgccctcttgtggtatgcaggcatctatgtagataaagcactcatacatagactaaataaatcttaaaaaaataatatattcaaacaactcaaaagaattcatttgtccttgtaatatgatgtagttcaaaagaatcttagcagctctacacaggaactgacaggtgattgccttagttctagaaaacatctttaggagttatgtaagatgtggctgatctccttccttgacttccatgatactcccttcatgggacctagtccagccacgtggacctctgttttagtttttaatgccctttatttgttttatttgaaaatttgttttatttgaaaaccgttattaacaagtatataatgcattcagggtacttccccttccccaccctatcttcatcagtagcccctcctccctacaggtctccttcccatattcctggtgttttgctttcttttgtgacccagagtgaccatggttttgaacctctgtctattggagccagttaggctgtccagggagagtacaactacagaccatgactgcccctacttcagaatcttctgtagttccctcccgcatcaacctctctcggatctgtgattgatctctgactggcagtcttatgcgatagtcccagctatggagagatcatgatacagtggctgtgtcagaccctagtgacatcatttcacagcctctcccctgtcttctagctcttacattctttctccaggcatctttgagggaaggatgtttttaaaaaatatatatatgtgtgtgtgtgtgtgtgtgtgtgtgtgtgtgtgtgtgtgtgtgtgtgtgtgtgtgtgtatagcattgagcatgcagtggtacttgttctcagcatgttaggtagccacaagcctccatatttactattatttattgcaaagagaaccatctgattaaagctggagttgtgagtcgtgtttgtctgtaggcataaaaacaaacatttagaaggcagtttgccagtcttctttttcaatttctcttttgtccactgtgtgcacatacatgcctgtgtcttgtggtggccagagtacaatttggtgtgttaatttgtcaggcacagtccccttgttttatttgaggcaggggctctccctgagctagactgaccttctaggccaggctgacaagccactgtgccctgcctccacttcccagaacagcatgccaccaggcctgacggtcacatgcagtcttacagatcagttcaggtcttcatgcttgctttgcagaaggtactctaccagcacagttactcctcagcgccagcttctccttctctaaatgcttgtcgttcagggcctgtctttcacattcttgcccttgctttactcattagacatttttgcaatcatgtgggtcccagcctctagtaaatgatgaactaattgttgtgctaatgttgtttgggggtttcttccccacctttgatcatttagttcacaaaagacacaaaacctttatgtttaaactaaaccttaaagtactagagctgggcagatatcaaccctctgctattttgtccatttccctgtcaataaccacaagatatcacttgccatgttatatctgccactctcactccaactactcctcatggtcatgtcctcacagttgacctagcccatggtgacgtcctcctcctcctcctcctcctcctcctcctcctcatcctcctcatcgtcttcctccccatcatccccaccgtccgcatctgcggcatccgctcctccataacttagagaactaggtgtacgaggccgacgctggtataggtgagaatgtggtcnnnnnnnnnnnnnnnnnnnnnnnnnnnnnnnnnnnnnnnnnnnnnnnnnntcctcctcctcctcttcttcctcttcctcctcttcctcctcctcctcctcttacggtctctatccaagaatccccagtctgggaacctttcccccacccccactgtctgcatctgtgttaaccagtcatcgataacttagtgagcaaggtttacacaacaaaagctggtatatgtgagaatctgctcatcttggagctaccagatcttggggttcagagtttagcattttgaatacaaacagcaccaagccattctcctatacccaatacccaccactttgtggggggctcttcctagatggttttctttccaagtcttctttgtgatttaataacctaagcatttttcacaacctctgaaattagaaactgaatcacttaatttcctaataaccttataattagactctctgaaatcaccccctaaaactggtgttcccctggaaagcatcagattctgtcgccactttatctctagctagcttttacctctgaaaagatactcatttaggcctccagttttcatgcctaaaactgtatgaagcaacatggagcccagcctgactacagtctgtgcccatggtgagcataagctttgctaatgagtgcttctgtgcagcgacagctgagccaggtgagtgacaggaagcatggcttacacgcctatgctgttcactctcaccctttctaggacaaacatacactaacctgtggatgagaagatctctcagctagttatccagagtctgctgatctctataacccttcatgataaagtgtagaaagacaagcgttgctggcctagaacttgctatgtggaccaaggatgaccttgaactcctgttgcttctgcttcctgagtgctggaattaaaggcaattgccatcacacccagcctagaaatacacattaatataaaatttgctccctaatgtttcatcataactggcttattaaaatcaccatgaccaacagcaaaaacatcctagaaatagctttgttttaagatgtaacaactaatcacgggagtgggaaaatgactctagttcactatttcatctggaacactttgtgatgttcagatcttttcgtcccattgaggatcctcacattcagggttgcagggctgtgagtaaagtgtgctgaggaatttcattatatgctttattggtttttaaaatgatacagtttttaatgacagcctgcaagtttataactaaagcttcgtaaatgtcgcaagtgcatgctctaaaagttaaaatcaaacaaaaaccccatcagttgacttccaaccaacactcatggctaaggagcctcattcatgaccgtgtaggctttgttccactgttacaaagtagcatgagtcagtgtccttcaggagactctgcagatgtaaaagctgagtgtgtgacaactcagtctatggcttccaaagaagcagagcatgtgctacttgtttttccatggctgacttagtttattacagtgtcctttataatgtccttttcacgatgctagtgacagaagtccgtttgtcttatggcagaataatactccattgtgtgtgcatgacatctttccctatatccggccattccaaggtctcatatattgtaaagctataagcctgcgagtgcagttgtctcttcagtattttgagaatcattagagttcctaacacagtgcacatatgttgaaaacatgtgttttataatcgtgtatctcagtctttgacttgagtatcttttgaagaaaaatctaattaaaaagtcaatgacattatgaaagttttaatgatgtctagagaattaaaaccaacaaaaaactaaggaatttggcttcgtcatgatgctgtgtgcttttaatcccagcactcaggagtcaggtacaggcagataatcagtgagttccaggccagcaaggcctacatagtaagactggttttttgtttctttttgctttttaaatccacttgttctgaatgtttgtagaactacacctgattgttggttgcaagcacaaattaaatatttaatattatattactctcctgtgaaactctcactaacttgtaaatagtgtaatattttatttcaacagatttgtttcagctggtagtaatgatacattaagcccagtactcagagcattgggaagatctctgaatttcagactagccagggctatctatggacatactgtctcagtaaacagacaagcccatttcatactgggctctacggtaatgaaagtaacgtgtaaaatcatgtgtgtttctgtatgtgtgtgcgtgtctgtgtctgtgcatagtgcttctccttagacctgtcttctgcggctctggaggcaatgaagtgctcagtttactttgcctggtattgtttgttttaataaaatgctatatgagttctcctgttctttatgtcttctctaggtcttcaggtagtttcttttatgtagcgatactctaaatgttcagtgattgcgtgaaatgatgaacctgccaccaacacccgaccacagcattgttaggcctcgccaacttgagtgtcacttttcacatgtagagcagctaagatcagtgttgggatacagtgctgggaaggaagctggggggttgctcagattttctaaaattatttattttatgtgcatggatgttttacagaactgtgtgagcatatgggagcgcttggtgccgtcaaagtctagttgatattataggatcttctgggacaagggttacagatgattgtgttcttaactagtatgttagttagtccctggaatctttcctagtaatatttttatgactcagatttttaacattcttatcctagaacattagtctggtattatagcatggttgtataaatgtaaactaaaaagctttcacaaaatccgttgagttgtttattttatctctgcttggaattcatattgtaaaaccagtgcatcaatgtatgaatagatacaaagttgacaatattttttttctcttctgtaggtcctaggaacaccaacaagggagcaaattagagaaatgaacccaaattatacagaattcaaattcccccaaatcaaggcacatccttggacgaaggtgagtgagaatgaatcagagtccaccagctaatagttaagtgactctcggggtttcactgtacttcagctggactattacagtggcctatgtcagctacaaatgcagagctgttctctgtgaggatagttaagtattttattgatggtactattattcctgttgattgcccaagggtaatttccagcctgggcttttcatttgcaagtaagaacaaagtatccttggcattaaaataatttatttagatttttttaaaatctaaggttctaaattttgtgatttaaatgggagatgcataattcaatattatcaaaataatatacctttacatttttttattcttttaaacatcaaaattgaccttctcatagttgttgcgtgtttttcttgcttactgtcctgttggtacttcagaagcagtatttttaaagcaggtataaccttgttggatacatactttgaaagtaatttataatgtttataagatgttactaaccattggaaaagcctcatcagaagttttcccaccctgcgttgtgttatgaggttagctatgcaatgaaaaataaaaagaatgtgtcggtatttacaaatggagtttatttgtagcagccttttttttttttttaatatatgtagattcagaaactctagccaaagctaggaatccagtgatccaaactgcacgggcagctgttaaactagcacttgctcttgtgtgcacaagagtgtgtgtccgatgctggatttttgcagtcacggtcatgttcgatgcatcattcacagagggagggagnnnnnnnnnnnnnnnnnnnnnnnnnnnnnnnnnnnnnnnnnnnnnnnnnntaggtaggtaggttggtaggtaggtagggaggtagggaggtaggtaggttagtaggtagatgggtaggtaggcaggttagtaggtagggagggagggaggtagggagggaggtaggtagatgggtaggtaggtaggttagtaggtaggtagggagggaggtaggtagatgggtaggtaggtaggtaggttagtagggaggtagggaggtagggaggtaggtaggttagtaggtagatgggtaggtaggcaggttagtaggtaggtaaccaaccatataataggttagaaacagtcaacttttaaattggtatttcaaagaagattattttaaaatgtccacatagcttgtttcatacatctaagtgtttaagaggtaaagtaatttaaggctgtagatgaaatgattcatactccatgtatgtagtcagtatgcctgcctgtcttagttaagattttattgtggtgaagagatcccatgttctcttcaactctcacaaaggagcatttgttgatgctggcttacagaacaggtttagaccattaccatatggcaggaagcatggcagcacgtgggaaggcatggtgctggggaggtagctaagagttctacatcttgacctgcatctagcaggaaaaggactgtgagcccacgatcctgggcttgagcatctgaaacctcaaatcctgcaccctgcaccctgcaccctggtgacacacttcctccaaggagaccacacccaccccagcaaggacacacctcctatagtgctactccctatgggccaagcatgcacacacatgagcttctgggagctgctccttttcaagccaccacactgcctccctttctcttcctgttctcttctcctaatcacataattccaggttcttcctttaacattttcgtaacccctcttaagaaaaatacttaacattcattatttctttcctatttaaacaaagacatcagtaatactttagtgccagaaatatagattgagactttcattctcctaagaggccaagtccccttcattttaaaattcatggtgaactctgtctttggtaaagtcatcttaggtctttgtatttaacacttaattttatcagtgtgatatgcttgggatctttgatgtgtccccaaacccagaggatcctaggtgacctgtttggattttgaagccaagtagactgaagctaccagtgtcttattgcgagtgatttataatatattgcaatccaactaagttaaggagaaaataatagagaattatgggtattaaatacagggatattttaagtagtcattattttataaagaaagaagggaggatattctaagccatcagtcagtcttagtgaatctgagtatatatcacatgtccttgatgctgacacacggtggagctggctgtgctgcactggaggtgacggaatctgttaacactcagcgcccactgacgctgatgtgtgtgttgtatcccttatccccacccgcagttttctctacagtggaaatatcagccagaataaaatatgaagaatggttaatttttaaatgtagcccacagaaagagctgttgagaattagtttaaaccttgctctgccttcgtgggttctgatgtttcccttataccctggatactttccaagtgcgaatgaagtataaaaccgtttatttagatctctgtttctatgccatgtacagaacagttgttcagttctcttaggtttcatattaaaactaccaggaacccttcgtttatatagtgtcctttgacaatccggctttctcttcatgcagtgtattccacaataaataaatttgtattgatgtctctttaatacacacaaatttagaaattccatggctccagttgagtaaatgatgagaatgtgctcgcattgaattaaaaaaatttttttccaaagtaaaatgtagaattggatcccaagaggtagcatagccccctcttccttaatcctcctgttccagtaatttacagcaacttgagaaggataagctgtactgggtagccagtagcgtcatttaaaaaccatacgtaatgttctctgaaaacaccgtgtcctttggatctggaaagacggagaagccagaacgcccttgttctcaatggtcttcctttagaatccacttccgccctcttccctaagaccatggggtgtgagccctggctcctcggctgcagggtgatcctcagtcgttactatccattccctcacatcaagttaaaaacacaccagaccaagtcgtggacccctacttaagtctccattctctttctaaaaacaaaccactcttgcacatgtactgttctgcgaaaatgagcgatttgtcttctcaaaagggtagacaacatgagaagactgccttcctcacaaaggatgcaggtgtataggaactagagaggagacgacattgtcactgtgccacattacaaggtttgtgggccatatgaacctattcagggtgcccttgactgttagatgtagctagttgggggctgaagccgttgttcaacccttaagacattcgttgctcttacagaggactgggggttcagttcctagaactcccaaggaatctgatcccctcttctgacctcttcagccaccaaacacacatgtgatacacataagaacatgcagggaaaacccacacacacataaaaggaaatatatctcagtaattataatagctagtgcaaaccaattcatcaataaagcatttaatgatttaagtggggaaggaaatggaggcttatgttttggtgtgtgttgtatatttcaaagaagaaatgccagaatgaaagctaaaggaaatgatacattgtagacagagaattaaatacagagatttgggtggggaaagagaaggaacttatcctgcctttagtacatggaggcagagttagctagcaggtcactaggagagaagtgggctttaaacagtttctgttttgtaagatagtgattatgtgccttgttttttcttcaggttcttttcaaagtacagctatactgattataaaggcttatttcagtcatggatggagaacattctacttccaatacacttccaccttgtcccttatttctgcctcatgtcctggccttggtttatgcctactcctcactggtttcacccctgcagcttcagaaatctcggtccaaccccagccttctcgttacattccaaatgagcctcctcacttccctgattcctcatagcctttaggacagcatgctatggaatgcatgttatgaaagagaggtcacgggttcttatttattatgatttccatataattctttcagtaagcagacttagttcttgtcttgcccccaagattaagattccctgagggacttagttactagaactctctttaccgagttgttctgcttccaccaacatgtgtgcgtgtgtgcatgtgcgtgtgtgtgtgtgtgtgtgtgtgcatacgagtatgcatgcgtgtgtgcgacgtgtgtgtgggatagctcgtcgaaaccttctgtagtatagttgtcgtaggaatctccagctcccactgttaagtagctggtgtcctttgtttaaatgactgtcacatgctcgcttttgtcttcttagcaaatgacccagaggtcagataagtaatgccaatgtctgtataattttacaaagtggtggaagtcacttaactggtttatggacagaagtagagcagaatccccactgtccccatctgttgtccctgtattacaccacttaataagttagctgtgtctgatgaggttgtctgtcttataccttaccttaaaggggcatgcatccatgtttaattttctgatctaatcaaatttccctaattgatacaggagaagccacgtttgtttctatatctttatcttcttgccagtgtctatccctctccacaatccacacacagtgttttccgttttcttctttgctaagcaattattaatctcagatcttctttcccctgtggaattttctcattgtccatgtaatctgcggtcagtcaccaccatgtaactactttagtccccctttagtcaaattgtatatatcacagcaattcctaatttccgattcatatcataacatgcgtcaggttgttttctgagagccagaaggatggcttaccaggttaaggcactgtcaccaaccctggatggactgactttgatcccaggaaccccccacatggtggaaggagggaaccagttccgtggtggctctttggtctctctgagcgtgcacacacaacacacagacaaatgtagggaagaacctttgttcttcaagacagccctagtcatcctggaacttgctacatctgtagaccagactgtcctcaaactaagagatacacttgcctctgctccctgagtgctggtataaaaacatgtttgttgccaagcttttgacatagttgtttaatatgcaacagctaagattaaatatatgcattaactcagtcattatagaggctgcctggaaactagtgtccagccaaagaagtcacgtgtgctgatttgttgagtgtgcttcgataggacgttgttgttccgtcttgctaacagaggaaacagaaagttgagtttgaagcacgttagctctcagcggaatcattttacactaagtaagttatactcttagcttaaaactaaaccaaaacctcactgtttgttttccaaggctcttgattgttgtaaaattatcaaagggctgtcttttacccaactagggccggcactacagtgccccaagatacctgctagacatcttttttttttttttttttttttttttttcggagctggggatcgaacccagggccttgcgcttcctaggcaagcgctctaccgctgagctaaatccccagcccctgtctgctagatatcttggcagaaacacacatcccaatcggtggtggcccagtgtccctgccgccacacactctcccaaattctccacaagaaagaacacaacacagtaacctccgatccaattgataagatataattgcccacctagacacacaaagccctgtacacagccatccctgaagaacattcgtaacaacctgtaaatacacagagaagaatctctccatcagcctccatgtcctctcggctagatatcggtggcggcttctcctgccgtctcttctctccttcctttccagtgtcctcctcttcccctaaacttttcttccgcccatccgttcttgtcatccaatgacagacctcattctatcctgtacccgccctcaccagtatgacatcctacacttgataaccttgcattttccatgaccgtttagtatggcagatttagccatgtcaactcttttgctgcattaatgagaagccttcagggagacccacaaagcaaaaactaagattgcagtcaaactttccttgcaaaccctatacctacctttttgtcttataacattcccatgtattccagaaaacataaagcctattgggcatatctgctagttcctaagattacctctgatctatgtcattgcttgtggaaaatcagactcgtgtttctcaacactctctgccttgaatactgttgtaaccagttaccacttgtgagtgtgtacaccatcactgaggagttttttctgcctcttttagtaccggcatcacaatctcctctcctttttctaatactcagtctccttagtgaataacttccttatttttctgtcttccaggctgtgtactttatttcatttctaaaagttgcacatataagagataactcttaacagtaggagcatgacttggtaattcagcatattcctagcatatgtactgcaccaggcttgatacccaacatgagacacacacatgcatgcacaacacccccccccccatgattaatctatatattggaggaatattttttctaattgtttaagatggaggacaaaggatgggattgggaattgattgaagaagaccattttttttaatttatcattgatttaaatattatgtatttattaccagtgctatgctgagtcccacaaaggatacaaaataaaatatgcagttcttgcctttgaagagcatagttaaagcaagtattaagtaatataaaattgtgtgttgtaaattttgtgttcatagctgtttacatctactgtgtgtcacaagccagacctttagggaaggcagcaaaagaagtctcctcaaagacgacataggacaagggctgttgaagtagaataaggttcgtttaagtgggacccatgaaggggctcagctgatgaagggtcttaccactaggtctgggtgacctgagtttgatctctggaacccacagaatggaagaaagttactgagtcacgtgagttgtcccctgacctccaagcatataaacacaaatgaattttcaaaggtgaaagaaagaaaggcgaagtcacgatttgctgaggtgtaggagaaaaggctaggcagacattttaagatgggagagaaggatagaaaggaaatgtgtactctgaggaaagccagtctgagtaccatagctgttttgtaaaaccatgaacagtcaactgggttagagccatttggtaaggcatgcctgtgtgtagtaatgtgtgcctgtgtgtagtaatgtcctacagtggggatgtgagaagaggaggactcacggtatagggaaatgttacttcctgtaactgtgtcggtacacgccgtttaattagggcagctggagctcagtaactgcagggccagccttgtcgtgactacagaaagggtctcgtgtttccttgttctcagctttccttacccgactttccaaccctcggaggttagaacgctgtgtggcttcttcatgaggcctgggctcctttcagtttgtgatccattctgaatggcagtgacttcagacatactgggcttgtggattctctgaaagctagttttcagtggcttcttagggggaattaataatttatagttgtgttagttagggttttattgctgtgaagagacatggtgacagttcttataaaggaaaacatttaattgggactggcttacaggttcagagcttcagtttgttaccattagagggtgctggagaaggagctgggagttctacatctgaattcacaggcacaaggaaaagactgagctgctaggccttgcttgagcaactgagacctcaaaacctgacccccacagtgacacacttctccaacaaagccacacctactctagcaaggccacagttcctgccagtgcccctccctgtgggagccactcttattctaaccaccgtccacagtagtaggttggattgccttgtgtttcagtgtatcagtagatgcatgtatgaaagcactccagacaccaacaaaggaaatgttaaaatatgagttacttaattattgttacctgcttagaaaaccaattagttggtttctttttgttgttaattaacataaataactattctcagatccatgataattatcttactttaatcttatatttaggtctcttaactgcagacagctttggcttacttgagagcctgtgttgaatggtgctactctttaacagtcatggtttgccagttttaaactgggggattatcttttgcctgtgaccaaaaattgagtctgtttcataagcctataatgggaagtaataggaggtagccatttgcagcaaacacataatttgttctgtgttctactaggttaatttccactatctatagttataaaaagtgggcatttttctattgctttggctgggtgcataatacagcagcattggtgagagctgactgcaggagaagcagctttacctggaggccttgcagtgtgtggggttcttcctcagtagccatcgggaagggcccgtggctcagctccaggagggatcttcacagccagtccagagatctgtctgttatctgagtcacccaaatgcaaatttattttaataattcttaaggatttgtttgcatctgttcagtaccatggagaccttgggggcacattaattttatgacagtatctcccccgcatggactagcaatctcgatgtctgcttgtttgaatttaaggcaatctgttactgcataggtgaggtagcaatgaagcactcaaacgttcatgtcagtgtttcggttcatttgattctttcttttcttttcttttttctttttatttcacctttatctcttttctttttctttattgttttgtttcattttgttttgttttattccttttttttttttttttaattttagcgtttaaaggggtgtctgtggccttgctgcatacttgcctggccatatttagtctgcatactaattgttagttggcccagttgaatcttactttacgcggttagttcttttggtatgcttttctgtttttgtccacctggctgagttttaacatttcaattctgacggggccggattttagtccaatgcactaaatttagtctgcagactaaaagtaatccgcggattatttatttttttgcaaaattcccccactggaaactatctaaaagaaagttccattctcttagctcagtcaagggagttcacagctaccggtgagttctgccaacttttgtgttctcaatgcctagacacaatagaccctgctaactcctgtgcacatttggctccgttctgcctaggtgttgtgtacattattctgcgtgtcacctgtaactgtgactgtcccttagttgttttgtttactgtatacattcttgtttagcttttggtgggtgaattgggcatactcgcagatgaagctgtgttcaatctgtactcaaagataaaggtttttttcagtgagtttttgctaaaaggaaagaatgctctatattaattttttttctaagtcatgggtgcaagggtatgagggggcaaagcccacaatagagagaagtaagtttatacccgcactcctgaggtgatacgtcagttttccggagagtcctcacaatgagactgtcagtaaacatctagcagggaagtcaatccaaacgaggagaataatccgcggtggcctgattctttcttagctcacacaaattaaaagtatctggcttgaaatttacagtaatataaattaaatatacctaatatttctctgtcatgaccaataatttagcaaaaaaaaatgtttatactgtataactgagacaaaataattgtgaaattttagcatattgggaacgtacatttttaaaagcaggccaataggcccctcccagccatgtctgtgatgatgaatccaggtcatggaagaccactttactttgagaactttacttctagatttcattttgattttaacttattttggttttgtatttttttaatgaacacactggcattggaacataataaattaaaaagaaatgtttctttactgtgcctcaccattgcacaaattaaaaaattttttgcatacacttgtggtaaaataatttttaaaatttggacttgctgctgtggagacgaatgcttacgtttataactgtataaacattttctatgtcccctacaccatggatttcttataacattggctatatatctttccttgctggactattctctcttgcattgacttcccctgtttggattatttcactgcattatcacctttaggatccatcaggaacaggacatttcaccccaggagcacgggtatgtactttttttttttttttttaaactttaggtccattttataacactagggaaaagggtgagaactgctgttggaatgtgctaatgaaatcagtacgggggattggtcgctggttctgtactgtataatacaggttttcacgtagcatagttcactggtcgatatccattgacatctagatggaaaagcatggattttaattattcttaacatgcagccaaaggtcctgtattttatttcagtggttaaggagcgaaatagatgcagaggcttggctagaggccacatttcagtagctgttggcttcctgtctttacagcagctagtgtccagttgggactatgcctttggaaggcgcatgctaccataccccatattatggatagactaggcctcgattcacatacttttcaacttaccctgtatccatagagtaccaaactaaggcttttctcatgaggactctcctaaagatttaaagtaaacgagtactttttaaaaggcgggtcttcaaaaaaatctattctaataaagactttggcatagattcttttttaactctggtacctcaagttaggatttcttcacaaaacaagctaatgtatatgccttagtacccaaatgacaggcggttcctggtagccaataaaagaccaaaaaagatacagggaactagttttttaaataatccaaaataaaatcataactctaacttcttaagacttttaacagaaaaattctcataactatagtacggtttaggtcgctcgttttgttttgtgtttaagacagagcctcactatgtgtcactggcttcatactcactgtgtaactcgggctggcctcgaactcatataatagacatctgcctgcctctgcctcccaagtgctggggttaaggcgtgcaccaccatgcacactcaagcatgaacctctatgacctctgtgcttggattttagcaggagttatatttatattcaagtgctgtctacttacctaataccaaaattggtaccaaaacattttattttagattagcaagtaaatccataaattgtcagttagatacaaagtgccaatcattttaatatttttcttacagttttgaatcacaatctcagtatgtacctcgggttgtcctggaagtcaacatgtagaccaggctgaccttgaactcataaagaattgcctgcctctgcttccagagtgctggatgtgggtcactatgcctggctaactatgatggttttcttttctgcattatggatttgattggcacttggaaacagggaagtgaccaaaaatggtggcaaaatgaaacatttaaaagtgtatagcagctttaagatttccttggctatggcaaattatagtagttaaaagtatgaagacaagaaagttttggctttgccacaggtctttttaaagtcgccaataagttgaccaagtcttaaaataagttcttaagatgctctccaggcacttactcaatttctgtaggaaataatccatttctgtaattaacagtagttctgtaaagggaaggaaaacaatccatccggttaccagtttctttagaaagtcctggggagtctcatgagtattaatgaaccctgtagtgaccaggacttgccctgtcagctgctgctaggaagccttggcctcctggggttttcctcgcccattaccatctgcacgcctccctggtccaccttctccagccccacccttgctctcctaccacttctttccatcaaagggtatggtttagtttctttacattcaagggttctgagtcccatctgctcctgtttacatcccccttaactactagctgtccttacagaataaggtctgtgtgcatcattttatgtgctggttcagtgtaagtcacagtgagtagaaagctatgtgttcttttaagcaaattagattaattgcttcaatttagacttaaattgacctttctgagattatggcccttgctgaaaaactaagttgttgttgtccaattaaaaactctggactattcactgaaagaaatccaaatgtgaatgatgactaacaaagtaggaagcctaggtactccagaatctttgaataataaaaggctctgtatcaagattatatggagccattagcatttttacaattaaaacagcttctcttttaaaaaactctgactgtgcagtccattgtcaacagttgttctgcttaaaagaataccaaggcaatctcatgttttagcagtaaaaactttaattaaaaaaaattattctagtcatgcttgcctccagatctcagcttttatatgcctgtagtagtgtctaggcatgcttaattaattatgccactttgcctaagaggtagcttttcttgcttctctgagcttttcttgcatagtttttgattagtgagcagttcacccattatggtctatcccatgcagttaactctctcattctagattttgtaaccgcggttttatatggtgagaattaactcagtactcaaagcattgtttgattgggttcttagctagagcttaattgttctttctgctgttttgtcctttcaggtctttcggccccgaactccaccagaggcaatcgcactgtgtagccgtctcctggagtacacgccgaccgcccggctaacaccactggaagcttgtgcacattcattttttgatgaattacgggacccaaatgtcaaactaccaaatgggcgagacacacctgccctcttcaactttaccactcaaggtaatcccagacacctcgtgtttgtctgttgagtgggacgcccttcttaagtctgcagctttcgaatactggtccccagtcgaggctgtttgagggagggtgaggaggtatggccttgctgaagtatgtcacttcatgtatcctgtgtgcttgttctcggcttcctttgtggtttaagatctcccaccccgcccccctccactatcaaggactctaatgagttttttggaaccatgcacccaaaataaacttttttcctataagttgctgtggtcatgatatgttatcacagagatatataagtaactaagacgccgatgaatagatgtgaaatgacactttgttggtaataacctaagacagacgtagtcacattaaggacctgcggaggttaagaagagcctgcactttattatatcagttttgagcggtaacgatagatgaaagtttgcgaactgtcctggtttgtccatttttttttttttgtccagatcgttgtaaatttggacatgtgaaatatatagtgatttcacagagtccgagtaaaaagattaagtaatagtgtaacccttagatttaaatctaaaagatagactattagtatttcttgctttccagcatttacagcaggtttttataacctagacatgagttttgcctattgtcttaattccatttgctgttgagagacaccatgacctttttatattttcttataaaaagaaaacacttaattggggctcacttacagtctcagaggttgagcccatcatgaaagggggcatgatagcatgtggcaaacatggtcctagagaagtgtccgagagctctacgttctgatccccaggcagcagggagagagacaaagaggcctgccttgggcttgtaaaacctcagtgacacatttcttccagcaaggccacacctccttaactgtttcctgtaatcctcactcctgatgactaagcattcaaatacatgagtctgtgggggtgcatctaagtcagaccacacctatatttctctaaactcaattatcttagttaaaaaataaaaacaagggagactagaaggcccacaaactgcaggaccaaacgcaagacgaatagagaaattaccgtcccttctggtctgggccccattgcttgctgctgagtaaacagtaatgggagaccgagtgcactcgttcattggtggtttccagtgagtcccaagagagccgtcatcttcttccctgagtaagactccgtgtcagtcttccactgtcttactactcctctgataggatcccctcccacccccaccaactaaacacaagaatagaacaaggttagtgtgctgactggctgagtggtaaggattggtcaggctaggaaatatgtgtgggagttgggagccttctgtgactgagaaagacatttcatctactgaatgtggagggttcattctaaaaccactccaatgttactacaacttccgtcttccaggctctataattcaagtgcttctttcaatattctaccctctgctcattagtgttctgcttaacatagggtaatcaggatcccaaacatttcagagttttggatcagcttttaacagagttaatagtctcaggacatctgagtactcttaaacatggtttaaaacccgcctcagcacagatgatagccgcataatgttgacagtcttaagccattactgttccctcaaatcttacatgtatccctccacattcaatttagaataacctaaatgtatagggagaatgttcattttcaaataaagaaaatgtctctgtgttggttagagagggctcctcacctccttagtgcatcgtaaagagggtaagcattgttctagttacaaagttgaaaaggatagtaggaaatcagcagatagcttgagtactcgcagacgacgggccggcgagagtccagttaacttgggagcaagttgtttttctgtgtgcccacccctgggcctggaggtaccagaaggaagctgtgtggttcgggtgttactgttgggcaactgcagtttgacaaaaggctaagctagaacgttgataaatggcctttttcttagtgctcttcacaaaggaaagttattggcatttagcacctcttcactgcgtgcgtgcgtgcgtgcgtgcgtgcgtgcgtgcgtgcgtgcgtgcgtgcgtgtgtgcgtgtgggtgtgtatgtgcaggcgcacacattcttgcgttaccaattcaatagatgcctaggttttttgtttttaaacactgataagaaattcacgaggataactttttaaaaagttaacagtgtcgtgtcatttttatatctctggtgctatgcagctaagacctttatcaacttccaaaacattttctttgctcctgaaatcatgtccccattaagcatgaattccccatagcctctctgcccctagcagcagccaatttgcttttcgtctccatggatttatccgttttggatattttgtataaatggaacccttaacgtatgatctgacttcctccacctaacctgttttcatccatagcacatgtatgagcccgccattccttcttgtggatgaagagcattctgtagggctgcagccatatttacgtaggcattcatctctgacggacttttgggccctgctgcaatggtttatatgctgatgcttgttccaataattggggcactgctcagtcacatgacaattctgtttacagaaactagcgcactatttccacagatgaacgtcttacagccctaccagcagggtaaagagttccgattcctccacagcatgtcagcacttgcctttgctgtcagtcatcttcctgatgggtgttagccttaacctgtttatctcactgtactgagtcaggattcagtgagtgctactgcctgcgtactatgtcctttggtggccttggggacttgggtccagtgagaatgttgaggaaatgaagagaggacagacagatggaaacacagacggggtctgggctcagtggtctgggcccttgctggaacagtgtctgcactctgaagtgcagcgtgtttgttgtgcagaggtgaaaggagaagtggggttgttgcatcccctggacaaggagctggggttcctgcctacagataacaagaaggcaggacttagggtgtacggctggagcaagcctacagtttagctaatcttggtaaagtcacctctgtagtagagcagacttcaggtcacagacatccagggagagaaagcactggtgggccttttccacacatgctatcaacatctgcacatagaccagaaggaaaagctttgtcatttttctgtggttgtcatcctgagaggtggggaggctttgtcacatcctgagaggtgggctgtggggtccttgacatggacagctcatgtcagcagcacattcactgaggacgtcacacactcagggcttccttcactccctgcatctgttctcttgttgtttgcaaccataagcaaaaaagaaaaaaagctttatgctttaaaatatgaccttgaaaatactgaaggtctttgggtcacttttagattccagtatctggtagaatcagctgcccttggtaattttcaagcttggactgagagtataactcaaccagtaacttgacatacatgaatgagggcctgagtctgatacccagagccacccctccaaaaaattttatctagtcaagtggactctgtaaatacatcctgggaaccatttttcatcaggctaactagattaagaaagacaagactagtggtgcgtacttgtaatcccaaaggcaaaatggcacccatggtttggtgtgtgtgcacacatacgtatgttcatatgcacctgcgcacgcacacacacacacacacaccctgcagtttcattataatgtttagctctggtgctatgttttaaaataaagtctcaattcgagaaaactaatatagttcaagcttttttaacaatgtacacacctaatattcacactttaaaaaaagactaagtattttatcttggtattgaaaaccattttatttgcccatatatcacacacaagcacaattctacaccatcctggtatggttatgttttcatggtttaaagagtacacaccctagttctctccccatttactgtgaaacggcacccggcaaaggcagtgtcacaggagaaagagtttatttgattcggctcacaactccaggtcctccttgcagggaggccagggtatcaggaacttgaagcagctagtaagatctacagtctggatcagagagagcaaatgcatgcacacctcctcctgctcagttcgcttcaattgtagtaccatccaggacttaaaccaggacagtgccacacttctaaagtgggtcttctcagatcagtttacataaaagattcctcacagttatacctcagctagactctctctctctctaagaatctcatacccatgagatcaatgctagattatatcaagttgagagttaaaactaaccattgcacatgtgtgtttggacataatatagatagataaatcagaggtagctatgttgtaattctgtgagaatcagagaaaattgtagtagttggaaaacctttctatctactcaagtggactctagaaagattctgggaatcgtttttcctgagactaaatatattagtgagagttcgtgagaatgcatgtgagtatatatgtatgtgttttgtcttaaaaattcctgtcggccattctgaaagtgaactgtcacagtagaggagtgccttctcccagtgatttgtaaaattacagtgatttctaaatcactgttactcttgagcagtatttctgcataaggacatggaattactgggtgctgttctatttgaatatataaatatatatatttaaagtgcagctttaaaatgcggagtttttaagagcagttggtaactaatgggttggagaactgtgtgtttcaagaggctaaaggaggaggcctagtcatccccttgactgtatatcgtagggagtatctggataaccactgttaggtgaaagaattgtgaagaaacagcaagctcccgctggtagcagtgacttctcctctctccaaacaaactcggtgacaccttgctctacacctcgtctccaaataaaaagaagccctgggcttcctgtctgaggtgctcactgacttgagttgaatgagtgctgcaacgtcactttacataaatacagccattttctgttaatgtaatggatatttatgtgcatttctagcttgcctctgattgctgtagctgttaacaattaaagtttacaacacagctaagttcggtctgctggttactgtacctcgaaagttagtgcacaaatgatacttgtgtttcttggattaatcataagaaatatgtgcaccctccactgggagcccttggggaggaatgctgtatcagtctgggtaaggccagtgtgcacacacactgaacaagtgatcagaaaagagaaagtcaacgaaagggaatgggcacccgggccgtgaggagagccactggacacctccaacccgtgtgtgtgtggaggtttgtcttcatgtagctttgtcaacatgcttccattacattccttccctctttcagatttcttctaggtgttgaacacatagggccctttgatccaaaagtgctgtcattctagcagacttgactgcctttttgttcatttggccagctctgttttctgttggtccagcaaagacactaaaagatctctgagacctagcaaactgattcacacccaatattattaaatgtgtgggtgggttttttctttgtattttacttgggataacaagtttaaatttctttcaggttaatgaagaataactatatacactgcataatcagccatggctgctaccagatacctctgctggcccagtcaggatattactgcttaacttttcttttaactttttattctttgagtttcacatgcatcccagtcccattatctctccattccctcatagctgccgtttgctcttgcaacctaaaatcacacacacataccaccaccatcaccaccaccaccaccccacatcagtaaacaaagcatagacatctcgttgtgaaacctgtagtatggcacagtatgtcccatagtatatttctctgtccacacatcttcacttggcaaatgttcattgcaatgaatcattggtcttgttcgagatctctgacttctgtgacaccatcaatattgggttctcatcataactcctcctggttaccctgttgttgccctgtctcctggggatcctgcagctttggatcagtaggacccactctttcacacatccctacaattcacagatgatatagattttggggtgggccaactcagatccctggatctggacctgttaaagtgcctggctgcaagcaaactagtgtggtatcctcttgttgctcagtgggagtaacgcctgcctcttacaccctccctagtagggtatagcttcccagaggaagacttctgtttgactaaatgctctcactcaccaaacactgactctttctctgcacttaaagcttttttcttccattaatttttcatgaatcttggtatcatttcattgcttttcttagttagactatagtcttatttagaatcttaatttagtataaaattagtatttagttgccaacctgtaaataaatcaccattggcaaaggttttgaccctcgaacatttttaaactgttacatctgaacactttgctttctagcaacttggggaaagagatttacaggggaataaaaggaaacctcaaactacttaatgggtttggggcttcctttatttcacaacttctcagttaatgccatgcatttaaattgtcaagatgttactctaggtgagaggttggtatcgtcctacaggcacatgctatttcaaatgtgactctggatggggggtggtgaaaagagaacccacagattataaggcagagtgccaggcatgactcgctccccagcaaactggaaggacagtcacacttcttgaaggcatcactgggtacttaagcaaactcagccccacttaccttaaggaatataaaatggagcgaaagcgaaagcattcctgtgttcactgctcctgtctgcccacccttcttggctgctgtctgttcatttacccatttctgtttgctgctgttctaagttacagtcaggagttacaggtgatggggggtggcaggaaggatgtatatacactcagcctcaaacttctcactgctgcctttgtccaagataaacttagttcttataatctgtcagctctgtgtcacatgactgtgcacttcccctctaacttaattactggctcactggtgcagtttataaactggtattttaatatcatgccttttatttttacctataaaggtatttcaggtaaagggtggagtctgaactttaatgtctgtctctgttactctagaactgtcaagtaacccacctctggccaccatccttatccctcctcacgctcggattcaggcagctgcttcacctcctgcaaacgccacagcagcctcaggtgagcacagcgcgtctttttggtccacttgtctaacccgctgatttccaaaggacactgttatctccattttctgtgctatgaacgtttaactccctttacgtatcaaagccagatagtacccaaatgagtgaaagagaataaaatactgctttccacttaaagcagttacaattaaaggttacaaaaaagtaaccgagaaatattataaacacacatgtacataacttatatgtaaggcatatccttttctggttttgggtttgttttttttttttttaagtacagaatagagtttatttagggcatggggaggggagttgaggtgggagtagagacaaagaaaggcagagggggggaggaaggggagggaggagagaggagtagaagccagccatgaacacatggagagaggactcgaggggattggggagagaagggacagagggggaagggagtaagagataccctggcttctcagtgtaagatgtttataggagaaaacagtctttataaatggaacattggagaatgcctcactttagttctcacttccagatgaactccactaataccctaaagaggatgatttaagaagagtggacgtacgttgatctaatcttaaaccatcgacagggctaagagtgcaacgaaaatacatttgattttgcatcagagcgccattaggctgtcctcctcctcacttccccaccccacccagaaaacaaaacaagaaaatctcacaaagaccccaggttgaagctagagcatagaatgaataagtgagaccctcatctccacatagacatagagaacacatttaggcatagacaaagcttttcttgtaagccatttccaaatctgcttcatgcttcaaagaaattgacagctaatctgcaaagctctagactttctaatatctgaacctagacatattttaaatattaaattttcaaggtcagaatttccaatcattcaacctaaatgagagcagcatatactagccccagcatgccccgggtctaatgagttagaatgtgagcctctgcattactacagtagttggtcttcttgatttaagtccctgtaatagaaactcggtatcagaaagaatttacaatttagaggctgagtcacaaagaaccaaatactgtcttcagcagcatgtcagtcaactaaaaaggtttggacaaaaatgaattcagaactaaggcataatcctgaaattaccgtccagcctgttacagctctgcacagtaccggtgagtttgtctacagcaaactcgttgggatacagaggcttccggtggcgcctcagaagtggtcctgtctgtagctggctagaaggaagattgggtttctgtggctttcctttaagcctgtgagcaaagctaagtactaagaacagaaacagaaaagcaagacaagaccatgatcatactcactgctatgaaacggactgatggccaaaactatcaagtggcttccagatacccagatcttgatgaattctttgtgttgctgcttctggaaacccaataaattttacttacctggatggagtcctaaaatcctgtcagtattattttgggaatatatagttagttcttgctacagaaactttgactaacacactttgtagtcacagtgtctcaccttttttgaaaatggttgagatagtgtgtctgctaatgagtgacgtaaacgcatgctgattacttctttgttcggttgtgtttagtgttcaggtggccactgcaggtgcagtgattaaagtgttacaatatacaaccatcaggttcaaactgtaaaatcatcaatcccattttagggctttaacttcctaatacatctagtagaagaggaaaaagctactacatacagagactgtaatcataaaataacaaagactgtagtgacagattttataaactggacaatcaaccttctatgagttcacacacgaacacttcagagatcatacgattaactagcagatgaattgaagtgagggtaggaggatgtcttactcagagcatctttctttgtagaggatgatggtctaaagaatatagatgattgttcagtaagtttcttgctgttgagaataactttgaagccagtgatttcagtttacattttcatcgtttactgagcacagatttatcgatctcatgattgttggtgtttgttcaggattgttccaataactggacccattactgacaaaagtgagcaaatttcatttttccatcacccaatataaattgatgcccctgccattctgcaagtgatttctgtatgtgttgctctcctcagtaagccacatgagatcatgctcagccaaccaaactgttgaggacagctctcagtactgatgaagatgacagactgtcctgggtatgttctaaaatgataaggcagttggtttttatagcttgtgtatcacatgcacactccataaatatgcctcttctacctaaatagagaaacgccatctaaggggatagacaaggctaagactgtttgactgtaccccgctgctgctgaggaactgggcaggcctcgttacagatgtcgcctctcgagactgcttcctgctcctgcttgcttagccataaaccaactttatatcgtagtcatttacacatgagtcagaacaacagcagaaactctcaggaaatacattctgttgtcgagaaaattctcagcccctttccaacttagaaaaataactctatcaaaaatggaaattaaactcgttctaatttgtttgggagagttaaagtcaggaaaatttctagtatcttcagtaattattaaatgaattctggagtccatctcagatttgtttctgaactgtgggctctgagtttgaagatatcttacgtgtttcttgccctttgttcagactgtgttttgttgactaggattcttgtcctttcctggttgaaaacctttctcagaaaaagggaatcgagggacttgactgagacggcttctctgtcgtttgacttggactgggctgaacttgtagatcctgccttgaaagctctcgatgtctgctgatgtagaccagttctggcctgccccttagatctaccttcttcttttcagttctttgactgtgtgtgtcatttagaaaattgtgtggcttttttttttaagctttttttttcttccactatcaatgcatagttaatctgggggacagtgtgttcaatgttcttgcttccagaggtaatcacagataatgttgttttgtgtattagtctgtacatgagtagccagaaaaatgagcttttcatttaaatgggcttacactattttgagtgtcaaggtatactactgtagtcacatgttgtatttttctattacttaatagtattgtattcctaaagctttgtggtaattcatctaatttgttccttttgaacatgagagatgtattttaaggattgtttggctttttttcttttgctcttctgaaaagggcaatgaaacttaccttttcataaagccctgcgtattctttcaaactagctaataaaaatagtttgggaagtaatgttgaaggaataaggaattttaaagtttttaatacaaactgtcaaattgccttccaaaaccctgttccagtttctactccccattgtagtgaaagtactttttttaatttcccagaagagtaggggtggggaaggttcactgtgcacatgtttatatatgttaccaatgtggccaaatgagtgtagctcttctcgggacctagatttctattttaaactattgcagtttaagagactcacaccagccagtctgtgtgcactgtatgttgggttaggtttacttcataaacttgtctgaatcactgcagtgaaacctgccaggttggtatggcgatgcatgccgtttccccagtcacacaggtctgacaaggaccaggagtttggccttcttgagtggtttaaaaccctggctcaggggttggggatttagctcagcggcagaatgcttgcctagcaagcacaaggccctgggttcgatccccagctccgaaaaaaagaaaaaaaaaaaaaaaaaaaaaaaaaaaccctggctcagtttgtaaagtgcttgccttgcaagcataagaccctacttcaatccccaaaatgctttcgatgccaagtgtggtgtacttgcttataatctagcagtaagaaggcagagacagggtcctgggggactgctggttctccattctagcctgataggtgagctccaggccagtgagagatcctgtcccatgttggtaggtaaggacacttgttgatacctgggatctatgtggtaaaaggaccaaactaaatccactaccaccaccaccccaggttttccatggcacgggtgtatgcatgcatgcatgtatgtatgtatgtatgtatgtatgtatgtatgtatgtatgtatgacaaagtattttaaagtagtaggatgcatttccgaggctgacacacaatcacgcagttcctgagcatgcccatacacagcatgcacattttcctgcacacatacaaattatgcatacacaaatgcctttttttaaaaaaggaaaattaacttagttacttttacaaaagcagtgagtggtaggtgctgcctctagaaaattgtgacttatgtgcgtacaaagcgtgtcctgtcctattaagtcaagattacctggaggtttccactgcctcctaaaggattcacctagtgaatgttttatagctcaccgtggatgtttcttaagtgttcttcagatttcacttccttgcctgaacttacgtgcttttatttatttttcgttcattatttccttaattagattttttttaacttaaaaaagaaaacatttcctataaccccatattctaccctgaacagtcaagccccttccttagaatgtgcttgcctctgcagtgtctctggtctcagcagatcagatacgggagctctcactgacaggctgtccgtgggccctggtgggtgggctagaccacaggcgtctgagttctgcttacattttcgcagtgactgatgccttccttggtttctgtgccaagtctctttctaggaatagcatggaaagaattgaagatgtgctgctaaggagggtttcctctcccttattacatattacattatatttatattaaatatactatatatttcacctattgtgaaaatcacagtaggtgttatgagagagacgcaaaaactggctgtgcaggtggagctttgagcttgctggtgtagatggatttagtcagggtgagtttaggatgcacttggattccactggtggtagctctgtttatccctgtgacttataacaaaagacatcgctttacattagggccttttctactgagtaggttgactaaattacttctgcagtataaataatatatttctggatgccagtattttttgtatgggacctgacatgtcatgagatttcttactaacctagtaagtacaagcacactggctgtggactggagttacagagcaaaataacagaaacgtgggaaatgaaatgcacacgagttctcaagtgcacacaacacacatttacacaggttaattcgactacccaaaatattcccttactaatatgtcacttgtagaaattaataaccgtagtttttggaaaactcggaaagtgtttagaacaaataagcactaaatgttgtttttaatttacagcctaaatagccacatgtagctcgtagtcactttggcttcgttggtcagtataaatagaacatttctgggtcgcagctagaccgtagtgtggattcctgtgggttagagccggtctctcttctagacacactttggaaatcctgatttcatggtggcagaaccatctcctccaagccagtgcgtcccacatgctgtgcctgtctaagaccctgtggccgcaggttaaaatgtggcccttcactgcctcccagtgcactcttggtaatcacactgggccccagaatgcataggaattgctgtacaaatgaggagtgaaggagaaaggagaactcaagatggtgactgttaagtctgtggcctgaaaagcttgactgaggaagtctccagtgttgagacagttgtcattaatccgtgactccagaatctagtgacttccacatttctgagaatctagggctagggcagcgttcacgtgcgcgtgcgcgttcacacacacacacacacacacacacacacacacacacacacactcctactattcccagaaaatgtctgtatgtctgtttgccagatatggacctttatgttcaaagttttgcaagctggtgtggtggcacagacttgtaatacagcatttggggagctgaggcaggaggctcaccaaaagctcaagaccagcctgtactgcataattttatgccaacttaggctgagaatgaggcaatctcaagaagtaaaggcaaacgtttaagaaatcaccagaggctgggcaggtggcttagtggttagtgcttcccacccaggcaagcaggaattgccagagattaagtccatgaaccatgtaagtgtcctgtggctgtgcctcctacctgtcatacccgaaagaaggagacggagcctcagagcaagctggctagggagacttttgagataggtgagtttgactgagagatcctgtctcagagaataaggtgaaagaacaacccaagaagattccccacataaacctcaaacttccacagttatacgtgcacaccccaccccacataccttagaaaacacatggaaacaaaacagtcacaaaagtattttagttcttttttttttttttttttttttaagatttatcttatgtggttacactgtcgctgtcttcagacacaccagaagagggcaccagatcccattacagatggctgtgagccaccatgtagttactgggaattgaactcagggcttccggaagagcactcattgctcttaaccactgagccatctctccggccatatttttaattcttataataattcttatagtagaatctagcacagaagccctggaactacaaatacaaacccttctcagaaaacaaggaagaagcagttgttttttttttttgtttttttgggttttttttctttttttgggggggggggagctggggactgaacccagggccttgcgcttgctaggcaagcgctctaccactaagctaaatccccaccccccagcagttgtttttctgaagtaattttgtttaccctaatgaagacagccctgcacagttagagacactccagtaccttagatggtacttggtcagtgatgctttcccaggcctctgagtgtaagcgcttcgtggcatttaagttcaacatacaaatgtgttcgttcttaccgctttgagttgccatcctgaattttagcaacttgtaacaggatggatgttctctggcatttcgtctgtaggttaggaatctagaagccatctggctatatgagagtttgaatgaggtggctaagatgtgcatatacatcatggcttccagtttagtgttggcgtggtattcctgagtatgcgaatgagtgagtctctgtgtctgtttctggtttgcatgctatttcttgggttctttccttctgtttgttttgtcctattctgatgtattaaactgtattttaccttattatttattttattattatcccttaggagactgttttctacgagacagaaaggcgtggatctgatgggaggggacgtgggaaggaactgggattagtagagggataggcaaccactatcaggatctattgtatgaggaaaacaatttaataaggggaggtgttcccgagggatacagttatctcatggcttgcctggggttagggctgggtcgtccatgtagttaacatgctttgacaggtctagccactcctcctgtggacttcttatttatctcagggctttcatcatatcagctgtcttctcttacaccaagtgatccagagagaatacggtggaagccatcttttatgtccttagaaatcacacaccataatctttgcagtgagcatgggtacccagaggcctgaatcaccactgttatcttgggaggtgctacagtgtattaggatgagcttaggcctgccttcctcttcccagggcaggttgtttgatggcgtctgtccatccctctcaactctgttctgggattggaggcaggatgtctgtaggctcttgagaagcggaacagtgggcgtgtttctaaagatggctgacctggggcagtgtcagctctgcagttgtttccagagtacttttgcctctgtccatcccacacgggtgtttgactgctctgtctcaacgtagtaaagatgagcgggtgtcctccaagcaccctctgcacagcagctcctacgccacattcagcctctagccatatcttcttttgtttgcttggttgttttatgctttaatacaaactaagagctgtaattttgcatataaaggatttgcttgtgtgtggaaccaggtctctctcactgtgtgggcaaggatggccagccaacacagcaggtaatcatgagtacctgttaaagtctccttatcaaatgattttgatacaatgctgagtaaaataatgagaccgttagattttaaatctgaggaacatgaatatccagcagattttatcgttcagcagtgctggttaaatgtggatgtggtgggtacctgtcagtcctgggctttaagatagaacagcagttcctcctggcatcttctgctgaaagtgggtgtcgtttggtcatgaccatgcatttggtgacaattgtgtggaaccaggaaatgcacgaggggatgcttttgtgaggaacattacagttctgaagtaactggttttccaaccttcttctgaaatgtttttaatgcagcctatgcctgaatcataaacatgaagaaaggcctgaagggctgtcatctagccaggaattcttgttgttaggagtacaccaaattcaggtgactgtgaacagaaaaacttccttttagtagggcatggctgagggagtcatggttgaagcctgagacctagccagctcttctgtgctgagaacggctgcctgctggctttggagaaaggtcctggtccatatgccatgtactcgtgagttagacgcttccgcaccctggcagcatggccctgcttcctttttcataaggaagtagaacgtagagtggttgtggaggtgataaacgaaacagcttacccttaaatggtaaggtgactatgtgtctttggtgtctgctgcgtcttcaccttagcaagtggcccttggagcatgactctggatctttacttagtgtcacttgttttcagtgagctgagtactgctgggcccagctatagcaaaagtcttgacagaagcaacctgaattcctgagtctgtgatacattaaatgtacacacgcacgcacgcacacacgcacgcacaaacgcaagcacgcacgcaggcacttcacttagctcctagcagaaaggaagtcctagtgctgggtgctggacccacataggagatgcagctccggaaggcctcagcactgtgtgggtgaagcggtgtacgtgtgttctttgcagtgttataacattgctcacgctctctcgggactttggatttgcagatactaatgctggagaccgtggacagaccaataacgccgcttctgcatcagcctccaactctacctgaacagccccaagtagccagctgcgcagggaagaccagcacttacttgagtgccactcagcaacactggtcacgtttggaaagaaaattaaaaagaggaaaacaaaaacaaaaacaaaaaacccctgttcattttagtgttcaatttttttattgttgttcttatttaaccttgtaaaatatctataaatacaaaccagattcattgtattctcacttttcaggagatccaagaggtggggagggttggaaggggaggggcagagcactaaacacacagtctctccctcgacaatctcttcctttcctttctttttaaaatcagaatccgctattgtataccgtaaccagactcctgcctcatggcccaccgccgcagaagacctgttctgtgctggtttactttctgaatttgttttcttttaaagtctggcgtaagaccgatacagtgcacagcttgaaattggttgggagctcggcaggcattactccgcagggacttcctgtcacttgtgaaatgtatgct

SEQ ID NO: 17 - DNA, 1474 bp, Rattus norvegicus
ttttttcttcgcgggagaacttaatgctgcatttattattaacctagtaccctaacataaaacaaaaggaagaaaaggaccaaggaaggaaaaggtgaatcgagaagagccatcatgtcggggcgaccgagaaccacctcctttgcggagagctgcaagccagtgcagcagccttcagcttttggtagcatgaaagttagcagagataaagatggcagcaaggtaaccacagtggtggcaactcctggacagggtcctgacaggccacaggaagtcagttacacagacactaaagtcattggaaatgggtcatttggtgtggtatatcaagccaaactttgtgactcaggagaactggtggccatcaagaaagttcttcaggacaagcgatttaagaaccgagagctccagatcatgagaaagctagatcactgtaacatagtccgattgcggtatttcttctactcgagtggcgagaagaaagatgaggtctaccttaacctggtgctggactatgttccggaaacagtgtacagagtcgccagacactatagtcgagccaagcagacactccctgtgatctatgtcaagttgtatatgtaccagctgttcagaagtctagcctatatccattcctttgggatctgccatcgagacattaaaccacagaacctcttgctggatcctgatacagctgtattaaaactctgcgactttggaagtgcaaagcagctggtccgaggagagcccaatgtttcatatatctgttctcggtactacagggcaccagagctgatctttggagccaccgattacacgtctagtatagatatgtggtctgcaggctgtgtgttggctgaattgttgctaggacaaccaatatttcctggggacagtggtgtggatcagttggtggaaataataaaggtcctaggaacaccaacaagggagcaaattagagaaatgaacccaaattatacagaattcaaattcccccaaatcaaggcacatccttggacgaaggtctttcggccccgaactccaccagaggcaatcgcactgtgtagccgtctcctggagtacacgccgaccgcccggctaacaccactggaagcttgtgcacattcattttttgatgaattacgggacccaaatgtcaaactaccaaatgggcgagacacacctgccctcttcaactttaccactcaagaactgtcaagtaacccacccctggccaccatccttatccctcctcacgctcggattcaggcagctgcttcacctcctgcaaacgccacagcagcctcagatactaatgctggagaccgtggacagaccaataacgccgcttctgcatcagcctccaactctacctgaacagccccaagtagccagctgcgcagggaagaccagcacttacttgagtgccactcagcaacactggtcacgtttggaaagaaaattaaaaaaaaaa

SEQ ID NO: 18 - DNA, 674 bp, Rattus norvegicus
gaggaccgagtgcccttcctccttctggcaccatgcagctctcactagccccttgccttgcctgcctgcttgtacatgcagccttcgttgctgtggagagccaggggtggcaagccttcaagaatgatgccacagaaatcatcccgggactcagagagtacccagagcctcctcaggaactagagaacaaccagaccatgaaccgggccgagaacggaggcagacccccccaccatccttatgacaccaaagacgtgtccgagtacagctgccgcgagctgcactacacccgcttcgtgaccgacggcccgtgccgcagtgccaagccggtcaccgagttggtgtgctcgggccagtgcggccccgcgcggctgctgcccaacgccatcgggcgcgtgaagtggtggcgcccgaacggacccgacttccgctgcatcccggatcgctaccgcgcgcagcgggtgcagctgctgtgccccggcggcgcggcgccgcgctcgcgcaaggtgcgtctggtggcctcgtgcaagtgcaagcgcctcacccgcttccacaaccagtcggagctcaaggacttcggacctgagaccgcgcggccgcagaagggtcgcaagccgcggccccgcgcccggggagccaaagccaaccaggcggagctggagaacgcctactag

SEQ ID NO: 19 - DNA, 475 bp, Rattus norvegicus
cgtgcgcgactcgtgcgagccggtcatgcagttcttcggcttctactggccggagatgctcaaatgtgacaagttccccgagggcgacgtctgcatcgccatgaccccgcccaatgccaccgaagcctcgaagccccaaggtacaacagtgtgtcctccgtgtgacaacgagttgaaatcggaggccatcatcgaacatctctgtgcaagcgagtttgcgctgagaatgaaaatcaaagaagtaaaaaaggaaaacggtgacaagaagattgtccccaagaagaagaaacccttgaagctggggcccatcaagaagaaggagctgaagcggctcgtgcttttcctaaagaatggcgccgactgtccctgccaccagctggacaacctcagccacaacttcctcatcatggggcgcaaggtgaagagccagtacttgctcacagccattcacaagtgggacaagaaaaacaaggag

SEQ ID NO: 20 - DNA, 1206 bp, Homo sapiens
gttgctgtcggggagttgaaacctaattttgtggcgtagagctatgcagcttgaaatccaagtagcactaaattttattatttcgtatttgtacaataagcttcccaggagacgtgtcaacatttttggtgaagaacttgaaagacttcttaagaagaaatatgaagggcactggtatcctgaaaagccatacaaaggatcggggtttagatgtatacacataggggagaaagtggacccagtgattgaacaagcatccaaagagagtggtttggacattgatgatgttcgtggcaatctgccacaggatcttagtgtttggatcgacccatttgaggtttcttaccaaattggtgaaaagggaccagtgaaggtgctttacgtggatgataataatgaaaatggatgtgagttggataaggagatcaaaaacagctttaacccagaggcccaggtttttatgcccataagtgacccagcctcatcagtgtccagctctccatcgcctccttttggtcactctgctgctgtaagccctaccttcatgccccggtccactcagcctttaacctttaccactgccacttttgctgccaccaagttcggctctaccaaaatgaagaatagtggccgtagcaacaaggttgcacgtacttctcccatcaacctcggcttgaatgtgaatgacctcttgaagcagaaagccatctcttcctcaatgcactctctgtatgggcttggcttgggtagccagcagcagccacagcaacagcagcagccagcccagccgccaccgccaccaccaccaccacagcagcaacaacagcagaaaacctctgctctttctcctaatgccaaggaatttatttttcctaatatgcagggtcaaggtagtagtaccaatggaatgttcccaggtgacagcccccttaacctcagtcctctccagtacagtaatgcctttgatgtgtttgcagcctatggaggcctcaatgagaagtcttttgtagatggcttgaattttagcttaaataacatgcagtattctaaccagcaattccagcctgttatggctaactaaaaaaaagaaaatgtatcgtacaagttaaaatgcacgggcccaagggggattttttttttcacctccttgagaatttttttttttttaagcttatagtaaggatacattcaagcttgggttaaaaa

SEQ ID NO: 21 - DNA, 2024 bp, Rattus norvegicus
gaccacggaaggttgaaggataatcttctagtaccagcactttgaatgagaatttgtttctctgccacaaactgatttttttttttaaattttatttttctggtggaggagttgaaacaaacctaccttttgtggcatcgcagctatgcagcttgaaattcaagtagcactaaattttattatttcctatttgtacaataagcttcccaggagacgtgtcaacatttttggtgaagaacttgaaagacttcttaaacagaaatatgaagggcattggtatcctgagaagccatacaaaggctcagggtttagatgcatacacgtaggggagaaggtggacccggtgatcgagcaagcgtccaaagagagtggtctggacattgacgatgttcgtggcaatctgccgcaggatctgagcgtgtggatcgacccgtttgaggtttcctaccaaatcggtgagaagggaccagtgaaggtgctgtacgtagatgacagtaatgagaacgggtgtgagctggataaggagatcaagaacagctttaaccctgaggcccaggttttcatgcccataagcgacccagcctcctctgtgtccagctcgccgtcgcctccctttggccattctgctgccgtcagccccaccttcatgccccggtccactcagcctttaacctttaccactgccacttttgctgccaccaagtttggctccaccaaaatgaagaacagtggccgtagcagcaaggtagcacgcacttctcccatcagcctgggcctgaatgtcaatgtgaacgacctcctgaagcagaaagccatctcttcctcaatgcactctctgtacgggctgggcctgggcagccagcagcagcctcagccgcagccgcagcagccgccatcccagccaccaccgccaccaccacctccacagcagcagcagcagcatcagcagcagcagcagcagcagcaacagcagcagcagcagccgcagcagcaaacctctgctctttctcccaatgccaaggaatttatttttcctaacatgcagggtcaaggtagtagtaccaatggaatgttcccaggtgacagcccccttaacctcagtcccctccagtacagtaatgcctttaatgtgtttgcggcctacggaggcctcaacgagaagtctttcgtagacggcttgaattttagcttaaataacattcagtattctaaccagcagttccagcccgtaatggctaactaaaaaaacaggaaaagagagaacatgtgtcgtacaagttaaaatgcatcggcccaagggggacttttttttttttttgcctccttgagattttttttttaagcttatagtaaggatacattcaagcttggtcacaaaataaaacatgcatcttttttcatttgccaaccaagcacaatgttattttatactgactgtatattttaaagtatactttcagatatggcctcttacagtatttaagatatagcaaggacatggctgatttttttttatatatatatataaaaaattggcactaataagtgggtttattggtcttttctaattgtataatttaatttagtacaaagtttgtaaactatcagaggatatatatatatatacatatatatattgtttctacgacacggtattgcatttctatctttttactacagtgatctgtggcagcggcttcatgttgtattttttttactgaaattgtaaaatatccatcttaaagacatcaactattctgaaaattgtgtacaggatattcctttagtggtggaattaaaatgtacgaatatttgctttttcaaaaaaatgtattttctgttaaaggtttaaagatttttgctatatattatggaagaaaatgtaatcgtaaatattaattttgtacctatattgtgcaatacttgaaaaaaaaacggtataaaagtattttgagtcagtgtcttacatgttaagagggactgaaatagtttatattaagtttgtattaaaattctttaaaatt

SEQ ID NO: 22 - DNA, 2828 bp, Rattus norvegicus
tgatgtttccaaaaggaactcgggagtcacttctaaaatggtgaccagcaaggaaactagtgaccaacttgtacaagtggagtgtgggggggtggatgagtgtgcagtacccgttagtaaacactagaaagacaggatacatactctggaggttccctaagtcctgctcccaaacggtgtgtaatcgcttagagtgctgttttaactataaacctgtcttgaactttagcaagagattgttttgtggtctgttcctttattctgtagcggtattttccagtctttctagcccagtagacactttcaaaaataaaactgcttcaggtaaaaagcttttggagctagtccattgagctcacttggatgtgtttgcttttgttttgttttcttaaaggaacaagaccacggaaggttgaaggataatcttctagtaccagcactttgaatgagaatttgtttctctgccacaaactgattttttttttaaattttatttttctggtggaggagttgaaacaaacctaccttttgtggcatcgcagctatgcagcttgaaattcaagtagcactaaattttattatttcctatttgtacaataagcttcccaggagacgtgtcaacatttttggtgaagaacttgaaagacttcttaaacagaaatatgaagggcattggtatcctgagaagccatacaaaggctcagggtttagatgcatacacgtaggggagaaggtggacccggtgatcgagcaagcgtccaaagagagtggtctggacattgacgatgttcgtggcaatctgccgcaggatctgagcgtgtggatcgacccgtttgaggtttcctaccaaatcggtgagaagggaccagtgaaggtgctgtacgtagatgacagtaatgagaacgggtgtgagctggataaggagatcaagaacagctttaaccctgaggcccaggttttcatgcccataagcgacccagcctcctctgtgtccagctcgccgtcgcctccctttggccattctgctgccgtcagccccaccttcatgccccggtccactcagcctttaacctttaccactgccacttttgctgccaccaagtttggctccaccaaaatgaagaacagtggccgtagcagcaaggtagcacgcacttctcccatcagcctgggcctgaatgtcaatgtgaacgacctcctgaagcagaaagccatctcttcctcaatgcactctctgtacgggctgggcctgggcagccagcagcagcctcagccgcagccgcagcagccgccatcccagccaccaccgccgccaccacctccacagcagcagcagcagcagcagcagcagcagcagcagcagcagcaacagcagcagcagcagccgcagcagcaaacctctgctctttctcccaatgccaaggaatttatttttcctaacatgcagggtcaaggtagtagtaccaatggaatgttcccaggtgacagcccccttaacctcagtcccctccagtacagtaatgcctttgatgtgtttgcggcctacggaggcctcaacgagaagtctttcgtagacggcttgaattttagcttaaataacattcagtattctaaccagcagttccagcccgtaatggctaactaaaaaaacaggaaaagagagaacatgtgtcgtacaagttaaaatgcatcggcccaagggggacttttttttttttttttgcctccttgagattttttttttaagcttatagtaaggatacattcaagcttggtcacaaaataaaacatgcatcttttttcatttgccaaccaagcacaatgttattttatactgactgtatattttaaagtatactttcagatatggcctcttacagtatttaagatatagcaaggacatggctgatttttttttatatatatatataaaaaattggcactaataagtgggtttattggtcttttctaattgtataatttaatttagtacaaagtttgtaaactatcagaggatatatatatatatacatatatatattgtttctacgacacggtattgcatttctatctttttactacagtgatctgtggcagcggcttcatgttgtattttttttactgaaattgtaaaatatccatcttaaagacatcaactattctgaaaattgtgtacaggatattcctttagtggtggaattaaaatgtacgaatatttgctttttcaaaaaaatgtattttctgttaaaggtttaaagatttttgctatatattatggaagaaaatgtaatcgtaaatattaattttgtacctatattgtgcaatacttgaaaaaaaaacggtataaaagtattttgagtcagtgtcttacatgttaagagggactgaaatagtttatattaagtttgtattaaaattctttaaaattaaaaatgcctatcatgtggtcttttgctaaaagcctgtgctcatgaagaaggaagatgtggctcgctgagtcagggagcagtaatggaaggcagggctctgccagagcatcggtggcgcttggtagagtgtttgcctcaaaagcagaaagccatgggttcaatccccagcaccctatgaatgtcagtggtacgtgcacacctctactccagcatccatctaggagagagccgtggggtaggaggacgtcgtgtaatccctcagcatctatttaaattgtgagaaggaagtcaggaaatgctagaaaacccttcaccttttacattcagccatggtctgtagctagaatttagggttaaacccatgcacagttgggaatgttcatgtctgtttcttttccc

SEQ ID NO: 23 - DNA, 20 bp, Artificial Sequence
  Antisense Compound
ccctactcagtaggcattgg

SEQ ID NO: 24 - DNA, 20 bp, Artificial Sequence
  Antisense Compound
gcccaagctggcatccgtca

SEQ ID NO: 25 - DNA, 20 bp, Artificial Sequence
  Antisense Compound
cgtgtgtctgtgctagtccc

SEQ ID NO: 26 - DNA, 20 bp, Artificial Sequence
  Antisense Compound
ggcaacgtgaacaggtccaa

SEQ ID NO: 27 - DNA, 20 bp, Artificial Sequence
  Antisense Compound
tattccacgaacgtaggctg

SEQ ID NO: 28 - DNA, 20 bp, Artificial Sequence
  Antisense Compound
aggtagctttgattatgtaa

SEQ ID NO: 29 - DNA, 18 bp, Artificial Sequence
  Antisense Compound
gcccattgctggacatgc

SEQ ID NO: 30 - DNA, 20 bp, Artificial Sequence
  Antisense Compound
agcccattgctggacatgca

SEQ ID NO: 31 - DNA, 20 bp, Artificial Sequence
  Antisense Compound
gcttgtgtgctctgctgtct

SEQ ID NO: 32 - DNA, 20 bp, Artificial Sequence
  Antisense Compound
ttgtcccagtcccaggcctc

SEQ ID NO: 33 - DNA, 20 bp, Artificial Sequence
  Antisense Compound
ctctctgtaggcccgcttgg

SEQ ID NO: 34 - DNA, 20 bp, Artificial Sequence
  Antisense Compound
ctttccgttggacccctggg

SEQ ID NO: 35 - DNA, 20 bp, Artificial Sequence
  Antisense Compound
ctgatcatagcgagtaagta

SEQ ID NO: 36 - DNA, 20 bp, Artificial Sequence
  Antisense Compound
gtgcgcgcgagcccgaaatc

SEQ ID NO: 37 - DNA, 20 bp, Artificial Sequence
  Antisense Compound
gctcagtggacatggatgag

SEQ ID NO: 38 - DNA, 20 bp, Artificial Sequence
  Antisense Compound
atccaagtgctactgtagta

SEQ ID NO: 39 - DNA, 22 bp, Artificial Sequence
  Antisense Compound
ataccgcgatcagtgcatcttt

SEQ ID NO: 40 - DNA, 22 bp, Artificial Sequence
  Antisense Compound
agactagcggtatctttatccc

SEQ ID NO: 41 - DNA, 20 bp, Artificial Sequence
  Antisense Compound
  misc_feature 1-20: n is a, c, g, or t
nnnnnnnnnnnnnnnnnnnn

SEQ ID NO: 42 - DNA, 20 bp, Artificial Sequence
  Antisense Compound
aatattcgcaccccactggt

SEQ ID NO: 43 - DNA, 20 bp, Artificial Sequence
  Antisense Compound
cgttattaacctccgttgaa

SEQ ID NO: 44 - DNA, 20 bp, Artificial Sequence
  Antisense Compound
acaagcgtcaaccgtattat

SEQ ID NO: 45 - DNA, 20 bp, Artificial Sequence
  Antisense Compound
ttcgcggctggacgattcag

SEQ ID NO: 46 - DNA, 20 bp, Artificial Sequence
  Antisense Compound
gaggtctcgacttacccgct

SEQ ID NO: 47 - DNA, 20 bp, Artificial Sequence
  Antisense Compound
ttagaatacgtcgcgttatg

SEQ ID NO: 48 - DNA, 20 bp, Artificial Sequence
  Antisense Compound
atgcatactacgaaaggccg

SEQ ID NO: 49 - DNA, 20 bp, Artificial Sequence
  Antisense Compound
acatgggcgcgcgactaagt

SEQ ID NO: 50 - DNA, 20 bp, Artificial Sequence
  Antisense Compound
gtacagttatgcgcggtaga

SEQ ID NO: 51 - DNA, 20 bp, Artificial Sequence
  Antisense Compound
ttctacctcgcgcgatttac

SEQ ID NO: 52 - DNA, 20 bp, Artificial Sequence
  Antisense Compound
attcgccagacaacactgac

SEQ ID NO: 53 - DNA, 20 bp, Artificial Sequence
  Antisense Compound
aataagtacgtactattgtc

SEQ ID NO: 54 - DNA, 20 bp, Artificial Sequence
  Antisense Compound
tttgatcgaggttagccgtg

SEQ ID NO: 55 - DNA, 20 bp, Artificial Sequence
  Antisense Compound
ggatagaacgcgaaagcttg

SEQ ID NO: 56 - DNA, 20 bp, Artificial Sequence
  Antisense Compound
tagtgcggacctacccacga

SEQ ID NO: 57 - DNA, 20 bp, Artificial Sequence
  Antisense Compound
gccctccatgctggcacagg

SEQ ID NO: 58 - DNA, 20 bp, Artificial Sequence
  Antisense Compound
ctcttactgtgctgtggaca

SEQ ID NO: 59 - DNA, 20 bp, Artificial Sequence
  Antisense Compound
agcaaaagatcaatccgtta

SEQ ID NO: 60 - DNA, 20 bp, Artificial Sequence
  Antisense Compound
ggaccccgaaagaccaccag

SEQ ID NO: 61 - DNA, 20 bp, Artificial Sequence
  Antisense Compound
ctgctagcctctggatttga

SEQ ID NO: 62 - DNA, 20 bp, Artificial Sequence
  Antisense Compound
ctgcctggatgggtgttttt

SEQ ID NO: 63 - DNA, 20 bp, Artificial Sequence
  Antisense Compound
tcccgcctgtgacatgcatt

SEQ ID NO: 64 - DNA, 20 bp, Artificial Sequence
  Antisense Compound
tacagaaggctgggccttga

SEQ ID NO: 65 - DNA, 20 bp, Artificial Sequence
  Antisense Compound
atgcattctgcccccaagga

SEQ ID NO: 66 - DNA, 18 bp, Artificial Sequence
  Antisense Compound
ccaacctcaaatgtccca

SEQ ID NO: 67 - DNA, 20 bp, Artificial Sequence
  Antisense Compound
aggcatggtctttgtcaata

SEQ ID NO: 68 - DNA, 18 bp, Artificial Sequence
  Antisense Compound
tgtgctattctgtgaatt

SEQ ID NO: 69 - DNA, 20 bp, Artificial Sequence
  Antisense Compound
tacgtccggaggcgtacgcc

SEQ ID NO: 70 - DNA, 21 bp, Artificial Sequence
  Forward Primer
caacggatttggtcgtattgg

SEQ ID NO: 71 - DNA, 26 bp, Artificial Sequence
  Reverse Primer
ggcaacaatatccactttaccagagt

SEQ ID NO: 72 - DNA, 21 bp, Artificial Sequence
  Probe
cgcctggtcaccagggctgct

SEQ ID NO: 73 - DNA, 19 bp, Artificial Sequence
  Forward Primer
gaaggtgaaggtcggagtc

SEQ ID NO: 74 - DNA, 20 bp, Artificial Sequence
  Reverse Primer
gaagatggtgatgggatttc

SEQ ID NO: 75 - DNA, 20 bp, Artificial Sequence
  Probe
caagcttcccgttctcagcc

SEQ ID NO: 76 - DNA, 20 bp, Artificial Sequence
  Probe
tggaatcatattggaacatg

SEQ ID NO: 77 - DNA, 20 bp, Artificial Sequence
  Forward Primer
ggcaaattcaacggcacagt

SEQ ID NO: 78 - DNA, 20 bp, Artificial Sequence
  Reverse Primer
gggtctcgctcctggaagat

SEQ ID NO: 79 - DNA, 27 bp, Artificial Sequence
  Probe
aaggccgagaatgggaagcttgtcatc

SEQ ID NO: 80 - DNA, 23 bp, Artificial Sequence
  Forward Primer
tgttctagagacagccgcatctt

SEQ ID NO: 81 - DNA, 21 bp, Artificial Sequence
  Reverse Primer
caccgaccttcaccatcttgt

SEQ ID NO: 82 - DNA, 24 bp, Artificial Sequence
  Probe
ttgtgcagtgccagcctcgtctca

SEQ ID NO: 83 - DNA, 18 bp, Artificial Sequence
  Forward Primer
ccgcacgcaattcaacag

SEQ ID NO: 84 - DNA, 20 bp, Artificial Sequence
  Reverse Primer
gacacaggccatcagcatgt

SEQ ID NO: 85 - DNA, 28 bp, Artificial Sequence
  Probe
ctgcagcagcttgtggcttactactcca

SEQ ID NO: 86 - DNA, 25 bp, Artificial Sequence
  Forward Primer
aagatcaccatcaagccagtaattc

SEQ ID NO: 87 - DNA, 23 bp, Artificial Sequence
  Reverse Primer
aaaaggagttcactgcatttgga

SEQ ID NO: 88 - DNA, 35 bp, Artificial Sequence
  Probe
ctaggcttcacacttgtcagagacactaaaccagc

SEQ ID NO: 89 - DNA, 27 bp, Artificial Sequence
  Forward Primer
caagtaccagactcttgacaactacca

SEQ ID NO: 90 - DNA, 18 bp, Artificial Sequence
  Reverse Primer
tgccgcactcctcatcct

SEQ ID NO: 91 - DNA, 15 bp, Artificial Sequence
  Probe
ccctacccttgcgcg

SEQ ID NO: 92 - DNA, 23 bp, Artificial Sequence
  Forward Primer
ggacccaaatgtcaaactaccaa

SEQ ID NO: 93 - DNA, 27 bp, Artificial Sequence
  Reverse Primer
tgacagttcttgagtggtaaagttgaa

SEQ ID NO: 94 - DNA, 21 bp, Artificial Sequence
  Probe
tgggcgagacacacctgccct

SEQ ID NO: 95 - DNA, 17 bp, Artificial Sequence
  Forward Primer
ctggtggcctcgtgcaa

SEQ ID NO: 96 - DNA, 22 bp, Artificial Sequence
  Reverse Primer
tctcaggtccgaagtccttgag

SEQ ID NO: 97 - DNA, 21 bp, Artificial Sequence
  Probe
ccgcttccacaaccagtcgga

SEQ ID NO: 98 - DNA, 21 bp, Artificial Sequence
  Forward Primer
tgcgctgagaatgaaaatcaa

SEQ ID NO: 99 - DNA, 23 bp, Artificial Sequence
  Reverse Primer
ccagcttcaagggtttcttcttc

SEQ ID NO: 100 - DNA, 36 bp, Artificial Sequence
  Probe
aagtaaaaaaggaaaacggtgacaagaagattgtcc

SEQ ID NO: 101 - DNA, 24 bp, Artificial Sequence
  Forward Primer
agagtggtttggacattgatgatg

SEQ ID NO: 102 - DNA, 21 bp, Artificial Sequence
  Reverse Primer
caaatgggtcgatccaaacac

SEQ ID NO: 103 - DNA, 25 bp, Artificial Sequence
  Probe
tcgtggcaatctgccacaggatctt

SEQ ID NO: 104 - DNA, 22 bp, Artificial Sequence
  Forward Primer
gcctgaatgtcaatgtgaacga

SEQ ID NO: 105 - DNA, 18 bp, Artificial Sequence
  Reverse Primer
gcccagcccgtacagaga

SEQ ID NO: 106 - DNA, 29 bp, Artificial Sequence
  Probe
aagcagaaagccatctcttcctcaatgca

SEQ ID NO: 107 - DNA, 20 bp, Artificial Sequence
  Antisense Compound
gtctccctctgtgttattga

SEQ ID NO: 108 - DNA, 20 bp, Artificial Sequence
  Antisense Compound
gttccacccagaagcctcca

SEQ ID NO: 109 - DNA, 20 bp, Artificial Sequence
  Antisense Compound
ttgctcttgttgctgcccat

SEQ ID NO: 110 - DNA, 20 bp, Artificial Sequence
  Antisense Compound
tccgaagagcttgggctcgg

SEQ ID NO: 111 - DNA, 20 bp, Artificial Sequence
  Antisense Compound
agcctccgaagagcttgggc

SEQ ID NO: 112 - DNA, 20 bp, Artificial Sequence
  Antisense Compound
gttgaagcctccgaagagct

SEQ ID NO: 113 - DNA, 20 bp, Artificial Sequence
  Antisense Compound
gagttgaagcctccgaagag

SEQ ID NO: 114 - DNA, 20 bp, Artificial Sequence
  Antisense Compound
cgaggagttgaagcctccga

SEQ ID NO: 115 - DNA, 20 bp, Artificial Sequence
  Antisense Compound
tccgaggagttgaagcctcc

SEQ ID NO: 116 - DNA, 20 bp, Artificial Sequence
  Antisense Compound
gtgtccgaggagttgaagcc

SEQ ID NO: 117 - DNA, 20 bp, Artificial Sequence
  Antisense Compound
gtcaccccacctgccagagg

SEQ ID NO: 118 - DNA, 20 bp, Artificial Sequence
  Antisense Compound
tcatagagggccacaaaggt

SEQ ID NO: 119 - DNA, 20 bp, Artificial Sequence
  Antisense Compound
tctctgtccgtgactcatag

SEQ ID NO: 120 - DNA, 20 bp, Artificial Sequence
  Antisense Compound
agtctctgtccgtgactcat

SEQ ID NO: 121 - DNA, 20 bp, Artificial Sequence
  Antisense Compound
aggacaggtcagtctctgtc

SEQ ID NO: 122 - DNA, 20 bp, Artificial Sequence
  Antisense Compound
aaggacaggtcagtctctgt

SEQ ID NO: 123 - DNA, 20 bp, Artificial Sequence
  Antisense Compound
cgctcccctttcttgaagga

SEQ ID NO: 124 - DNA, 20 bp, Artificial Sequence
  Antisense Compound
cagccgctcccctttcttga

SEQ ID NO: 125 - DNA, 20 bp, Artificial Sequence
  Antisense Compound
ttgacaatctgcagccgctc

SEQ ID NO: 126 - DNA, 20 bp, Artificial Sequence
  Antisense Compound
cgtgttattgacaatctgca

SEQ ID NO: 127 - DNA, 20 bp, Artificial Sequence
  Antisense Compound
acatccaccttcctcgtgtt

SEQ ID NO: 128 - DNA, 20 bp, Artificial Sequence
  Antisense Compound
gagtgtgccagccaccagtc

SEQ ID NO: 129 - DNA, 20 bp, Artificial Sequence
  Antisense Compound
gctcagcgagtgtgccagcc

SEQ ID NO: 130 - DNA, 20 bp, Artificial Sequence
  Antisense Compound
tgctcagcgagtgtgccagc

SEQ ID NO: 131 - DNA, 20 bp, Artificial Sequence
  Antisense Compound
tcagcctggatggagtcgga

SEQ ID NO: 132 - DNA, 20 bp, Artificial Sequence
  Antisense Compound
ctcagcctggatggagtcgg

SEQ ID NO: 133 - DNA, 20 bp, Artificial Sequence
  Antisense Compound
ccgtctagtgatcttgccaa

SEQ ID NO: 134 - DNA, 20 bp, Artificial Sequence
  Antisense Compound
ctctgattcccgtctagtga

SEQ ID NO: 135 - DNA, 20 bp, Artificial Sequence
  Antisense Compound
agccgctctgattcccgtct

SEQ ID NO: 136 - DNA, 20 bp, Artificial Sequence
  Antisense Compound
cctcacgaggaaggtccctc

SEQ ID NO: 137 - DNA, 20 bp, Artificial Sequence
  Antisense Compound
ggtctcactctccctcacga

SEQ ID NO: 138 - DNA, 20 bp, Artificial Sequence
  Antisense Compound
atacagagaggcagtaggca

SEQ ID NO: 139 - DNA, 20 bp, Artificial Sequence
  Antisense Compound
gtcggatacagagaggcagt

SEQ ID NO: 140 - DNA, 20 bp, Artificial Sequence
  Antisense Compound
tttaggcccttggcattgtc

SEQ ID NO: 141 - DNA, 20 bp, Artificial Sequence
  Antisense Compound
atttaggcccttggcattgt

SEQ ID NO: 142 - DNA, 20 bp, Artificial Sequence
  Antisense Compound
gtgtttcacatttaggccct

SEQ ID NO: 143 - DNA, 20 bp, Artificial Sequence
  Antisense Compound
atgtttggagtagtaagcca

SEQ ID NO: 144 - DNA, 20 bp, Artificial Sequence
  Antisense Compound
aggccatcagcatgtttgga

SEQ ID NO: 145 - DNA, 20 bp, Artificial Sequence
  Antisense Compound
cggtgacacaggccatcagc

SEQ ID NO: 146 - DNA, 20 bp, Artificial Sequence
  Antisense Compound
tgaggcttggatgtgggaca

SEQ ID NO: 147 - DNA, 20 bp, Artificial Sequence
  Antisense Compound
ccctgggtctgaggcttgga

SEQ ID NO: 148 - DNA, 20 bp, Artificial Sequence
  Antisense Compound
acctccagccgcagggactc

SEQ ID NO: 149 - DNA, 20 bp, Artificial Sequence
  Antisense Compound
cagcttgacctccagccgca

SEQ ID NO: 150 - DNA, 20 bp, Artificial Sequence
  Antisense Compound
ctggcccagcttgacctcca

SEQ ID NO: 151 - DNA, 20 bp, Artificial Sequence
  Antisense Compound
gcaaccctggcccagcttga

SEQ ID NO: 152 - DNA, 20 bp, Artificial Sequence
  Antisense Compound
acctctccgaagcaaccctg

SEQ ID NO: 153 - DNA, 20 bp, Artificial Sequence
  Antisense Compound
tccacacctctccgaagcaa

SEQ ID NO: 154 - DNA, 20 bp, Artificial Sequence
  Antisense Compound
cgtggtgccgttccaggtcc

SEQ ID NO: 155 - DNA, 20 bp, Artificial Sequence
  Antisense Compound
atggcaaccctcgtggtgcc

SEQ ID NO: 156 - DNA, 20 bp, Artificial Sequence
  Antisense Compound
tgatggcaaccctcgtggtg

SEQ ID NO: 157 - DNA, 20 bp, Artificial Sequence
  Antisense Compound
cttgggcctcctgcaggaag

SEQ ID NO: 158 - DNA, 20 bp, Artificial Sequence
  Antisense Compound
tcagtttcttcatgacttgg

SEQ ID NO: 159 - DNA, 20 bp, Artificial Sequence
  Antisense Compound
ccttgttcatgtactctgtc

SEQ ID NO: 160 - DNA, 20 bp, Artificial Sequence
  Antisense Compound
gcagactccccttgttcatg

SEQ ID NO: 161 - DNA, 20 bp, Artificial Sequence
  Antisense Compound
cagcagactccccttgttca

SEQ ID NO: 162 - DNA, 20 bp, Artificial Sequence
  Antisense Compound
cgtttcccccttgagaaagt

SEQ ID NO: 163 - DNA, 20 bp, Artificial Sequence
  Antisense Compound
tatttgcccgtttccccctt

SEQ ID NO: 164 - DNA, 20 bp, Artificial Sequence
  Antisense Compound
agacatgtccaccagctggg

SEQ ID NO: 165 - DNA, 20 bp, Artificial Sequence
  Antisense Compound
tcatccgctccacataggcc

SEQ ID NO: 166 - DNA, 20 bp, Artificial Sequence
  Antisense Compound
ccggtgcacatagttcatcc

SEQ ID NO: 167 - DNA, 20 bp, Artificial Sequence
  Antisense Compound
cactttgcacaccaggttct

SEQ ID NO: 168 - DNA, 20 bp, Artificial Sequence
  Antisense Compound
gtcggccactttgcacacca

SEQ ID NO: 169 - DNA, 20 bp, Artificial Sequence
  Antisense Compound
cccaaagtcggccactttgc

SEQ ID NO: 170 - DNA, 20 bp, Artificial Sequence
  Antisense Compound
gtgagctcagtcagcaggat

SEQ ID NO: 171 - DNA, 20 bp, Artificial Sequence
  Antisense Compound
gacaaggcatccggtagccc

SEQ ID NO: 172 - DNA, 20 bp, Artificial Sequence
  Antisense Compound
cagcactggcacatgaggtc

SEQ ID NO: 173 - DNA, 20 bp, Artificial Sequence
  Antisense Compound
gccagcactggcacatgagg

SEQ ID NO: 174 - DNA, 20 bp, Artificial Sequence
  Antisense Compound
ttccgccagcactggcacat

SEQ ID NO: 175 - DNA, 20 bp, Artificial Sequence
  Antisense Compound
tccttccgccagcactggca

SEQ ID NO: 176 - DNA, 20 bp, Artificial Sequence
  Antisense Compound
ggctccttccgccagcactg

SEQ ID NO: 177 - DNA, 20 bp, Artificial Sequence
  Antisense Compound
ggccgctcctcaggctcctt

SEQ ID NO: 178 - DNA, 20 bp, Artificial Sequence
  Antisense Compound
gggccgctcctcaggctcct

SEQ ID NO: 179 - DNA, 20 bp, Artificial Sequence
  Antisense Compound
actcgaaggtgggccgctcc

SEQ ID NO: 180 - DNA, 20 bp, Artificial Sequence
  Antisense Compound
ctataggttctccccgggct

SEQ ID NO: 181 - DNA, 20 bp, Artificial Sequence
  Antisense Compound
acacagttcctataggttct

SEQ ID NO: 182 - DNA, 20 bp, Artificial Sequence
  Antisense Compound
caccccctgtacagacggaa

SEQ ID NO: 183 - DNA, 20 bp, Artificial Sequence
  Antisense Compound
tcagcatgttctggagacgg

SEQ ID NO: 184 - DNA, 20 bp, Artificial Sequence
  Antisense Compound
acaccagccgtagtgtccgg

SEQ ID NO: 185 - DNA, 20 bp, Artificial Sequence
  Antisense Compound
gtcctgactgcagggagcac

SEQ ID NO: 186 - DNA, 20 bp, Artificial Sequence
  Antisense Compound
ctacgaaggagaagacagta

SEQ ID NO: 187 - DNA, 20 bp, Artificial Sequence
  Antisense Compound
ccggttcggctgcagagtca

SEQ ID NO: 188 - DNA, 20 bp, Artificial Sequence
  Antisense Compound
gcaagcctgggtccccagga

SEQ ID NO: 189 - DNA, 20 bp, Artificial Sequence
  Antisense Compound
actttgcaagcctgggtccc

SEQ ID NO: 190 - DNA, 20 bp, Artificial Sequence
  Antisense Compound
accgtcactttgcaagcctg

SEQ ID NO: 191 - DNA, 20 bp, Artificial Sequence
  Antisense Compound
agaaggactcaagagggaga

SEQ ID NO: 192 - DNA, 20 bp, Artificial Sequence
  Antisense Compound
cagagccatcatctcagaag

SEQ ID NO: 193 - DNA, 20 bp, Artificial Sequence
  Antisense Compound
agacccgggtagctcccgct

SEQ ID NO: 194 - DNA, 20 bp, Artificial Sequence
  Antisense Compound
ccagcagagggtggccgccg

SEQ ID NO: 195 - DNA, 20 bp, Artificial Sequence
  Antisense Compound
ggaattgagaaccgagttca

SEQ ID NO: 196 - DNA, 20 bp, Artificial Sequence
  Antisense Compound
gcctgggtgccccgcagcgc

SEQ ID NO: 197 - DNA, 20 bp, Artificial Sequence
  Antisense Compound
gctgactgcagagcctgggt

SEQ ID NO: 198 - DNA, 20 bp, Artificial Sequence
  Antisense Compound
cccgggtacaggattcccgg

SEQ ID NO: 199 - DNA, 20 bp, Artificial Sequence
  Antisense Compound
gtacttattcccgcccgggt

SEQ ID NO: 200 - DNA, 20 bp, Artificial Sequence
  Antisense Compound
ttgtcaatggtctggtactt

SEQ ID NO: 201 - DNA, 20 bp, Artificial Sequence
  Antisense Compound
acgggtacggctggtagttg

SEQ ID NO: 202 - DNA, 20 bp, Artificial Sequence
  Antisense Compound
agtgccgcactcctcgtcct

SEQ ID NO: 203 - DNA, 20 bp, Artificial Sequence
  Antisense Compound
ctgcaggcgagacagatttg

SEQ ID NO: 204 - DNA, 20 bp, Artificial Sequence
  Antisense Compound
tttttgcagtaattcccggg

SEQ ID NO: 205 - DNA, 20 bp, Artificial Sequence
  Antisense Compound
catattccatttttgcagta

SEQ ID NO: 206 - DNA, 20 bp, Artificial Sequence
  Antisense Compound
agaagacacacatattccat

SEQ ID NO: 207 - DNA, 20 bp, Artificial Sequence
  Antisense Compound
tcctcaatttctcctcggaa

SEQ ID NO: 208 - DNA, 20 bp, Artificial Sequence
  Antisense Compound
tttcagtgatggtttcctca

SEQ ID NO: 209 - DNA, 20 bp, Artificial Sequence
  Antisense Compound
cattaccaaagctttcagtg

SEQ ID NO: 210 - DNA, 20 bp, Artificial Sequence
  Antisense Compound
cttctggaatacccatccaa

SEQ ID NO: 211 - DNA, 20 bp, Artificial Sequence
  Antisense Compound
atacatttttgaagacaagg

SEQ ID NO: 212 - DNA, 20 bp, Artificial Sequence
  Antisense Compound
agaaccttcttgtcctttgg

SEQ ID NO: 213 - DNA, 20 bp, Artificial Sequence
  Antisense Compound
cggagacaaacagaaccttc

SEQ ID NO: 214 - DNA, 20 bp, Artificial Sequence
  Antisense Compound
gatgaccggagacaaacaga

SEQ ID NO: 215 - DNA, 20 bp, Artificial Sequence
  Antisense Compound
cacaatcctgaggcacagtc

SEQ ID NO: 216 - DNA, 20 bp, Artificial Sequence
  Antisense Compound
agaagtgtctagcacaacac

SEQ ID NO: 217 - DNA, 20 bp, Artificial Sequence
  Antisense Compound
atcttggaccagaagtgtct

SEQ ID NO: 218 - DNA, 20 bp, Artificial Sequence
  Antisense Compound
tacagatcttggaccagaag

SEQ ID NO: 219 - DNA, 20 bp, Artificial Sequence
  Antisense Compound
aggtttacagatcttggacc

SEQ ID NO: 220 - DNA, 20 bp, Artificial Sequence
  Antisense Compound
ggacaggtttacagatcttg

SEQ ID NO: 221 - DNA, 20 bp, Artificial Sequence
  Antisense Compound
tatgcttggtacacacttga

SEQ ID NO: 222 - DNA, 20 bp, Artificial Sequence
  Antisense Compound
ctagtccatgagagcctttt

SEQ ID NO: 223 - DNA, 20 bp, Artificial Sequence
  Antisense Compound
cttctccacagtaacaacgc

SEQ ID NO: 224 - DNA, 20 bp, Artificial Sequence
  Antisense Compound
tctgtatccggcaagacaga

SEQ ID NO: 225 - DNA, 20 bp, Artificial Sequence
  Antisense Compound
gatctttctgtatccggcaa

SEQ ID NO: 226 - DNA, 20 bp, Artificial Sequence
  Antisense Compound
atggtgatctttctgtatcc

SEQ ID NO: 227 - DNA, 20 bp, Artificial Sequence
  Antisense Compound
gcttgatggtgatctttctg

SEQ ID NO: 228 - DNA, 20 bp, Artificial Sequence
  Antisense Compound
agaattactggcttgatggt

SEQ ID NO: 229 - DNA, 20 bp, Artificial Sequence
  Antisense Compound
tgtgaagcctagaagaatta

SEQ ID NO: 230 - DNA, 20 bp, Artificial Sequence
  Antisense Compound
acaagtgtgaagcctagaag

SEQ ID NO: 231 - DNA, 20 bp, Artificial Sequence
  Antisense Compound
tagctggtttagtgtctctg

SEQ ID NO: 232 - DNA, 20 bp, Artificial Sequence
  Antisense Compound
gttcactgcatttggatagc

SEQ ID NO: 233 - DNA, 20 bp, Artificial Sequence
  Antisense Compound
ttcatagcatctattatata

SEQ ID NO: 234 - DNA, 20 bp, Artificial Sequence
  Antisense Compound
tcataaaaggttttcatagc

SEQ ID NO: 235 - DNA, 20 bp, Artificial Sequence
  Antisense Compound
tccttaggattgagttgatg

SEQ ID NO: 236 - DNA, 20 bp, Artificial Sequence
  Antisense Compound
gcttaactgaaaccacagaa

SEQ ID NO: 237 - DNA, 20 bp, Artificial Sequence
  Antisense Compound
aggtgttattggaatgctta

SEQ ID NO: 238 - DNA, 20 bp, Artificial Sequence
  Antisense Compound
cactccaggtttttggaagg

SEQ ID NO: 239 - DNA, 20 bp, Artificial Sequence
  Antisense Compound
acaaagctcttacactccag

SEQ ID NO: 240 - DNA, 20 bp, Artificial Sequence
  Antisense Compound
gggagttccataaagaaaca

SEQ ID NO: 241 - DNA, 20 bp, Artificial Sequence
  Antisense Compound
aatttactgcaatcacaggg

SEQ ID NO: 242 - DNA, 20 bp, Artificial Sequence
  Antisense Compound
acagtaatttactgcaatca

SEQ ID NO: 243 - DNA, 20 bp, Artificial Sequence
  Antisense Compound
actgagaatttacaatacag

SEQ ID NO: 244 - DNA, 20 bp, Artificial Sequence
  Antisense Compound
caggtaagtgccacactgag

SEQ ID NO: 245 - DNA, 20 bp, Artificial Sequence
  Antisense Compound
atttacaggtaagtgccaca

SEQ ID NO: 246 - DNA, 20 bp, Artificial Sequence
  Antisense Compound
tgcagcacctttagaaaaat

SEQ ID NO: 247 - DNA, 20 bp, Artificial Sequence
  Antisense Compound
aaaataggcagtgcagcacc

SEQ ID NO: 248 - DNA, 20 bp, Artificial Sequence
  Antisense Compound
atatagaatatttgtcagtc

SEQ ID NO: 249 - DNA, 20 bp, Artificial Sequence
  Antisense Compound
atgatttacttcagttcaat

SEQ ID NO: 250 - DNA, 20 bp, Artificial Sequence
  Antisense Compound
tttaagaactataagctgaa

SEQ ID NO: 251 - DNA, 20 bp, Artificial Sequence
  Antisense Compound
ttctagactctagaattaaa

SEQ ID NO: 252 - DNA, 20 bp, Artificial Sequence
  Antisense Compound
aggtacctatcatttgtcat

SEQ ID NO: 253 - DNA, 20 bp, Artificial Sequence
  Antisense Compound
taatttaagcattaagatag

SEQ ID NO: 254 - DNA, 20 bp, Artificial Sequence
  Antisense Compound
aaactatcacagcctaaagg

SEQ ID NO: 255 - DNA, 20 bp, Artificial Sequence
  Antisense Compound
cccctctcacctggtagttg

SEQ ID NO: 256 - DNA, 20 bp, Artificial Sequence
  Antisense Compound
tctcttcttcccgattaaac

SEQ ID NO: 257 - DNA, 20 bp, Artificial Sequence
  Antisense Compound
tatctgtatgatcccatcgt

SEQ ID NO: 258 - DNA, 20 bp, Artificial Sequence
  Antisense Compound
ctaagttaagcaaatgcaat

SEQ ID NO: 259 - DNA, 20 bp, Artificial Sequence
  Antisense Compound
tcagtcataggtaatatccc

SEQ ID NO: 260 - DNA, 20 bp, Artificial Sequence
  Antisense Compound
acacatattcctaaggaagc

SEQ ID NO: 261 - DNA, 20 bp, Artificial Sequence
  Antisense Compound
acatccttacctttggtgtg

SEQ ID NO: 262 - DNA, 20 bp, Artificial Sequence
  Antisense Compound
ccttcttgtcctacgaagga

SEQ ID NO: 263 - DNA, 20 bp, Artificial Sequence
  Antisense Compound
caggactcaccgtttttgca

SEQ ID NO: 264 - DNA, 20 bp, Artificial Sequence
  Antisense Compound
tagctttgacagaaccagag

SEQ ID NO: 265 - DNA, 20 bp, Artificial Sequence
  Antisense Compound
taaactttcaaatgctcaaa

SEQ ID NO: 266 - DNA, 20 bp, Artificial Sequence
  Antisense Compound
gagagaagagagtttggcaa

SEQ ID NO: 267 - DNA, 20 bp, Artificial Sequence
  Antisense Compound
tctcacccgctagttacttt

SEQ ID NO: 268 - DNA, 20 bp, Artificial Sequence
  Antisense Compound
atgcatattcctaagaaagc

SEQ ID NO: 269 - DNA, 20 bp, Artificial Sequence
  Antisense Compound
ccttcttgccctgtaaagga

SEQ ID NO: 270 - DNA, 20 bp, Artificial Sequence
  Antisense Compound
aggctgttggttttagtgtc

SEQ ID NO: 271 - DNA, 20 bp, Artificial Sequence
  Antisense Compound
cttgggattgagctgacaaa

SEQ ID NO: 272 - DNA, 20 bp, Artificial Sequence
  Antisense Compound
acatccttgggattgagctg

SEQ ID NO: 273 - DNA, 20 bp, Artificial Sequence
  Antisense Compound
gaagattcctacatccttgg

SEQ ID NO: 274 - DNA, 20 bp, Artificial Sequence
  Antisense Compound
tacacactgaagattcctac

SEQ ID NO: 275 - DNA, 20 bp, Artificial Sequence
  Antisense Compound
atgcttaattacacactgaa

SEQ ID NO: 276 - DNA, 20 bp, Artificial Sequence
  Antisense Compound
tcggaatgcttaattacaca

SEQ ID NO: 277 - DNA, 20 bp, Artificial Sequence
  Antisense Compound
caaagtccttacactccaga

SEQ ID NO: 278 - DNA, 20 bp, Artificial Sequence
  Antisense Compound
ggacagctgccactgcacgc

SEQ ID NO: 279 - DNA, 20 bp, Artificial Sequence
  Antisense Compound
agggaggctgcagagagcca

SEQ ID NO: 280 - DNA, 20 bp, Artificial Sequence
  Antisense Compound
ggaattgatgagaactgagt

SEQ ID NO: 281 - DNA, 20 bp, Artificial Sequence
  Antisense Compound
gcgttggaattgatgagaac

SEQ ID NO: 282 - DNA, 20 bp, Artificial Sequence
  Antisense Compound
atcgcgttggaattgatgag

SEQ ID NO: 283 - DNA, 20 bp, Artificial Sequence
  Antisense Compound
tgatcgcgttggaattgatg

SEQ ID NO: 284 - DNA, 20 bp, Artificial Sequence
  Antisense Compound
gttcttgatcgcgttggaat

SEQ ID NO: 285 - DNA, 20 bp, Artificial Sequence
  Antisense Compound
ggcaggttcttgatcgcgtt

SEQ ID NO: 286 - DNA, 20 bp, Artificial Sequence
  Antisense Compound
ttgttcccgccctcatagag

SEQ ID NO: 287 - DNA, 20 bp, Artificial Sequence
  Antisense Compound
ggtacttgttcccgccctca

SEQ ID NO: 288 - DNA, 20 bp, Artificial Sequence
  Antisense Compound
agagtctggtacttgttccc

SEQ ID NO: 289 - DNA, 20 bp, Artificial Sequence
  Antisense Compound
tggtagttgtcaagagtctg

SEQ ID NO: 290 - DNA, 20 bp, Artificial Sequence
  Antisense Compound
tagggctggtagttgtcaag

SEQ ID NO: 291 - DNA, 20 bp, Artificial Sequence
  Antisense Compound
caggcagatttgtacacctc

SEQ ID NO: 292 - DNA, 20 bp, Artificial Sequence
  Antisense Compound
ctgcgctttcggcaagccag

SEQ ID NO: 293 - DNA, 20 bp, Artificial Sequence
  Antisense Compound
catagcgtgcctcatgcagc

SEQ ID NO: 294 - DNA, 20 bp, Artificial Sequence
  Antisense Compound
tgcatattccgtttttgcag

SEQ ID NO: 295 - DNA, 20 bp, Artificial Sequence
  Antisense Compound
tggctgtggtcagagggcat

SEQ ID NO: 296 - DNA, 20 bp, Artificial Sequence
  Antisense Compound
aatggctgtggtcagagggc

SEQ ID NO: 297 - DNA, 20 bp, Artificial Sequence
  Antisense Compound
gtaaatggctgtggtcagag

SEQ ID NO: 298 - DNA, 20 bp, Artificial Sequence
  Antisense Compound
tttcccctcgaggtaaatgg

SEQ ID NO: 299 - DNA, 20 bp, Artificial Sequence
  Antisense Compound
atgatgccttcctcgatttc

SEQ ID NO: 300 - DNA, 20 bp, Artificial Sequence
  Antisense Compound
tcaatgatgccttcctcgat

SEQ ID NO: 301 - DNA, 20 bp, Artificial Sequence
  Antisense Compound
gttttcaatgatgccttcct

SEQ ID NO: 302 - DNA, 20 bp, Artificial Sequence
  Antisense Compound
ccaaggttttcaatgatgcc

SEQ ID NO: 303 - DNA, 20 bp, Artificial Sequence
  Antisense Compound
tgccaaggttttcaatgatg

SEQ ID NO: 304 - DNA, 20 bp, Artificial Sequence
  Antisense Compound
cggcaccgtggtcattgcca

SEQ ID NO: 305 - DNA, 20 bp, Artificial Sequence
  Antisense Compound
atccatccccggcaccgtgg

SEQ ID NO: 306 - DNA, 20 bp, Artificial Sequence
  Antisense Compound
cttctgggatatccatcccc

SEQ ID NO: 307 - DNA, 20 bp, Artificial Sequence
  Antisense Compound
tggttcttctgggatatcca

SEQ ID NO: 308 - DNA, 20 bp, Artificial Sequence
  Antisense Compound
cagtgtggttcttctgggat

SEQ ID NO: 309 - DNA, 20 bp, Artificial Sequence
  Antisense Compound
atatttttgaagtcagtgtg

SEQ ID NO: 310 - DNA, 20 bp, Artificial Sequence
  Antisense Compound
gtgatatatttttgaagtca

SEQ ID NO: 311 - DNA, 20 bp, Artificial Sequence
  Antisense Compound
tcttgccctttggtgtgata

SEQ ID NO: 312 - DNA, 20 bp, Artificial Sequence
  Antisense Compound
gagccttcttgccctttggt

SEQ ID NO: 313 - DNA, 20 bp, Artificial Sequence
  Antisense Compound
tctgatgatcggaggcagac

SEQ ID NO: 314 - DNA, 20 bp, Artificial Sequence
  Antisense Compound
gccctgtggcgcagtctgat

SEQ ID NO: 315 - DNA, 20 bp, Artificial Sequence
  Antisense Compound
cacagccctgtggcgcagtc

SEQ ID NO: 316 - DNA, 20 bp, Artificial Sequence
  Antisense Compound
cagaaatgtcttgcacaaca

SEQ ID NO: 317 - DNA, 20 bp, Artificial Sequence
  Antisense Compound
accttctttaaggacaggtt

SEQ ID NO: 318 - DNA, 20 bp, Artificial Sequence
  Antisense Compound
tgaccttctttaaggacagg

SEQ ID NO: 319 - DNA, 20 bp, Artificial Sequence
  Antisense Compound
acctgaccttctttaaggac

SEQ ID NO: 320 - DNA, 20 bp, Artificial Sequence
  Antisense Compound
gtgcttggtgcatacctgac

SEQ ID NO: 321 - DNA, 20 bp, Artificial Sequence
  Antisense Compound
cagcccgtgggagcctttcc

SEQ ID NO: 322 - DNA, 20 bp, Artificial Sequence
  Antisense Compound
ctccagcccgtgggagcctt

SEQ ID NO: 323 - DNA, 20 bp, Artificial Sequence
  Antisense Compound
aacagcgctggaatatctcc

SEQ ID NO: 324 - DNA, 20 bp, Artificial Sequence
  Antisense Compound
gtaacagcgctggaatatct

SEQ ID NO: 325 - DNA, 20 bp, Artificial Sequence
  Antisense Compound
cagaccttccccacagtaac

SEQ ID NO: 326 - DNA, 20 bp, Artificial Sequence
  Antisense Compound
ttctgtatcctgcaagccag

SEQ ID NO: 327 - DNA, 20 bp, Artificial Sequence
  Antisense Compound
tctttctgtatcctgcaagc

SEQ ID NO: 328 - DNA, 20 bp, Artificial Sequence
  Antisense Compound
gatctttctgtatcctgcaa

SEQ ID NO: 329 - DNA, 20 bp, Artificial Sequence
  Antisense Compound
gtggagcctggaagaattgc

SEQ ID NO: 330 - DNA, 20 bp, Artificial Sequence
  Antisense Compound
gtgtctctggcaggtgtgga

SEQ ID NO: 331 - DNA, 20 bp, Artificial Sequence
  Antisense Compound
tagtgtctctggcaggtgtg

SEQ ID NO: 332 - DNA, 20 bp, Artificial Sequence
  Antisense Compound
tgaaatgtcctgttcctgac

SEQ ID NO: 333 - DNA, 20 bp, Artificial Sequence
  Antisense Compound
gccgaaagacccttgctcct

SEQ ID NO: 334 - DNA, 20 bp, Artificial Sequence
  Antisense Compound
aaagacttcgttctcttggc

SEQ ID NO: 335 - DNA, 20 bp, Artificial Sequence
  Antisense Compound
ttaagttctcccgcaagaaa

SEQ ID NO: 336 - DNA, 20 bp, Artificial Sequence
  Antisense Compound
atgcagcattaagttctccc

SEQ ID NO: 337 - DNA, 20 bp, Artificial Sequence
  Antisense Compound
cccgacatgatggctcttct

SEQ ID NO: 338 - DNA, 20 bp, Artificial Sequence
  Antisense Compound
tcgccccgacatgatggctc

SEQ ID NO: 339 - DNA, 20 bp, Artificial Sequence
  Antisense Compound
tccgcaaaggaggtggttct

SEQ ID NO: 340 - DNA, 20 bp, Artificial Sequence
  Antisense Compound
agctctccgcaaaggaggtg

SEQ ID NO: 341 - DNA, 20 bp, Artificial Sequence
  Antisense Compound
cttgcagctctccgcaaagg

SEQ ID NO: 342 - DNA, 20 bp, Artificial Sequence
  Antisense Compound
aaagctgaaggctgctgcac

SEQ ID NO: 343 - DNA, 20 bp, Artificial Sequence
  Antisense Compound
tctctgctaactttcatgct

SEQ ID NO: 344 - DNA, 20 bp, Artificial Sequence
  Antisense Compound
accttgctgccatctttatc

SEQ ID NO: 345 - DNA, 20 bp, Artificial Sequence
  Antisense Compound
ccaggagttgccaccactgt

SEQ ID NO: 346 - DNA, 20 bp, Artificial Sequence
  Antisense Compound
cttcctgtggcctgtcagga

SEQ ID NO: 347 - DNA, 20 bp, Artificial Sequence
  Antisense Compound
tgatataccacaccaaatga

SEQ ID NO: 348 - DNA, 20 bp, Artificial Sequence
  Antisense Compound
tggcttgatataccacacca

SEQ ID NO: 349 - DNA, 20 bp, Artificial Sequence
  Antisense Compound
aagtttggcttgatatacca

SEQ ID NO: 350 - DNA, 20 bp, Artificial Sequence
  Antisense Compound
tcacaaagtttggcttgata

SEQ ID NO: 351 - DNA, 20 bp, Artificial Sequence
  Antisense Compound
ttcttaaatcgcttgtcctg

SEQ ID NO: 352 - DNA, 20 bp, Artificial Sequence
  Antisense Compound
ctcatgatctggagctctcg

SEQ ID NO: 353 - DNA, 20 bp, Artificial Sequence
  Antisense Compound
gctttctcatgatctggagc

SEQ ID NO: 354 - DNA, 20 bp, Artificial Sequence
  Antisense Compound
atctagctttctcatgatct

SEQ ID NO: 355 - DNA, 20 bp, Artificial Sequence
  Antisense Compound
acagtgatctagctttctca

SEQ ID NO: 356 - DNA, 20 bp, Artificial Sequence
  Antisense Compound
ggactatgttacagtgatct

SEQ ID NO: 357 - DNA, 20 bp, Artificial Sequence
  Antisense Compound
ccactcgagtagaagaaata

SEQ ID NO: 358 - DNA, 20 bp, Artificial Sequence
  Antisense Compound
tagacctcatctttcttctc

SEQ ID NO: 359 - DNA, 20 bp, Artificial Sequence
  Antisense Compound
ggaacatagtccagcaccag

SEQ ID NO: 360 - DNA, 20 bp, Artificial Sequence
  Antisense Compound
actgtttccggaacatagtc

SEQ ID NO: 361 - DNA, 20 bp, Artificial Sequence
  Antisense Compound
agtgtctggcgactctgtac

SEQ ID NO: 362 - DNA, 20 bp, Artificial Sequence
  Antisense Compound
gctcgactatagtgtctggc

SEQ ID NO: 363 - DNA, 20 bp, Artificial Sequence
  Antisense Compound
tcacagggagtgtctgcttg

SEQ ID NO: 364 - DNA, 20 bp, Artificial Sequence
  Antisense Compound
tacatatacaacttgacata

SEQ ID NO: 365 - DNA, 20 bp, Artificial Sequence
  Antisense Compound
aaaggaatggatataggcta

SEQ ID NO: 366 - DNA, 20 bp, Artificial Sequence
  Antisense Compound
ttaatgtctcgatggcagat

SEQ ID NO: 367 - DNA, 20 bp, Artificial Sequence
  Antisense Compound
agaggttctgtggtttaatg

SEQ ID NO: 368 - DNA, 20 bp, Artificial Sequence
  Antisense Compound
tacagctgtatcaggatcca

SEQ ID NO: 369 - DNA, 20 bp, Artificial Sequence
  Antisense Compound
tgctttgcacttccaaagtc

SEQ ID NO: 370 - DNA, 20 bp, Artificial Sequence
  Antisense Compound
ccagctgctttgcacttcca

SEQ ID NO: 371 - DNA, 20 bp, Artificial Sequence
  Antisense Compound
tcggaccagctgctttgcac

SEQ ID NO: 372 - DNA, 20 bp, Artificial Sequence
  Antisense Compound
tctcctcggaccagctgctt

SEQ ID NO: 373 - DNA, 20 bp, Artificial Sequence
  Antisense Compound
tagtaccgagaacagatata

SEQ ID NO: 374 - DNA, 20 bp, Artificial Sequence
  Antisense Compound
tgccctgtagtaccgagaac

SEQ ID NO: 375 - DNA, 20 bp, Artificial Sequence
  Antisense Compound
cctagcaacaattcagccaa

SEQ ID NO: 376 - DNA, 20 bp, Artificial Sequence
  Antisense Compound
ggaaatattggttgtcctag

SEQ ID NO: 377 - DNA, 20 bp, Artificial Sequence
  Antisense Compound
actgtccccaggaaatattg

SEQ ID NO: 378 - DNA, 20 bp, Artificial Sequence
  Antisense Compound
accaactgatccacaccact

SEQ ID NO: 379 - DNA, 20 bp, Artificial Sequence
  Antisense Compound
cctaggacctttattatttc

SEQ ID NO: 380 - DNA, 20 bp, Artificial Sequence
  Antisense Compound
gaattctgtataatttgggt

SEQ ID NO: 381 - DNA, 20 bp, Artificial Sequence
  Antisense Compound
caaggatgtgccttgatttg

SEQ ID NO: 382 - DNA, 20 bp, Artificial Sequence
  Antisense Compound
caagcttccagtggtgttag

SEQ ID NO: 383 - DNA, 20 bp, Artificial Sequence
  Antisense Compound
gtgcacaagcttccagtggt

SEQ ID NO: 384 - DNA, 20 bp, Artificial Sequence
  Antisense Compound
tgaatgtgcacaagcttcca

SEQ ID NO: 385 - DNA, 20 bp, Artificial Sequence
  Antisense Compound
aaaaatgaatgtgcacaagc

SEQ ID NO: 386 - DNA, 20 bp, Artificial Sequence
  Antisense Compound
taattcatcaaaaaatgaat

SEQ ID NO: 387 - DNA, 20 bp, Artificial Sequence
  Antisense Compound
ttgggtcccgtaattcatca

SEQ ID NO: 388 - DNA, 20 bp, Artificial Sequence
  Antisense Compound
agtttgacatttgggtcccg

SEQ ID NO: 389 - DNA, 20 bp, Artificial Sequence
  Antisense Compound
ttggtagtttgacatttggg

SEQ ID NO: 390 - DNA, 20 bp, Artificial Sequence
  Antisense Compound
cccatttggtagtttgacat

SEQ ID NO: 391 - DNA, 20 bp, Artificial Sequence
  Antisense Compound
tctcgcccatttggtagttt

SEQ ID NO: 392 - DNA, 20 bp, Artificial Sequence
  Antisense Compound
gtgtgtctcgcccatttggt

SEQ ID NO: 393 - DNA, 20 bp, Artificial Sequence
  Antisense Compound
cttgacagttcttgagtggt

SEQ ID NO: 394 - DNA, 20 bp, Artificial Sequence
  Antisense Compound
ttagtatctgaggctgctgt

SEQ ID NO: 395 - DNA, 20 bp, Artificial Sequence
  Antisense Compound
ggtctgtccacggtctccag

SEQ ID NO: 396 - DNA, 20 bp, Artificial Sequence
  Antisense Compound
ttattggtctgtccacggtc

SEQ ID NO: 397 - DNA, 20 bp, Artificial Sequence
  Antisense Compound
tggcactcaagtaagtgctg

SEQ ID NO: 398 - DNA, 20 bp, Artificial Sequence
  Antisense Compound
gtgaccagtgttgctgagtg

SEQ ID NO: 399 - DNA, 20 bp, Artificial Sequence
  Antisense Compound
caaacgtgaccagtgttgct

SEQ ID NO: 400 - DNA, 20 bp, Artificial Sequence
  Antisense Compound
ctttccaaacgtgaccagtg

SEQ ID NO: 401 - DNA, 20 bp, Artificial Sequence
  Antisense Compound
taattttctttccaaacgtg

SEQ ID NO: 402 - DNA, 20 bp, Artificial Sequence
  Antisense Compound
cgatactcactgctaacttt

SEQ ID NO: 403 - DNA, 20 bp, Artificial Sequence
  Antisense Compound
ccatacttttggaataacaa

SEQ ID NO: 404 - DNA, 20 bp, Artificial Sequence
  Antisense Compound
aatttgcagactcagaactg

SEQ ID NO: 405 - DNA, 20 bp, Artificial Sequence
  Antisense Compound
ctgctttgcactgatgaaaa

SEQ ID NO: 406 - DNA, 20 bp, Artificial Sequence
  Antisense Compound
acagtttttcacctactagc

SEQ ID NO: 407 - DNA, 20 bp, Artificial Sequence
  Antisense Compound
aagtacatacccgtgctcct

SEQ ID NO: 408 - DNA, 20 bp, Artificial Sequence
  Antisense Compound
aaaatgaatgtgcacaagct

SEQ ID NO: 409 - DNA, 20 bp, Artificial Sequence
  Antisense Compound
gcagaccatacatctatact

SEQ ID NO: 410 - DNA, 20 bp, Artificial Sequence
  Antisense Compound
aggaggaagggcactcggtc

SEQ ID NO: 411 - DNA, 20 bp, Artificial Sequence
  Antisense Compound
tgagagctgcatggtgccag

SEQ ID NO: 412 - DNA, 20 bp, Artificial Sequence
  Antisense Compound
ctgcatgtacaagcaggcag

SEQ ID NO: 413 - DNA, 20 bp, Artificial Sequence
  Antisense Compound
gaaggctgcatgtacaagca

SEQ ID NO: 414 - DNA, 20 bp, Artificial Sequence
  Antisense Compound
gcaacgaaggctgcatgtac

SEQ ID NO: 415 - DNA, 20 bp, Artificial Sequence
  Antisense Compound
tggctctccacagcaacgaa

SEQ ID NO: 416 - DNA, 20 bp, Artificial Sequence
  Antisense Compound
gaaggcttgccacccctggc

SEQ ID NO: 417 - DNA, 20 bp, Artificial Sequence
  Antisense Compound
ttcttgaaggcttgccaccc

SEQ ID NO: 418 - DNA, 20 bp, Artificial Sequence
  Antisense Compound
catcattcttgaaggcttgc

SEQ ID NO: 419 - DNA, 20 bp, Artificial Sequence
  Antisense Compound
tgtggcatcattcttgaagg

SEQ ID NO: 420 - DNA, 20 bp, Artificial Sequence
  Antisense Compound
agtcccgggatgatttctgt

SEQ ID NO: 421 - DNA, 20 bp, Artificial Sequence
  Antisense Compound
gtactctctgagtcccggga

SEQ ID NO: 422 - DNA, 20 bp, Artificial Sequence
  Antisense Compound
ctctgggtactctctgagtc

SEQ ID NO: 423 - DNA, 20 bp, Artificial Sequence
  Antisense Compound
ggaggctctgggtactctct

SEQ ID NO: 424 - DNA, 20 bp, Artificial Sequence
  Antisense Compound
gttcctgaggaggctctggg

SEQ ID NO: 425 - DNA, 20 bp, Artificial Sequence
  Antisense Compound
ctctagttcctgaggaggct

SEQ ID NO: 426 - DNA, 20 bp, Artificial Sequence
  Antisense Compound
ttgttctctagttcctgagg

SEQ ID NO: 427 - DNA, 20 bp, Artificial Sequence
  Antisense Compound
ggttcatggtctggttgttc

SEQ ID NO: 428 - DNA, 20 bp, Artificial Sequence
  Antisense Compound
cggttcatggtctggttgtt

SEQ ID NO: 429 - DNA, 20 bp, Artificial Sequence
  Antisense Compound
ccggttcatggtctggttgt

SEQ ID NO: 430 - DNA, 20 bp, Artificial Sequence
  Antisense Compound
ggcccggttcatggtctggt

SEQ ID NO: 431 - DNA, 20 bp, Artificial Sequence
  Antisense Compound
tcggcccggttcatggtctg

SEQ ID NO: 432 - DNA, 20 bp, Artificial Sequence
  Antisense Compound
tctcggcccggttcatggtc

SEQ ID NO: 433 - DNA, 20 bp, Artificial Sequence
  Antisense Compound
ctttggtgtcataaggatgg

SEQ ID NO: 434 - DNA, 20 bp, Artificial Sequence
  Antisense Compound
gtctttggtgtcataaggat

SEQ ID NO: 435 - DNA, 20 bp, Artificial Sequence
  Antisense Compound
cgtctttggtgtcataagga

SEQ ID NO: 436 - DNA, 20 bp, Artificial Sequence
  Antisense Compound
cggacacgtctttggtgtca

SEQ ID NO: 437 - DNA, 20 bp, Artificial Sequence
  Antisense Compound
ctcggacacgtctttggtgt

SEQ ID NO: 438 - DNA, 20 bp, Artificial Sequence
  Antisense Compound
gtactcggacacgtctttgg

SEQ ID NO: 439 - DNA, 20 bp, Artificial Sequence
  Antisense Compound
gctgtactcggacacgtctt

SEQ ID NO: 440 - DNA, 20 bp, Artificial Sequence
  Antisense Compound
ggcagctgtactcggacacg

SEQ ID NO: 441 - DNA, 20 bp, Artificial Sequence
  Antisense Compound
cggcagctgtactcggacac

SEQ ID NO: 442 - DNA, 20 bp, Artificial Sequence
  Antisense Compound
agctcgcggcagctgtactc

SEQ ID NO: 443 - DNA, 20 bp, Artificial Sequence
  Antisense Compound
gcagctcgcggcagctgtac

SEQ ID NO: 444 - DNA, 20 bp, Artificial Sequence
  Antisense Compound
tgcagctcgcggcagctgta

SEQ ID NO: 445 - DNA, 20 bp, Artificial Sequence
  Antisense Compound
gtgcagctcgcggcagctgt

SEQ ID NO: 446 - DNA, 20 bp, Artificial Sequence
  Antisense Compound
tagtgcagctcgcggcagct

SEQ ID NO: 447 - DNA, 20 bp, Artificial Sequence
  Antisense Compound
tgtagtgcagctcgcggcag

SEQ ID NO: 448 - DNA, 20 bp, Artificial Sequence
  Antisense Compound
ggtgtagtgcagctcgcggc

SEQ ID NO: 449 - DNA, 20 bp, Artificial Sequence
  Antisense Compound
gcgggtgtagtgcagctcgc

SEQ ID NO: 450 - DNA, 20 bp, Artificial Sequence
  Antisense Compound
aagcgggtgtagtgcagctc

SEQ ID NO: 451 - DNA, 20 bp, Artificial Sequence
  Antisense Compound
cacgaagcgggtgtagtgca

SEQ ID NO: 452 - DNA, 20 bp, Artificial Sequence
  Antisense Compound
gtcacgaagcgggtgtagtg

SEQ ID NO: 453 - DNA, 20 bp, Artificial Sequence
  Antisense Compound
gaccggcttggcactgcggc

SEQ ID NO: 454 - DNA, 20 bp, Artificial Sequence
  Antisense Compound
actcggtgaccggcttggca

SEQ ID NO: 455 - DNA, 20 bp, Artificial Sequence
  Antisense Compound
aactcggtgaccggcttggc

SEQ ID NO: 456 - DNA, 20 bp, Artificial Sequence
  Antisense Compound
caactcggtgaccggcttgg

SEQ ID NO: 457 - DNA, 20 bp, Artificial Sequence
  Antisense Compound
ccaactcggtgaccggcttg

SEQ ID NO: 458 - DNA, 20 bp, Artificial Sequence
  Antisense Compound
acaccaactcggtgaccggc

SEQ ID NO: 459 - DNA, 20 bp, Artificial Sequence
  Antisense Compound
gagcacaccaactcggtgac

SEQ ID NO: 460 - DNA, 20 bp, Artificial Sequence
  Antisense Compound
gcccgagcacaccaactcgg

SEQ ID NO: 461 - DNA, 20 bp, Artificial Sequence
  Antisense Compound
actggcccgagcacaccaac

SEQ ID NO: 462 - DNA, 20 bp, Artificial Sequence
  Antisense Compound
gatgcagcggaagtcgggtc

SEQ ID NO: 463 - DNA, 20 bp, Artificial Sequence
  Antisense Compound
gtagcgatccgggatgcagc

SEQ ID NO: 464 - DNA, 20 bp, Artificial Sequence
  Antisense Compound
agcagctgcacccgctgcgc

SEQ ID NO: 465 - DNA, 20 bp, Artificial Sequence
  Antisense Compound
acagcagctgcacccgctgc

SEQ ID NO: 466 - DNA, 20 bp, Artificial Sequence
  Antisense Compound
ggcacagcagctgcacccgc

SEQ ID NO: 467 - DNA, 20 bp, Artificial Sequence
  Antisense Compound
gccaccagacgcaccttgcg

SEQ ID NO: 468 - DNA, 20 bp, Artificial Sequence
  Antisense Compound
cgaggccaccagacgcacct

SEQ ID NO: 469 - DNA, 20 bp, Artificial Sequence
  Antisense Compound
tgcacgaggccaccagacgc

SEQ ID NO: 470 - DNA, 20 bp, Artificial Sequence
  Antisense Compound
ttgcacgaggccaccagacg

SEQ ID NO: 471 - DNA, 20 bp, Artificial Sequence
  Antisense Compound
gcacttgcacgaggccacca

SEQ ID NO: 472 - DNA, 20 bp, Artificial Sequence
  Antisense Compound
ttgcacttgcacgaggccac

SEQ ID NO: 473 - DNA, 20 bp, Artificial Sequence
  Antisense Compound
cgcttgcacttgcacgaggc

SEQ ID NO: 474 - DNA, 20 bp, Artificial Sequence
  Antisense Compound
ccgactggttgtggaagcgg

SEQ ID NO: 475 - DNA, 20 bp, Artificial Sequence
  Antisense Compound
ctccgactggttgtggaagc

SEQ ID NO: 476 - DNA, 20 bp, Artificial Sequence
  Antisense Compound
gagctccgactggttgtgga

SEQ ID NO: 477 - DNA, 20 bp, Artificial Sequence
  Antisense Compound
tccttgagctccgactggtt

SEQ ID NO: 478 - DNA, 20 bp, Artificial Sequence
  Antisense Compound
gtccttgagctccgactggt

SEQ ID NO: 479 - DNA, 20 bp, Artificial Sequence
  Antisense Compound
cgaagtccttgagctccgac

SEQ ID NO: 480 - DNA, 20 bp, Artificial Sequence
  Antisense Compound
ggtccgaagtccttgagctc

SEQ ID NO: 481 - DNA, 20 bp, Artificial Sequence
  Antisense Compound
cttgcgacccttctgcggcc

SEQ ID NO: 482 - DNA, 20 bp, Artificial Sequence
  Antisense Compound
gcttgcgacccttctgcggc

SEQ ID NO: 483 - DNA, 20 bp, Artificial Sequence
  Antisense Compound
gcggcttgcgacccttctgc

SEQ ID NO: 484 - DNA, 20 bp, Artificial Sequence
  Antisense Compound
agctccgcctggttggcttt

SEQ ID NO: 485 - DNA, 20 bp, Artificial Sequence
  Antisense Compound
cagctccgcctggttggctt

SEQ ID NO: 486 - DNA, 20 bp, Artificial Sequence
  Antisense Compound
tctccagctccgcctggttg

SEQ ID NO: 487 - DNA, 20 bp, Artificial Sequence
  Antisense Compound
ttctccagctccgcctggtt

SEQ ID NO: 488 - DNA, 20 bp, Artificial Sequence
  Antisense Compound
aagaactgcatgaccggctc

SEQ ID NO: 489 - DNA, 20 bp, Artificial Sequence
  Antisense Compound
cgaagaactgcatgaccggc

SEQ ID NO: 490 - DNA, 20 bp, Artificial Sequence
  Antisense Compound
gccgaagaactgcatgaccg

SEQ ID NO: 491 - DNA, 20 bp, Artificial Sequence
  Antisense Compound
gaagccgaagaactgcatga

SEQ ID NO: 492 - DNA, 20 bp, Artificial Sequence
  Antisense Compound
gtagaagccgaagaactgca

SEQ ID NO: 493 - DNA, 20 bp, Artificial Sequence
  Antisense Compound
cggccagtagaagccgaaga

SEQ ID NO: 494 - DNA, 20 bp, Artificial Sequence
  Antisense Compound
tctccggccagtagaagccg

SEQ ID NO: 495 - DNA, 20 bp, Artificial Sequence
  Antisense Compound
gagcatctccggccagtaga

SEQ ID NO: 496 - DNA, 20 bp, Artificial Sequence
  Antisense Compound
cacatttgagcatctccggc

SEQ ID NO: 497 - DNA, 20 bp, Artificial Sequence
  Antisense Compound
gtcacatttgagcatctccg

SEQ ID NO: 498 - DNA, 20 bp, Artificial Sequence
  Antisense Compound
acttgtcacatttgagcatc

SEQ ID NO: 499 - DNA, 20 bp, Artificial Sequence
  Antisense Compound
gggaacttgtcacatttgag

SEQ ID NO: 500 - DNA, 20 bp, Artificial Sequence
  Antisense Compound
aggcttcggtggcattgggc

SEQ ID NO: 501 - DNA, 20 bp, Artificial Sequence
  Antisense Compound
ttcgaggcttcggtggcatt

SEQ ID NO: 502 - DNA, 20 bp, Artificial Sequence
  Antisense Compound
ggcttcgaggcttcggtggc

SEQ ID NO: 503 - DNA, 20 bp, Artificial Sequence
  Antisense Compound
ggacacactgttgtaccttg

SEQ ID NO: 504 - DNA, 20 bp, Artificial Sequence
  Antisense Compound
cacacggaggacacactgtt

SEQ ID NO: 505 - DNA, 20 bp, Artificial Sequence
  Antisense Compound
gtcacacggaggacacactg

SEQ ID NO: 506 - DNA, 20 bp, Artificial Sequence
  Antisense Compound
tcgttgtcacacggaggaca

SEQ ID NO: 507 - DNA, 20 bp, Artificial Sequence
  Antisense Compound
ttcaactcgttgtcacacgg

SEQ ID NO: 508 - DNA, 20 bp, Artificial Sequence
  Antisense Compound
cgatttcaactcgttgtcac

SEQ ID NO: 509 - DNA, 20 bp, Artificial Sequence
  Antisense Compound
cctccgatttcaactcgttg

SEQ ID NO: 510 - DNA, 20 bp, Artificial Sequence
  Antisense Compound
ggcctccgatttcaactcgt

SEQ ID NO: 511 - DNA, 20 bp, Artificial Sequence
  Antisense Compound
atggcctccgatttcaactc

SEQ ID NO: 512 - DNA, 20 bp, Artificial Sequence
  Antisense Compound
tgatggcctccgatttcaac

SEQ ID NO: 513 - DNA, 20 bp, Artificial Sequence
  Antisense Compound
tcgatgatggcctccgattt

SEQ ID NO: 514 - DNA, 20 bp, Artificial Sequence
  Antisense Compound
gttcgatgatggcctccgat

SEQ ID NO: 515 - DNA, 20 bp, Artificial Sequence
  Antisense Compound
gatgttcgatgatggcctcc

SEQ ID NO: 516 - DNA, 20 bp, Artificial Sequence
  Antisense Compound
acagagatgttcgatgatgg

SEQ ID NO: 517 - DNA, 20 bp, Artificial Sequence
  Antisense Compound
gcacagagatgttcgatgat

SEQ ID NO: 518 - DNA, 20 bp, Artificial Sequence
  Antisense Compound
ttgcacagagatgttcgatg

SEQ ID NO: 519 - DNA, 20 bp, Artificial Sequence
  Antisense Compound
actcgcttgcacagagatgt

SEQ ID NO: 520 - DNA, 20 bp, Artificial Sequence
  Antisense Compound
aactcgcttgcacagagatg

SEQ ID NO: 521 - DNA, 20 bp, Artificial Sequence
  Antisense Compound
gcaaactcgcttgcacagag

SEQ ID NO: 522 - DNA, 20 bp, Artificial Sequence
  Antisense Compound
cagcgcaaactcgcttgcac

SEQ ID NO: 523 - DNA, 20 bp, Artificial Sequence
  Antisense Compound
tctcagcgcaaactcgcttg

SEQ ID NO: 524 - DNA, 20 bp, Artificial Sequence
  Antisense Compound
tcattctcagcgcaaactcg

SEQ ID NO: 525 - DNA, 20 bp, Artificial Sequence
  Antisense Compound
attttcattctcagcgcaaa

SEQ ID NO: 526 - DNA, 20 bp, Artificial Sequence
  Antisense Compound
ttgattttcattctcagcgc

SEQ ID NO: 527 - DNA, 20 bp, Artificial Sequence
  Antisense Compound
ggacaatcttcttgtcaccg

SEQ ID NO: 528 - DNA, 20 bp, Artificial Sequence
  Antisense Compound
cagcttcaagggtttcttct

SEQ ID NO: 529 - DNA, 20 bp, Artificial Sequence
  Antisense Compound
cccagcttcaagggtttctt

SEQ ID NO: 530 - DNA, 20 bp, Artificial Sequence
  Antisense Compound
ggccccagcttcaagggttt

SEQ ID NO: 531 - DNA, 20 bp, Artificial Sequence
  Antisense Compound
gatgggccccagcttcaagg

SEQ ID NO: 532 - DNA, 20 bp, Artificial Sequence
  Antisense Compound
tcttgatgggccccagcttc

SEQ ID NO: 533 - DNA, 20 bp, Artificial Sequence
  Antisense Compound
ctccttcttcttgatgggcc

SEQ ID NO: 534 - DNA, 20 bp, Artificial Sequence
  Antisense Compound
gctccttcttcttgatgggc

SEQ ID NO: 535 - DNA, 20 bp, Artificial Sequence
  Antisense Compound
gccgcttcagctccttcttc

SEQ ID NO: 536 - DNA, 20 bp, Artificial Sequence
  Antisense Compound
gagccgcttcagctccttct

SEQ ID NO: 537 - DNA, 20 bp, Artificial Sequence
  Antisense Compound
gcacgagccgcttcagctcc

SEQ ID NO: 538 - DNA, 20 bp, Artificial Sequence
  Antisense Compound
aaagcacgagccgcttcagc

SEQ ID NO: 539 - DNA, 20 bp, Artificial Sequence
  Antisense Compound
gaaaagcacgagccgcttca

SEQ ID NO: 540 - DNA, 20 bp, Artificial Sequence
  Antisense Compound
tttaggaaaagcacgagccg

SEQ ID NO: 541 - DNA, 20 bp, Artificial Sequence
  Antisense Compound
tccagctggtggcagggaca

SEQ ID NO: 542 - DNA, 20 bp, Artificial Sequence
  Antisense Compound
gttgtccagctggtggcagg

SEQ ID NO: 543 - DNA, 20 bp, Artificial Sequence
  Antisense Compound
tgaggttgtccagctggtgg

SEQ ID NO: 544 - DNA, 20 bp, Artificial Sequence
  Antisense Compound
tgtggctgaggttgtccagc

SEQ ID NO: 545 - DNA, 20 bp, Artificial Sequence
  Antisense Compound
aagttgtggctgaggttgtc

SEQ ID NO: 546 - DNA, 20 bp, Artificial Sequence
  Antisense Compound
aggaagttgtggctgaggtt

SEQ ID NO: 547 - DNA, 20 bp, Artificial Sequence
  Antisense Compound
tgatgaggaagttgtggctg

SEQ ID NO: 548 - DNA, 20 bp, Artificial Sequence
  Antisense Compound
catgatgaggaagttgtggc

SEQ ID NO: 549 - DNA, 20 bp, Artificial Sequence
  Antisense Compound
cccatgatgaggaagttgtg

SEQ ID NO: 550 - DNA, 20 bp, Artificial Sequence
  Antisense Compound
gccccatgatgaggaagttg

SEQ ID NO: 551 - DNA, 20 bp, Artificial Sequence
  Antisense Compound
gcgccccatgatgaggaagt

SEQ ID NO: 552 - DNA, 20 bp, Artificial Sequence
  Antisense Compound
ttgcgccccatgatgaggaa

SEQ ID NO: 553 - DNA, 20 bp, Artificial Sequence
  Antisense Compound
ccttgcgccccatgatgagg

SEQ ID NO: 554 - DNA, 20 bp, Artificial Sequence
  Antisense Compound
caccttgcgccccatgatga

SEQ ID NO: 555 - DNA, 20 bp, Artificial Sequence
  Antisense Compound
cttcaccttgcgccccatga

SEQ ID NO: 556 - DNA, 20 bp, Artificial Sequence
  Antisense Compound
tggctcttcaccttgcgccc

SEQ ID NO: 557 - DNA, 20 bp, Artificial Sequence
  Antisense Compound
actggctcttcaccttgcgc

SEQ ID NO: 558 - DNA, 20 bp, Artificial Sequence
  Antisense Compound
gtactggctcttcaccttgc

SEQ ID NO: 559 - DNA, 20 bp, Artificial Sequence
  Antisense Compound
gtgagcaagtactggctctt

SEQ ID NO: 560 - DNA, 20 bp, Artificial Sequence
  Antisense Compound
atggctgtgagcaagtactg

SEQ ID NO: 561 - DNA, 20 bp, Artificial Sequence
  Antisense Compound
gaatggctgtgagcaagtac

SEQ ID NO: 562 - DNA, 20 bp, Artificial Sequence
  Antisense Compound
cccacttgtgaatggctgtg

SEQ ID NO: 563 - DNA, 20 bp, Artificial Sequence
  Antisense Compound
gtcccacttgtgaatggctg

SEQ ID NO: 564 - DNA, 20 bp, Artificial Sequence
  Antisense Compound
ttgtcccacttgtgaatggc

SEQ ID NO: 565 - DNA, 20 bp, Artificial Sequence
  Antisense Compound
ttcttgtcccacttgtgaat

SEQ ID NO: 566 - DNA, 20 bp, Artificial Sequence
  Antisense Compound
ctctacgccacaaaattagg

SEQ ID NO: 567 - DNA, 20 bp, Artificial Sequence
  Antisense Compound
catagctctacgccacaaaa

SEQ ID NO: 568 - DNA, 20 bp, Artificial Sequence
  Antisense Compound
gaagcttattgtacaaatac

SEQ ID NO: 569 - DNA, 20 bp, Artificial Sequence
  Antisense Compound
cgtctcctgggaagcttatt

SEQ ID NO: 570 - DNA, 20 bp, Artificial Sequence
  Antisense Compound
aaaaatgttgacacgtctcc

SEQ ID NO: 571 - DNA, 20 bp, Artificial Sequence
  Antisense Compound
cccgatcctttgtatggctt

SEQ ID NO: 572 - DNA, 20 bp, Artificial Sequence
  Antisense Compound
atacatctaaaccccgatcc

SEQ ID NO: 573 - DNA, 20 bp, Artificial Sequence
  Antisense Compound
ctatgtgtatacatctaaac

SEQ ID NO: 574 - DNA, 20 bp, Artificial Sequence
  Antisense Compound
tggatgcttgttcaatcact

SEQ ID NO: 575 - DNA, 20 bp, Artificial Sequence
  Antisense Compound
atgtccaaaccactctcttt

SEQ ID NO: 576 - DNA, 20 bp, Artificial Sequence
  Antisense Compound
aatgtccaaaccactctctt

SEQ ID NO: 577 - DNA, 20 bp, Artificial Sequence
  Antisense Compound
gatctccttatccaactcac

SEQ ID NO: 578 - DNA, 20 bp, Artificial Sequence
  Antisense Compound
agctggacactgatgaggct

SEQ ID NO: 579 - DNA, 20 bp, Artificial Sequence
  Antisense Compound
cgatggagagctggacactg

SEQ ID NO: 580 - DNA, 20 bp, Artificial Sequence
  Antisense Compound
gcgatggagagctggacact

SEQ ID NO: 581 - DNA, 20 bp, Artificial Sequence
  Antisense Compound
tacagcagcagagtgaccaa

SEQ ID NO: 582 - DNA, 20 bp, Artificial Sequence
  Antisense Compound
gcatgaaggtagggcttaca

SEQ ID NO: 583 - DNA, 20 bp, Artificial Sequence
  Antisense Compound
cagcaaaagtggcagtggta

SEQ ID NO: 584 - DNA, 20 bp, Artificial Sequence
  Antisense Compound
ttttggtagagccgaacttg

SEQ ID NO: 585 - DNA, 20 bp, Artificial Sequence
  Antisense Compound
tcttcattttggtagagccg

SEQ ID NO: 586 - DNA, 20 bp, Artificial Sequence
  Antisense Compound
gaagtacgtgcaaccttgtt

SEQ ID NO: 587 - DNA, 20 bp, Artificial Sequence
  Antisense Compound
cacattcaagccgaggttga

SEQ ID NO: 588 - DNA, 20 bp, Artificial Sequence
  Antisense Compound
aagagatggctttctgcttc

SEQ ID NO: 589 - DNA, 20 bp, Artificial Sequence
  Antisense Compound
tgaggaagagatggctttct

SEQ ID NO: 590 - DNA, 20 bp, Artificial Sequence
  Antisense Compound
gctggctacccaagccaagc

SEQ ID NO: 591 - DNA, 20 bp, Artificial Sequence
  Antisense Compound
ctgctggctacccaagccaa

SEQ ID NO: 592 - DNA, 20 bp, Artificial Sequence
  Antisense Compound
gctgctggctacccaagcca

SEQ ID NO: 593 - DNA, 20 bp, Artificial Sequence
  Antisense Compound
aggaaaaataaattccttgg

SEQ ID NO: 594 - DNA, 20 bp, Artificial Sequence
  Antisense Compound
ccctgcatattaggaaaaat

SEQ ID NO: 595 - DNA, 20 bp, Artificial Sequence
  Antisense Compound
cattccattggtactactac

SEQ ID NO: 596 - DNA, 20 bp, Artificial Sequence
  Antisense Compound
ctgtcacctgggaacattcc

SEQ ID NO: 597 - DNA, 20 bp, Artificial Sequence
  Antisense Compound
ttactgtactggagaggact

SEQ ID NO: 598 - DNA, 20 bp, Artificial Sequence
  Antisense Compound
attcaagccatctacaaaag

SEQ ID NO: 599 - DNA, 20 bp, Artificial Sequence
  Antisense Compound
actgcatgttatttaagcta

SEQ ID NO: 600 - DNA, 20 bp, Artificial Sequence
  Antisense Compound
aggctggaattgctggttag

SEQ ID NO: 601 - DNA, 20 bp, Artificial Sequence
  Antisense Compound
ttttagttagccataacagg

SEQ ID NO: 602 - DNA, 20 bp, Artificial Sequence
  Antisense Compound
cccaagcttgaatgtatcct

SEQ ID NO: 603 - DNA, 20 bp, Artificial Sequence
  Antisense Compound
gaagattatccttcaacctt

SEQ ID NO: 604 - DNA, 20 bp, Artificial Sequence
  Antisense Compound
ttctcattcaaagtgctggt

SEQ ID NO: 605 - DNA, 20 bp, Artificial Sequence
  Antisense Compound
tgtggcagagaaacaaattc

SEQ ID NO: 606 - DNA, 20 bp, Artificial Sequence
  Antisense Compound
gtttcaactcctccaccaga

SEQ ID NO: 607 - DNA, 20 bp, Artificial Sequence
  Antisense Compound
aaaggtaggtttgtttcaac

SEQ ID NO: 608 - DNA, 20 bp, Artificial Sequence
  Antisense Compound
tcaagctgcatagctgcgat

SEQ ID NO: 609 - DNA, 20 bp, Artificial Sequence
  Antisense Compound
ataaaatttagtgctacttg

SEQ ID NO: 610 - DNA, 20 bp, Artificial Sequence
  Antisense Compound
ctgggaagcttattgtacaa

SEQ ID NO: 611 - DNA, 20 bp, Artificial Sequence
  Antisense Compound
gtctcctgggaagcttattg

SEQ ID NO: 612 - DNA, 20 bp, Artificial Sequence
  Antisense Compound
gacacgtctcctgggaagct

SEQ ID NO: 613 - DNA, 20 bp, Artificial Sequence
  Antisense Compound
atgttgacacgtctcctggg

SEQ ID NO: 614 - DNA, 20 bp, Artificial Sequence
  Antisense Compound
caaaaatgttgacacgtctc

SEQ ID NO: 615 - DNA, 20 bp, Artificial Sequence
  Antisense Compound
ttcaccaaaaatgttgacac

SEQ ID NO: 616 - DNA, 20 bp, Artificial Sequence
  Antisense Compound
caagttcttcaccaaaaatg

SEQ ID NO: 617 - DNA, 20 bp, Artificial Sequence
  Antisense Compound
tctttcaagttcttcaccaa

SEQ ID NO: 618 - DNA, 20 bp, Artificial Sequence
  Antisense Compound
agaagtctttcaagttcttc

SEQ ID NO: 619 - DNA, 20 bp, Artificial Sequence
  Antisense Compound
cctttgtatggcttctcagg

SEQ ID NO: 620 - DNA, 20 bp, Artificial Sequence
  Antisense Compound
cctgagcctttgtatggctt

SEQ ID NO: 621 - DNA, 20 bp, Artificial Sequence
  Antisense Compound
taaaccctgagcctttgtat

SEQ ID NO: 622 - DNA, 20 bp, Artificial Sequence
  Antisense Compound
ccagaccactctctttggac

SEQ ID NO: 623 - DNA, 20 bp, Artificial Sequence
  Antisense Compound
acgaacatcgtcaatgtcca

SEQ ID NO: 624 - DNA, 20 bp, Artificial Sequence
  Antisense Compound
ttgccacgaacatcgtcaat

SEQ ID NO: 625 - DNA, 20 bp, Artificial Sequence
  Antisense Compound
ggcagattgccacgaacatc

SEQ ID NO: 626 - DNA, 20 bp, Artificial Sequence
  Antisense Compound
aacctcaaacgggtcgatcc

SEQ ID NO: 627 - DNA, 20 bp, Artificial Sequence
  Antisense Compound
cgtacagcaccttcactggt

SEQ ID NO: 628 - DNA, 20 bp, Artificial Sequence
  Antisense Compound
tcattactgtcatctacgta

SEQ ID NO: 629 - DNA, 20 bp, Artificial Sequence
  Antisense Compound
cgttctcattactgtcatct

SEQ ID NO: 630 - DNA, 20 bp, Artificial Sequence
  Antisense Compound
ctcacacccgttctcattac

SEQ ID NO: 631 - DNA, 20 bp, Artificial Sequence
  Antisense Compound
ttatccagctcacacccgtt

SEQ ID NO: 632 - DNA, 20 bp, Artificial Sequence
  Antisense Compound
atctccttatccagctcaca

SEQ ID NO: 633 - DNA, 20 bp, Artificial Sequence
  Antisense Compound
gacacagaggaggctgggtc

SEQ ID NO: 634 - DNA, 20 bp, Artificial Sequence
  Antisense Compound
gacggcagcagaatggccaa

SEQ ID NO: 635 - DNA, 20 bp, Artificial Sequence
  Antisense Compound
taaaggctgagtggaccggg

SEQ ID NO: 636 - DNA, 20 bp, Artificial Sequence
  Antisense Compound
aaggttaaaggctgagtgga

SEQ ID NO: 637 - DNA, 20 bp, Artificial Sequence
  Antisense Compound
gtggtaaaggttaaaggctg

SEQ ID NO: 638 - DNA, 20 bp, Artificial Sequence
  Antisense Compound
tggcagtggtaaaggttaaa

SEQ ID NO: 639 - DNA, 20 bp, Artificial Sequence
  Antisense Compound
aaaagtggcagtggtaaagg

SEQ ID NO: 640 - DNA, 20 bp, Artificial Sequence
  Antisense Compound
accttgctgctacggccact

SEQ ID NO: 641 - DNA, 20 bp, Artificial Sequence
  Antisense Compound
gggagaagtgcgtgctacct

SEQ ID NO: 642 - DNA, 20 bp, Artificial Sequence
  Antisense Compound
acattgacattcaggcccag

SEQ ID NO: 643 - DNA, 20 bp, Artificial Sequence
  Antisense Compound
gcttcaggaggtcgttcaca

SEQ ID NO: 644 - DNA, 20 bp, Artificial Sequence
  Antisense Compound
ggctttctgcttcaggaggt

SEQ ID NO: 645 - DNA, 20 bp, Artificial Sequence
  Antisense Compound
agagatggctttctgcttca

SEQ ID NO: 646 - DNA, 20 bp, Artificial Sequence
  Antisense Compound
gaggaagagatggctttctg

SEQ ID NO: 647 - DNA, 20 bp, Artificial Sequence
  Antisense Compound
gcattgaggaagagatggct

SEQ ID NO: 648 - DNA, 20 bp, Artificial Sequence
  Antisense Compound
agagtgcattgaggaagaga

SEQ ID NO: 649 - DNA, 20 bp, Artificial Sequence
  Antisense Compound
tacagagagtgcattgagga

SEQ ID NO: 650 - DNA, 20 bp, Artificial Sequence
  Antisense Compound
gctggctgcccaggcccagc

SEQ ID NO: 651 - DNA, 20 bp, Artificial Sequence
  Antisense Compound
ctgctgctggctgcccaggc

SEQ ID NO: 652 - DNA, 20 bp, Artificial Sequence
  Antisense Compound
ctgcggctgcggctgaggct

SEQ ID NO: 653 - DNA, 20 bp, Artificial Sequence
  Antisense Compound
tccgtaggccgcaaacacat

SEQ ID NO: 654 - DNA, 20 bp, Artificial Sequence
  Antisense Compound
aggcctccgtaggccgcaaa

SEQ ID NO: 655 - DNA, 20 bp, Artificial Sequence
  Antisense Compound
cgttgaggcctccgtaggcc

SEQ ID NO: 656 - DNA, 20 bp, Artificial Sequence
  Antisense Compound
tttttagttagccattacgg

SEQ ID NO: 657 - DNA, 20 bp, Artificial Sequence
  Antisense Compound
cgacacatgttctctctttt

SEQ ID NO: 658 - DNA, 20 bp, Artificial Sequence
  Antisense Compound
cttgtacgacacatgttctc

SEQ ID NO: 659 - DNA, 20 bp, Artificial Sequence
  Antisense Compound
gatgcattttaacttgtacg

SEQ ID NO: 660 - DNA, 20 bp, Artificial Sequence
  Antisense Compound
cttgggccgatgcattttaa

SEQ ID NO: 661 - DNA, 20 bp, Artificial Sequence
  Antisense Compound
tcccccttgggccgatgcat

SEQ ID NO: 662 - DNA, 20 bp, Artificial Sequence
  Antisense Compound
ccttactataagcttaaaaa

SEQ ID NO: 663 - DNA, 20 bp, Artificial Sequence
  Antisense Compound
tgtatccttactataagctt

SEQ ID NO: 664 - DNA, 20 bp, Artificial Sequence
  Antisense Compound
ttgaatgtatccttactata

SEQ ID NO: 665 - DNA, 20 bp, Artificial Sequence
  Antisense Compound
caagcttgaatgtatcctta

SEQ ID NO: 666 - DNA, 20 bp, Artificial Sequence
  Antisense Compound
cttggttggcaaatgaaaaa

SEQ ID NO: 667 - DNA, 20 bp, Artificial Sequence
  Antisense Compound
taaaataacattgtgcttgg

SEQ ID NO: 668 - DNA, 20 bp, Artificial Sequence
  Antisense Compound
gtatactttaaaatatacag

SEQ ID NO: 669 - DNA, 20 bp, Artificial Sequence
  Antisense Compound
atatctgaaagtatacttta

SEQ ID NO: 670 - DNA, 20 bp, Artificial Sequence
  Antisense Compound
gtccttgctatatcttaaat

SEQ ID NO: 671 - DNA, 20 bp, Artificial Sequence
  Antisense Compound
cccacttattagtgccaatt

SEQ ID NO: 672 - DNA, 20 bp, Artificial Sequence
  Antisense Compound
ctttgtactaaattaaatta

SEQ ID NO: 673 - DNA, 20 bp, Artificial Sequence
  Antisense Compound
ttacaaactttgtactaaat

SEQ ID NO: 674 - DNA, 20 bp, Artificial Sequence
  Antisense Compound
gcaataccgtgtcgtagaaa

SEQ ID NO: 675 - DNA, 20 bp, Artificial Sequence
  Antisense Compound
gctgccacagatcactgtag

SEQ ID NO: 676 - DNA, 20 bp, Artificial Sequence
  Antisense Compound
catgaagccgctgccacaga

SEQ ID NO: 677 - DNA, 20 bp, Artificial Sequence
  Antisense Compound
ctttaagatggatattttac

SEQ ID NO: 678 - DNA, 20 bp, Artificial Sequence
  Antisense Compound
gatgtctttaagatggatat

SEQ ID NO: 679 - DNA, 20 bp, Artificial Sequence
  Antisense Compound
tgtacacaattttcagaata

SEQ ID NO: 680 - DNA, 20 bp, Artificial Sequence
  Antisense Compound
ccactaaaggaatatcctgt